SEQUENCE LISTING (496 sequences)

SEQ ID NO: 1 - protein, 107 aa, Synthetic construct
DIQMTQSPSSLSASVGDRVTITCRASQDIRNDLGWYQQKPGKAPKRLIYATSSLQSGVPSRFSGSRSGAEFTLTVSSLQPEDFATYYCLQHNTYPYTFGQGTKLEIK

SEQ ID NO: 2 - protein, 119 aa, Synthetic construct
QVQLVESGGGVVQPGRSLRLSCAASGFTFSIYGMHWVRQAPGKGLEWVAVISYDGNNKDYTDSVKGRFTISRDNSKNTLYLQMNSLTAEDTAVYYCAKRGYGSSSLEFWGQGTLVTVSS

SEQ ID NO: 3 - skipped

SEQ ID NO: 4 - protein, 107 aa, Synthetic construct
DIQMTQSPSSLSASVGDRVTITCRSSQDIRNDLGWYQQKPGKAPKRLIYASSNLQSGVPSRFSGSGSGTEFTLTISSLQPDDFATYYCLQHNTYPYTFGQGTKLEIK

SEQ ID NO: 5 - protein, 119 aa, Synthetic construct
QVQLVESGGGVVLPGRSLRLSCAASGFTFNIYGMHWVRQAPGKGLEWVAVISYDGNNKEYADSVKGRFTISRDSSRNTLYLQMNSLRAEDTAVYYCAKRGYGSSSLEFWGQGTLVTVSS

SEQ ID NO: 6 - protein, 112 aa, Synthetic construct
DIVMTQSPLSLPVTPGEPASISCRSSQSLLHSNGYSYLAWYLQKPGQSPQLLIYLVSNRASGVPDRFTGSGSGTDFTLKISRVEAEDVGVYYCMQALQTPWTFGQGTKVEIK

SEQ ID NO: 7 - protein, 118 aa, Synthetic construct
EVQLVESGGGLVQPGRSLRLSCAASGFMFDDYVMHWVRQAPGKGLEWVSGINWNSDNIGYADSVKGRFTISRDNAKNSLFLQMNSLRVEDTALYYCAKDIDAAAFDYWGQGTLVTVSS

SEQ ID NO: 8 - skipped

SEQ ID NO: 9 - protein, 108 aa, Synthetic construct
EIVLTQSPGTLSLSPGERATLSCRASQSVISGYLAWYQQKPGQAPRLLIYGASRRATGITDRFSGSGSGTDFTLIISRLEPEDSAVYYCQQYDSSPRTFGQGTKVEIK

SEQ ID NO: 10 - protein, 113 aa, Synthetic construct
EVQLVESGGGLVKPGESLRLSCAASGFTFSNAWMSWVRQAPGKGLEWVGRDGGARDYAAPVKGRFTISRDDSKNTLYLQMNTLKTEDTAVYYCTKGAFFFDYWGQGTLVTVSS

SEQ ID NO: 11-12 - skipped (2 entries)

SEQ ID NO: 13 - protein, 107 aa, Synthetic construct
DIQMTQSPSSLSASVGDRVTITCRPSQNISSYLNWYQQKPGKAPKLLIYAASSLRSGVPSRFSGSGSGTDFTLTVSSLQPEDFANYYCQQSYSTPYTFGRGTKLEIK

SEQ ID NO: 14 - protein, 121 aa, Synthetic construct
QVQLVQSGSELKKPGASVKVSCKTSGYTFTSYAMNWVRQAPGQGLEWMGWINTNTGNPTYAQGFRGRFVFSLDTSVSTAYLQISSLKAEDTAVYYCARDKYSSSWYWFDSWGQGTLVTVSS

SEQ ID NO: 15 - protein, 108 aa, Synthetic construct
EIVLTQSPGTLSLSPGERATLSCRASQSVSSSYLAWYQQKPGQAPRLLIYGASTRGTGIPDRFSGSGSGTDFTLTISRLEPEDFALYYCQQYGSSPYTFGQGTKLEIK

SEQ ID NO: 16 - protein, 125 aa, Synthetic construct
QVQLVQSGTEVKYPGASVKVSCKASGYTFIDYGISWVRQAPGQGLEWMGWISAYNGNTDYAQKLQGRVTMTTDTSTSTAYMDLRSLRSDDTAVYYCAREGEGYCTYGVCPSFDYWGQGTLVTVSS

SEQ ID NO: 17 - protein, 111 aa, Synthetic construct
QSVLTQPPSVSGAPGQRVSFSCTGTSSNIGTGFDVHWYRQLPGTAPKVLIYNNNNRPSGVPDRFSASKSGTSASLAITGLQAEDEADYYCQSYDSSLSGSVFGGGTRLTVL

SEQ ID NO: 18 - protein, 121 aa, Synthetic construct
QVQLQESGPGLVKPSETLSLTCTVSGASISGYYWNWIRQPPGKGLEWIGYIHYSGTTNYNPSLKSRITISLDTSENQFSLRLSSVTAADTAVYYCARDWYTGYDWEGFDYWGQGTLVTVSS

SEQ ID NO: 19 - protein, 107 aa, Synthetic construct
DIQMTQSPSSLSASVGDRVTITCRASQNIITYLNWYQQKPGKAPKLLIYAASSLQSGVPSRFSGSGSGTDFTLTISSLQPEDFAIYHCQQSYNVPTTFGQGTKLEIK

SEQ ID NO: 20 - protein, 122 aa, Synthetic construct
EVQLVESGGDLVKSGGSLRLSCAASGFTFSDAWMSWVRQAPGKGLEWVGRIQSKTDGGTTDYFAPVKGRFTISRDDSKDTLYLHMNSLTTEDTAVYYCTTDLYYYGSGSYFLGQGTLVTVSS

SEQ ID NO: 21 - protein, 112 aa, Synthetic construct
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFPLTFGGGTKVEIK

SEQ ID NO: 22 - protein, 127 aa, Synthetic construct
EVQLVESGGGVVRPGGSLRLSCAASGFTLDDYGMSWVRQAPGKGLEWVSGINWNGATTDYADSVKGRFSISRDNAKNSVYLQMNSLRAEDTALYYCAREGITMVRGVIIEDWYFDLWGRGTLVTVSS

SEQ ID NO: 23 - protein, 122 aa, Synthetic construct
EVQLVESGGDLVKSGGSLRLSCAASGFTFSDAWMSWVRQAPGKGLEWVGRIQSKTDGGTTDYVAPVKGRFTISRDDSKDTLYLHMNSLTTEDTAVYYCTTDLYYYGSGSYFLGQGTLVTVSS

SEQ ID NO: 24 - protein, 127 aa, Synthetic construct
EVQLVESGGGVVRPGGSLRLSCAASGFTFDDYGMSWVRQAPGKGLEWVSGINWNGGSTGYADSVKGRFSISRDNAKNSLYLQMNSLRAEDTALYYCAREGITILRGVIIEDWYFDLWGRGTLVIVSS

SEQ ID NO: 25 - skipped

SEQ ID NO: 26 - protein, 11 aa, Synthetic construct
RASQDIRNDLG

SEQ ID NO: 27 - protein, 8 aa, Synthetic construct
YATSSLQS

SEQ ID NO: 28 - protein, 9 aa, Synthetic construct
LQHNTYPYT

SEQ ID NO: 29 - protein, 13 aa, Synthetic construct
AASGFTFSIYGMH

SEQ ID NO: 30 - protein, 10 aa, Synthetic construct
VISYDGNNKD

SEQ ID NO: 31 - protein, 12 aa, Synthetic construct
AKRGYGSSSLEF

SEQ ID NO: 32-33 - skipped (2 entries)

SEQ ID NO: 34 - protein, 11 aa, Synthetic construct
RSSQDIRNDLG

SEQ ID NO: 35 - protein, 8 aa, Synthetic construct
YASSNLQS

SEQ ID NO: 36 - protein, 13 aa, Synthetic construct
AASGFTFNIYGMH

SEQ ID NO: 37 - protein, 10 aa, Synthetic construct
VISYDGNNKE

SEQ ID NO: 38 - protein, 16 aa, Synthetic construct
RSSQSLLHSNGYSYLA

SEQ ID NO: 39 - protein, 8 aa, Synthetic construct
YLVSNRAS

SEQ ID NO: 40 - protein, 9 aa, Synthetic construct
MQALQTPWT

SEQ ID NO: 41 - protein, 13 aa, Synthetic construct
AASGFMFDDYVMH

SEQ ID NO: 42 - protein, 10 aa, Synthetic construct
GINWNSDNIG

SEQ ID NO: 43 - protein, 11 aa, Synthetic construct
AKDIDAAAFDY

SEQ ID NO: 44 - skipped

SEQ ID NO: 45 - protein, 12 aa, Synthetic construct
RASQSVISGYLA

SEQ ID NO: 46 - protein, 8 aa, Synthetic construct
YGASRRAT

SEQ ID NO: 47 - protein, 9 aa, Synthetic construct
QQYDSSPRT

SEQ ID NO: 48 - protein, 13 aa, Synthetic construct
AASGFTFSNAWMS

SEQ ID NO: 49 - protein, 7 aa, Synthetic construct
RDGGARD

SEQ ID NO: 50 - protein, 9 aa, Synthetic construct
TKGAFFFDY

SEQ ID NO: 51 - protein, 11 aa, Synthetic construct
RPSQNISSYLN

SEQ ID NO: 52 - protein, 8 aa, Synthetic construct
YAASSLRS

SEQ ID NO: 53 - protein, 9 aa, Synthetic construct
QQSYSTPYT

SEQ ID NO: 54 - protein, 13 aa, Synthetic construct
KTSGYTFTSYAMN

SEQ ID NO: 55 - protein, 10 aa, Synthetic construct
WINTNTGNPT

SEQ ID NO: 56 - protein, 14 aa, Synthetic construct
ARDKYSSSWYWFDS

SEQ ID NO: 57 - protein, 12 aa, Synthetic construct
RASQSVSSSYLA

SEQ ID NO: 58 - protein, 8 aa, Synthetic construct
YGASTRGT

SEQ ID NO: 59 - protein, 9 aa, Synthetic construct
QQYGSSPYT

SEQ ID NO: 60 - protein, 13 aa, Synthetic construct
KASGYTFIDYGIS

SEQ ID NO: 61 - protein, 10 aa, Synthetic construct
WISAYNGNTD

SEQ ID NO: 62 - protein, 18 aa, Synthetic construct
AREGEGYCTYGVCPSFDY

SEQ ID NO: 63 - protein, 14 aa, Synthetic construct
TGTSSNIGTGFDVH

SEQ ID NO: 64 - protein, 8 aa, Synthetic construct
YNNNNRPS

SEQ ID NO: 65 - protein, 11 aa, Synthetic construct
QSYDSSLSGSV

SEQ ID NO: 66 - protein, 13 aa, Synthetic construct
TVSGASISGYYWN

SEQ ID NO: 67 - protein, 9 aa, Synthetic construct
YIHYSGTTN

SEQ ID NO: 68 - protein, 15 aa, Synthetic construct
ARDWYTGYDWEGFDY

SEQ ID NO: 69 - protein, 11 aa, Synthetic construct
RASQNIITYLN

SEQ ID NO: 70 - protein, 8 aa, Synthetic construct
YAASSLQS

SEQ ID NO: 71 - protein, 9 aa, Synthetic construct
QQSYNVPTT

SEQ ID NO: 72 - protein, 13 aa, Synthetic construct
AASGFTFSDAWMS

SEQ ID NO: 73 - protein, 12 aa, Synthetic construct
RIQSKTDGGTTD

SEQ ID NO: 74 - protein, 13 aa, Synthetic construct
TTDLYYYGSGSYF

SEQ ID NO: 75 - protein, 16 aa, Synthetic construct
RSSQSLVHSDGNTYLS

SEQ ID NO: 76 - protein, 8 aa, Synthetic construct
YKISNRFS

SEQ ID NO: 77 - protein, 9 aa, Synthetic construct
MQATQFPLT

SEQ ID NO: 78 - protein, 13 aa, Synthetic construct
AASGFTLDDYGMS

SEQ ID NO: 79 - protein, 10 aa, Synthetic construct
GINWNGATTD

SEQ ID NO: 80 - protein, 20 aa, Synthetic construct
AREGITMVRGVIIEDWYFDL

SEQ ID NO: 81 - protein, 13 aa, Synthetic construct
AASGFTFDDYGMS

SEQ ID NO: 82 - protein, 10 aa, Synthetic construct
GINWNGGSTG

SEQ ID NO: 83 - protein, 20 aa, Synthetic construct
AREGITILRGVIIEDWYFDL

SEQ ID NO: 84 - protein, 327 aa, Synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 85 - protein, 18 aa, Synthetic construct
GSTSGSGKPGSGEGSTKG

SEQ ID NO: 86 - protein, 6 aa, Synthetic construct
QDIRND

SEQ ID NO: 87 - protein, 8 aa, Synthetic construct
GFTFSIYG

SEQ ID NO: 88 - protein, 8 aa, Synthetic construct
ISYDGNNK

SEQ ID NO: 89 - skipped

SEQ ID NO: 90 - protein, 8 aa, Synthetic construct
GFTFNIYG

SEQ ID NO: 91 - protein, 11 aa, Synthetic construct
QSLLHSNGYSY

SEQ ID NO: 92 - protein, 8 aa, Synthetic construct
GFMFDDYV

SEQ ID NO: 93 - protein, 8 aa, Synthetic construct
INWNSDNI

SEQ ID NO: 94 - skipped

SEQ ID NO: 95 - protein, 7 aa, Synthetic construct
QSVISGY

SEQ ID NO: 96 - protein, 8 aa, Synthetic construct
GFTFSNAW

SEQ ID NO: 97 - protein, 6 aa, Synthetic construct
RDGGAR

SEQ ID NO: 98 - protein, 6 aa, Synthetic construct
QNISSY

SEQ ID NO: 99 - protein, 8 aa, Synthetic construct
GYTFTSYA

SEQ ID NO: 100 - protein, 8 aa, Synthetic construct
INTNTGNP

SEQ ID NO: 101 - protein, 7 aa, Synthetic construct
QSVSSSY

SEQ ID NO: 102 - protein, 8 aa, Synthetic construct
GYTFIDYG

SEQ ID NO: 103 - protein, 8 aa, Synthetic construct
ISAYNGNT

SEQ ID NO: 104 - protein, 9 aa, Synthetic construct
SSNIGTGFD

SEQ ID NO: 105 - protein, 8 aa, Synthetic construct
GASISGYY

SEQ ID NO: 106 - protein, 7 aa, Synthetic construct
IHYSGTT

SEQ ID NO: 107 - protein, 6 aa, Synthetic construct
QNIITY

SEQ ID NO: 108 - protein, 8 aa, Synthetic construct
GFTFSDAW

SEQ ID NO: 109 - protein, 10 aa, Synthetic construct
IQSKTDGGTT

SEQ ID NO: 110 - protein, 11 aa, Synthetic construct
QSLVHSDGNTY

SEQ ID NO: 111 - protein, 8 aa, Synthetic construct
GFTLDDYG

SEQ ID NO: 112 - protein, 8 aa, Synthetic construct
INWNGATT

SEQ ID NO: 113 - protein, 8 aa, Synthetic construct
GFTFDDYG

SEQ ID NO: 114 - protein, 8 aa, Synthetic construct
INWNGGST

SEQ ID NO: 115 - skipped

SEQ ID NO: 116 - protein, 7 aa, Synthetic construct
ATSSLQS

SEQ ID NO: 117 - protein, 5 aa, Synthetic construct
IYGMH

SEQ ID NO: 118 - protein, 17 aa, Synthetic construct
VISYDGNNKDYTDSVKG

SEQ ID NO: 119 - protein, 10 aa, Synthetic construct
RGYGSSSLEF

SEQ ID NO: 120 - skipped

SEQ ID NO: 121 - protein, 7 aa, Synthetic construct
ASSNLQS

SEQ ID NO: 122 - protein, 17 aa, Synthetic construct
VISYDGNNKEYADSVKG

SEQ ID NO: 123 - protein, 7 aa, Synthetic construct
LVSNRAS

SEQ ID NO: 124 - protein, 5 aa, Synthetic construct
DYVMH

SEQ ID NO: 125 - protein, 17 aa, Synthetic construct
GINWNSDNIGYADSVKG

SEQ ID NO: 126 - protein, 9 aa, Synthetic construct
DIDAAAFDY

SEQ ID NO: 127 - protein, 7 aa, Synthetic construct
GASRRAT

SEQ ID NO: 128 - protein, 5 aa, Synthetic construct
NAWMS

SEQ ID NO: 129 - protein, 14 aa, Synthetic construct
RDGGARDYAAPVKG

SEQ ID NO: 130 - protein, 7 aa, Synthetic construct
GAFFFDY

SEQ ID NO: 131 - protein, 7 aa, Synthetic construct
AASSLRS

SEQ ID NO: 132 - protein, 5 aa, Synthetic construct
SYAMN

SEQ ID NO: 133 - protein, 17 aa, Synthetic construct
WINTNTGNPTYAQGFRG

SEQ ID NO: 134 - protein, 12 aa, Synthetic construct
DKYSSSWYWFDS

SEQ ID NO: 135 - protein, 7 aa, Synthetic construct
GASTRGT

SEQ ID NO: 136 - protein, 5 aa, Synthetic construct
DYGIS

SEQ ID NO: 137 - protein, 17 aa, Synthetic construct
WISAYNGNTDYAQKLQG

SEQ ID NO: 138 - protein, 16 aa, Synthetic construct
EGEGYCTYGVCPSFDY

SEQ ID NO: 139 - protein, 7 aa, Synthetic construct
NNNNRPS

SEQ ID NO: 140 - protein, 6 aa, Synthetic construct
SGYYWN

SEQ ID NO: 141 - protein, 16 aa, Synthetic construct
YIHYSGTTNYNPSLKS

SEQ ID NO: 142 - protein, 13 aa, Synthetic construct
DWYTGYDWEGFDY

SEQ ID NO: 143 - protein, 7 aa, Synthetic construct
AASSLQS

SEQ ID NO: 144 - protein, 5 aa, Synthetic construct
DAWMS

SEQ ID NO: 145 - protein, 19 aa, Synthetic construct
RIQSKTDGGTTDYFAPVKG

SEQ ID NO: 146 - protein, 11 aa, Synthetic construct
DLYYYGSGSYF

SEQ ID NO: 147 - protein, 7 aa, Synthetic construct
KISNRFS

SEQ ID NO: 148 - protein, 5 aa, Synthetic construct
DYGMS

SEQ ID NO: 149 - protein, 17 aa, Synthetic construct
GINWNGATTDYADSVKG

SEQ ID NO: 150 - protein, 18 aa, Synthetic construct
EGITMVRGVIIEDWYFDL

SEQ ID NO: 151 - protein, 19 aa, Synthetic construct
RIQSKTDGGTTDYVAPVKG

SEQ ID NO: 152 - protein, 17 aa, Synthetic construct
GINWNGGSTGYADSVKG

SEQ ID NO: 153 - protein, 18 aa, Synthetic construct
EGITILRGVIIEDWYFDL

SEQ ID NO: 154-155 - skipped (2 entries)

SEQ ID NO: 156 - protein, 7 aa, Synthetic construct
GFTFSIY

SEQ ID NO: 157 - protein, 6 aa, Synthetic construct
SYDGNN

SEQ ID NO: 158 - protein, 7 aa, Synthetic construct
GFTFNIY

SEQ ID NO: 159 - protein, 7 aa, Synthetic construct
GFMFDDY

SEQ ID NO: 160 - protein, 6 aa, Synthetic construct
NWNSDN

SEQ ID NO: 161 - protein, 7 aa, Synthetic construct
GFTFSNA

SEQ ID NO: 162 - protein, 4 aa, Synthetic construct
DGGA

SEQ ID NO: 163 - protein, 7 aa, Synthetic construct
GYTFTSY

SEQ ID NO: 164 - protein, 6 aa, Synthetic construct
NTNTGN

SEQ ID NO: 165 - protein, 7 aa, Synthetic construct
GYTFIDY

SEQ ID NO: 166 - protein, 6 aa, Synthetic construct
SAYNGN

SEQ ID NO: 167 - protein, 7 aa, Synthetic construct
GASISGY

SEQ ID NO: 168 - protein, 5 aa, Synthetic construct
HYSGT

SEQ ID NO: 169 - protein, 7 aa, Synthetic construct
GFTFSDA

SEQ ID NO: 170 - protein, 8 aa, Synthetic construct
QSKTDGGT

SEQ ID NO: 171 - protein, 7 aa, Synthetic construct
GFTLDDY

SEQ ID NO: 172 - protein, 6 aa, Synthetic construct
NWNGAT

SEQ ID NO: 173 - protein, 7 aa, Synthetic construct
GFTFDDY

SEQ ID NO: 174 - protein, 6 aa, Synthetic construct
NWNGGS

SEQ ID NO: 175 - protein, 7 aa, Synthetic construct
RNDLGWY

SEQ ID NO: 176 - protein, 10 aa, Synthetic construct
RLIYATSSLQ

SEQ ID NO: 177 - protein, 8 aa, Synthetic construct
LQHNTYPY

SEQ ID NO: 178 - protein, 6 aa, Synthetic construct
SIYGMH

SEQ ID NO: 179 - protein, 13 aa, Synthetic construct
WVAVISYDGNNKD

SEQ ID NO: 180 - protein, 11 aa, Synthetic construct
AKRGYGSSSLE

SEQ ID NO: 181-182 - skipped (2 entries)

SEQ ID NO: 183 - protein, 10 aa, Synthetic construct
RLIYASSNLQ

SEQ ID NO: 184 - protein, 6 aa, Synthetic construct
NIYGMH

SEQ ID NO: 185 - protein, 13 aa, Synthetic construct
WVAVISYDGNNKE

SEQ ID NO: 186 - protein, 7 aa, Synthetic construct
YSYLAWY

SEQ ID NO: 187 - protein, 10 aa, Synthetic construct
LLIYLVSNRA

SEQ ID NO: 188 - protein, 8 aa, Synthetic construct
MQALQTPW

SEQ ID NO: 189 - protein, 6 aa, Synthetic construct
DDYVMH

SEQ ID NO: 190 - protein, 13 aa, Synthetic construct
WVSGINWNSDNIG

SEQ ID NO: 191 - protein, 10 aa, Synthetic construct
AKDIDAAAFD

SEQ ID NO: 192 - protein, 7 aa, Synthetic construct
SGYLAWY

SEQ ID NO: 193 - protein, 10 aa, Synthetic construct
LLIYGASRRA

SEQ ID NO: 194 - protein, 8 aa, Synthetic construct
QQYDSSPR

SEQ ID NO: 195 - protein, 6 aa, Synthetic construct
SNAWMS

SEQ ID NO: 196 - protein, 10 aa, Synthetic construct
WVGRDGGARD

SEQ ID NO: 197 - protein, 8 aa, Synthetic construct
TKGAFFFD

SEQ ID NO: 198 - protein, 7 aa, Synthetic construct
SSYLNWY

SEQ ID NO: 199 - protein, 10 aa, Synthetic construct
LLIYAASSLR

SEQ ID NO: 200 - protein, 8 aa, Synthetic construct
QQSYSTPY

SEQ ID NO: 201 - protein, 6 aa, Synthetic construct
TSYAMN

SEQ ID NO: 202 - protein, 13 aa, Synthetic construct
WMGWINTNTGNPT

SEQ ID NO: 203 - protein, 13 aa, Synthetic construct
ARDKYSSSWYWFD

SEQ ID NO: 204 - protein, 7 aa, Synthetic construct
SSYLAWY

SEQ ID NO: 205 - protein, 10 aa, Synthetic construct
LLIYGASTRG

SEQ ID NO: 206 - protein, 8 aa, Synthetic construct
QQYGSSPY

SEQ ID NO: 207 - protein, 6 aa, Synthetic construct
IDYGIS

SEQ ID NO: 208 - protein, 13 aa, Synthetic construct
WMGWISAYNGNTD

SEQ ID NO: 209 - protein, 17 aa, Synthetic construct
AREGEGYCTYGVCPSFD

SEQ ID NO: 210 - protein, 7 aa, Synthetic construct
GFDVHWY

SEQ ID NO: 211 - protein, 10 aa, Synthetic construct
VLIYNNNNRP

SEQ ID NO: 212 - protein, 10 aa, Synthetic construct
QSYDSSLSGS

SEQ ID NO: 213 - protein, 12 aa, Synthetic construct
WIGYIHYSGTTN

SEQ ID NO: 214 - protein, 14 aa, Synthetic construct
ARDWYTGYDWEGFD

SEQ ID NO: 215 - protein, 7 aa, Synthetic construct
ITYLNWY

SEQ ID NO: 216 - protein, 10 aa, Synthetic construct
LLIYAASSLQ

SEQ ID NO: 217 - protein, 8 aa, Synthetic construct
QQSYNVPT

SEQ ID NO: 218 - protein, 6 aa, Synthetic construct
SDAWMS

SEQ ID NO: 219 - protein, 15 aa, Synthetic construct
WVGRIQSKTDGGTTD

SEQ ID NO: 220 - protein, 12 aa, Synthetic construct
TTDLYYYGSGSY

SEQ ID NO: 221 - protein, 7 aa, Synthetic construct
NTYLSWL

SEQ ID NO: 222 - protein, 10 aa, Synthetic construct
LLIYKISNRF

SEQ ID NO: 223 - protein, 8 aa, Synthetic construct
MQATQFPL

SEQ ID NO: 224 - protein, 6 aa, Synthetic construct
DDYGMS

SEQ ID NO: 225 - protein, 13 aa, Synthetic construct
WVSGINWNGATTD

SEQ ID NO: 226 - protein, 19 aa, Synthetic construct
AREGITMVRGVIIEDWYFD

SEQ ID NO: 227 - protein, 13 aa, Synthetic construct
WVSGINWNGGSTG

SEQ ID NO: 228 - protein, 19 aa, Synthetic construct
AREGITILRGVIIEDWYFD

SEQ ID NO: 229-230 - skipped (2 entries)

SEQ ID NO: 231 - protein, 22 aa, Synthetic construct
MDMRVPAQLLGLLLLWFPGARC

SEQ ID NO: 232 - protein, 19 aa, Synthetic construct
MEFGLSWVFLVALLRGVQC

SEQ ID NO: 233 - skipped

SEQ ID NO: 234 - protein, 20 aa, Synthetic construct
MRVPAQLLGLLLLWFPGARC

SEQ ID NO: 235 - protein, 20 aa, Synthetic construct
MRLPAQLLGLLMLWVSGSSG

SEQ ID NO: 236 - protein, 19 aa, Synthetic construct
MELGLSWIFLLAILKGVQC

SEQ ID NO: 237 - skipped

SEQ ID NO: 238 - protein, 20 aa, Synthetic construct
METPAQLLFLLLLWLPDTTG

SEQ ID NO: 239 - protein, 19 aa, Synthetic construct
MEFGLNWIFLAAILKGVQC

SEQ ID NO: 240 - protein, 22 aa, Synthetic construct
MDMRVPAQLLGLLLLWLRGARC

SEQ ID NO: 241 - protein, 19 aa, Synthetic construct
MDWTWRILFLVAAGTGAHS

SEQ ID NO: 242 - protein, 19 aa, Synthetic construct
MDWTWSILFLVAAATGAHS

SEQ ID NO: 243 - protein, 19 aa, Synthetic construct
MAWSPLLLTLLAHCTGSWA

SEQ ID NO: 244 - protein, 19 aa, Synthetic construct
MKHLWFFLLLVAAPRWVLS

SEQ ID NO: 245 - protein, 22 aa, Synthetic construct
MDMRVPAQLLGLLLLWLRGASC

SEQ ID NO: 246 - protein, 19 aa, Synthetic construct
MEFGLSWIFLAAILKGVQC

SEQ ID NO: 247 - protein, 20 aa, Synthetic construct
MRLLAQLLGLLMLWVPGSSG

SEQ ID NO: 248 - protein, 19 aa, Synthetic construct
MEFGLSWVFLVAILKGVQC

SEQ ID NO: 249 - protein, 20 aa, Synthetic construct
MRVPAQLLGLLLLWLRGASC

SEQ ID NO: 250 - skipped

SEQ ID NO: 251 - protein, 499 aa, Homo sapiens
MLLLLLLLPLLWGTKGMEGDRQYGDGYLLQVQELVTVQEGLCVHVPCSFSYPQDGWTDSDPVHGYWFRAGDRPYQDAPVATNNPDREVQAETQGRFQLLGDIWSNDCSLSIRDARKRDKGSYFFRLERGSMKWSYKSQLNYKTKQLSVFVTALTHRPDILILGTLESGHSRNLTCSVPWACKQGTPPMISWIGASVSSPGPTTARSSVLTLTPKPQDHGTSLTCQVTLPGTGVTTTSTVRLDVSYPPWNLTMTVFQGDATASTALGNGSSLSVLEGQSLRLVCAVNSNPPARLSWTRGSLTLCPSRSSNPGLLELPRVHVRDEGEFTCRAQNAQGSQHISLSLSLQNEGTGTSRPVSQVTLAAVGGAGATALAFLSFCIIFIIVRSCRKKSARPAAGVGDTGMEDAKAIRGSASQGPLTESWKDGNPLKKPPPAVAPSSGEEGELHYATLSFHKVKPQDPQGQEATDSEYSEIKIHKRETAETQACLRNHNPSSKEVRG

SEQ ID NO: 252 - DNA, 1500 bp, Homo sapiens
  source 1..1500: mol_type = genomic DNA
atgctgctgctgctcctgctgctgcccctgctctgggggacaaaggggatggagggagacagacaatatggggatggttacttgctgcaagtgcaggagctggtgacggtgcaggagggcctgtgtgtccatgtgccctgctccttctcctacccccaggatggctggactgactctgacccagttcatggctactggttccgggcaggagacagaccataccaagacgctccagtggccacaaacaacccagacagagaagtgcaggcagagacccagggccgattccaactccttggggacatttggagcaacgactgctccctgagcatcagagacgccaggaagagggataaggggtcatatttctttcggctagagagaggaagcatgaaatggagttacaaatcacagttgaattacaaaactaagcagctgtctgtgtttgtgacagccctgacccataggcctgacatcctcatcctagggaccctagagtctggccactccaggaacctgacctgctctgtgccctgggcctgtaagcaggggacaccccccatgatctcctggattggggcctccgtgtcctccccgggccccactactgcccgctcctcagtgctcacccttaccccaaagccccaggaccacggcaccagcctcacctgtcaggtgaccttgcctgggacaggtgtgaccacgaccagtaccgtccgcctcgatgtgtcctaccctccttggaacttgaccatgactgtcttccaaggagatgccacagcatccacagccctgggaaatggctcatctctttcagtccttgagggccagtctctgcgcctggtctgtgctgtcaacagcaatccccctgccaggctgagctggacccgggggagcctgaccctgtgcccctcacggtcctcaaaccctgggctgctggagctgcctcgagtgcacgtgagggatgaaggggaattcacctgccgagctcagaacgctcagggctcccagcacatttccctgagcctctccctgcagaatgagggcacaggcacctcaagacctgtatcacaagtgacactggcagcagtcgggggagctggagccacagccctggccttcctgtccttctgcatcatcttcatcatagtgaggtcctgcaggaagaaatcggcaaggccagcagcgggcgtgggggatacaggcatggaagatgcaaaggccatcaggggctcggcctctcagggacccctgactgaatcctggaaagatggcaaccccctgaagaagcctcccccagctgttgccccctcgtcaggggaggaaggagagctccattatgcaaccctcagcttccataaagtgaagcctcaggacccgcagggacaggaggccactgacagtgaatactcggagatcaagatccacaagcgagaaactgcagagactcaggcctgtttgaggaatcacaacccctccagcaaagaagtcagaggctga

SEQ ID NO: 253 - protein, 406 aa, Homo sapiens
MLLLLLLLPLLWGTKGMEGDRQYGDGYLLQVQELVTVQEGLCVHVPCSFSYPQDGWTDSDPVHGYWFRAGDRPYQDAPVATNNPDREVQAETQGRFQLLGDIWSNDCSLSIRDARKRDKGSYFFRLERGSMKWSYKSQLNYKTKQLSVFVTDPPWNLTMTVFQGDATASTALGNGSSLSVLEGQSLRLVCAVNSNPPARLSWTRGSLTLCPSRSSNPGLLELPRVHVRDEGEFTCRAQNAQGSQHISLSLSLQNEGTGTSRPVSQVTLAAVGGAGATALAFLSFCIIFIIVRSCRKKSARPAAGVGDTGMEDAKAIRGSASQGPLTESWKDGNPLKKPPPAVAPSSGEEGELHYATLSFHKVKPQDPQGQEATDSEYSEIKIHKRETAETQACLRNHNPSSKEVRG

SEQ ID NO: 254 - DNA, 1221 bp, Homo sapiens
  source 1..1221: mol_type = genomic DNA
atgctgttactgctcttactgctgcccctgctctgggggacaaaggggatggagggagacagacaatatggggatggttacttgctgcaagtgcaggagctggtgacggtgcaggagggcctgtgtgtccatgtgccctgctccttctcctacccccaggatggctggactgactctgacccagttcatggctactggttccgggcaggagacagaccataccaagacgctccagtggccacaaacaacccagacagagaagtgcaggcagagacccagggccgattccaactccttggggacatttggagcaacgactgctccctgagcatcagagacgccaggaagagggataaggggtcatatttctttcggctagagagaggaagcatgaaatggagttacaaatcacagttgaattacaaaactaagcagctgtctgtgtttgtgacagaccctccttggaacttgaccatgactgtcttccaaggagatgccacagcatccacagccctgggaaatggctcatctctttcagtccttgagggccagtctctgcgcctggtctgtgctgtcaacagcaatccccctgccaggctgagctggacccgggggagcctgaccctgtgcccctcacggtcctcaaaccctgggctgctggagctgcctcgagtgcacgtgagggatgaaggggaattcacctgccgagctcagaacgctcagggctcccagcacatttccctgagcctctccctgcagaatgagggcacaggcacctcaagacctgtatcacaagtgacactggcagcagtcgggggagctggagccacagccctggccttcctgtccttctgcatcatcttcatcatagtgaggtcctgcaggaagaaatcggcaaggccagcagcgggcgtgggggatacaggcatggaagatgcaaaggccatcaggggctcggcctctcagggacccctgactgaatcctggaaagatggcaaccccctgaagaagcctcccccagctgttgccccctcgtcaggggaggaaggagagctccattatgcaaccctcagcttccataaagtgaagcctcaggacccgcagggacaggaggccactgacagtgaatactcggagatcaagatccacaagcgagaaactgcagagactcaggcctgtttgaggaatcacaacccctccagcaaagaagtcagaggctga

SEQ ID NO: 255 - protein, 300 aa, Synthetic construct
MLLLLLLLPLLWGTKGMEGDRQYGDGYLLQVQELVTVQEGLCVHVPCSFSYPQDGWTDSDPVHGYWFRAGDRPYQDAPVATNNPDREVQAETQGRFQLLGDIWSNDCSLSIRDARKRDKGSYFFRLERGSMKWSYKSQLNYKTKQLSVFVTATSRPVSQVTLAAVGGAGATALAFLSFCIIFIIVRSCRKKSARPAAGVGDTGMEDAKAIRGSASQGPLTESWKDGNPLKKPPPAVAPSSGEEGELHYATLSFHKVKPQDPQGQEATDSEYSEIKIHKRETAETQACLRNHNPSSKEVRG

SEQ ID NO: 256 - DNA, 903 bp, Synthetic construct
  source 1..903: mol_type = other DNA
atgctgttactgctcttactgctgcccctgctctgggggacaaaggggatggagggagacagacaatatggggatggttacttgctgcaagtgcaggagctggtgacggtgcaggagggcctgtgtgtccatgtgccctgctccttctcctacccccaggatggctggactgactctgacccagttcatggctactggttccgggcaggagacagaccataccaagacgctccagtggccacaaacaacccagacagagaagtgcaggcagagacccagggccgattccaactccttggggacatttggagcaacgactgctccctgagcatcagagacgccaggaagagggataaggggtcatatttctttcggctagagagaggaagcatgaaatggagttacaaatcacagttgaattacaaaactaagcagctgtctgtgtttgtgacagcaacctcaagacctgtatcacaagtgacactggcagcagtcgggggagctggagccacagccctggccttcctgtccttctgcatcatcttcatcatagtgaggtcctgcaggaagaaatcggcaaggccagcagcgggcgtgggggatacaggcatggaagatgcaaaggccatcaggggctcggcctctcagggacccctgactgaatcctggaaagatggcaaccccctgaagaagcctcccccagctgttgccccctcgtcaggggaggaaggagagctccattatgcaaccctcagcttccataaagtgaagcctcaggacccgcagggacaggaggccactgacagtgaatactcggagatcaagatccacaagcgagaaactgcagagactcaggcctgtttgaggaatcacaacccctccagcaaagaagtcagaggctga

SEQ ID NO: 257 - protein, 487 aa, Synthetic construct
MLWPLPLFLLCAGSLAQDLEFQLVAPESVTVEEGLCVHVPCSVFYPSIKLTLGPVTGSWLRKGVSLHEDSPVATSDPRQLVQKATQGRFQLLGDPQKHDCSLFIRDAQKNDTGMYFFRVVREPFVRYSYKKSQLSLHVTSLSRTPDIIIPGTLEAGYPSNLTCSVPWACEQGTPPTFSWMSTALTSLSSRTTDSSVLTFTPQPQDHGTKLTCLVTFSGAGVTVERTIQLNVTRPPWNLTMTVFQGDATASTALGNGSSLSVLEGQSLRLVCAVNSNPPARLSWTRGSLTLCPSRSSNPGLLELPRVHVRDEGEFTCRAQNAQGSQHISLSLSLQNEGTGTSRPVSQVTLAAVGGAGATALAFLSFCIIFIIVRSCRKKSARPAAGVGDTGMEDAKAIRGSASQGPLTESWKDGNPLKKPPPAVAPSSGEEGELHYATLSFHKVKPQDPQGQEATDSEYSEIKIHKRETAETQACLRNHNPSSKEVRG

SEQ ID NO: 258 - DNA, 1464 bp, Synthetic construct
  source 1..1464: mol_type = other DNA
atgctttggccactgcctctcttcctcttgtgtgcaggaagtttggcacaggatctcgaatttcaacttgtcgccccagaatcagtaacggtagaagaaggtctctgcgtgcacgttccctgttccgtcttctatccctcaataaagcttactctgggacctgtgacaggttcctggttgcgcaaaggagttagtctgcacgaggactcaccagtcgctaccagcgacccccgccagcttgtgcagaaggcgacccaaggcagatttcaactgcttggagaccctcagaaacatgactgtagtctcttcataagggacgcacagaagaatgacacaggtatgtattttttccgggtcgtgcgagagccattcgtccgatattcctataagaaatcccagctcagtttgcatgttactagtctgagtcgcaccccagatataattattccaggtacattggaggccggttatccgtccaacctcacatgctccgtgccctgggcatgtgaacaggggacgcctcccacgttctcttggatgagtacagcgctgacctccttgagttcccgaaccacggacagctctgttctcacatttacaccgcagccacaagatcacggaaccaagctcacatgccttgttacattctctggagctggtgttaccgtcgaacgaaccatccaacttaatgtcacccgcccaccgtggaatctcactatgacggtttttcaaggggatgccactgcatctacggccctgggtaatgggagttcattgtcagtgttggaggggcaatccctcaggcttgtttgcgctgtaaacagtaatccacctgctagactttcttggactcggggcagccttactctctgtccgtctcgatcatccaaccccggcctcctcgaacttccgcgagttcacgttcgagatgaaggggagtttacttgtcgggcacagaatgcccaaggttctcaacatattagtttgtcattgtctctgcagaatgaggggaccgggacgagtcgcccggtttcccaggtcacgctggccgcagtaggtggagccggggcgacagccctcgccttcctttccttctgcattattttcattatagttcgatcctgtcggaaaaagtccgcgagaccggcagcaggtgttggggatacgggcatggaggacgccaaagcaatacggggaagtgcgagccaaggtcccctgacggaatcatggaaagatggcaaccctctcaaaaagcctccaccggcagttgcgccctcttctggtgaagagggggagttgcactatgccacgctttcttttcataaagtgaaacctcaggacccgcaaggacaagaagctactgattccgaatattcagaaataaagatccataagagggagactgctgagacacaagcatgccttagaaatcataatccgtccagcaaagaagtcagaggctga

SEQ ID NO: 259 - protein, 573 aa, Synthetic construct
MRWAWLLPLLWAGCLATDGYSLSVTGSVTVQEGLCVFVACQVQYPNSKGPVFGYWFREGANIFSGSPVATNDPQRSVLKEAQGRFYLMGKENSHNCSLDIRDAQKIDTGTYFFRLDGSVKYSFQKSMLSVLVIALTEVPNIQVTSTLVSGNSTKLLCSVPWACEQGTPPIFSWMSSALTSLGHRTTLSSELNLTPRPQDNGTNLTCQVNLPGTGVTVERTQQLSVIALTHRPDILILGTLESGHSRNLTCSVPWACKQGTPPMISWIGASVSSPGPTTARSSVLTLTPKPQDHGTSLTCQVTLPGTGVTTTSTVRLDVSPPWNLTMTVFQGDATASTALGNGSSLSVLEGQSLRLVCAVNSNPPARLSWTRGSLTLCPSRSSNPGLLELPRVHVRDEGEFTCRAQNAQGSQHISLSLSLQNEGTGTSRPVSQVTLAAVGGAGATALAFLSFCIIFIIVRSCRKKSARPAAGVGDTGMEDAKAIRGSASQGPLTESWKDGNPLKKPPPAVAPSSGEEGELHYATLSFHKVKPQDPQGQEATDSEYSEIKIHKRETAETQACLRNHNPSSKEVRG

SEQ ID NO: 260 - DNA, 1722 bp, Synthetic construct
  source 1..1722: mol_type = other DNA
atgagatgggcgtggttattgccgcttttgtgggctggttgcctcgctactgatggctactccctgtcagtcacgggttccgttactgttcaggaaggtctgtgtgtgttcgttgcctgtcaagtccaatatcccaattccaaggggccagttttcggatactggttccgggagggtgccaacatattctccggaagcccagttgctaccaatgatccacagcggagtgtccttaaggaggcacagggtagattctacctcatgggtaaagaaaatagtcacaattgtagcttggacataagagatgcacaaaaaatagacacggggacctattttttccgcttggacggctccgtgaagtattcatttcaaaagagcatgctctcagttcttgttattgcacttacggaggtccctaacatacaggtcacgagtacccttgtgtctgggaatagtacaaagctgctctgcagcgtgccttgggcgtgcgagcagggcacccctccgattttttcatggatgtcctctgctcttacttccctgggtcatagaactaccctgagctctgaacttaacctgacgccacgacctcaggataatggcacaaaccttacgtgccaggtaaacttgccgggcacaggtgtcactgtggaaaggacccagcaactttccgtaattgccctgacccataggcctgacatcctcatcctagggaccctagagtctggccactccaggaacctgacctgctctgtgccctgggcctgtaagcaggggacaccccccatgatctcctggattggggcctccgtgtcctccccgggccccactactgcccgctcctcagtgctcacccttaccccaaagccccaggaccacggcaccagcctcacctgtcaggtgaccttgcctgggacaggtgtgaccacgaccagtaccgtccgcctcgatgtgtcccctccttggaacttgaccatgactgtcttccaaggagatgccacagcatccacagccctgggaaatggctcatctctttcagtccttgagggccagtctctgcgcctggtctgtgctgtcaacagcaatccccctgccaggctgagctggacccgggggagcctgaccctgtgcccctcacggtcctcaaaccctgggctgctggagctgcctcgagtgcacgtgagggatgaaggggaattcacctgccgagctcagaacgctcagggctcccagcacatttccctgagcctctccctgcagaatgagggcacaggcacctcaagacctgtatcacaagtgacactggcagcagtcgggggagctggagccacagccctggccttcctgtccttctgcatcatcttcatcatagtgaggtcctgcaggaagaaatcggcaaggccagcagcgggcgtgggggatacaggcatggaagatgcaaaggccatcaggggctcggcctctcagggacccctgactgaatcctggaaagatggcaaccccctgaagaagcctcccccagctgttgccccctcgtcaggggaggaaggagagctccattatgcaaccctcagcttccataaagtgaagcctcaggacccgcagggacaggaggccactgacagtgaatactcggagatcaagatccacaagcgagaaactgcagagactcaggcctgtttgaggaatcacaacccctccagcaaagaagtcagaggctga

SEQ ID NO: 261 - protein, 508 aa, Synthetic construct
MLLLLLLLPLLWGTKGHHHHHHGSGMEGDRQYGDGYLLQVQELVTVQEGLCVHVPCSFSYPQDGWTDSDPVHGYWFRAGDRPYQDAPVATNNPDREVQAETQGRFQLLGDIWSNDCSLSIRDARKRDKGSYFFRLERGSMKWSYKSQLNYKTKQLSVFVTALTHRPDILILGTLESGHSRNLTCSVPWACKQGTPPMISWIGASVSSPGPTTARSSVLTLTPKPQDHGTSLTCQVTLPGTGVTTTSTVRLDVSYPPWNLTMTVFQGDATASTALGNGSSLSVLEGQSLRLVCAVNSNPPARLSWTRGSLTLCPSRSSNPGLLELPRVHVRDEGEFTCRAQNAQGSQHISLSLSLQNEGTGTSRPVSQVTLAAVGGAGATALAFLSFCIIFIIVRSCRKKSARPAAGVGDTGMEDAKAIRGSASQGPLTESWKDGNPLKKPPPAVAPSSGEEGELHYATLSFHKVKPQDPQGQEATDSEYSEIKIHKRETAETQACLRNHNPSSKEVRG

SEQ ID NO: 262 - DNA, 1527 bp, Synthetic construct
  source 1..1527: mol_type = other DNA
atgctgcttctcctgctgcttctgcctctcttgtggggcaccaaaggccatcatcaccatcatcacggatccggaatggaaggagatagacaatacggggacggttatcttctgcaagttcaggaacttgttaccgtccaagaaggactctgcgttcacgttccgtgtagtttttcttacccacaagatggctggacagactccgacccggttcacgggtattggtttagagcgggcgacaggccgtatcaggatgccccggtggccaccaataacccggaccgcgaagtacaagcggaaacgcaaggacggtttcaactgctgggcgacatttggagtaacgactgtagtctcagcattcgcgacgccaggaagcgcgataagggctcatatttctttcggctggaacgcggatcaatgaagtggagttacaagtcacagctcaactataaaactaaacagctttcagttttcgtcaccgcgcttacccaccggcctgacattctcattctggggactcttgaaagtggacacagtcggaacctgacctgcagcgtgccttgggcttgcaagcaaggcactcctcccatgatttcttggattggggcctcagtaagcagtccgggtccgaccacagcccgctcatcagtacttacactgacgccaaagccgcaggatcacggtacttccctcacgtgtcaagtgactctgcctggaacgggagttacgacaacctcaacagtcagattggacgtatcctaccccccgtggaacctcacaatgaccgtgttccaaggagatgctactgcgtcaactgctctcggtaacgggtcttcattgagcgtgcttgaaggccaatctctgcggctggtatgtgcggtgaactccaaccctccggcgcgcctttcatggacgcgaggaagtttgacgctctgcccttcaagatcatccaaccctggccttcttgaattgccgcgcgttcacgtgcgcgatgagggtgaatttacatgccgcgcgcagaacgcacaaggtagtcaacacatcagcttgagtctgagcttgcaaaatgaaggcaccggaactagtaggccggtaagccaagtaacactggctgcagtgggaggggctggagcgactgcccttgcctttctgtccttttgtatcattttcatcattgtccgctcatgtaggaagaaatcagcacgaccggctgctggtgtcggtgacaccgggatggaagatgctaaagcgatccggggctctgccagccagggccccctcacagaaagttggaaggatggtaatcccctgaagaaacctccaccagcggtcgccccctcatctggagaagaaggagaacttcactacgcgactctctcctttcataaggtcaagccgcaggacccccaggggcaggaggccactgattccgaatacagcgagataaaaatacacaaacgggagacagccgagactcaggcttgtttgcgcaaccacaacccctcatccaaggaggtgcgagggtga

SEQ ID NO: 263 - protein, 589 aa, Synthetic construct
MLLLLLLLPLLWGTKGMEGDRQYGDGYLLQVQELVTVQEGLCVHVPCSFSYPQDGWTDSDPVHGYWFRAGDRPYQDAPVATNNPDREVQAETQGRFQLLGDIWSNDCSLSIRDARKRDKGSYFFRLERGSMKWSYKSQLNYKTKQLSVFVTALTHRPDILILGTLESGHSRNLTCSVPWACKQGTPPMISWIGASVSSPGPTTARSSVLTLTPKPQDHGTSLTCQVTLPGTGVTTTSTVRLDVSYPPWNLTMTVFQGDATASTALGNGSSLSVLEGQSLRLVCAVNSNPPARLSWTRGSLTLCPSRSSNPGLLELPRVHVRDEGEFTCRAQNAQGSQHISLSLSLQNEGTGTSRPVSQVTLAAGGGSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 264 - DNA, 1770 bp, Synthetic construct
  source 1..1770: mol_type = other DNA
atgctgctgctgctcctgctgctgcccctgctctgggggacaaaggggatggagggagacagacaatatggggatggttacttgctgcaagtgcaggagctggtgacggtgcaggagggcctgtgtgtccatgtgccctgctccttctcctacccccaggatggctggactgactctgacccagttcatggctactggttccgggcaggagacagaccataccaagacgctccagtggccacaaacaacccagacagagaagtgcaggcagagacccagggccgattccaactccttggggacatttggagcaacgactgctccctgagcatcagagacgccaggaagagggataaggggtcatatttctttcggctagagagaggaagcatgaaatggagttacaaatcacagttgaattacaaaactaagcagctgtctgtgtttgtgacagccctgacccataggcctgacatcctcatcctagggaccctagagtctggccactccaggaacctgacctgctctgtgccctgggcctgtaagcaggggacaccccccatgatctcctggattggggcctccgtgtcctccccgggccccactactgcccgctcctcagtgctcacccttaccccaaagccccaggaccacggcaccagcctcacctgtcaggtgaccttgcctgggacaggtgtgaccacgaccagtaccgtccgcctcgatgtgtcctaccctccttggaacttgaccatgactgtcttccaaggagatgccacagcatccacagccctgggaaatggctcatctctttcagtccttgagggccagtctctgcgcctggtctgtgctgtcaacagcaatccccctgccaggctgagctggacccgggggagcctgaccctgtgcccctcacggtcctcaaaccctgggctgctggagctgcctcgagtgcacgtgagggatgaaggggaattcacctgccgagctcagaacgctcagggctcccagcacatttccctgagcctctccctgcagaatgagggcacaggcacctcaagacctgtatcacaagtgacactggcagcaggaggtggatccggttgtaagccctgtatttgcacagtaccagaggtgtcttctgtttttatatttcctccaaagcctaaggatgtactcactataactcttacaccaaaggttacttgtgtggttgtcgatatcagcaaggatgacccggaggtacaatttagttggtttgtagatgatgtggaagtccacaccgcgcaaacgcagccacgagaagaacagtttaactctacatttaggagcgttagcgagctcccaatcatgcaccaggattggcttaacggaaaggagtttaaatgccgggtcaactcagcggcttttccagccccgattgagaagacaatttcaaaaaccaagggacgcccaaaagccccgcaagtatatactatcccaccaccaaaggaacaaatggctaaggataaagttagtctgacttgtatgattacagattttttcccagaggatataacagtcgaatggcagtggaatggtcagccggcagaaaactataaaaacacccagccgatcatgaatacaaacggttcctactttgtttatagcaagttgaatgtccaaaaaagcaactgggaagcgggcaatacctttacctgttccgtgctgcacgagggactgcataaccatcacaccgaaaagagtctgtcacatagtcctggaaaatga

SEQ ID NO: 265 - protein, 571 aa, Synthetic construct
MRWAWLLPLLWAGCLATDGYSLSVTGSVTVQEGLCVFVACQVQYPNSKGPVFGYWFREGANIFSGSPVATNDPQRSVLKEAQGRFYLMGKENSHNCSLDIRDAQKIDTGTYFFRLDGSVKYSFQKSMLSVLVIALTEVPNIQVTSTLVSGNSTKLLCSVPWACEQGTPPIFSWMSSALTSLGHRTTLSSELNLTPRPQDNGTNLTCQVNLPGTGVTVERTQQLSVIYPPWNLTMTVFQGDATASTALGNGSSLSVLEGQSLRLVCAVNSNPPARLSWTRGSLTLCPSRSSNPGLLELPRVHVRDEGEFTCRAQNAQGSQHISLSLSLQNEGTGTSRPVSQVTLAAGGGSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 266 - DNA, 1716 bp, Synthetic construct
  source 1..1716: mol_type = other DNA
atgagatgggcgtggctcctcccgcttttgtgggctggttgcctcgctactgatggctactccctgtcagtcacgggttccgttactgttcaggaaggtctgtgtgtgttcgttgcctgtcaagtccaatatcccaattccaaggggccagttttcggatactggttccgggagggtgccaacatattctccggaagcccagttgctaccaatgatccacagcggagtgtccttaaggaggcacagggtagattctacctcatgggtaaagaaaatagtcacaattgtagcttggacataagagatgcacaaaaaatagacacggggacctattttttccgcttggacggctccgtgaagtattcatttcaaaagagcatgctctcagttcttgttattgcacttacggaggtccctaacatacaggtcacgagtacccttgtgtctgggaatagtacaaagctgctctgcagcgtgccttgggcgtgcgagcagggcacccctccgattttttcatggatgtcctctgctcttacttccctgggtcatagaactaccctgagctctgaacttaacctgacgccacgacctcaggataatggcacaaaccttacgtgccaggtaaacttgccgggcacaggtgtcactgtggaaaggacccagcaactttccgtaatttatccgccctggaacctgacaatgaccgtatttcagggcgatgcgacagcgagtactgcactgggaaatggtagttctctttccgttctggagggccagtcactcaggttggtttgtgcggtaaactcaaaccccccggcacgcctctcatggacgcgcggctccctcaccctgtgtccatccagaagcagcaacccaggcttgctcgaattgcccagggtacatgttcgggacgaaggtgaattcacgtgcagagctcagaatgctcagggctcacagcatattagtctgtcactgtctcttcagaacgaaggcacggggacgtctcgcccggtaagccaagtgacgctggccgcgggaggtggatccggttgtaagccctgtatttgcacagtaccagaggtgtcttctgtttttatatttcctccaaagcctaaggatgtactcactataactcttacaccaaaggttacttgtgtggttgtcgatatcagcaaggatgacccggaggtacaatttagttggtttgtagatgatgtggaagtccacaccgcgcaaacgcagccacgagaagaacagtttaactctacatttaggagcgttagcgagctcccaatcatgcaccaggattggcttaacggaaaggagtttaaatgccgggtcaactcagcggcttttccagccccgattgagaagacaatttcaaaaaccaagggacgcccaaaagccccgcaagtatatactatcccaccaccaaaggaacaaatggctaaggataaagttagtctgacttgtatgattacagattttttcccagaggatataacagtcgaatggcagtggaatggtcagccggcagaaaactataaaaacacccagccgatcatgaatacaaacggttcctactttgtttatagcaagttgaatgtccaaaaaagcaactgggaagcgggcaatacctttacctgttccgtgctgcacgagggactgcataaccatcacaccgaaaagagtctgtcacatagtcctggaaaatga

SEQ ID NO: 267 - protein, 577 aa, Synthetic construct
MLWPLPLFLLCAGSLAQDLEFQLVAPESVTVEEGLCVHVPCSVFYPSIKLTLGPVTGSWLRKGVSLHEDSPVATSDPRQLVQKATQGRFQLLGDPQKHDCSLFIRDAQKNDTGMYFFRVVREPFVRYSYKKSQLSLHVTSLSRTPDIIIPGTLEAGYPSNLTCSVPWACEQGTPPTFSWMSTALTSLSSRTTDSSVLTFTPQPQDHGTKLTCLVTFSGAGVTVERTIQLNVTRPPWNLTMTVFQGDATASTALGNGSSLSVLEGQSLRLVCAVNSNPPARLSWTRGSLTLCPSRSSNPGLLELPRVHVRDEGEFTCRAQNAQGSQHISLSLSLQNEGTGTSRPVSQVTLAAGGGSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 268 - DNA, 1734 bp, Synthetic construct
  source 1..1734: mol_type = other DNA
atgctttggccactgcctctcttcctcttgtgtgcaggaagtttggcacaggatctcgaatttcaacttgtcgccccagaatcagtaacggtagaagaaggtctctgcgtgcacgttccctgttccgtcttctatccctcaataaagcttactctgggacctgtgacaggttcctggttgcgcaaaggagttagtctgcacgaggactcaccagtcgctaccagcgacccccgccagcttgtgcagaaggcgacccaaggcagatttcaactgcttggagaccctcagaaacatgactgtagtctcttcataagggacgcacagaagaatgacacaggtatgtattttttccgggtcgtgcgagagccattcgtccgatattcctataagaaatcccagctcagtttgcatgttactagtctgagtcgcaccccagatataattattccaggtacattggaggccggttatccgtccaacctcacatgctccgtgccctgggcatgtgaacaggggacgcctcccacgttctcttggatgagtacagcgctgacctccttgagttcccgaaccacggacagctctgttctcacatttacaccgcagccacaagatcacggaaccaagctcacatgccttgttacattctctggagctggtgttaccgtcgaacgaaccatccaacttaatgtcacccgcccaccgtggaatctcactatgacggtttttcaaggggatgccactgcatctacggccctgggtaatgggagttcattgtcagtgttggaggggcaatccctcaggcttgtttgcgctgtaaacagtaatccacctgctagactttcttggactcggggcagccttactctctgtccgtctcgatcatccaaccccggcctcctcgaacttccgcgagttcacgttcgagatgaaggggagtttacttgtcgggcacagaatgcccaaggttctcaacatattagtttgtcattgtctctgcagaatgaggggaccgggacgagtcgcccggtttcccaggtcacgctggccgcaggaggtggatccggttgtaagccctgtatttgcacagtaccagaggtgtcttctgtttttatatttcctccaaagcctaaggatgtactcactataactcttacaccaaaggttacttgtgtggttgtcgatatcagcaaggatgacccggaggtacaatttagttggtttgtagatgatgtggaagtccacaccgcgcaaacgcagccacgagaagaacagtttaactctacatttaggagcgttagcgagctcccaatcatgcaccaggattggcttaacggaaaggagtttaaatgccgggtcaactcagcggcttttccagccccgattgagaagacaatttcaaaaaccaagggacgcccaaaagccccgcaagtatatactatcccaccaccaaaggaacaaatggctaaggataaagttagtctgacttgtatgattacagattttttcccagaggatataacagtcgaatggcagtggaatggtcagccggcagaaaactataaaaacacccagccgatcatgaatacaaacggttcctactttgtttatagcaagttgaatgtccaaaaaagcaactgggaagcgggcaatacctttacctgttccgtgctgcacgagggactgcataaccatcacaccgaaaagagtctgtcacatagtcctggaaaatga

SEQ ID NO: 269 - protein, 388 aa, Synthetic construct
MLLLLLLLPLLWGTKGMEGDRQYGDGYLLQVQELVTVQEGLCVHVPCSFSYPQDGWTDSDPVHGYWFRAGDRPYQDAPVATNNPDREVQAETQGRFQLLGDIWSNDCSLSIRDARKRDKGSYFFRLERGSMKWSYKSQLNYKTKQLSVFVTALTHRPDILILGTLESGHSRNLTCSVPWACKQGTPPMISWIGASVSSPGPTTARSSVLTLTPKPQDHGTSLTCQVTLPGTGVTTTSTVRLDVSYPPWNLTMTVFQGDATASTALGNGSSLSVLEGQSLRLVCAVNSNPPARLSWTRGSLTLCPSRSSNPGLLELPRVHVRDEGEFTCRAQNAQGSQHISLSLSLQNEGTGTSRPVSQVTLAAGSHHHHHHGSGLNDIFEAQKIEWHE

SEQ ID NO: 270 - DNA, 1167 bp, Synthetic construct
  source 1..1167: mol_type = other DNA
atgctgctgctgctcctgctgctgcccctgctctgggggacaaaggggatggagggagacagacaatatggggatggttacttgctgcaagtgcaggagctggtgacggtgcaggagggcctgtgtgtccatgtgccctgctccttctcctacccccaggatggctggactgactctgacccagttcatggctactggttccgggcaggagacagaccataccaagacgctccagtggccacaaacaacccagacagagaagtgcaggcagagacccagggccgattccaactccttggggacatttggagcaacgactgctccctgagcatcagagacgccaggaagagggataaggggtcatatttctttcggctagagagaggaagcatgaaatggagttacaaatcacagttgaattacaaaactaagcagctgtctgtgtttgtgacagccctgacccataggcctgacatcctcatcctagggaccctagagtctggccactccaggaacctgacctgctctgtgccctgggcctgtaagcaggggacaccccccatgatctcctggattggggcctccgtgtcctccccgggccccactactgcccgctcctcagtgctcacccttaccccaaagccccaggaccacggcaccagcctcacctgtcaggtgaccttgcctgggacaggtgtgaccacgaccagtaccgtccgcctcgatgtgtcctaccctccttggaacttgaccatgactgtcttccaaggagatgccacagcatccacagccctgggaaatggctcatctctttcagtccttgagggccagtctctgcgcctggtctgtgctgtcaacagcaatccccctgccaggctgagctggacccgggggagcctgaccctgtgcccctcacggtcctcaaaccctgggctgctggagctgcctcgagtgcacgtgagggatgaaggggaattcacctgccgagctcagaacgctcagggctcccagcacatttccctgagcctctccctgcagaatgagggcacaggcacctcaagacctgtatcacaagtgacactggcagcaggatcccatcatcaccatcaccacggaagtggtttgaacgacatttttgaagcgcagaaaattgaatggcatgaatga

SEQ ID NO: 271 - protein, 471 aa, Synthetic construct
METDTLLLWVLLLWVPGSTGQVQLQESGPGLVKPSETLSLTCTVSGASISGYYWNWIRQPPGKGLEWIGYIHYSGTTNYNPSLKSRITISLDTSENQFSLRLSSVTAADTAVYYCARDWYTGYDWEGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 272 - DNA, 1416 bp, Synthetic construct
  source 1..1416: mol_type = other DNA
atggagacagatactttgctcctttgggttttgttgctgtgggttccaggatccacggggcaggtgcaacttcaggagtcaggtccaggcttggttaaaccaagtgagacactgtccctgacttgcacagtatccggggcatctatctctggttattattggaactggattaggcaaccacctgggaagggtcttgaatggattggttacatacattactcagggacgacgaattataatccatccctcaagtccagaataactatcagtctcgacacttcagaaaatcagtttagcctgagactgtcaagtgtcaccgctgcggatactgccgtatattactgcgctagagactggtataccggctacgactgggaaggattcgattattggggccaaggaaccttggtaacagtgtcttcagctagcaccaaaggaccctccgtctttccgctcgcgccgagcagcaagtccacgagcggggggacagccgcccttgggtgtctggtcaaggattattttcctgagcccgttactgtctcctggaatagcggtgccttgacttctggggtacataccttcccggcggttttgcaatcatcaggactgtatagcctctctagcgttgtaacggtaccatcttcatcacttggcacacaaacatatatttgtaatgtaaatcacaaaccttcaaatactaaagttgacaaaaaagtggagccgaagagctgtgataaaacgcatacttgcccgccttgccccgctccagaattgctgggcgggccgtctgtgttcctcttcccaccgaaaccgaaggatacgctgatgataagtcgaacgcccgaagtaacatgcgtggttgttgatgtaagccacgaagatccggaagtaaagttcaactggtacgtagatggagtagaagtgcataacgcgaaaactaagccccgcgaggagcaatataattcaacctatcgggttgttagtgtgctcaccgttctccatcaggattggcttaacgggaaagagtataagtgcaaggttagcaataaagcattgccagcacctatagagaagacaattagcaaagcgaagggacagccacgagagccccaagtctacacgctgcctcccagccgcgacgagttgacgaagaatcaagtgtcactcacatgtcttgtcaagggattctatccctcagatattgcagtagaatgggagtctaatggacaaccagagaacaactataaaactactccaccggtgcttgatagtgacggtagttttttcctttatagtaagctgactgtcgataagagtcgctggcaacagggaaatgttttttcttgtagtgttatgcatgaagcacttcataaccattacacacaaaagtctctgtcactttcccctggcaaatga

SEQ ID NO: 273 - protein, 468 aa, Synthetic construct
METDTLLLWVLLLWVPGSTGQVQLQESGPGLVKPSETLSLTCTVSGASISGYYWNWIRQPPGKGLEWIGYIHYSGTTNYNPSLKSRITISLDTSENQFSLRLSSVTAADTAVYYCARDWYTGYDWEGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 274 - DNA, 1407 bp, Synthetic construct
  source 1..1407: mol_type = other DNA
atggagacagatactttgctcctttgggttttgttgctgtgggttccaggatccacggggcaggtgcaacttcaggagtcaggtccaggcttggttaaaccaagtgagacactgtccctgacttgcacagtatccggggcatctatctctggttattattggaactggattaggcaaccacctgggaagggtcttgaatggattggttacatacattactcagggacgacgaattataatccatccctcaagtccagaataactatcagtctcgacacttcagaaaatcagtttagcctgagactgtcaagtgtcaccgctgcggatactgccgtatattactgcgctagagactggtataccggctacgactgggaaggattcgattattggggccaaggaaccttggtaacagtgtcttcagctagcaccaaaggaccctccgtctttccgctcgcgccgtgctctcgatccacgagcgagtctacggcggccttgggctgtctcgtgaaagactatttcccagagccagtgaccgtttcctggaactccggagcgctcacatcaggtgtgcatacctttccagccgtacttcaatcttcagggctgtatagcttgtcctctgtcgtgaccgtaccctccagttctctcgggacaaagacctatacctgtaacgtagaccataagccatccaataccaaagtagataagagggtggaatctaaatatggaccgccttgtccgagctgcccggctcctgagttccttggagggccgtccgtctttctgtttccccctaaacctaaggacaccctgatgataagtaggacgccggaagtaacctgtgtcgtggttgatgtgtcacaggaagacccggaggtacaatttaattggtacgtcgatggagtcgaggtgcataatgcgaaaaccaagccacgagaggagcaattcaacagcacctaccgagtagtttcagtcttgactgtgctgcaccaagactggctgaatggcaaggagtacaaatgcaaggtcagtaataaaggattgccgtcttcaatagagaagactatcagtaaggcgaagggacaacctcgcgagcctcaggtatacactttgccgcccagccaagaagaaatgacgaagaaccaagtctctttgacttgcctggtcaaaggcttctacccctctgatatagcagtcgaatgggagtccaacggtcaaccggagaacaattataagacaactccacctgtgcttgattcagacggctcttttttcttgtatagccgccttaccgtagacaagagtcgatggcaggaaggtaatgtattttcttgtagtgtaatgcacgaggcacttcataatcactacacacaaaagtctctgtcactttccctgggcaaatga

SEQ ID NO: 275 - protein, 237 aa, Synthetic construct
METDTLLLWVLLLWVPGSTGQSVLTQPPSVSGAPGQRVSFSCTGTSSNIGTGFDVHWYRQLPGTAPKVLIYNNNNRPSGVPDRFSASKSGTSASLAITGLQAEDEADYYCQSYDSSLSGSVFGGGTRLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 276 - DNA, 717 bp, Synthetic construct
  source 1..717: mol_type = other DNA
atggagacagatactttgctcctttgggttttgttgctgtgggttccaggatccacggggcagagcgtacttacacagcccccttcagtcagtggagcgccaggtcaaagggtctctttttcatgtactggaacgtcaagcaacatcgggacaggatttgatgtccactggtataggcagctcccaggaactgcccccaaagtcttgatctataacaataacaatcgcccttctggcgtacctgatcgattcagcgcatcaaaatccgggacctcagccagcctcgccattacgggcttgcaggcagaagacgaggccgattactactgtcaaagttatgatagctcattgagcgggtcagtctttggcggcggtacgagattgaccgttctgggacagcctaaagcaaatccaaccgtaaccttgtttccaccgtccagcgaagagcttcaggctaacaaagctacactcgtatgcctgatcagtgacttctatcctggggccgtaacagtagcgtggaaagcagatgggtcacccgtcaaagcaggggtggagacgaccaaaccctccaagcagtccaacaataagtacgcagcctcaagttatttgtcacttactcctgaacaatggaaatcacatcgatcctattcctgtcaagttacccatgaggggagcactgttgaaaaaactgtcgccccaactgaatgtagttaatga

SEQ ID NO: 277 - protein, 475 aa, Synthetic construct
METDTLLLWVLLLWVPGSTGQVQLVQSGTEVKYPGASVKVSCKASGYTFIDYGISWVRQAPGQGLEWMGWISAYNGNTDYAQKLQGRVTMTTDTSTSTAYMDLRSLRSDDTAVYYCAREGEGYCTYGVCPSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 278 - DNA, 1428 bp, Synthetic construct
  source 1..1428: mol_type = other DNA
atggagacagatactttgctcctttgggttttgttgctgtgggttccaggatccacggggcaggtacaattggtccagtccgggacagaagtcaagtatccgggggcttctgttaaggtatcctgtaaagcgtccggttacacattcatcgactacggcataagctgggtaagacaagcaccgggccaagggcttgaatggatgggatggatctccgcatacaatggtaacacggactatgcccaaaagctgcaaggaagagtaactatgactacagatacaagcaccagcacggcctatatggatcttagaagtcttcggagtgacgacacggcggtttactactgcgcaagggagggggagggatattgcacatatggagtgtgtccatcattcgattattggggacaaggaaccttggtaacagtgtcttcagctagcaccaaaggaccctccgtctttccgctcgcgccgagcagcaagtccacgagcggggggacagccgcccttgggtgtctggtcaaggattattttcctgagcccgttactgtctcctggaatagcggtgccttgacttctggggtacataccttcccggcggttttgcaatcatcaggactgtatagcctctctagcgttgtaacggtaccatcttcatcacttggcacacaaacatatatttgtaatgtaaatcacaaaccttcaaatactaaagttgacaaaaaagtggagccgaagagctgtgataaaacgcatacttgcccgccttgccccgctccagaattgctgggcgggccgtctgtgttcctcttcccaccgaaaccgaaggatacgctgatgataagtcgaacgcccgaagtaacatgcgtggttgttgatgtaagccacgaagatccggaagtaaagttcaactggtacgtagatggagtagaagtgcataacgcgaaaactaagccccgcgaggagcaatataattcaacctatcgggttgttagtgtgctcaccgttctccatcaggattggcttaacgggaaagagtataagtgcaaggttagcaataaagcattgccagcacctatagagaagacaattagcaaagcgaagggacagccacgagagccccaagtctacacgctgcctcccagccgcgacgagttgacgaagaatcaagtgtcactcacatgtcttgtcaagggattctatccctcagatattgcagtagaatgggagtctaatggacaaccagagaacaactataaaactactccaccggtgcttgatagtgacggtagttttttcctttatagtaagctgactgtcgataagagtcgctggcaacagggaaatgttttttcttgtagtgttatgcatgaagcacttcataaccattacacacaaaagtctctgtcactttcccctggcaaatga

SEQ ID NO: 279 - protein, 472 aa, Synthetic construct
METDTLLLWVLLLWVPGSTGQVQLVQSGTEVKYPGASVKVSCKASGYTFIDYGISWVRQAPGQGLEWMGWISAYNGNTDYAQKLQGRVTMTTDTSTSTAYMDLRSLRSDDTAVYYCAREGEGYCTYGVCPSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 280 - DNA, 1419 bp, Synthetic construct
  source 1..1419: mol_type = other DNA
atggagacagatactttgctcctttgggttttgttgctgtgggttccaggatccacggggcaggtacaattggtccagtccgggacagaagtcaagtatccgggggcttctgttaaggtatcctgtaaagcgtccggttacacattcatcgactacggcataagctgggtaagacaagcaccgggccaagggcttgaatggatgggatggatctccgcatacaatggtaacacggactatgcccaaaagctgcaaggaagagtaactatgactacagatacaagcaccagcacggcctatatggatcttagaagtcttcggagtgacgacacggcggtttactactgcgcaagggagggggagggatattgcacatatggagtgtgtccatcattcgattattggggacaaggaaccttggtaacagtgtcttcagctagcaccaaaggaccctccgtctttccgctcgcgccgtgctctcgatccacgagcgagtctacggcggccttgggctgtctcgtgaaagactatttcccagagccagtgaccgtttcctggaactccggagcgctcacatcaggtgtgcatacctttccagccgtacttcaatcttcagggctgtatagcttgtcctctgtcgtgaccgtaccctccagttctctcgggacaaagacctatacctgtaacgtagaccataagccatccaataccaaagtagataagagggtggaatctaaatatggaccgccttgtccgagctgcccggctcctgagttccttggagggccgtccgtctttctgtttccccctaaacctaaggacaccctgatgataagtaggacgccggaagtaacctgtgtcgtggttgatgtgtcacaggaagacccggaggtacaatttaattggtacgtcgatggagtcgaggtgcataatgcgaaaaccaagccacgagaggagcaattcaacagcacctaccgagtagtttcagtcttgactgtgctgcaccaagactggctgaatggcaaggagtacaaatgcaaggtcagtaataaaggattgccgtcttcaatagagaagactatcagtaaggcgaagggacaacctcgcgagcctcaggtatacactttgccgcccagccaagaagaaatgacgaagaaccaagtctctttgacttgcctggtcaaaggcttctacccctctgatatagcagtcgaatgggagtccaacggtcaaccggagaacaattataagacaactccacctgtgcttgattcagacggctcttttttcttgtatagccgccttaccgtagacaagagtcgatggcaggaaggtaatgtattttcttgtagtgtaatgcacgaggcacttcataatcactacacacaaaagtctctgtcactttccctgggcaaatga

SEQ ID NO: 281 - protein, 235 aa, Synthetic construct
METDTLLLWVLLLWVPGSTGEIVLTQSPGTLSLSPGERATLSCRASQSVSSSYLAWYQQKPGQAPRLLIYGASTRGTGIPDRFSGSGSGTDFTLTISRLEPEDFALYYCQQYGSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 282 - DNA, 711 bp, Synthetic construct
  source 1..711: mol_type = other DNA
atggagacagatactttgctcctttgggttttgttgctgtgggttccaggatccacgggggaaattgttctgacgcagtctccgggtaccctctcactgagtcctggtgaaagggcaacccttagttgccgcgcatcacagtcagtttcatccagttatctcgcctggtaccagcaaaagcctggtcaagccccgcggttgcttatctacggggcatccacccgaggtaccggcattcctgaccgattctccggttcagggtcagggactgatttcactctcactataagccgacttgagccggaggatttcgccttgtactattgccagcagtacgggtccagcccatatactttcggccaaggtacaaagttggagataaagcgaactgttgcagctccaagtgttttcatatttccgccgtcagatgaacaactgaaatcaggcacagcctcagtggtgtgtctgcttaataatttctaccctcgagaggcaaaggtacagtggaaggtagacaacgccctgcaaagcgggaatagtcaagaaagcgttacggaacaagatagcaaagattccacctactcactcagttctaccttgaccctcagcaaggctgattacgagaagcataaagtgtatgcgtgcgaagtgacccatcaaggactctcctcacctgttacaaagtcctttaatcgcggggagtgttaatga

SEQ ID NO: 283 - protein, 469 aa, Synthetic construct
METDTLLLWVLLLWVPGSTGQVQLVESGGGVVLPGRSLRLSCAASGFTFNIYGMHWVRQAPGKGLEWVAVISYDGNNKEYADSVKGRFTISRDSSRNTLYLQMNSLRAEDTAVYYCAKRGYGSSSLEFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 284 - DNA, 1410 bp, Synthetic construct
  source 1..1410: mol_type = other DNA
atggagacagatactttgctcctttgggttttgttgctgtgggttccaggatccacggggcaagtccaacttgttgaaagtggagggggagttgtcctgccgggtcgatctcttcgccttagctgcgcggcgagtgggttcacatttaacatatacggaatgcactgggtgcgccaagcccctggaaaggggctcgaatgggttgcggtcattagttacgatgggaacaacaaagagtacgctgacagtgtgaagggacgatttacgatttcacgcgactcatctcgcaacacattgtacttgcaaatgaactcacttagagcagaagatacggctgtttattattgtgcaaaaagaggctatggatcatcctcacttgaattttggggtcaaggaaccttggtaacagtgtcttcagctagcaccaaaggaccctccgtctttccgctcgcgccgagcagcaagtccacgagcggggggacagccgcccttgggtgtctggtcaaggattattttcctgagcccgttactgtctcctggaatagcggtgccttgacttctggggtacataccttcccggcggttttgcaatcatcaggactgtatagcctctctagcgttgtaacggtaccatcttcatcacttggcacacaaacatatatttgtaatgtaaatcacaaaccttcaaatactaaagttgacaaaaaagtggagccgaagagctgtgataaaacgcatacttgcccgccttgccccgctccagaattgctgggcgggccgtctgtgttcctcttcccaccgaaaccgaaggatacgctgatgataagtcgaacgcccgaagtaacatgcgtggttgttgatgtaagccacgaagatccggaagtaaagttcaactggtacgtagatggagtagaagtgcataacgcgaaaactaagccccgcgaggagcaatataattcaacctatcgggttgttagtgtgctcaccgttctccatcaggattggcttaacgggaaagagtataagtgcaaggttagcaataaagcattgccagcacctatagagaagacaattagcaaagcgaagggacagccacgagagccccaagtctacacgctgcctcccagccgcgacgagttgacgaagaatcaagtgtcactcacatgtcttgtcaagggattctatccctcagatattgcagtagaatgggagtctaatggacaaccagagaacaactataaaactactccaccggtgcttgatagtgacggtagttttttcctttatagtaagctgactgtcgataagagtcgctggcaacagggaaatgttttttcttgtagtgttatgcatgaagcacttcataaccattacacacaaaagtctctgtcactttcccctggcaaatga

SEQ ID NO: 285 - protein, 466 aa, Synthetic construct
METDTLLLWVLLLWVPGSTGQVQLVESGGGVVLPGRSLRLSCAASGFTFNIYGMHWVRQAPGKGLEWVAVISYDGNNKEYADSVKGRFTISRDSSRNTLYLQMNSLRAEDTAVYYCAKRGYGSSSLEFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 286 - DNA, 1401 bp, Synthetic construct
  source 1..1401: mol_type = other DNA
atggagacagatactttgctcctttgggttttgttgctgtgggttccaggatccacggggcaagtccaacttgttgaaagtggagggggagttgtcctgccgggtcgatctcttcgccttagctgcgcggcgagtgggttcacatttaacatatacggaatgcactgggtgcgccaagcccctggaaaggggctcgaatgggttgcggtcattagttacgatgggaacaacaaagagtacgctgacagtgtgaagggacgatttacgatttcacgcgactcatctcgcaacacattgtacttgcaaatgaactcacttagagcagaagatacggctgtttattattgtgcaaaaagaggctatggatcatcctcacttgaattttggggtcaaggaaccttggtaacagtgtcttcagctagcaccaaaggaccctccgtctttccgctcgcgccgtgctctcgatccacgagcgagtctacggcggccttgggctgtctcgtgaaagactatttcccagagccagtgaccgtttcctggaactccggagcgctcacatcaggtgtgcatacctttccagccgtacttcaatcttcagggctgtatagcttgtcctctgtcgtgaccgtaccctccagttctctcgggacaaagacctatacctgtaacgtagaccataagccatccaataccaaagtagataagagggtggaatctaaatatggaccgccttgtccgagctgcccggctcctgagttccttggagggccgtccgtctttctgtttccccctaaacctaaggacaccctgatgataagtaggacgccggaagtaacctgtgtcgtggttgatgtgtcacaggaagacccggaggtacaatttaattggtacgtcgatggagtcgaggtgcataatgcgaaaaccaagccacgagaggagcaattcaacagcacctaccgagtagtttcagtcttgactgtgctgcaccaagactggctgaatggcaaggagtacaaatgcaaggtcagtaataaaggattgccgtcttcaatagagaagactatcagtaaggcgaagggacaacctcgcgagcctcaggtatacactttgccgcccagccaagaagaaatgacgaagaaccaagtctctttgacttgcctggtcaaaggcttctacccctctgatatagcagtcgaatgggagtccaacggtcaaccggagaacaattataagacaactccacctgtgcttgattcagacggctcttttttcttgtatagccgccttaccgtagacaagagtcgatggcaggaaggtaatgtattttcttgtagtgtaatgcacgaggcacttcataatcactacacacaaaagtctctgtcactttccctgggcaaatga

SEQ ID NO: 287 - protein, 234 aa, Synthetic construct
METDTLLLWVLLLWVPGSTGDIQMTQSPSSLSASVGDRVTITCRSSQDIRNDLGWYQQKPGKAPKRLIYASSNLQSGVPSRFSGSGSGTEFTLTISSLQPDDFATYYCLQHNTYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 288 - DNA, 708 bp, Synthetic construct
  source 1..708: mol_type = other DNA
atggagacagatactttgctcctttgggttttgttgctgtgggttccaggatccacgggggacatccagatgacacaatcaccgtctagcctttctgcgtcagtcggagaccgcgtgaccataacgtgcagatcctcccaggatattcggaacgatttgggctggtatcagcagaagccgggcaaagcgccaaagcgcctgatctacgcctcttccaacttgcagtccggtgtacctagcaggttttctggtagcggcagtggtactgaatttaccttgacaatatccagtcttcagccagacgactttgccacctattactgtcttcaacataacacttacccatacacctttggacaaggtacaaaattggagattaaacgaactgttgcagctccaagtgttttcatatttccgccgtcagatgaacaactgaaatcaggcacagcctcagtggtgtgtctgcttaataatttctaccctcgagaggcaaaggtacagtggaaggtagacaacgccctgcaaagcgggaatagtcaagaaagcgttacggaacaagatagcaaagattccacctactcactcagttctaccttgaccctcagcaaggctgattacgagaagcataaagtgtatgcgtgcgaagtgacccatcaaggactctcctcacctgttacaaagtcctttaatcgcggggagtgttaatga

SEQ ID NO: 289 - protein, 472 aa, Synthetic construct
METDTLLLWVLLLWVPGSTGEVQLVESGGDLVKSGGSLRLSCAASGFTFSDAWMSWVRQAPGKGLEWVGRIQSKTDGGTTDYVAPVKGRFTISRDDSKDTLYLHMNSLTTEDTAVYYCTTDLYYYGSGSYFLGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 290 - DNA, 1419 bp, Synthetic construct
  source 1..1419: mol_type = other DNA
atggagacagatactttgctcctttgggttttgttgctgtgggttccaggatccacgggggaagtccaactcgtcgagagcgggggtgaccttgtcaagtccggcggaagcctcagactgtcttgtgcagccagcgggtttacgttctctgatgcctggatgtcatgggtaagacaggcccctggaaaagggcttgagtgggtaggccgcatacagagcaaaactgacggggggacaacggattacgtcgctccagttaaagggcggttcactataagccgagacgacagtaaggacacgctttatcttcacatgaatagccttaccactgaggacacggctgtctattattgcacgacagacctgtactattacggtagcggatcatatttcctcggacaaggaaccttggtaacagtgtcttcagctagcaccaaaggaccctccgtctttccgctcgcgccgagcagcaagtccacgagcggggggacagccgcccttgggtgtctggtcaaggattattttcctgagcccgttactgtctcctggaatagcggtgccttgacttctggggtacataccttcccggcggttttgcaatcatcaggactgtatagcctctctagcgttgtaacggtaccatcttcatcacttggcacacaaacatatatttgtaatgtaaatcacaaaccttcaaatactaaagttgacaaaaaagtggagccgaagagctgtgataaaacgcatacttgcccgccttgccccgctccagaattgctgggcgggccgtctgtgttcctcttcccaccgaaaccgaaggatacgctgatgataagtcgaacgcccgaagtaacatgcgtggttgttgatgtaagccacgaagatccggaagtaaagttcaactggtacgtagatggagtagaagtgcataacgcgaaaactaagccccgcgaggagcaatataattcaacctatcgggttgttagtgtgctcaccgttctccatcaggattggcttaacgggaaagagtataagtgcaaggttagcaataaagcattgccagcacctatagagaagacaattagcaaagcgaagggacagccacgagagccccaagtctacacgctgcctcccagccgcgacgagttgacgaagaatcaagtgtcactcacatgtcttgtcaagggattctatccctcagatattgcagtagaatgggagtctaatggacaaccagagaacaactataaaactactccaccggtgcttgatagtgacggtagttttttcctttatagtaagctgactgtcgataagagtcgctggcaacagggaaatgttttttcttgtagtgttatgcatgaagcacttcataaccattacacacaaaagtctctgtcactttcccctggcaaatga

SEQ ID NO: 291 - protein, 469 aa, Synthetic construct
METDTLLLWVLLLWVPGSTGEVQLVESGGDLVKSGGSLRLSCAASGFTFSDAWMSWVRQAPGKGLEWVGRIQSKTDGGTTDYVAPVKGRFTISRDDSKDTLYLHMNSLTTEDTAVYYCTTDLYYYGSGSYFLGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 292 - DNA, 1410 bp, Synthetic construct
  source 1..1410: mol_type = other DNA
atggagacagatactttgctcctttgggttttgttgctgtgggttccaggatccacgggggaagtccaactcgtcgagagcgggggtgaccttgtcaagtccggcggaagcctcagactgtcttgtgcagccagcgggtttacgttctctgatgcctggatgtcatgggtaagacaggcccctggaaaagggcttgagtgggtaggccgcatacagagcaaaactgacggggggacaacggattacgtcgctccagttaaagggcggttcactataagccgagacgacagtaaggacacgctttatcttcacatgaatagccttaccactgaggacacggctgtctattattgcacgacagacctgtactattacggtagcggatcatatttcctcggacaaggaaccttggtaacagtgtcttcagctagcaccaaaggaccctccgtctttccgctcgcgccgtgctctcgatccacgagcgagtctacggcggccttgggctgtctcgtgaaagactatttcccagagccagtgaccgtttcctggaactccggagcgctcacatcaggtgtgcatacctttccagccgtacttcaatcttcagggctgtatagcttgtcctctgtcgtgaccgtaccctccagttctctcgggacaaagacctatacctgtaacgtagaccataagccatccaataccaaagtagataagagggtggaatctaaatatggaccgccttgtccgccgtgcccggctcctgagttccttggagggccgtccgtctttctgtttccccctaaacctaaggacaccctgatgataagtaggacgccggaagtaacctgtgtcgtggttgatgtgtcacaggaagacccggaggtacaatttaattggtacgtcgatggagtcgaggtgcataatgcgaaaaccaagccacgagaggagcaattcaacagcacctaccgagtagtttcagtcttgactgtgctgcaccaagactggctgaatggcaaggagtacaaatgcaaggtcagtaataaaggattgccgtcttcaatagagaagactatcagtaaggcgaagggacaacctcgcgagcctcaggtatacactttgccgcccagccaagaagaaatgacgaagaaccaagtctctttgacttgcctggtcaaaggcttctacccctctgatatagcagtcgaatgggagtccaacggtcaaccggagaacaattataagacaactccacctgtgcttgattcagacggctcttttttcttgtatagccgccttaccgtagacaagagtcgatggcaggaaggtaatgtattttcttgtagtgtaatgcacgaggcacttcataatcactacacacaaaagtctctgtcactttccctgggcaaatga

SEQ ID NO: 293 - protein, 234 aa, Synthetic construct
METDTLLLWVLLLWVPGSTGDIQMTQSPSSLSASVGDRVTITCRASQNIITYLNWYQQKPGKAPKLLIYAASSLQSGVPSRFSGSGSGTDFTLTISSLQPEDFAIYHCQQSYNVPTTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 294 - DNA, 708 bp, Synthetic construct
  source 1..708: mol_type = other DNA
atggagacagatactttgctcctttgggttttgttgctgtgggttccaggatccacgggggacattcaaatgactcaaagtccaagtagccttagtgcgagcgtcggagatcgagtcactataacctgtcgggccagccaaaatatcatcacctacctcaattggtatcagcagaaaccaggtaaggcaccaaaactgcttatatatgctgcttcatctcttcaatccggtgtgccctctcgattctctgggagtgggtctggcacagatttcactcttactatatcatctctgcaacccgaggactttgcgatatatcattgccagcagtcatacaacgtcccaactacgtttgggcaggggactaagctggagataaaacgaactgttgcagctccaagtgttttcatatttccgccgtcagatgaacaactgaaatcaggcacagcctcagtggtgtgtctgcttaataatttctaccctcgagaggcaaaggtacagtggaaggtagacaacgccctgcaaagcgggaatagtcaagaaagcgttacggaacaagatagcaaagattccacctactcactcagttctaccttgaccctcagcaaggctgattacgagaagcataaagtgtatgcgtgcgaagtgacccatcaaggactctcctcacctgttacaaagtcctttaatcgcggggagtgttaatga

SEQ ID NO: 295 - protein, 214 aa, Synthetic construct
DIQMTQSPSSLSASVGDRVTITCRASQNIITYLNWYQQKPGKAPKLLIYAASSLQSGVPSRFSGSGSGTDFTLTISSLQPEDFAIYHCQQSYNVPTTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 296 - protein, 469 aa, Synthetic construct
METDTLLLWVLLLWVPGSTGQVQLVESGGGVVQPGRSLRLSCAASGFTFSIYGMHWVRQAPGKGLEWVAVISYDGNNKDYTDSVKGRFTISRDNSKNTLYLQMNSLTAEDTAVYYCAKRGYGSSSLEFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 297 - DNA, 1410 bp, Synthetic construct
  source 1..1410: mol_type = other DNA
atggagacagatactttgctcctttgggtactcttgctgtgggttcccggtagcaccggccaagtccagcttgtagagtccggcggcggcgtagtccaaccgggtcggtcacttcggttgtcctgtgcggcgagtggtttcaccttcagcatttatggtatgcactgggtaagacaagcccctggcaaaggattggaatgggtggcagtaatttcctacgatggtaacaacaaggactacacggacagtgtgaaaggtcggttcactatatcacgggacaatagcaagaacactctctacttgcagatgaactcactgacggccgaggatactgcagtatattactgcgccaagcgcgggtacggttctagttcccttgagttttggggtcagggcactctggtaacggtgtctagcgctagcaccaaaggaccctccgtctttccgctcgcgccgagcagcaagtccacgagcggggggacagccgcccttgggtgtctggtcaaggattattttcctgagcccgttactgtctcctggaatagcggtgccttgacttctggggtacataccttcccggcggttttgcaatcatcaggactgtatagcctctctagcgttgtaacggtaccatcttcatcacttggcacacaaacatatatttgtaatgtaaatcacaaaccttcaaatactaaagttgacaaaaaagtggagccgaagagctgtgataaaacgcatacttgcccgccttgccccgctccagaattgctgggcgggccgtctgtgttcctcttcccaccgaaaccgaaggatacgctgatgataagtcgaacgcccgaagtaacatgcgtggttgttgatgtaagccacgaagatccggaagtaaagttcaactggtacgtagatggagtagaagtgcataacgcgaaaactaagccccgcgaggagcaatataattcaacctatcgggttgttagtgtgctcaccgttctccatcaggattggcttaacgggaaagagtataagtgcaaggttagcaataaagcattgccagcacctatagagaagacaattagcaaagcgaagggacagccacgagagccccaagtctacacgctgcctcccagccgcgacgagttgacgaagaatcaagtgtcactcacatgtcttgtcaagggattctatccctcagatattgcagtagaatgggagtctaatggacaaccagagaacaactataaaactactccaccggtgcttgatagtgacggtagttttttcctttatagtaagctgactgtcgataagagtcgctggcaacagggaaatgttttttcttgtagtgttatgcatgaagcacttcataaccattacacacaaaagtctctgtcactttcccctggcaaatga

SEQ ID NO: 298 - protein, 449 aa, Synthetic construct
QVQLVESGGGVVQPGRSLRLSCAASGFTFSIYGMHWVRQAPGKGLEWVAVISYDGNNKDYTDSVKGRFTISRDNSKNTLYLQMNSLTAEDTAVYYCAKRGYGSSSLEFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 299 - protein, 529 aa, Synthetic construct
METDTLLLWVLLLWVPGSTGQVQLVQSGTEVKYPGASVKVSCKASGYTFIDYGISWVRQAPGQGLEWMGWISAYNGNTDYAQKLQGRVTMTTDTSTSTAYMDLRSLRSDDTAVYYCAREGEGYCTYGVCPSFDYWGQGTLVTVSSGGGGSGGGGSGGGGSEIVLTQSPGTLSLSPGERATLSCRASQSVSSSYLAWYQQKPGQAPRLLIYGASTRGTGIPDRFSGSGSGTDFTLTISRLEPEDFALYYCQQYGSSPYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 300 - DNA, 1590 bp, Synthetic construct
  source 1..1590: mol_type = other DNA
atggagacagatactttgctcctttgggtattgctgttgtgggttccaggaagtacaggccaggtacaattggtccagtccgggacagaagtcaagtatccgggggcttctgttaaggtatcctgtaaagcgtccggttacacattcatcgactacggcataagctgggtaagacaagcaccgggccaagggcttgaatggatgggatggatctccgcatacaatggtaacacggactatgcccaaaagctgcaaggaagagtaactatgactacagatacaagcaccagcacggcctatatggatcttagaagtcttcggagtgacgacacggcggtttactactgcgcaagggagggggagggatattgcacatatggagtgtgtccatcattcgattattggggacaaggaaccttggtaacagtgtcttcaggggggggtggatcaggtggtggagggtccgggggaggaggatctgaaattgttctgacgcagtctccgggtaccctctcactgagtcctggtgaaagggcaacccttagttgccgcgcatcacagtcagtttcatccagttatctcgcctggtaccagcaaaagcctggtcaagccccgcggttgcttatctacggggcatccacccgaggtaccggcattcctgaccgattctccggttcagggtcagggactgatttcactctcactataagccgacttgagccggaggatttcgccttgtactattgccagcagtacgggtccagcccatatactttcggccaaggtacaaagttggagataaagagcggaggaggcggatccgaggtacagttggtcgagagtggagggggactcgttcaacccggcggttccctgaagctgtcctgcgcagcgagcggtttcactttcaataaatacgcgatgaactgggtaaggcaagctccaggcaaaggtcttgagtgggttgcaagaatcagaagcaagtataataactacgctacttactacgctgattcagtgaaagacagatttaccatatcccgggatgactcaaaaaacaccgcctacctgcaaatgaataacttgaagaccgaggataccgctgtatactattgtgtgcgccatggaaattttggcaacagttatatatcatactgggcctactggggccagggaactctggtgaccgtaagtagtggaggtggtgggtccggaggtgggggatctgggggtggaggttcccaaactgttgttacgcaagagccatcacttacggtctcccccgggggaacagttacgttgacatgtggttcttctactggtgcggttacgagcggtaactaccctaattgggtccagcagaagcccggacaagctccgcggggacttatcggcggaactaagtttcttgcacctggcaccccagcacgatttagtggtagccttttgggtggcaaggcagctctcaccttgtccggcgttcagcctgaggatgaagcggagtactactgcgtgctgtggtattcaaataggtgggttttcggtggaggtacgaaactgactgtgcttcatcaccaccaccatcactga

SEQ ID NO: 301 - protein, 525 aa, Synthetic construct
METDTLLLWVLLLWVPGSTGEVQLVESGGDLVKSGGSLRLSCAASGFTFSDAWMSWVRQAPGKGLEWVGRIQSKTDGGTTDYVAPVKGRFTISRDDSKDTLYLHMNSLTTEDTAVYYCTTDLYYYGSGSYFLGQGTLVTVSSGGGGSGGGGSGGGGSDIQMTQSPSSLSASVGDRVTITCRASQNIITYLNWYQQKPGKAPKLLIYAASSLQSGVPSRFSGSGSGTDFTLTISSLQPEDFAIYHCQQSYNVPTTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 302 - DNA, 1578 bp, Synthetic construct
  source 1..1578: mol_type = other DNA
atggagacagatactttgctcctttgggtattgctgttgtgggttccaggaagtacaggcgaagtccaactcgtcgagagcgggggtgaccttgtcaagtccggcggaagcctcagactgtcttgtgcagccagcgggtttacgttctctgatgcctggatgtcatgggtaagacaggcccctggaaaagggcttgagtgggtaggccgcatacagagcaaaactgacggggggacaacggattacgtcgctccagttaaagggcggttcactataagccgagacgacagtaaggacacgctttatcttcacatgaatagccttaccactgaggacacggctgtctattattgcacgacagacctgtactattacggtagcggatcatatttcctcggacaaggaaccttggtaacagtgtcttcaggggggggtggatcaggtggtggagggtccgggggaggaggatctgacattcaaatgactcaaagtccaagtagccttagtgcgagcgtcggagatcgagtcactataacctgtcgggccagccaaaatatcatcacctacctcaattggtatcagcagaaaccaggtaaggcaccaaaactgcttatatatgctgcttcatctcttcaatccggtgtgccctctcgattctctgggagtgggtctggcacagatttcactcttactatatcatctctgcaacccgaggactttgcgatatatcattgccagcagtcatacaacgtcccaactacgtttgggcaggggactaagctggagataaaaagcggaggaggcggatccgaggtacagttggtcgagagtggagggggactcgttcaacccggcggttccctgaagctgtcctgcgcagcgagcggtttcactttcaataaatacgcgatgaactgggtaaggcaagctccaggcaaaggtcttgagtgggttgcaagaatcagaagcaagtataataactacgctacttactacgctgattcagtgaaagacagatttaccatatcccgggatgactcaaaaaacaccgcctacctgcaaatgaataacttgaagaccgaggataccgctgtatactattgtgtgcgccatggaaattttggcaacagttatatatcatactgggcctactggggccagggaactctggtgaccgtaagtagtggaggtggtgggtccggaggtgggggatctgggggtggaggttcccaaactgttgttacgcaagagccatcacttacggtctcccccgggggaacagttacgttgacatgtggttcttctactggtgcggttacgagcggtaactaccctaattgggtccagcagaagcccggacaagctccgcggggacttatcggcggaactaagtttcttgcacctggcaccccagcacgatttagtggtagccttttgggtggcaaggcagctctcaccttgtccggcgttcagcctgaggatgaagcggagtactactgcgtgctgtggtattcaaataggtgggttttcggtggaggtacgaaactgactgtgcttcatcaccaccaccatcactga

SEQ ID NO: 303 - protein, 528 aa, Synthetic construct
METDTLLLWVLLLWVPGSTGQVQLQESGPGLVKPSETLSLTCTVSGASISGYYWNWIRQPPGKGLEWIGYIHYSGTTNYNPSLKSRITISLDTSENQFSLRLSSVTAADTAVYYCARDWYTGYDWEGFDYWGQGTLVTVSSGGGGSGGGGSGGGGSQSVLTQPPSVSGAPGQRVSFSCTGTSSNIGTGFDVHWYRQLPGTAPKVLIYNNNNRPSGVPDRFSASKSGTSASLAITGLQAEDEADYYCQSYDSSLSGSVFGGGTRLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 304 - DNA, 1587 bp, Synthetic construct
  source 1..1587: mol_type = other DNA
atggagacagatactttgctcctttgggttttgttgctgtgggttccaggatccacggggcaggtgcaacttcaggagtcaggtccaggcttggttaaaccaagtgagacactgtccctgacttgcacagtatccggggcatctatctctggttattattggaactggattaggcaaccacctgggaagggtcttgaatggattggttacatacattactcagggacgacgaattataatccatccctcaagtccagaataactatcagtctcgacacttcagaaaatcagtttagcctgagactgtcaagtgtcaccgctgcggatactgccgtatattactgcgctagagactggtataccggctacgactgggaaggattcgattattggggccaaggaaccttggtaacagtgtcttcaggggggggtggatcaggtggtggagggtccgggggaggaggatctcagagcgtacttacacagcccccttcagtcagtggagcgccaggtcaaagggtctctttttcatgtactggaacgtcaagcaacatcgggacaggatttgatgtccactggtataggcagctcccaggaactgcccccaaagtcttgatctataacaataacaatcgcccttctggcgtacctgatcgattcagcgcatcaaaatccgggacctcagccagcctcgccattacgggcttgcaggcagaagacgaggccgattactactgtcaaagttatgatagctcattgagcgggtcagtctttggcggcggtacgagattgaccgttctgagcggaggaggcggatccgaggtacagttggtcgagagtggagggggactcgttcaacccggcggttccctgaagctgtcctgcgcagcgagcggtttcactttcaataaatacgcgatgaactgggtaaggcaagctccaggcaaaggtcttgagtgggttgcaagaatcagaagcaagtataataactacgctacttactacgctgattcagtgaaagacagatttaccatatcccgggatgactcaaaaaacaccgcctacctgcaaatgaataacttgaagaccgaggataccgctgtatactattgtgtgcgccatggaaattttggcaacagttatatatcatactgggcctactggggccagggaactctggtgaccgtaagtagtggaggtggtgggtccggaggtgggggatctgggggtggaggttcccaaactgttgttacgcaagagccatcacttacggtctcccccgggggaacagttacgttgacatgtggttcttctactggtgcggttacgagcggtaactaccctaattgggtccagcagaagcccggacaagctccgcggggacttatcggcggaactaagtttcttgcacctggcaccccagcacgatttagtggtagccttttgggtggcaaggcagctctcaccttgtccggcgttcagcctgaggatgaagcggagtactactgcgtgctgtggtattcaaataggtgggttttcggtggaggtacgaaactgactgtgcttcatcaccaccaccatcactga

SEQ ID NO: 305 - protein, 522 aa, Synthetic construct
METDTLLLWVLLLWVPGSTGQVQLVESGGGVVLPGRSLRLSCAASGFTFNIYGMHWVRQAPGKGLEWVAVISYDGNNKEYADSVKGRFTISRDSSRNTLYLQMNSLRAEDTAVYYCAKRGYGSSSLEFWGQGTLVTVSSGGGGSGGGGSGGGGSDIQMTQSPSSLSASVGDRVTITCRSSQDIRNDLGWYQQKPGKAPKRLIYASSNLQSGVPSRFSGSGSGTEFTLTISSLQPDDFATYYCLQHNTYPYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 306 - DNA, 1569 bp, Synthetic construct
  source 1..1569: mol_type = other DNA
atggagacagatactttgctcctttgggttttgttgctgtgggttccaggatccacggggcaagtccaacttgttgaaagtggagggggagttgtcctgccgggtcgatctcttcgccttagctgcgcggcgagtgggttcacatttaacatatacggaatgcactgggtgcgccaagcccctggaaaggggctcgaatgggttgcggtcattagttacgatgggaacaacaaagagtacgctgacagtgtgaagggacgatttacgatttcacgcgactcatctcgcaacacattgtacttgcaaatgaactcacttagagcagaagatacggctgtttattattgtgcaaaaagaggctatggatcatcctcacttgaattttggggtcaaggaaccttggtaacagtgtcttcaggggggggtggatcaggtggtggagggtccgggggaggaggatctgacatccagatgacacaatcaccgtctagcctttctgcgtcagtcggagaccgcgtgaccataacgtgcagatcctcccaggatattcggaacgatttgggctggtatcagcagaagccgggcaaagcgccaaagcgcctgatctacgcctcttccaacttgcagtccggtgtacctagcaggttttctggtagcggcagtggtactgaatttaccttgacaatatccagtcttcagccagacgactttgccacctattactgtcttcaacataacacttacccatacacctttggacaaggtacaaaattggagattaaaagcggaggaggcggatccgaggtacagttggtcgagagtggagggggactcgttcaacccggcggttccctgaagctgtcctgcgcagcgagcggtttcactttcaataaatacgcgatgaactgggtaaggcaagctccaggcaaaggtcttgagtgggttgcaagaatcagaagcaagtataataactacgctacttactacgctgattcagtgaaagacagatttaccatatcccgggatgactcaaaaaacaccgcctacctgcaaatgaataacttgaagaccgaggataccgctgtatactattgtgtgcgccatggaaattttggcaacagttatatatcatactgggcctactggggccagggaactctggtgaccgtaagtagtggaggtggtgggtccggaggtgggggatctgggggtggaggttcccaaactgttgttacgcaagagccatcacttacggtctcccccgggggaacagttacgttgacatgtggttcttctactggtgcggttacgagcggtaactaccctaattgggtccagcagaagcccggacaagctccgcggggacttatcggcggaactaagtttcttgcacctggcaccccagcacgatttagtggtagccttttgggtggcaaggcagctctcaccttgtccggcgttcagcctgaggatgaagcggagtactactgcgtgctgtggtattcaaataggtgggttttcggtggaggtacgaaactgactgtgcttcatcaccaccaccatcactga

SEQ ID NO: 307 - protein, 495 aa, Synthetic construct
MRAWIFFLLCLAGRALADIQMTQSPSSLSASVGDRVTITCRASQNIITYLNWYQQKPGKAPKLLIYAASSLQSGVPSRFSGSGSGTDFTLTISSLQPEDFAIYHCQQSYNVPTTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 308 - DNA, 1488 bp, Synthetic construct
  source 1..1488: mol_type = other DNA
atgagagcctggatcttcttcctgctttgtttggcaggacgagcgctagccgacattcaaatgactcaaagtccaagtagccttagtgcgagcgtcggagatcgagtcactataacctgtcgggccagccaaaatatcatcacctacctcaattggtatcagcagaaaccaggtaaggcaccaaaactgcttatatatgctgcttcatctcttcaatccggtgtgccctctcgattctctgggagtgggtctggcacagatttcactcttactatatcatctctgcaacccgaggactttgcgatatatcattgccagcagtcatacaacgtcccaactacgtttgggcaggggactaagctggagataaaacgaacagtggcggcaccgtccgtatttattttccctccaagtgatgagcagctcaaaagtggcacagcgagcgttgtatgtctcctcaataacttttaccccagagaggcgaaagtacaatggaaggttgataacgcactccaaagcggtaactctcaggagagtgtcacagaacaggatagtaaagactccacctactccctcagttctacccttactctttcaaaagcggattatgagaaacacaaggtttacgcctgtgaagtcactcatcagggactgagttccccggtcacgaagtcatttaatcgcggtgagtgcggggggggtggatcaggtggtggagggtccgggggaggaggatccgaggtacagttggtcgagagtggagggggactcgttcaacccggcggttccctgaagctgtcctgcgcagcgagcggtttcactttcaataaatacgcgatgaactgggtaaggcaagctccaggcaaaggtcttgagtgggttgcaagaatcagaagcaagtataataactacgctacttactacgctgattcagtgaaagacagatttaccatatcccgggatgactcaaaaaacaccgcctacctgcaaatgaataacttgaagaccgaggataccgctgtatactattgtgtgcgccatggaaattttggcaacagttatatatcatactgggcctactggggccagggaactctggtgaccgtaagtagtggaggtggtgggtccggaggtgggggatctgggggtggaggttcccaaactgttgttacgcaagagccatcacttacggtctcccccgggggaacagttacgttgacatgtggttcttctactggtgcggttacgagcggtaactaccctaattgggtccagcagaagcccggacaagctccgcggggacttatcggcggaactaagtttcttgcacctggcaccccagcacgatttagtggtagccttttgggtggcaaggcagctctcaccttgtccggcgttcagcctgaggatgaagcggagtactactgcgtgctgtggtattcaaataggtgggttttcggtggaggtacgaaactgactgtgctttga

SEQ ID NO: 309 - protein, 498 aa, Synthetic construct
MRAWIFFLLCLAGRALAQSVLTQPPSVSGAPGQRVSFSCTGTSSNIGTGFDVHWYRQLPGTAPKVLIYNNNNRPSGVPDRFSASKSGTSASLAITGLQAEDEADYYCQSYDSSLSGSVFGGGTRLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECSGGGGSGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 310 - DNA, 1497 bp, Synthetic construct
  source 1..1497: mol_type = other DNA
atgagagcctggatcttcttcctgctttgtttggcaggacgagcgctagcccagagcgtacttacacagcccccttcagtcagtggagcgccaggtcaaagggtctctttttcatgtactggaacgtcaagcaacatcgggacaggatttgatgtccactggtataggcagctcccaggaactgcccccaaagtcttgatctataacaataacaatcgcccttctggcgtacctgatcgattcagcgcatcaaaatccgggacctcagccagcctcgccattacgggcttgcaggcagaagacgaggccgattactactgtcaaagttatgatagctcattgagcgggtcagtctttggcggcggtacgagattgaccgttctgggacagcctaaagcaaatccaaccgtaaccttgtttccaccgtccagcgaagagcttcaggctaacaaagctacactcgtatgcctgatcagtgacttctatcctggggccgtaacagtagcgtggaaagcagatgggtcacccgtcaaagcaggggtggagacgaccaaaccctccaagcagtccaacaataagtacgcagcctcaagttatttgtcacttactcctgaacaatggaaatcacatcgatcctattcctgtcaagttacccatgaggggagcactgttgaaaaaactgtcgccccaactgaatgtagtggggggggtggatcaggtggtggagggtccgggggaggaggatccgaggtacagttggtcgagagtggagggggactcgttcaacccggcggttccctgaagctgtcctgcgcagcgagcggtttcactttcaataaatacgcgatgaactgggtaaggcaagctccaggcaaaggtcttgagtgggttgcaagaatcagaagcaagtataataactacgctacttactacgctgattcagtgaaagacagatttaccatatcccgggatgactcaaaaaacaccgcctacctgcaaatgaataacttgaagaccgaggataccgctgtatactattgtgtgcgccatggaaattttggcaacagttatatatcatactgggcctactggggccagggaactctggtgaccgtaagtagtggaggtggtgggtccggaggtgggggatctgggggtggaggttcccaaactgttgttacgcaagagccatcacttacggtctcccccgggggaacagttacgttgacatgtggttcttctactggtgcggttacgagcggtaactaccctaattgggtccagcagaagcccggacaagctccgcggggacttatcggcggaactaagtttcttgcacctggcaccccagcacgatttagtggtagccttttgggtggcaaggcagctctcaccttgtccggcgttcagcctgaggatgaagcggagtactactgcgtgctgtggtattcaaataggtgggttttcggtggaggtacgaaactgactgtgctttga

SEQ ID NO: 311 - protein, 495 aa, Synthetic construct
MRAWIFFLLCLAGRALADIQMTQSPSSLSASVGDRVTITCRSSQDIRNDLGWYQQKPGKAPKRLIYASSNLQSGVPSRFSGSGSGTEFTLTISSLQPDDFATYYCLQHNTYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 312 - DNA, 1488 bp, Synthetic construct
  source 1..1488: mol_type = other DNA
atgagagcctggatcttcttcctgctttgtttggcaggacgagcgctagccgacatccagatgacacaatcaccgtctagcctttctgcgtcagtcggagaccgcgtgaccataacgtgcagatcctcccaggatattcggaacgatttgggctggtatcagcagaagccgggcaaagcgccaaagcgcctgatctacgcctcttccaacttgcagtccggtgtacctagcaggttttctggtagcggcagtggtactgaatttaccttgacaatatccagtcttcagccagacgactttgccacctattactgtcttcaacataacacttacccatacacctttggacaaggtacaaaattggagattaaacgaacagtggcggcaccgtccgtatttattttccctccaagtgatgagcagctcaaaagtggcacagcgagcgttgtatgtctcctcaataacttttaccccagagaggcgaaagtacaatggaaggttgataacgcactccaaagcggtaactctcaggagagtgtcacagaacaggatagtaaagactccacctactccctcagttctacccttactctttcaaaagcggattatgagaaacacaaggtttacgcctgtgaagtcactcatcagggactgagttccccggtcacgaagtcatttaatcgcggtgagtgcggggggggtggatcaggtggtggagggtccgggggaggaggatccgaggtacagttggtcgagagtggagggggactcgttcaacccggcggttccctgaagctgtcctgcgcagcgagcggtttcactttcaataaatacgcgatgaactgggtaaggcaagctccaggcaaaggtcttgagtgggttgcaagaatcagaagcaagtataataactacgctacttactacgctgattcagtgaaagacagatttaccatatcccgggatgactcaaaaaacaccgcctacctgcaaatgaataacttgaagaccgaggataccgctgtatactattgtgtgcgccatggaaattttggcaacagttatatatcatactgggcctactggggccagggaactctggtgaccgtaagtagtggaggtggtgggtccggaggtgggggatctgggggtggaggttcccaaactgttgttacgcaagagccatcacttacggtctcccccgggggaacagttacgttgacatgtggttcttctactggtgcggttacgagcggtaactaccctaattgggtccagcagaagcccggacaagctccgcggggacttatcggcggaactaagtttcttgcacctggcaccccagcacgatttagtggtagccttttgggtggcaaggcagctctcaccttgtccggcgttcagcctgaggatgaagcggagtactactgcgtgctgtggtattcaaataggtgggttttcggtggaggtacgaaactgactgtgctttga

SEQ ID NO: 313 - protein, 496 aa, Synthetic construct
MRAWIFFLLCLAGRALAEIVLTQSPGTLSLSPGERATLSCRASQSVSSSYLAWYQQKPGQAPRLLIYGASTRGTGIPDRFSGSGSGTDFTLTISRLEPEDFALYYCQQYGSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 314 - DNA, 1491 bp, Synthetic construct
  source 1..1491: mol_type = other DNA
atgagagcctggatcttcttcctgctttgtttggcaggacgagcgctagccgaaattgttctgacgcagtctccgggtaccctctcactgagtcctggtgaaagggcaacccttagttgccgcgcatcacagtcagtttcatccagttatctcgcctggtaccagcaaaagcctggtcaagccccgcggttgcttatctacggggcatccacccgaggtaccggcattcctgaccgattctccggttcagggtcagggactgatttcactctcactataagccgacttgagccggaggatttcgccttgtactattgccagcagtacgggtccagcccatatactttcggccaaggtacaaagttggagataaagcgaacagtggcggcaccgtccgtatttattttccctccaagtgatgagcagctcaaaagtggcacagcgagcgttgtatgtctcctcaataacttttaccccagagaggcgaaagtacaatggaaggttgataacgcactccaaagcggtaactctcaggagagtgtcacagaacaggatagtaaagactccacctactccctcagttctacccttactctttcaaaagcggattatgagaaacacaaggtttacgcctgtgaagtcactcatcagggactgagttccccggtcacgaagtcatttaatcgcggtgagtgcggggggggtggatcaggtggtggagggtccgggggaggaggatccgaggtacagttggtcgagagtggagggggactcgttcaacccggcggttccctgaagctgtcctgcgcagcgagcggtttcactttcaataaatacgcgatgaactgggtaaggcaagctccaggcaaaggtcttgagtgggttgcaagaatcagaagcaagtataataactacgctacttactacgctgattcagtgaaagacagatttaccatatcccgggatgactcaaaaaacaccgcctacctgcaaatgaataacttgaagaccgaggataccgctgtatactattgtgtgcgccatggaaattttggcaacagttatatatcatactgggcctactggggccagggaactctggtgaccgtaagtagtggaggtggtgggtccggaggtgggggatctgggggtggaggttcccaaactgttgttacgcaagagccatcacttacggtctcccccgggggaacagttacgttgacatgtggttcttctactggtgcggttacgagcggtaactaccctaattgggtccagcagaagcccggacaagctccgcggggacttatcggcggaactaagtttcttgcacctggcaccccagcacgatttagtggtagccttttgggtggcaaggcagctctcaccttgtccggcgttcagcctgaggatgaagcggagtactactgcgtgctgtggtattcaaataggtgggttttcggtggaggtacgaaactgactgtgctttga

SEQ ID NO: 315 - protein, 451 aa, Synthetic construct
QVQLQESGPGLVKPSETLSLTCTVSGASISGYYWNWIRQPPGKGLEWIGYIHYSGTTNYNPSLKSRITISLDTSENQFSLRLSSVTAADTAVYYCARDWYTGYDWEGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 316 - protein, 448 aa, Synthetic construct
QVQLQESGPGLVKPSETLSLTCTVSGASISGYYWNWIRQPPGKGLEWIGYIHYSGTTNYNPSLKSRITISLDTSENQFSLRLSSVTAADTAVYYCARDWYTGYDWEGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 317 - protein, 217 aa, Synthetic construct
QSVLTQPPSVSGAPGQRVSFSCTGTSSNIGTGFDVHWYRQLPGTAPKVLIYNNNNRPSGVPDRFSASKSGTSASLAITGLQAEDEADYYCQSYDSSLSGSVFGGGTRLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 318 - protein, 455 aa, Synthetic construct
QVQLVQSGTEVKYPGASVKVSCKASGYTFIDYGISWVRQAPGQGLEWMGWISAYNGNTDYAQKLQGRVTMTTDTSTSTAYMDLRSLRSDDTAVYYCAREGEGYCTYGVCPSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 319 - protein, 452 aa, Synthetic construct
QVQLVQSGTEVKYPGASVKVSCKASGYTFIDYGISWVRQAPGQGLEWMGWISAYNGNTDYAQKLQGRVTMTTDTSTSTAYMDLRSLRSDDTAVYYCAREGEGYCTYGVCPSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 320 - protein, 215 aa, Synthetic construct
EIVLTQSPGTLSLSPGERATLSCRASQSVSSSYLAWYQQKPGQAPRLLIYGASTRGTGIPDRFSGSGSGTDFTLTISRLEPEDFALYYCQQYGSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 321 - protein, 449 aa, Synthetic construct
QVQLVESGGGVVLPGRSLRLSCAASGFTFNIYGMHWVRQAPGKGLEWVAVISYDGNNKEYADSVKGRFTISRDSSRNTLYLQMNSLRAEDTAVYYCAKRGYGSSSLEFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 322 - protein, 446 aa, Synthetic construct
QVQLVESGGGVVLPGRSLRLSCAASGFTFNIYGMHWVRQAPGKGLEWVAVISYDGNNKEYADSVKGRFTISRDSSRNTLYLQMNSLRAEDTAVYYCAKRGYGSSSLEFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 323 - protein, 214 aa, Synthetic construct
DIQMTQSPSSLSASVGDRVTITCRSSQDIRNDLGWYQQKPGKAPKRLIYASSNLQSGVPSRFSGSGSGTEFTLTISSLQPDDFATYYCLQHNTYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 324 - protein, 452 aa, Synthetic construct
EVQLVESGGDLVKSGGSLRLSCAASGFTFSDAWMSWVRQAPGKGLEWVGRIQSKTDGGTTDYVAPVKGRFTISRDDSKDTLYLHMNSLTTEDTAVYYCTTDLYYYGSGSYFLGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 325 - protein, 449 aa, Synthetic construct
EVQLVESGGDLVKSGGSLRLSCAASGFTFSDAWMSWVRQAPGKGLEWVGRIQSKTDGGTTDYVAPVKGRFTISRDDSKDTLYLHMNSLTTEDTAVYYCTTDLYYYGSGSYFLGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 326 - protein, 511 aa, Synthetic construct
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 327 - protein, 523 aa, Synthetic construct
MGWSCIILFLVATATGVHSDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 328 - protein, 272 aa, Synthetic construct
MAEVQLVESGGSLVKPGGSLRLSCAASGFTFSNYSMNWVRQAPGKGLEWISSISGSSRYIYYADFVKGRFTISRDNATNSLYLQMNSLRAEDTAVYYCVRSSITIFGGGMDVWGRGTLVTVSSGGGGSGGGGSGGGGSQSVLTQPASVSGSPGQSITISCAGTSSDVGGYNYVSWYQQHPGKAPKLMIYEDSKRPSGVSNRFSGSKSGNTASLTISGLQAEDEADYYCSSYTTRSTRVFGGGTKLAVLGAAAEQKLISEEDLNGAAHHHHHH

SEQ ID NO: 329 - protein, 509 aa, Synthetic construct
QVQLVQSGTEVKYPGASVKVSCKASGYTFIDYGISWVRQAPGQGLEWMGWISAYNGNTDYAQKLQGRVTMTTDTSTSTAYMDLRSLRSDDTAVYYCAREGEGYCTYGVCPSFDYWGQGTLVTVSSGGGGSGGGGSGGGGSEIVLTQSPGTLSLSPGERATLSCRASQSVSSSYLAWYQQKPGQAPRLLIYGASTRGTGIPDRFSGSGSGTDFTLTISRLEPEDFALYYCQQYGSSPYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 330 - protein, 525 aa, Synthetic construct; identical to SEQ ID NO: 301

SEQ ID NO: 331 - protein, 508 aa, Synthetic construct
QVQLQESGPGLVKPSETLSLTCTVSGASISGYYWNWIRQPPGKGLEWIGYIHYSGTTNYNPSLKSRITISLDTSENQFSLRLSSVTAADTAVYYCARDWYTGYDWEGFDYWGQGTLVTVSSGGGGSGGGGSGGGGSQSVLTQPPSVSGAPGQRVSFSCTGTSSNIGTGFDVHWYRQLPGTAPKVLIYNNNNRPSGVPDRFSASKSGTSASLAITGLQAEDEADYYCQSYDSSLSGSVFGGGTRLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 332 - protein, 502 aa, Synthetic construct
QVQLVESGGGVVLPGRSLRLSCAASGFTFNIYGMHWVRQAPGKGLEWVAVISYDGNNKEYADSVKGRFTISRDSSRNTLYLQMNSLRAEDTAVYYCAKRGYGSSSLEFWGQGTLVTVSSGGGGSGGGGSGGGGSDIQMTQSPSSLSASVGDRVTITCRSSQDIRNDLGWYQQKPGKAPKRLIYASSNLQSGVPSRFSGSGSGTEFTLTISSLQPDDFATYYCLQHNTYPYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 333 - protein, 478 aa, Synthetic construct
DIQMTQSPSSLSASVGDRVTITCRASQNIITYLNWYQQKPGKAPKLLIYAASSLQSGVPSRFSGSGSGTDFTLTISSLQPEDFAIYHCQQSYNVPTTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 334 - protein, 481 aa, Synthetic construct
QSVLTQPPSVSGAPGQRVSFSCTGTSSNIGTGFDVHWYRQLPGTAPKVLIYNNNNRPSGVPDRFSASKSGTSASLAITGLQAEDEADYYCQSYDSSLSGSVFGGGTRLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECSGGGGSGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 335 - protein, 478 aa, Synthetic construct
DIQMTQSPSSLSASVGDRVTITCRSSQDIRNDLGWYQQKPGKAPKRLIYASSNLQSGVPSRFSGSGSGTEFTLTISSLQPDDFATYYCLQHNTYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 336 - protein, 479 aa, Synthetic construct
EIVLTQSPGTLSLSPGERATLSCRASQSVSSSYLAWYQQKPGQAPRLLIYGASTRGTGIPDRFSGSGSGTDFTLTISRLEPEDFALYYCQQYGSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 337 - protein, 107 aa, Homo sapiens [sequence omitted]

SEQ ID NO: 338 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 339 - protein, 330 aa, Homo sapiens [sequence omitted]

SEQ ID NO: 340 - protein, 225 aa, Homo sapiens
THTCPPCPAPEFFGGPSVFFFPPKPKDTFMISRTPEVTCVVVDVSHEDPEVKFNWYVDGVEVHNAKTKPREEQYNSTYRVVSVETVFHQDWENGKEYKCKVSNKAFPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHNHYTQKSLSLSPGK

SEQ ID NO: 341 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 342 - protein, 217 aa, Homo sapiens
PAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 343 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 344 - protein, 218 aa, Homo sapiens
PAPELLGGPSVFLFPPKPKDTLMISRTPEVTCVVVDVSHEDPEVQFKWYVDGVEVHNAKTKPREEQFNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 345 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 346 - protein, 218 aa, Homo sapiens
PAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 347 - protein, 5 aa, Synthetic construct
  REPEAT 1..5: note = (GGGGS) can be repeated n times, wherein n is an integer of 1, 2, 3, 4, 5, 6, 7, 8, 9 or 10
GGGGS

SEQ ID NO: 348 - protein, 9 aa, Synthetic construct
  REPEAT 1..4: note = (GGGS) can be repeated n times, wherein n is an integer of 1, 2, 3, 4, 5, 6, 7, 8, 9 or 10
  REPEAT 5..9: note = (GGGGS) can be repeated n times, wherein n is an integer of 1, 2, 3, 4, 5, 6, 7, 8, 9 or 10
GGGSGGGGS

SEQ ID NO: 349 - protein, 7 aa, Synthetic construct
  REPEAT 1..4: note = (GGGS) can be repeated n times, wherein n is an integer of 1, 2, 3, 4, 5, 6, 7, 8, 9 or 10
  REPEAT 5..7: note = (GGS) can be repeated n times, wherein n is an integer of 1, 2, 3, 4, 5, 6, 7, 8, 9 or 10
GGGSGGS

SEQ ID NO: 350 - protein, 9 aa, Synthetic construct
  REPEAT 1..4: note = (GGGS) can be repeated n times, wherein n is an integer of 1, 2, 3, 4, 5, 6, 7, 8, 9 or 10
GGGSGGGGS

SEQ ID NO: 351 - protein, 20 aa, Synthetic construct
GGGGSGGGGSGGGGSGGGGS

SEQ ID NO: 352 - protein, 15 aa, Synthetic construct
GGGGSGGGGSGGGGS

SEQ ID NO: 353 - protein, 10 aa, Synthetic construct
GGGGSGGGGS

SEQ ID NO: 354 - protein, 5 aa, Synthetic construct
GGGGS

SEQ ID NO: 355 - protein, 8 aa, Synthetic construct
GGGSGGGS

SEQ ID NO: 356 - protein, 4 aa, Synthetic construct
GGGS

SEQ ID NO: 357 - protein, 6 aa, Synthetic construct
GGSGGS

SEQ ID NO: 358 - protein, 11 aa, Synthetic construct
GGSGGGSGGSG

SEQ ID NO: 359 - protein, 10 aa, Synthetic construct
GGSGGGSGSG

SEQ ID NO: 360 - protein, 8 aa, Synthetic construct
GGSGGGSG

SEQ ID NO: 361 - skipped

SEQ ID NO: 362 - protein, 39 aa, Synthetic construct
RIARLEEKVKTLKAQNSELASTANMLREQVAQLKQKVMN

SEQ ID NO: 363 - protein, 39 aa, Synthetic construct
LTDTLQAETDQLEDKKSALQTEIANLLKEKEKLEFILAA

SEQ ID NO: 364 - protein, 59 aa, Synthetic construct
WREFCESHARAAALDFARRFRLYLASHPQYAGPGAEAAFSRRFAELFLQHFEAEVARAS

SEQ ID NO: 365 - protein, 55 aa, Synthetic construct
THRAIFRFVPRHEDELELEVDDPLLVELQAEDYWYEAYNMRTGARGVFPAYYAIE

SEQ ID NO: 366 - protein, 101 aa, Oryctolagus cuniculus
LGEVHRFHVTVAPGTKLESGPATLHLCNDVLVLARDIPPAVTGQWKLSDLRRYGAVPSGFIFEGGTRCGYWAGVFFLSSAEGEQISFLFDCIVRGISPTKG

SEQ ID NO: 367 - protein, 115 aa, Homo sapiens
DCKEEHAEFVLVRKDMLFNQLIEMALLSLGYSHSSAAQAKGLIQVGKWNPVPLSYVTDAPDATVADMLQDVYHVVTLKIQLHSCPKLEDLPPEQWSHTTVRNALKDLLKDMNQSS

SEQ ID NO: 368 - protein, 427 aa, Homo sapiens
CAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGSQLFSYAAFSDSNIITVVVDTALYIAKQNSPVVEVWDKKTEKLCGLIDCVHFLREVMVKETKIFSFSNDFTIQKLIETRTNKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 369 - protein, 75 aa, Synthetic construct
DQELKHLILEAADGFLFIVSCETGRVVYVSDSVTPVLNQQQSEWFGSTLYDQVHPDDVDKLREQLSTSENALTGR

SEQ ID NO: 370 - protein, 33 aa, Homo sapiens
LSAKETKMLMAAGDKDGDGKIGVDEFSTLVAES

SEQ ID NO: 371 - protein, 44 aa, Synthetic construct
SHIQIPPGLTELLQGYTVEVLRQQPPDLVEFAVEYFTRLREARA

SEQ ID NO: 372 - protein, 45 aa, Synthetic construct
CGHIQIPPGLTELLQGYTVEVLRQQPPDLVEFAVEYFTRLREARA

SEQ ID NO: 373 - protein, 17 aa, Synthetic construct
QIEYLAKQIVDNAIQQA

SEQ ID NO: 374 - protein, 21 aa, Synthetic construct
CGQIEYLAKQIVDNAIQQAGC

SEQ ID NO: 375 - protein, 106 aa, Synthetic construct
MGVQVETISPGDGRTFPKRGQTCWHYTGMLEDGKKFDSSRDRNPFKFMLGKQEVIRGWEEGVAQMSVGQRAKLTISPDYAYGATGHPGIIPPHATLVFDVELLKLE

SEQ ID NO: 376 - protein, 100 aa, Synthetic construct
MASRILWHEMWHEGLEEASRLYFGERNVKGMFEVLEPLHAMMERGPQTLKETSFNQAYGRDLMEAQEWCRKYMKSGNVKDLTQAWDLYYHVFRRISKLES

SEQ ID NO: 377 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 378 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 379 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 380 - protein, 764 aa, Synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 381 - protein, 585 aa, Synthetic construct
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPR

SEQ ID NO: 382 - protein, 349 aa, Homo sapiens
VIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 383 - protein, 18 aa, Homo sapiens
VEPKSSDKTHTCPPCPAP

SEQ ID NO: 384 - protein, 255 aa, Homo sapiens
VEPKSSDKTHTCPPCPAPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 385 - protein, 453 aa, Homo sapiens [sequence omitted]

SEQ ID NO: 386 - protein, 453 aa, Synthetic construct
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 387 - protein, 453 aa, Synthetic construct
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLASSLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 388 - protein, 453 aa, Synthetic construct
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVAEEEWNTGETYTCVVAHEALPNRVTERTVDKSTGKPTLYNVSLVMSDTAGTCY

SEQ ID NO: 389 - protein, 453 aa, Synthetic construct
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSAEEWNTGETYTCVVAHEALPNRVTERTVDKSTGKPTLYNVSLVMSDTAGTCY

SEQ ID NO: 390 - protein, 453 aa, Synthetic construct
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEAEWNTGETYTCVVAHEALPNRVTERTVDKSTGKPTLYNVSLVMSDTAGTCY

SEQ ID NO: 391 - protein, 453 aa, Synthetic construct
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLAESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 392 - protein, 453 aa, Synthetic construct
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRASATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 393 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 394 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 395 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCSTYDRNKCYTAVVPLVYGGETKMVETALTPDACYPD

SEQ ID NO: 396 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCRTYDRNKCYTAVVPLVYGGETKMVETALTPDACYPD

SEQ ID NO: 397 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYAYDRNKCYTAVVPLVYGGETKMVETALTPDACYPD

SEQ ID NO: 398 - protein, 137 aa, Homo sapiens
QEDERIVLVDNKCKCARITSRIIRSSEDPNEDIVERNIRIIVPLNNREAISDPTSPLRTRFVYHLSDLCKKCDPTEVELDNQIVTATQSNICDEDSATETCYTYDRNKCYTAVVPLVYGGETKMVETALTPDACYPD

SEQ ID NO: 399 - protein, 136 aa, Homo sapiens
EDERIVLVDNKCKCARITSRIIRSSEDPNEDIVERNIRIIVPLNNRENIADPTSPLRTRFVYHLSDLCKKCDPTEVELDNQIVTATQSNICDEDSATETCYTYDRNKCYTAVVPLVYGGETKMVETALTPDACYPD

SEQ ID NO: 400 - skipped

SEQ ID NO: 401 - protein, 6 aa, Synthetic construct
HHHHHH

SEQ ID NO: 402 - protein, 7 aa, Synthetic construct
DYKDDDD

SEQ ID NO: 403 - protein, 8 aa, Synthetic construct
DLYDDDDK

SEQ ID NO: 404 - protein, 15 aa, Synthetic construct
GLNDIFEAQKIEWHE

SEQ ID NO: 405 - protein, 26 aa, Synthetic construct
KRRWKKNFIAVSAANRFKKISSSGAL

SEQ ID NO: 406 - protein, 6 aa, Synthetic construct
EEEEEE

SEQ ID NO: 407 - protein, 9 aa, Synthetic construct
YPYDVPDYA

SEQ ID NO: 408 - protein, 10 aa, Synthetic construct
EQKLISEEDL

SEQ ID NO: 409 - protein, 8 aa, Synthetic construct
WRHPQFGG

SEQ ID NO: 410 - protein, 8 aa, Synthetic construct
WSHPQFEK

SEQ ID NO: 411 - protein, 13 aa, Synthetic construct
SLAELLNAGLGGS

SEQ ID NO: 412 - protein, 8 aa, Synthetic construct
TQDPSRVG

SEQ ID NO: 413 - protein, 14 aa, Synthetic construct
GKPIPNPLLGLDST

SEQ ID NO: 414 - RNA, 20 nt, Synthetic construct
  source 1..20: mol_type = other RNA
ctgggggtaggagaaggagc

SEQ ID NO: 415 - RNA, 20 nt, Synthetic construct
  source 1..20: mol_type = other RNA
cttgctgcaagtgcaggagc

SEQ ID NO: 416 - RNA, 20 nt, Synthetic construct
  source 1..20: mol_type = other RNA
cccctttgtcccccagagca

SEQ ID NO: 417 - DNA, 20 bp, Synthetic construct
  source 1..20: mol_type = other DNA
gctccaaatgtccccaagga

SEQ ID NO: 418 - DNA, 21 bp, Synthetic construct
  source 1..21: mol_type = other DNA
acttttctgacttcaccccca

SEQ ID NO: 419 - DNA, 23 bp, Synthetic construct
  source 1..23: mol_type = other DNA
ctccaaatgtccccaaggagttg

SEQ ID NO: 420 - protein, 347 aa, Synthetic construct [sequence omitted]

SEQ ID NO: 421 - protein, 262 aa, Synthetic construct
MLRLLLALNLFPSIQVTGGSSQVQLVESGGGVVLPGRSLRLSCAASGFTFNIYGMHWVRQAPGKGLEWVAVISYDGNNKEYADSVKGRFTISRDSSRNTLYLQMNSLRAEDTAVYYCAKRGYGSSSLEFWGQGTLVTVSSGGGGSGGGGSGGGGSDIQMTQSPSSLSASVGDRVTITCRSSQDIRNDLGWYQQKPGKAPKRLIYASSNLQSGVPSRFSGSGSGTEFTLTISSLQPDDFATYYCLQHNTYPYTFGQGTKLEIK

SEQ ID NO: 422 - DNA, 786 bp, Synthetic construct
  source 1..786: mol_type = other DNA
atgctcaggctgctcttggctctcaacttattcccttcaattcaagtaacaggagggtcttcgcaagtccaacttgttgaaagtggagggggagttgtcctgccgggtcgatctcttcgccttagctgcgcggcgagtgggttcacatttaacatatacggaatgcactgggtgcgccaagcccctggaaaggggctcgaatgggttgcggtcattagttacgatgggaacaacaaagagtacgctgacagtgtgaagggacgatttacgatttcacgcgactcatctcgcaacacattgtacttgcaaatgaactcacttagagcagaagatacggctgtttattattgtgcaaaaagaggctatggatcatcctcacttgaattttggggtcaaggaaccttggtaacagtgtcttcaggaggcggaggatctggcggagggggctctggaggaggaggatctgacatccagatgacacaatcaccgtctagcctttctgcgtcagtcggagaccgcgtgaccataacgtgcagatcctcccaggatattcggaacgatttgggctggtatcagcagaagccgggcaaagcgccaaagcgcctgatctacgcctcttccaacttgcagtccggtgtacctagcaggttttctggtagcggcagtggtactgaatttaccttgacaatatccagtcttcagccagacgactttgccacctattactgtcttcaacataacacttacccatacacctttggacaaggtacaaaattggagattaaa

SEQ ID NO: 423 - protein, 264 aa, Synthetic construct
MLRLLLALNLFPSIQVTGGSSQVQLVQSGSELKKPGASVKVSCKTSGYTFTSYAMNWVRQAPGQGLEWMGWINTNTGNPTYAQGFRGRFVFSLDTSVSTAYLQISSLKAEDTAVYYCARDKYSSSWYWFDSWGQGTLVTVSSGGGGSGGGGSGGGGSDIQMTQSPSSLSASVGDRVTITCRPSQNISSYLNWYQQKPGKAPKLLIYAASSLRSGVPSRFSGSGSGTDFTLTVSSLQPEDFANYYCQQSYSTPYTFGRGTKLEIK

SEQ ID NO: 424 - DNA, 792 bp, Synthetic construct
  source 1..792: mol_type = other DNA
atgctcaggctgctcttggctctcaacttattcccttcaattcaagtaacaggagggtcttcgcaggtacagttggtccagagcggttctgagctgaaaaaaccaggtgcatcagtaaaagtatcctgtaagacatcaggttatacgttcacgagctacgccatgaactgggttcgccaagcgccgggacaaggacttgaatggatgggttggataaacactaacactgggaatccaacttacgcacaggggtttcgagggaggttcgtttttagcctggatacgagtgtgagcacggcataccttcagatcagtagccttaaagcggaagatactgcggtgtattactgtgcaagggataagtactcaagctcctggtattggtttgattcatggggtcagggaacactcgtaactgttagctcaggaggcggaggatctggcggagggggctctggaggaggaggatctgatatacaaatgacccagtccccgagcagcctgagcgcgtcagtaggggatagagtcacaatcacctgtagaccaagtcaaaacatttcatcatacctgaactggtatcagcaaaaaccaggcaaagccccgaaattgcttatatatgctgcctcttcactccgctcaggcgttccttccagattctctggctccgggagcgggaccgactttacgctcacggtgtctagccttcaacccgaagacttcgcgaactactattgtcaacaatcttactcaacaccatatacgtttgggcgagggacaaaactcgaaattaag

SEQ ID NO: 425 - protein, 269 aa, Synthetic construct
MLRLLLALNLFPSIQVTGGSSQVQLVQSGTEVKYPGASVKVSCKASGYTFIDYGISWVRQAPGQGLEWMGWISAYNGNTDYAQKLQGRVTMTTDTSTSTAYMDLRSLRSDDTAVYYCAREGEGYCTYGVCPSFDYWGQGTLVTVSSGGGGSGGGGSGGGGSEIVLTQSPGTLSLSPGERATLSCRASQSVSSSYLAWYQQKPGQAPRLLIYGASTRGTGIPDRFSGSGSGTDFTLTISRLEPEDFALYYCQQYGSSPYTFGQGTKLEIK

SEQ ID NO: 426 - DNA, 807 bp, Synthetic construct
  source 1..807: mol_type = other DNA
atgctcaggctgctcttggctctcaacttattcccttcaattcaagtaacaggagggtcttcgcaggtacaattggtccagtccgggacagaagtcaagtatccgggggcttctgttaaggtatcctgtaaagcgtccggttacacattcatcgactacggcataagctgggtaagacaagcaccgggccaagggcttgaatggatgggatggatctccgcatacaatggtaacacggactatgcccaaaagctgcaaggaagagtaactatgactacagatacaagcaccagcacggcctatatggatcttagaagtcttcggagtgacgacacggcggtttactactgcgcaagggagggggagggatattgcacatatggagtgtgtccatcattcgattattggggacaaggaaccttggtaacagtgtcttcaggaggcggaggatctggcggagggggctctggaggaggaggatctgaaattgttctgacgcagtctccgggtaccctctcactgagtcctggtgaaagggcaacccttagttgccgcgcatcacagtcagtttcatccagttatctcgcctggtaccagcaaaagcctggtcaagccccgcggttgcttatctacggggcatccacccgaggtaccggcattcctgaccgattctccggttcagggtcagggactgatttcactctcactataagccgacttgagccggaggatttcgccttgtactattgccagcagtacgggtccagcccatatactttcggccaaggtacaaagttggagataaag

SEQ ID NO: 427 - protein, 268 aa, Synthetic construct
MLRLLLALNLFPSIQVTGGSSQVQLQESGPGLVKPSETLSLTCTVSGASISGYYWNWIRQPPGKGLEWIGYIHYSGTTNYNPSLKSRITISLDTSENQFSLRLSSVTAADTAVYYCARDWYTGYDWEGFDYWGQGTLVTVSSGGGGSGGGGSGGGGSQSVLTQPPSVSGAPGQRVSFSCTGTSSNIGTGFDVHWYRQLPGTAPKVLIYNNNNRPSGVPDRFSASKSGTSASLAITGLQAEDEADYYCQSYDSSLSGSVFGGGTRLTVL

SEQ ID NO: 428 - DNA, 804 bp, Synthetic construct
  source 1..804: mol_type = other DNA
atgctcaggctgctcttggctctcaacttattcccttcaattcaagtaacaggagggtcttcgcaggtgcaacttcaggagtcaggtccaggcttggttaaaccaagtgagacactgtccctgacttgcacagtatccggggcatctatctctggttattattggaactggattaggcaaccacctgggaagggtcttgaatggattggttacatacattactcagggacgacgaattataatccatccctcaagtccagaataactatcagtctcgacacttcagaaaatcagtttagcctgagactgtcaagtgtcaccgctgcggatactgccgtatattactgcgctagagactggtataccggctacgactgggaaggattcgattattggggccaaggaaccttggtaacagtgtcttcaggggggggtggatcaggtggtggagggtccgggggaggaggatctcagagcgtacttacacagcccccttcagtcagtggagcgccaggtcaaagggtctctttttcatgtactggaacgtcaagcaacatcgggacaggatttgatgtccactggtataggcagctcccaggaactgcccccaaagtcttgatctataacaataacaatcgcccttctggcgtacctgatcgattcagcgcatcaaaatccgggacctcagccagcctcgccattacgggcttgcaggcagaagacgaggccgattactactgtcaaagttatgatagctcattgagcgggtcagtctttggcggcggtacgagattgaccgttctg

SEQ ID NO: 429 - protein, 265 aa, Synthetic construct
MLRLLLALNLFPSIQVTGGSSEVQLVESGGDLVKSGGSLRLSCAASGFTFSDAWMSWVRQAPGKGLEWVGRIQSKTDGGTTDYVAPVKGRFTISRDDSKDTLYLHMNSLTTEDTAVYYCTTDLYYYGSGSYFLGQGTLVTVSSGGGGSGGGGSGGGGSDIQMTQSPSSLSASVGDRVTITCRASQNIITYLNWYQQKPGKAPKLLIYAASSLQSGVPSRFSGSGSGTDFTLTISSLQPEDFAIYHCQQSYNVPTTFGQGTKLEIK

SEQ ID NO: 430 - DNA, 795 bp, Synthetic construct
  source 1..795: mol_type = other DNA
atgctcaggctgctcttggctctcaacttattcccttcaattcaagtaacaggagggtcttcggaagtccaactcgtcgagagcgggggtgaccttgtcaagtccggcggaagcctcagactgtcttgtgcagccagcgggtttacgttctctgatgcctggatgtcatgggtaagacaggcccctggaaaagggcttgagtgggtaggccgcatacagagcaaaactgacggggggacaacggattacgtcgctccagttaaagggcggttcactataagccgagacgacagtaaggacacgctttatcttcacatgaatagccttaccactgaggacacggctgtctattattgcacgacagacctgtactattacggtagcggatcatatttcctcggacaaggaaccttggtaacagtgtcttcaggggggggtggatcaggtggtggagggtccgggggaggaggatctgacattcaaatgactcaaagtccaagtagccttagtgcgagcgtcggagatcgagtcactataacctgtcgggccagccaaaatatcatcacctacctcaattggtatcagcagaaaccaggtaaggcaccaaaactgcttatatatgctgcttcatctcttcaatccggtgtgccctctcgattctctgggagtgggtctggcacagatttcactcttactatatcatctctgcaacccgaggactttgcgatatatcattgccagcagtcatacaacgtcccaactacgtttgggcaggggactaagctggagataaaa

SEQ ID NO: 431 - protein, 466 aa, Synthetic construct
METDTLLLWVLLLWVPGSTGQVQLVESGGGVVQPGRSLRLSCAASGFTFSIYGMHWVRQAPGKGLEWVAVISYDGNNKDYTDSVKGRFTISRDNSKNTLYLQMNSLTAEDTAVYYCAKRGYGSSSLEFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 432 - DNA, 1401 bp, Synthetic construct
  source 1..1401: mol_type = other DNA
atggagacagatactttgctcctttgggtactcttgctgtgggttcccggtagcaccggccaagtccagcttgtagagtccggcggcggcgtagtccaaccgggtcggtcacttcggttgtcctgtgcggcgagtggtttcaccttcagcatttatggtatgcactgggtaagacaagcccctggcaaaggattggaatgggtggcagtaatttcctacgatggtaacaacaaggactacacggacagtgtgaaaggtcggttcactatatcacgggacaatagcaagaacactctctacttgcagatgaactcactgacggccgaggatactgcagtatattactgcgccaagcgcgggtacggttctagttcccttgagttttggggtcagggcactctggtaacggtgtctagcgctagcaccaaaggaccctccgtctttccgctcgcgccgtgctctcgatccacgagcgagtctacggcggccttgggctgtctcgtgaaagactatttcccagagccagtgaccgtttcctggaactccggagcgctcacatcaggtgtgcatacctttccagccgtacttcaatcttcagggctgtatagcttgtcctctgtcgtgaccgtaccctccagttctctcgggacaaagacctatacctgtaacgtagaccataagccatccaataccaaagtagataagagggtggaatctaaatatggaccgccttgtccgccgtgcccggctcctgagttccttggagggccgtccgtctttctgtttccccctaaacctaaggacaccctgatgataagtaggacgccggaagtaacctgtgtcgtggttgatgtgtcacaggaagacccggaggtacaatttaattggtacgtcgatggagtcgaggtgcataatgcgaaaaccaagccacgagaggagcaattcaacagcacctaccgagtagtttcagtcttgactgtgctgcaccaagactggctgaatggcaaggagtacaaatgcaaggtcagtaataaaggattgccgtcttcaatagagaagactatcagtaaggcgaagggacaacctcgcgagcctcaggtatacactttgccgcccagccaagaagaaatgacgaagaaccaagtctctttgacttgcctggtcaaaggcttctacccctctgatatagcagtcgaatgggagtccaacggtcaaccggagaacaattataagacaactccacctgtgcttgattcagacggctcttttttcttgtatagccgccttaccgtagacaagagtcgatggcaggaaggtaatgtattttcttgtagtgtaatgcacgaggcacttcataatcactacacacaaaagtctctgtcactttccctgggcaaatga

SEQ ID NO: 433 - protein, 446 aa, Synthetic construct
QVQLVESGGGVVQPGRSLRLSCAASGFTFSIYGMHWVRQAPGKGLEWVAVISYDGNNKDYTDSVKGRFTISRDNSKNTLYLQMNSLTAEDTAVYYCAKRGYGSSSLEFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 434 - protein, 231 aa, Synthetic construct
MRAWIFFLLCLAGRALADIQMTQSPSSLSASVGDRVTITCRASQDIRNDLGWYQQKPGKAPKRLIYATSSLQSGVPSRFSGSRSGAEFTLTVSSLQPEDFATYYCLQHNTYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 435 - DNA, 696 bp, Synthetic construct
  source 1..696: mol_type = other DNA
atgagagcctggatcttcttcctgctttgtttggcaggacgagcgctagccgacatacaaatgacgcaatcaccatccagtctctctgcgtctgtcggcgaccgcgtgacgataacttgtagggccagccaggacattcggaatgatcttggttggtatcagcaaaagcccggaaaagctccaaaacgattgatctacgcgaccagctcactgcaatctggcgttccatcaagatttagcggtagccggtcaggcgctgaatttacactcacggtctcctctcttcaacctgaagactttgcaacatattattgtcttcaacataatacatatccctatactttcgggcaaggcactaagctcgaaattaagcgtacggtagctgcgcctagcgtgtttatttttccgccctctgacgaacaactcaaaagtggcactgcctctgtagtctgtctcctgaacaatttttatccaagggaagctaaggtacagtggaaagtcgataacgcgctccaaagtggcaacagtcaagaatctgtaacggagcaagatagtaaagacagcacttacagcttgagcagcacactcacattgtccaaggccgactacgaaaaacataaagtgtacgcctgtgaggtaacacaccagggtctgagcagcccagtgacgaagtcatttaatcgcggtgagtgctga

SEQ ID NO: 436 - protein, 214 aa, Synthetic construct
DIQMTQSPSSLSASVGDRVTITCRASQDIRNDLGWYQQKPGKAPKRLIYATSSLQSGVPSRFSGSRSGAEFTLTVSSLQPEDFATYYCLQHNTYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 437 - protein, 468 aa, Synthetic construct
METDTLLLWVLLLWVPGSTGEVQLVESGGGLVQPGRSLRLSCAASGFMFDDYVMHWVRQAPGKGLEWVSGINWNSDNIGYADSVKGRFTISRDNAKNSLFLQMNSLRVEDTALYYCAKDIDAAAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 438 - DNA, 1407 bp, Synthetic construct
  source 1..1407: mol_type = other DNA
atggagacagatactttgctcctttgggtactcttgctgtgggttcccggtagcaccggcgaagtccaactcgtcgagtcaggaggggggttggtacagccaggcagatccctgcggctgagttgtgcagcttccgggtttatgtttgatgactatgttatgcactgggtccgccaagcgccaggtaagggacttgaatgggtttccggtataaactggaactccgacaatatcggctatgccgactccgtgaaaggtcggttcacaataagtcgggacaatgcaaagaactctttgtttctccagatgaacagtcttcgagtggaggacactgcgttgtattattgcgctaaggatatagacgcggctgctttcgactattggggtcagggcactctggtaacggtgtctagcgctagcaccaaaggaccctccgtctttccgctcgcgccgagcagcaagtccacgagcggggggacagccgcccttgggtgtctggtcaaggattattttcctgagcccgttactgtctcctggaatagcggtgccttgacttctggggtacataccttcccggcggttttgcaatcatcaggactgtatagcctctctagcgttgtaacggtaccatcttcatcacttggcacacaaacatatatttgtaatgtaaatcacaaaccttcaaatactaaagttgacaaaaaagtggagccgaagagctgtgataaaacgcatacttgcccgccttgccccgctccagaattgctgggcgggccgtctgtgttcctcttcccaccgaaaccgaaggatacgctgatgataagtcgaacgcccgaagtaacatgcgtggttgttgatgtaagccacgaagatccggaagtaaagttcaactggtacgtagatggagtagaagtgcataacgcgaaaactaagccccgcgaggagcaatataattcaacctatcgggttgttagtgtgctcaccgttctccatcaggattggcttaacgggaaagagtataagtgcaaggttagcaataaagcattgccagcacctatagagaagacaattagcaaagcgaagggacagccacgagagccccaagtctacacgctgcctcccagccgcgacgagttgacgaagaatcaagtgtcactcacatgtcttgtcaagggattctatccctcagatattgcagtagaatgggagtctaatggacaaccagagaacaactataaaactactccaccggtgcttgatagtgacggtagttttttcctttatagtaagctgactgtcgataagagtcgctggcaacagggaaatgttttttcttgtagtgttatgcatgaagcacttcataaccattacacacaaaagtctctgtcactttcccctggcaaatga

SEQ ID NO: 439 - protein, 448 aa, Synthetic construct
EVQLVESGGGLVQPGRSLRLSCAASGFMFDDYVMHWVRQAPGKGLEWVSGINWNSDNIGYADSVKGRFTISRDNAKNSLFLQMNSLRVEDTALYYCAKDIDAAAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 440 - protein, 465 aa, Synthetic construct
METDTLLLWVLLLWVPGSTGEVQLVESGGGLVQPGRSLRLSCAASGFMFDDYVMHWVRQAPGKGLEWVSGINWNSDNIGYADSVKGRFTISRDNAKNSLFLQMNSLRVEDTALYYCAKDIDAAAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 441 - DNA, 1398 bp, Synthetic construct
  source 1..1398: mol_type = other DNA
atggagacagatactttgctcctttgggtactcttgctgtgggttcccggtagcaccggcgaagtccaactcgtcgagtcaggaggggggttggtacagccaggcagatccctgcggctgagttgtgcagcttccgggtttatgtttgatgactatgttatgcactgggtccgccaagcgccaggtaagggacttgaatgggtttccggtataaactggaactccgacaatatcggctatgccgactccgtgaaaggtcggttcacaataagtcgggacaatgcaaagaactctttgtttctccagatgaacagtcttcgagtggaggacactgcgttgtattattgcgctaaggatatagacgcggctgctttcgactattggggtcagggcactctggtaacggtgtctagcgctagcaccaaaggaccctccgtctttccgctcgcgccgtgctctcgatccacgagcgagtctacggcggccttgggctgtctcgtgaaagactatttcccagagccagtgaccgtttcctggaactccggagcgctcacatcaggtgtgcatacctttccagccgtacttcaatcttcagggctgtatagcttgtcctctgtcgtgaccgtaccctccagttctctcgggacaaagacctatacctgtaacgtagaccataagccatccaataccaaagtagataagagggtggaatctaaatatggaccgccttgtccgccgtgcccggctcctgagttccttggagggccgtccgtctttctgtttccccctaaacctaaggacaccctgatgataagtaggacgccggaagtaacctgtgtcgtggttgatgtgtcacaggaagacccggaggtacaatttaattggtacgtcgatggagtcgaggtgcataatgcgaaaaccaagccacgagaggagcaattcaacagcacctaccgagtagtttcagtcttgactgtgctgcaccaagactggctgaatggcaaggagtacaaatgcaaggtcagtaataaaggattgccgtcttcaatagagaagactatcagtaaggcgaagggacaacctcgcgagcctcaggtatacactttgccgcccagccaagaagaaatgacgaagaaccaagtctctttgacttgcctggtcaaaggcttctacccctctgatatagcagtcgaatgggagtccaacggtcaaccggagaacaattataagacaactccacctgtgcttgattcagacggctcttttttcttgtatagccgccttaccgtagacaagagtcgatggcaggaaggtaatgtattttcttgtagtgtaatgcacgaggcacttcataatcactacacacaaaagtctctgtcactttccctgggcaaatga

SEQ ID NO: 442 - protein, 445 aa, Synthetic construct
EVQLVESGGGLVQPGRSLRLSCAASGFMFDDYVMHWVRQAPGKGLEWVSGINWNSDNIGYADSVKGRFTISRDNAKNSLFLQMNSLRVEDTALYYCAKDIDAAAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 443 - protein, 236 aa, Synthetic construct
MRAWIFFLLCLAGRALADIVMTQSPLSLPVTPGEPASISCRSSQSLLHSNGYSYLAWYLQKPGQSPQLLIYLVSNRASGVPDRFTGSGSGTDFTLKISRVEAEDVGVYYCMQALQTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 444 - DNA, 711 bp, Synthetic construct
  source 1..711: mol_type = other DNA
atgagagcctggatcttcttcctgctttgtttggcaggacgagcgctagccgacatcgttatgactcaatcaccactctctttgcccgtcacccctggggaaccagcgagcataagttgtaggagttcccagtccctccttcactctaacggatatagttacctggcttggtatttgcagaaaccagggcagtctcctcagttgctcatatatctggtctccaatcgcgcttctggagtgcctgacagatttaccggatctggaagtggaaccgacttcacactgaaaatttcccgagtagaggctgaggatgttggcgtctattattgcatgcaggctctccagactccttggacgttcggccagggtactaaagtggagataaaacgtacggtagctgcgcctagcgtgtttatttttccgccctctgacgaacaactcaaaagtggcactgcctctgtagtctgtctcctgaacaatttttatccaagggaagctaaggtacagtggaaagtcgataacgcgctccaaagtggcaacagtcaagaatctgtaacggagcaagatagtaaagacagcacttacagcttgagcagcacactcacattgtccaaggccgactacgaaaaacataaagtgtacgcctgtgaggtaacacaccagggtctgagcagcccagtgacgaagtcatttaatcgcggtgagtgctga

SEQ ID NO: 445 - protein, 219 aa, Synthetic construct
DIVMTQSPLSLPVTPGEPASISCRSSQSLLHSNGYSYLAWYLQKPGQSPQLLIYLVSNRASGVPDRFTGSGSGTDFTLKISRVEAEDVGVYYCMQALQTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 446 - protein, 463 aa, Synthetic construct
METDTLLLWVLLLWVPGSTGEVQLVESGGGLVKPGESLRLSCAASGFTFSNAWMSWVRQAPGKGLEWVGRDGGARDYAAPVKGRFTISRDDSKNTLYLQMNTLKTEDTAVYYCTKGAFFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 447 - DNA, 1392 bp, Synthetic construct
  source 1..1392: mol_type = other DNA
atggagacagatactttgctcctttgggtactcttgctgtgggttcccggtagcaccggcgaggtgcagcttgtcgaaagcggaggaggccttgttaagccgggagagtcacttcgactctcctgcgcggcttctgggtttacgtttagtaatgcgtggatgagctgggtgagacaagctcccggtaaaggcttggagtgggtgggtcgagacggcggcgcacgagattacgcagctccggtcaaggggcgcttcaccatttctcgcgacgattctaaaaataccttgtacttgcaaatgaatactcttaaaactgaagatacagcggtctattattgtaccaagggtgccttcttttttgactactgggggcagggcactctggtaacggtgtctagcgctagcaccaaaggaccctccgtctttccgctcgcgccgagcagcaagtccacgagcggggggacagccgcccttgggtgtctggtcaaggattattttcctgagcccgttactgtctcctggaatagcggtgccttgacttctggggtacataccttcccggcggttttgcaatcatcaggactgtatagcctctctagcgttgtaacggtaccatcttcatcacttggcacacaaacatatatttgtaatgtaaatcacaaaccttcaaatactaaagttgacaaaaaagtggagccgaagagctgtgataaaacgcatacttgcccgccttgccccgctccagaattgctgggcgggccgtctgtgttcctcttcccaccgaaaccgaaggatacgctgatgataagtcgaacgcccgaagtaacatgcgtggttgttgatgtaagccacgaagatccggaagtaaagttcaactggtacgtagatggagtagaagtgcataacgcgaaaactaagccccgcgaggagcaatataattcaacctatcgggttgttagtgtgctcaccgttctccatcaggattggcttaacgggaaagagtataagtgcaaggttagcaataaagcattgccagcacctatagagaagacaattagcaaagcgaagggacagccacgagagccccaagtctacacgctgcctcccagccgcgacgagttgacgaagaatcaagtgtcactcacatgtcttgtcaagggattctatccctcagatattgcagtagaatgggagtctaatggacaaccagagaacaactataaaactactccaccggtgcttgatagtgacggtagttttttcctttatagtaagctgactgtcgataagagtcgctggcaacagggaaatgttttttcttgtagtgttatgcatgaagcacttcataaccattacacacaaaagtctctgtcactttcccctggcaaatga

SEQ ID NO: 448 - protein, 443 aa, Synthetic construct
EVQLVESGGGLVKPGESLRLSCAASGFTFSNAWMSWVRQAPGKGLEWVGRDGGARDYAAPVKGRFTISRDDSKNTLYLQMNTLKTEDTAVYYCTKGAFFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 449 - protein, 460 aa, Synthetic construct
METDTLLLWVLLLWVPGSTGEVQLVESGGGLVKPGESLRLSCAASGFTFSNAWMSWVRQAPGKGLEWVGRDGGARDYAAPVKGRFTISRDDSKNTLYLQMNTLKTEDTAVYYCTKGAFFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 450 - DNA, 1383 bp, Synthetic construct
  source 1..1383: mol_type = other DNA
atggagacagatactttgctcctttgggtactcttgctgtgggttcccggtagcaccggcgaggtgcagcttgtcgaaagcggaggaggccttgttaagccgggagagtcacttcgactctcctgcgcggcttctgggtttacgtttagtaatgcgtggatgagctgggtgagacaagctcccggtaaaggcttggagtgggtgggtcgagacggcggcgcacgagattacgcagctccggtcaaggggcgcttcaccatttctcgcgacgattctaaaaataccttgtacttgcaaatgaatactcttaaaactgaagatacagcggtctattattgtaccaagggtgccttcttttttgactactgggggcagggcactctggtaacggtgtctagcgctagcaccaaaggaccctccgtctttccgctcgcgccgtgctctcgatccacgagcgagtctacggcggccttgggctgtctcgtgaaagactatttcccagagccagtgaccgtttcctggaactccggagcgctcacatcaggtgtgcatacctttccagccgtacttcaatcttcagggctgtatagcttgtcctctgtcgtgaccgtaccctccagttctctcgggacaaagacctatacctgtaacgtagaccataagccatccaataccaaagtagataagagggtggaatctaaatatggaccgccttgtccgccgtgcccggctcctgagttccttggagggccgtccgtctttctgtttccccctaaacctaaggacaccctgatgataagtaggacgccggaagtaacctgtgtcgtggttgatgtgtcacaggaagacccggaggtacaatttaattggtacgtcgatggagtcgaggtgcataatgcgaaaaccaagccacgagaggagcaattcaacagcacctaccgagtagtttcagtcttgactgtgctgcaccaagactggctgaatggcaaggagtacaaatgcaaggtcagtaataaaggattgccgtcttcaatagagaagactatcagtaaggcgaagggacaacctcgcgagcctcaggtatacactttgccgcccagccaagaagaaatgacgaagaaccaagtctctttgacttgcctggtcaaaggcttctacccctctgatatagcagtcgaatgggagtccaacggtcaaccggagaacaattataagacaactccacctgtgcttgattcagacggctcttttttcttgtatagccgccttaccgtagacaagagtcgatggcaggaaggtaatgtattttcttgtagtgtaatgcacgaggcacttcataatcactacacacaaaagtctctgtcactttccctgggcaaatga

SEQ ID NO: 451 - protein, 440 aa, Synthetic construct
EVQLVESGGGLVKPGESLRLSCAASGFTFSNAWMSWVRQAPGKGLEWVGRDGGARDYAAPVKGRFTISRDDSKNTLYLQMNTLKTEDTAVYYCTKGAFFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 452 - protein, 232 aa, Synthetic construct
MRAWIFFLLCLAGRALAEIVLTQSPGTLSLSPGERATLSCRASQSVISGYLAWYQQKPGQAPRLLIYGASRRATGITDRFSGSGSGTDFTLIISRLEPEDSAVYYCQQYDSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 453 - DNA, 699 bp, Synthetic construct
  source 1..699: mol_type = other DNA
atgagagcctggatcttcttcctgctttgtttggcaggacgagcgctagccgaaatcgtacttacgcaaagtccgggcacgctgagtcttagtccaggagaaagagctactctctcttgtcgcgcgagtcagagtgtaattagcggatacctcgcgtggtaccagcaaaaacctgggcaggctcctagattgttgatatatggtgcaagtcggcgagcaactggaatcacggatagattttcaggttcagggtccggtacagactttacattgattatatctaggctcgaacccgaggacagtgcggtttattattgtcagcaatatgactcatcaccccggacatttggccagggtactaaagtggagataaaacgtacggtagctgcgcctagcgtgtttatttttccgccctctgacgaacaactcaaaagtggcactgcctctgtagtctgtctcctgaacaatttttatccaagggaagctaaggtacagtggaaagtcgataacgcgctccaaagtggcaacagtcaagaatctgtaacggagcaagatagtaaagacagcacttacagcttgagcagcacactcacattgtccaaggccgactacgaaaaacataaagtgtacgcctgtgaggtaacacaccagggtctgagcagcccagtgacgaagtcatttaatcgcggtgagtgctga

SEQ ID NO: 454 - protein, 215 aa, Synthetic construct
EIVLTQSPGTLSLSPGERATLSCRASQSVISGYLAWYQQKPGQAPRLLIYGASRRATGITDRFSGSGSGTDFTLIISRLEPEDSAVYYCQQYDSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 455 - protein, 471 aa, Synthetic construct
METDTLLLWVLLLWVPGSTGQVQLVQSGSELKKPGASVKVSCKTSGYTFTSYAMNWVRQAPGQGLEWMGWINTNTGNPTYAQGFRGRFVFSLDTSVSTAYLQISSLKAEDTAVYYCARDKYSSSWYWFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 456 - DNA, 1416 bp, Synthetic construct
  source 1..1416: mol_type = other DNA
atggagacagatactttgctcctttgggtactcttgctgtgggttcccggtagcaccggccaggtccaacttgttcaatccgggtccgaattgaagaaacctggtgcttcagttaaagtgtcttgtaagacaagtggttatacgttcacctcttatgcaatgaattgggtccgccaggcgcctggccaaggtctggagtggatgggctggataaataccaacacgggtaatccaacgtatgcccagggcttccgaggtcgctttgttttcagtctggacacttcagtcagcactgcctatctccagatatcttctcttaaagctgaggatactgcggtttattattgtgcgagggataagtacagtagttcatggtattggttcgactcttggggccagggcactctggtaacggtgtctagcgctagcaccaaaggaccctccgtctttccgctcgcgccgagcagcaagtccacgagcggggggacagccgcccttgggtgtctggtcaaggattattttcctgagcccgttactgtctcctggaatagcggtgccttgacttctggggtacataccttcccggcggttttgcaatcatcaggactgtatagcctctctagcgttgtaacggtaccatcttcatcacttggcacacaaacatatatttgtaatgtaaatcacaaaccttcaaatactaaagttgacaaaaaagtggagccgaagagctgtgataaaacgcatacttgcccgccttgccccgctccagaattgctgggcgggccgtctgtgttcctcttcccaccgaaaccgaaggatacgctgatgataagtcgaacgcccgaagtaacatgcgtggttgttgatgtaagccacgaagatccggaagtaaagttcaactggtacgtagatggagtagaagtgcataacgcgaaaactaagccccgcgaggagcaatataattcaacctatcgggttgttagtgtgctcaccgttctccatcaggattggcttaacgggaaagagtataagtgcaaggttagcaataaagcattgccagcacctatagagaagacaattagcaaagcgaagggacagccacgagagccccaagtctacacgctgcctcccagccgcgacgagttgacgaagaatcaagtgtcactcacatgtcttgtcaagggattctatccctcagatattgcagtagaatgggagtctaatggacaaccagagaacaactataaaactactccaccggtgcttgatagtgacggtagttttttcctttatagtaagctgactgtcgataagagtcgctggcaacagggaaatgttttttcttgtagtgttatgcatgaagcacttcataaccattacacacaaaagtctctgtcactttcccctggcaaatga

SEQ ID NO: 457 - protein, 451 aa, Synthetic construct
QVQLVQSGSELKKPGASVKVSCKTSGYTFTSYAMNWVRQAPGQGLEWMGWINTNTGNPTYAQGFRGRFVFSLDTSVSTAYLQISSLKAEDTAVYYCARDKYSSSWYWFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 458 - protein, 468 aa, Synthetic construct
METDTLLLWVLLLWVPGSTGQVQLVQSGSELKKPGASVKVSCKTSGYTFTSYAMNWVRQAPGQGLEWMGWINTNTGNPTYAQGFRGRFVFSLDTSVSTAYLQISSLKAEDTAVYYCARDKYSSSWYWFDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 459 - DNA, 1407 bp, Synthetic construct
  source 1..1407: mol_type = other DNA
atggagacagatactttgctcctttgggtactcttgctgtgggttcccggtagcaccggccaggtccaacttgttcaatccgggtccgaattgaagaaacctggtgcttcagttaaagtgtcttgtaagacaagtggttatacgttcacctcttatgcaatgaattgggtccgccaggcgcctggccaaggtctggagtggatgggctggataaataccaacacgggtaatccaacgtatgcccagggcttccgaggtcgctttgttttcagtctggacacttcagtcagcactgcctatctccagatatcttctcttaaagctgaggatactgcggtttattattgtgcgagggataagtacagtagttcatggtattggttcgactcttggggccagggcactctggtaacggtgtctagcgctagcaccaaaggaccctccgtctttccgctcgcgccgtgctctcgatccacgagcgagtctacggcggccttgggctgtctcgtgaaagactatttcccagagccagtgaccgtttcctggaactccggagcgctcacatcaggtgtgcatacctttccagccgtacttcaatcttcagggctgtatagcttgtcctctgtcgtgaccgtaccctccagttctctcgggacaaagacctatacctgtaacgtagaccataagccatccaataccaaagtagataagagggtggaatctaaatatggaccgccttgtccgccgtgcccggctcctgagttccttggagggccgtccgtctttctgtttccccctaaacctaaggacaccctgatgataagtaggacgccggaagtaacctgtgtcgtggttgatgtgtcacaggaagacccggaggtacaatttaattggtacgtcgatggagtcgaggtgcataatgcgaaaaccaagccacgagaggagcaattcaacagcacctaccgagtagtttcagtcttgactgtgctgcaccaagactggctgaatggcaaggagtacaaatgcaaggtcagtaataaaggattgccgtcttcaatagagaagactatcagtaaggcgaagggacaacctcgcgagcctcaggtatacactttgccgcccagccaagaagaaatgacgaagaaccaagtctctttgacttgcctggtcaaaggcttctacccctctgatatagcagtcgaatgggagtccaacggtcaaccggagaacaattataagacaactccacctgtgcttgattcagacggctcttttttcttgtatagccgccttaccgtagacaagagtcgatggcaggaaggtaatgtattttcttgtagtgtaatgcacgaggcacttcataatcactacacacaaaagtctctgtcactttccctgggcaaatga

SEQ ID NO: 460 - protein, 448 aa, Synthetic construct
QVQLVQSGSELKKPGASVKVSCKTSGYTFTSYAMNWVRQAPGQGLEWMGWINTNTGNPTYAQGFRGRFVFSLDTSVSTAYLQISSLKAEDTAVYYCARDKYSSSWYWFDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 461 - protein, 231 aa, Synthetic construct
MRAWIFFLLCLAGRALADIQMTQSPSSLSASVGDRVTITCRPSQNISSYLNWYQQKPGKAPKLLIYAASSLRSGVPSRFSGSGSGTDFTLTVSSLQPEDFANYYCQQSYSTPYTFGRGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 462 - DNA, 696 bp, Synthetic construct
  source 1..696: mol_type = other DNA
atgagagcctggatcttcttcctgctttgtttggcaggacgagcgctagccgacattcagatgactcagtccccatcctcattgagtgctagtgttggcgacagagttactattacatgtcggccatctcagaacattagcagttatcttaattggtaccaacagaagcctggtaaagcacccaagctgcttatttacgctgcaagttccctcaggtcaggggttcctagccgatttagcggcagtggtagcgggactgacttcacgctgaccgtttcaagtcttcaacctgaagactttgccaattattattgccagcaatcatatagtacgccttatactttcggaaggggcaccaaactggaaattaaacgtacggtagctgcgcctagcgtgtttatttttccgccctctgacgaacaactcaaaagtggcactgcctctgtagtctgtctcctgaacaatttttatccaagggaagctaaggtacagtggaaagtcgataacgcgctccaaagtggcaacagtcaagaatctgtaacggagcaagatagtaaagacagcacttacagcttgagcagcacactcacattgtccaaggccgactacgaaaaacataaagtgtacgcctgtgaggtaacacaccagggtctgagcagcccagtgacgaagtcatttaatcgcggtgagtgctga

SEQ ID NO: 463 - protein, 214 aa, Synthetic construct
DIQMTQSPSSLSASVGDRVTITCRPSQNISSYLNWYQQKPGKAPKLLIYAASSLRSGVPSRFSGSGSGTDFTLTVSSLQPEDFANYYCQQSYSTPYTFGRGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 464 - protein, 472 aa, Synthetic construct
METDTLLLWVLLLWVPGSTGEVQLVESGGDLVKSGGSLRLSCAASGFTFSDAWMSWVRQAPGKGLEWVGRIQSKTDGGTTDYFAPVKGRFTISRDDSKDTLYLHMNSLTTEDTAVYYCTTDLYYYGSGSYFLGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 465 - DNA, 1419 bp, Synthetic construct
  source 1..1419: mol_type = other DNA
atggagacagatactttgctcctttgggtactcttgctgtgggttcccggtagcaccggcgaggtacagttggtcgaaagcgggggcgatttggtaaaaagtggaggttcactgcggttgtcatgcgcggcatccggcttcacgtttagcgatgcctggatgtcttgggtacgccaagcacctggaaaaggactggagtgggtaggcagaatccagagcaaaactgatggcggaacaacagattacttcgcccccgttaaaggccggtttaccatctctcgagacgattccaaagatacgctttaccttcacatgaacagcttgaccactgaggacaccgccgtgtactactgtaccactgatctttactactacggcagtggatcttattttttgggtcaaggcactctggtaacggtgtctagcgctagcaccaaaggaccctccgtctttccgctcgcgccgagcagcaagtccacgagcggggggacagccgcccttgggtgtctggtcaaggattattttcctgagcccgttactgtctcctggaatagcggtgccttgacttctggggtacataccttcccggcggttttgcaatcatcaggactgtatagcctctctagcgttgtaacggtaccatcttcatcacttggcacacaaacatatatttgtaatgtaaatcacaaaccttcaaatactaaagttgacaaaaaagtggagccgaagagctgtgataaaacgcatacttgcccgccttgccccgctccagaattgctgggcgggccgtctgtgttcctcttcccaccgaaaccgaaggatacgctgatgataagtcgaacgcccgaagtaacatgcgtggttgttgatgtaagccacgaagatccggaagtaaagttcaactggtacgtagatggagtagaagtgcataacgcgaaaactaagccccgcgaggagcaatataattcaacctatcgggttgttagtgtgctcaccgttctccatcaggattggcttaacgggaaagagtataagtgcaaggttagcaataaagcattgccagcacctatagagaagacaattagcaaagcgaagggacagccacgagagccccaagtctacacgctgcctcccagccgcgacgagttgacgaagaatcaagtgtcactcacatgtcttgtcaagggattctatccctcagatattgcagtagaatgggagtctaatggacaaccagagaacaactataaaactactccaccggtgcttgatagtgacggtagttttttcctttatagtaagctgactgtcgataagagtcgctggcaacagggaaatgttttttcttgtagtgttatgcatgaagcacttcataaccattacacacaaaagtctctgtcactttcccctggcaaatga

SEQ ID NO: 466 - protein, 452 aa, Synthetic construct
EVQLVESGGDLVKSGGSLRLSCAASGFTFSDAWMSWVRQAPGKGLEWVGRIQSKTDGGTTDYFAPVKGRFTISRDDSKDTLYLHMNSLTTEDTAVYYCTTDLYYYGSGSYFLGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 467 - protein, 469 aa, Synthetic construct
METDTLLLWVLLLWVPGSTGEVQLVESGGDLVKSGGSLRLSCAASGFTFSDAWMSWVRQAPGKGLEWVGRIQSKTDGGTTDYFAPVKGRFTISRDDSKDTLYLHMNSLTTEDTAVYYCTTDLYYYGSGSYFLGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 468 - DNA, 1410 bp, Synthetic construct
  source 1..1410: mol_type = other DNA
atggagacagatactttgctcctttgggtactcttgctgtgggttcccggtagcaccggcgaggtacagttggtcgaaagcgggggcgatttggtaaaaagtggaggttcactgcggttgtcatgcgcggcatccggcttcacgtttagcgatgcctggatgtcttgggtacgccaagcacctggaaaaggactggagtgggtaggcagaatccagagcaaaactgatggcggaacaacagattacttcgcccccgttaaaggccggtttaccatctctcgagacgattccaaagatacgctttaccttcacatgaacagcttgaccactgaggacaccgccgtgtactactgtaccactgatctttactactacggcagtggatcttattttttgggtcaaggcactctggtaacggtgtctagcgctagcaccaaaggaccctccgtctttccgctcgcgccgtgctctcgatccacgagcgagtctacggcggccttgggctgtctcgtgaaagactatttcccagagccagtgaccgtttcctggaactccggagcgctcacatcaggtgtgcatacctttccagccgtacttcaatcttcagggctgtatagcttgtcctctgtcgtgaccgtaccctccagttctctcgggacaaagacctatacctgtaacgtagaccataagccatccaataccaaagtagataagagggtggaatctaaatatggaccgccttgtccgccgtgcccggctcctgagttccttggagggccgtccgtctttctgtttccccctaaacctaaggacaccctgatgataagtaggacgccggaagtaacctgtgtcgtggttgatgtgtcacaggaagacccggaggtacaatttaattggtacgtcgatggagtcgaggtgcataatgcgaaaaccaagccacgagaggagcaattcaacagcacctaccgagtagtttcagtcttgactgtgctgcaccaagactggctgaatggcaaggagtacaaatgcaaggtcagtaataaaggattgccgtcttcaatagagaagactatcagtaaggcgaagggacaacctcgcgagcctcaggtatacactttgccgcccagccaagaagaaatgacgaagaaccaagtctctttgacttgcctggtcaaaggcttctacccctctgatatagcagtcgaatgggagtccaacggtcaaccggagaacaattataagacaactccacctgtgcttgattcagacggctcttttttcttgtatagccgccttaccgtagacaagagtcgatggcaggaaggtaatgtattttcttgtagtgtaatgcacgaggcacttcataatcactacacacaaaagtctctgtcactttccctgggcaaatga

SEQ ID NO: 469 - protein, 449 aa, Synthetic construct
EVQLVESGGDLVKSGGSLRLSCAASGFTFSDAWMSWVRQAPGKGLEWVGRIQSKTDGGTTDYFAPVKGRFTISRDDSKDTLYLHMNSLTTEDTAVYYCTTDLYYYGSGSYFLGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 470 - protein, 477 aa, Synthetic construct
METDTLLLWVLLLWVPGSTGEVQLVESGGGVVRPGGSLRLSCAASGFTLDDYGMSWVRQAPGKGLEWVSGINWNGATTDYADSVKGRFSISRDNAKNSVYLQMNSLRAEDTALYYCAREGITMVRGVIIEDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 471 - DNA, 1434 bp, Synthetic construct
  source 1..1434: mol_type = other DNA
atggagacagatactttgctcctttgggtactcttgctgtgggttcccggtagcaccggcgaggtgcagcttgtcgaatcaggtggcggcgttgttcgccctggcgggagtcttcggctgtcatgcgctgcttccgggttcactcttgatgattatgggatgtcttgggtgagacaagcaccaggaaaaggattggaatgggtgtctggaattaactggaacggcgcgactacggactatgcggatagtgttaagggtagatttagtatttcccgagacaatgctaagaatagcgtatatctgcagatgaacagcttgagggccgaagataccgctttgtactattgtgcaagagaaggtattacgatggtaagaggggtgattatagaagactggtactttgacttgtggggacggggcactctggtaacggtgtctagcgctagcaccaaaggaccctccgtctttccgctcgcgccgagcagcaagtccacgagcggggggacagccgcccttgggtgtctggtcaaggattattttcctgagcccgttactgtctcctggaatagcggtgccttgacttctggggtacataccttcccggcggttttgcaatcatcaggactgtatagcctctctagcgttgtaacggtaccatcttcatcacttggcacacaaacatatatttgtaatgtaaatcacaaaccttcaaatactaaagttgacaaaaaagtggagccgaagagctgtgataaaacgcatacttgcccgccttgccccgctccagaattgctgggcgggccgtctgtgttcctcttcccaccgaaaccgaaggatacgctgatgataagtcgaacgcccgaagtaacatgcgtggttgttgatgtaagccacgaagatccggaagtaaagttcaactggtacgtagatggagtagaagtgcataacgcgaaaactaagccccgcgaggagcaatataattcaacctatcgggttgttagtgtgctcaccgttctccatcaggattggcttaacgggaaagagtataagtgcaaggttagcaataaagcattgccagcacctatagagaagacaattagcaaagcgaagggacagccacgagagccccaagtctacacgctgcctcccagccgcgacgagttgacgaagaatcaagtgtcactcacatgtcttgtcaagggattctatccctcagatattgcagtagaatgggagtctaatggacaaccagagaacaactataaaactactccaccggtgcttgatagtgacggtagttttttcctttatagtaagctgactgtcgataagagtcgctggcaacagggaaatgttttttcttgtagtgttatgcatgaagcacttcataaccattacacacaaaagtctctgtcactttcccctggcaaatga

SEQ ID NO: 472 - protein, 457 aa, Synthetic construct
EVQLVESGGGVVRPGGSLRLSCAASGFTLDDYGMSWVRQAPGKGLEWVSGINWNGATTDYADSVKGRFSISRDNAKNSVYLQMNSLRAEDTALYYCAREGITMVRGVIIEDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 473 - protein, 474 aa, Synthetic construct
METDTLLLWVLLLWVPGSTGEVQLVESGGGVVRPGGSLRLSCAASGFTLDDYGMSWVRQAPGKGLEWVSGINWNGATTDYADSVKGRFSISRDNAKNSVYLQMNSLRAEDTALYYCAREGITMVRGVIIEDWYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 474 - DNA, 1425 bp, Synthetic construct
  source 1..1425: mol_type = other DNA
atggagacagatactttgctcctttgggtactcttgctgtgggttcccggtagcaccggcgaggtgcagcttgtcgaatcaggtggcggcgttgttcgccctggcgggagtcttcggctgtcatgcgctgcttccgggttcactcttgatgattatgggatgtcttgggtgagacaagcaccaggaaaaggattggaatgggtgtctggaattaactggaacggcgcgactacggactatgcggatagtgttaagggtagatttagtatttcccgagacaatgctaagaatagcgtatatctgcagatgaacagcttgagggccgaagataccgctttgtactattgtgcaagagaaggtattacgatggtaagaggggtgattatagaagactggtactttgacttgtggggacggggcactctggtaacggtgtctagcgctagcaccaaaggaccctccgtctttccgctcgcgccgtgctctcgatccacgagcgagtctacggcggccttgggctgtctcgtgaaagactatttcccagagccagtgaccgtttcctggaactccggagcgctcacatcaggtgtgcatacctttccagccgtacttcaatcttcagggctgtatagcttgtcctctgtcgtgaccgtaccctccagttctctcgggacaaagacctatacctgtaacgtagaccataagccatccaataccaaagtagataagagggtggaatctaaatatggaccgccttgtccgccgtgcccggctcctgagttccttggagggccgtccgtctttctgtttccccctaaacctaaggacaccctgatgataagtaggacgccggaagtaacctgtgtcgtggttgatgtgtcacaggaagacccggaggtacaatttaattggtacgtcgatggagtcgaggtgcataatgcgaaaaccaagccacgagaggagcaattcaacagcacctaccgagtagtttcagtcttgactgtgctgcaccaagactggctgaatggcaaggagtacaaatgcaaggtcagtaataaaggattgccgtcttcaatagagaagactatcagtaaggcgaagggacaacctcgcgagcctcaggtatacactttgccgcccagccaagaagaaatgacgaagaaccaagtctctttgacttgcctggtcaaaggcttctacccctctgatatagcagtcgaatgggagtccaacggtcaaccggagaacaattataagacaactccacctgtgcttgattcagacggctcttttttcttgtatagccgccttaccgtagacaagagtcgatggcaggaaggtaatgtattttcttgtagtgtaatgcacgaggcacttcataatcactacacacaaaagtctctgtcactttccctgggcaaatga

SEQ ID NO: 475 - protein, 454 aa, Synthetic construct
EVQLVESGGGVVRPGGSLRLSCAASGFTLDDYGMSWVRQAPGKGLEWVSGINWNGATTDYADSVKGRFSISRDNAKNSVYLQMNSLRAEDTALYYCAREGITMVRGVIIEDWYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 476 - protein, 236 aa, Synthetic construct
MRAWIFFLLCLAGRALADIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 477 - DNA, 711 bp, Synthetic construct
  source 1..711: mol_type = other DNA
atgagagcctggatcttcttcctgctttgtttggcaggacgagcgctagccgatatagtgatgacacaaacccctctttcttcacctgttacgcttggacaaccggcttctattagttgtaggtcatcccaatccctggtccactctgatggaaatacatatttgagctggctccaacagcggcccggtcaacccccgcgattgctcatctataagataagtaatcgcttctccggtgtgccagacaggttttccggttcaggagccggtacggactttactctcaagatatcccgcgttgaggcagaagacgtgggcgtgtactactgtatgcaggctactcaattccccctgactttcggcgggggaactaaggttgaaatcaaacgtacggtagctgcgcctagcgtgtttatttttccgccctctgacgaacaactcaaaagtggcactgcctctgtagtctgtctcctgaacaatttttatccaagggaagctaaggtacagtggaaagtcgataacgcgctccaaagtggcaacagtcaagaatctgtaacggagcaagatagtaaagacagcacttacagcttgagcagcacactcacattgtccaaggccgactacgaaaaacataaagtgtacgcctgtgaggtaacacaccagggtctgagcagcccagtgacgaagtcatttaatcgcggtgagtgctga

SEQ ID NO: 478 - protein, 219 aa, Synthetic construct
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 479 - protein, 477 aa, Synthetic construct
METDTLLLWVLLLWVPGSTGEVQLVESGGGVVRPGGSLRLSCAASGFTFDDYGMSWVRQAPGKGLEWVSGINWNGGSTGYADSVKGRFSISRDNAKNSLYLQMNSLRAEDTALYYCAREGITILRGVIIEDWYFDLWGRGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 480 - DNA, 1434 bp, Synthetic construct
  source 1..1434: mol_type = other DNA
atggagacagatactttgctcctttgggtactcttgctgtgggttcccggtagcaccggcgaagtacagctcgtagaaagcggcggcggagttgtacgcccaggaggttccttgcgcctctcatgtgccgcaagcggctttacgttcgatgattacggcatgtcttgggtacgccaggcccccggaaaaggtttggagtgggtatcaggaataaactggaacggtggaagtacaggatatgctgactctgtcaaaggacgcttttccatatcacgcgataatgctaagaatagcctgtatctgcaaatgaacagtctgagggcggaagacactgctctctactattgtgcgagggaaggcataaccatacttcggggcgtaatcatagaagattggtactttgatctttggggaaggggtacacttgtaatcgtctcctccgctagcaccaaaggaccctccgtctttccgctcgcgccgagcagcaagtccacgagcggggggacagccgcccttgggtgtctggtcaaggattattttcctgagcccgttactgtctcctggaatagcggtgccttgacttctggggtacataccttcccggcggttttgcaatcatcaggactgtatagcctctctagcgttgtaacggtaccatcttcatcacttggcacacaaacatatatttgtaatgtaaatcacaaaccttcaaatactaaagttgacaaaaaagtggagccgaagagctgtgataaaacgcatacttgcccgccttgccccgctccagaattgctgggcgggccgtctgtgttcctcttcccaccgaaaccgaaggatacgctgatgataagtcgaacgcccgaagtaacatgcgtggttgttgatgtaagccacgaagatccggaagtaaagttcaactggtacgtagatggagtagaagtgcataacgcgaaaactaagccccgcgaggagcaatataattcaacctatcgggttgttagtgtgctcaccgttctccatcaggattggcttaacgggaaagagtataagtgcaaggttagcaataaagcattgccagcacctatagagaagacaattagcaaagcgaagggacagccacgagagccccaagtctacacgctgcctcccagccgcgacgagttgacgaagaatcaagtgtcactcacatgtcttgtcaagggattctatccctcagatattgcagtagaatgggagtctaatggacaaccagagaacaactataaaactactccaccggtgcttgatagtgacggtagttttttcctttatagtaagctgactgtcgataagagtcgctggcaacagggaaatgttttttcttgtagtgttatgcatgaagcacttcataaccattacacacaaaagtctctgtcactttcccctggcaaatga

SEQ ID NO: 481 - protein, 457 aa, Synthetic construct
EVQLVESGGGVVRPGGSLRLSCAASGFTFDDYGMSWVRQAPGKGLEWVSGINWNGGSTGYADSVKGRFSISRDNAKNSLYLQMNSLRAEDTALYYCAREGITILRGVIIEDWYFDLWGRGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 482 - protein, 474 aa, Synthetic construct
METDTLLLWVLLLWVPGSTGEVQLVESGGGVVRPGGSLRLSCAASGFTFDDYGMSWVRQAPGKGLEWVSGINWNGGSTGYADSVKGRFSISRDNAKNSLYLQMNSLRAEDTALYYCAREGITILRGVIIEDWYFDLWGRGTLVIVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 483 - DNA, 1425 bp, Synthetic construct
  source 1..1425: mol_type = other DNA
atggagacagatactttgctcctttgggtactcttgctgtgggttcccggtagcaccggcgaagtacagctcgtagaaagcggcggcggagttgtacgcccaggaggttccttgcgcctctcatgtgccgcaagcggctttacgttcgatgattacggcatgtcttgggtacgccaggcccccggaaaaggtttggagtgggtatcaggaataaactggaacggtggaagtacaggatatgctgactctgtcaaaggacgcttttccatatcacgcgataatgctaagaatagcctgtatctgcaaatgaacagtctgagggcggaagacactgctctctactattgtgcgagggaaggcataaccatacttcggggcgtaatcatagaagattggtactttgatctttggggaaggggtacacttgtaatcgtctcctccgctagcaccaaaggaccctccgtctttccgctcgcgccgtgctctcgatccacgagcgagtctacggcggccttgggctgtctcgtgaaagactatttcccagagccagtgaccgtttcctggaactccggagcgctcacatcaggtgtgcatacctttccagccgtacttcaatcttcagggctgtatagcttgtcctctgtcgtgaccgtaccctccagttctctcgggacaaagacctatacctgtaacgtagaccataagccatccaataccaaagtagataagagggtggaatctaaatatggaccgccttgtccgccgtgcccggctcctgagttccttggagggccgtccgtctttctgtttccccctaaacctaaggacaccctgatgataagtaggacgccggaagtaacctgtgtcgtggttgatgtgtcacaggaagacccggaggtacaatttaattggtacgtcgatggagtcgaggtgcataatgcgaaaaccaagccacgagaggagcaattcaacagcacctaccgagtagtttcagtcttgactgtgctgcaccaagactggctgaatggcaaggagtacaaatgcaaggtcagtaataaaggattgccgtcttcaatagagaagactatcagtaaggcgaagggacaacctcgcgagcctcaggtatacactttgccgcccagccaagaagaaatgacgaagaaccaagtctctttgacttgcctggtcaaaggcttctacccctctgatatagcagtcgaatgggagtccaacggtcaaccggagaacaattataagacaactccacctgtgcttgattcagacggctcttttttcttgtatagccgccttaccgtagacaagagtcgatggcaggaaggtaatgtattttcttgtagtgtaatgcacgaggcacttcataatcactacacacaaaagtctctgtcactttccctgggcaaatga

SEQ ID NO: 484 - protein, 454 aa, Synthetic construct
EVQLVESGGGVVRPGGSLRLSCAASGFTFDDYGMSWVRQAPGKGLEWVSGINWNGGSTGYADSVKGRFSISRDNAKNSLYLQMNSLRAEDTALYYCAREGITILRGVIIEDWYFDLWGRGTLVIVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 485 - protein, 60 aa, Synthetic construct
AAAGTTTTPAPRPPTPAPTIASQPLSLRPEACRPAAGGAVHTRGLDFACDRRPPSKPFWV

SEQ ID NO: 486 - DNA, 180 bp, Synthetic construct
  source 1..180: mol_type = other DNA
gcggccgcaggtaccaccacaacgcccgctcctcggccaccgacgccagcgccaactattgcgagtcagcctctcagtctgcgacctgaggcttgtcgaccagcagccggaggcgcagtgcacacgagggggctggacttcgcctgtgatagaagacctccttctaagcccttttgggtg

SEQ ID NO: 487 - protein, 23 aa, Synthetic construct
LVVVGGVLACYSLLVTVAFIIFW

SEQ ID NO: 488 - DNA, 69 bp, Synthetic construct
  source 1..69: mol_type = other DNA
ctggtggtggttggtggagtcctggcttgctatagcttgctagtaacagtggcctttattattttctgg

SEQ ID NO: 489 - protein, 162 aa, Synthetic construct
VRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 490 - DNA, 486 bp, Synthetic construct
  source 1..486: mol_type = other DNA
gtgaggaaacggggcagaaagaaactcctgtatatattcaaacaaccatttatgagaccagtacaaactactcaagaggaagatggctgtagctgccgatttccagaagaagaagaaggaggatgtgaactggctagcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgctagc

SEQ ID NO: 491 - protein, 241 aa, Synthetic construct
QVQLVESGGGVVLPGRSLRLSCAASGFTFNIYGMHWVRQAPGKGLEWVAVISYDGNNKEYADSVKGRFTISRDSSRNTLYLQMNSLRAEDTAVYYCAKRGYGSSSLEFWGQGTLVTVSSGGGGSGGGGSGGGGSDIQMTQSPSSLSASVGDRVTITCRSSQDIRNDLGWYQQKPGKAPKRLIYASSNLQSGVPSRFSGSGSGTEFTLTISSLQPDDFATYYCLQHNTYPYTFGQGTKLEIK

SEQ ID NO: 492 - protein, 243 aa, Synthetic construct
QVQLVQSGSELKKPGASVKVSCKTSGYTFTSYAMNWVRQAPGQGLEWMGWINTNTGNPTYAQGFRGRFVFSLDTSVSTAYLQISSLKAEDTAVYYCARDKYSSSWYWFDSWGQGTLVTVSSGGGGSGGGGSGGGGSDIQMTQSPSSLSASVGDRVTITCRPSQNISSYLNWYQQKPGKAPKLLIYAASSLRSGVPSRFSGSGSGTDFTLTVSSLQPEDFANYYCQQSYSTPYTFGRGTKLEIK

SEQ ID NO: 493 - protein, 248 aa, Synthetic construct
QVQLVQSGTEVKYPGASVKVSCKASGYTFIDYGISWVRQAPGQGLEWMGWISAYNGNTDYAQKLQGRVTMTTDTSTSTAYMDLRSLRSDDTAVYYCAREGEGYCTYGVCPSFDYWGQGTLVTVSSGGGGSGGGGSGGGGSEIVLTQSPGTLSLSPGERATLSCRASQSVSSSYLAWYQQKPGQAPRLLIYGASTRGTGIPDRFSGSGSGTDFTLTISRLEPEDFALYYCQQYGSSPYTFGQGTKLEIK

SEQ ID NO: 494 - protein, 247 aa, Synthetic construct
QVQLQESGPGLVKPSETLSLTCTVSGASISGYYWNWIRQPPGKGLEWIGYIHYSGTTNYNPSLKSRITISLDTSENQFSLRLSSVTAADTAVYYCARDWYTGYDWEGFDYWGQGTLVTVSSGGGGSGGGGSGGGGSQSVLTQPPSVSGAPGQRVSFSCTGTSSNIGTGFDVHWYRQLPGTAPKVLIYNNNNRPSGVPDRFSASKSGTSASLAITGLQAEDEADYYCQSYDSSLSGSVFGGGTRLTVL

SEQ ID NO: 495 - protein, 244 aa, Synthetic construct
EVQLVESGGDLVKSGGSLRLSCAASGFTFSDAWMSWVRQAPGKGLEWVGRIQSKTDGGTTDYVAPVKGRFTISRDDSKDTLYLHMNSLTTEDTAVYYCTTDLYYYGSGSYFLGQGTLVTVSSGGGGSGGGGSGGGGSDIQMTQSPSSLSASVGDRVTITCRASQNIITYLNWYQQKPGKAPKLLIYAASSLQSGVPSRFSGSGSGTDFTLTISSLQPEDFAIYHCQQSYNVPTTFGQGTKLEIK

SEQ ID NO: 496 - protein, 249 aa, Synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

(19) United States
(12) Patent Application Publication
(10) Pub. No.: US 2025/0034245 A1
(43) Pub. Date: Jan. 30, 2025

(54) SIGLEC-8 ANTIBODIES AND USES THEREOF

(71) Applicant: Fred Hutchison Cancer Center, Seattle, WA (US)

(72) Inventors: Roland B. Walter, Seattle, WA (US); George S. Laszlo, Seattle, WA (US)

(73) Assignee: Fred Hutchinson Cancer Center, Seattle, WA (US)

(21) Appl. No.: 18/785,723

(22) Filed: Jul. 26, 2024

Related U.S. Application Data
(60) Provisional application No. 63/516,048, filed on Jul. 27, 2023.

(51) Int. Cl. C07K 16/28; A61P 37/04

(52) U.S. Cl. CPC C07K 16/2803; A61P 37/04; C07K 2317/21; C07K 2317/565; C07K 2317/732

(57) ABSTRACT

Siglec-8 antibodies and uses thereof are described. The Siglec-8 antibodies can be fully human and can be used for a variety of purposes, including in the research, diagnosis, and treatment of eosinophil and mast cell related disorders, such as asthma, rhinitis, dermatitis, eosinophilic gastrointestinal disorders, eosinophilic pneumonia, anaphylaxis, urticaria, eosinophilic cellulitis and fasciitis, Churg-Strauss syndrome, hematologic malignancies involving eosinophils or mast cells, and mastocytosis.

### CROSS-REFERENCE TO RELATED APPLICATION

This application claims priority to U.S. Provisional Patent Application No. 63/516,048 filed Jul. 27, 2023, the contents of which are incorporated herein by reference in their entirety as if fully set forth herein.

### STATEMENT REGARDING FEDERALLY SPONSORED RESEARCH OR DEVELOPMENT

This invention was made with government support under AI150566 awarded by the National Institutes of Health. The government has certain rights in the invention.

### REFERENCE TO SEQUENCE LISTING

The Sequence Listing associated with this application is provided in XML format in lieu of a paper copy and is hereby incorporated by reference into the specification. The name of the file containing the Sequence Listing is 39S8217.xml. The file is 541,679 bytes, was created on Jul. 24, 2024, and is being submitted electronically via Patent Center.

### FIELD OF THE DISCLOSURE

The current disclosure provides Siglec-8 antibodies and uses thereof. The Siglec-8 antibodies can be fully human and can be used for a variety of purposes, including in the research, diagnosis, and treatment of eosinophil and mast cell related disorders.

### BACKGROUND OF THE DISCLOSURE

In a wide variety of human diseases, many of which are orphan diseases, eosinophils and/or mast cells play an important pathogenetic role. Examples of eosinophil and mast cell-related disorders include asthma, chronic or allergic rhinitis with nasal polyposis, atopic dermatitis, eosinophilic gastrointestinal disorders (e.g., eosinophilic esophagitis, gastritis, and colitis), chronic eosinophilic pneumonia, hypereosinophilic syndromes, anaphylaxis, chronic idiopathic urticaria, eosinophilic cellulitis and fasciitis, Churg-Strauss syndrome, hematologic malignancies involving eosinophils or mast cells (e.g., eosinophilic leukemia) and mastocytosis.

Targeting eosinophils with monoclonal antibodies (mAbs) blocking interleukin-5 (e.g. mepolizumab or reslizumab), targeting eosinophils and basophils with mAbs blocking the interleukin-5 receptor (e.g. benralizumab), or targeting mast cells and basophils with mAbs that bind free IgE (e.g. omalizumab) are some strategies to treat these disorders. Oftentimes, however, these and other existing therapeutics are insufficiently effective, and patients suffer severe, life-threatening, or fatal complications from the underlying disease. Thus, there is a need for novel, more effective treatments.

One promising target to improve outcomes of eosinophil and/or mast cell-related disorders is sialic acid immunoglobulin (Ig)-like lectin 8 (Siglec-8; also known as SAF2). Siglecs are members of the Ig gene family implicated in promoting cell-cell interactions and regulating functions of cells in the innate and adaptive immune systems through glycan recognition. They are primarily found on hematopoietic and immune cells, mostly in a highly cell type-restricted pattern. For example, Siglec-8, particularly, is expressed late in eosinophil and mast cell maturation and is not displayed on hematopoietic stem cells.

Siglec-8 is characterized by a membrane-distal Ig-like V-set domain, a type 1 Ig-like C2-set domain, and a membrane-proximal type 2 Ig-like C2-set domain in its extracellular portion. In addition to this full-length Siglec-8 (Siglec-8^{FL}), alternative splicing leads to shorter variants, including one lacking the exon 2-encoded type 1 Ig-like C2-set domain (Siglec-8^{ΔE2}).

Unconjugated Siglec-8 mAbs cause caspase and/or reactive oxygen species-dependent apoptosis and inhibit mast cell degranulation and activation. A humanized non-fucosylated IgG1 Siglec-8 mAb (AK002, lirentelimab) was tested in healthy subjects and showed drug tolerability and rapid depletion of eosinophils from the peripheral blood. Data from a randomized phase 2 trial in adults with symptomatic eosinophilic gastritis and/or eosinophilic duodenitis showed responses in 63% of cases with lirentelimab (vs. 5% with placebo), thereby validating Siglec-8 as a therapeutic drug target. This notion is supported by findings from additional, uncontrolled trials reporting beneficial effects of this unconjugated Siglec-8 mAb in patients with indolent systemic mastocytosis, chronic urticaria, and severe allergic conjunctivitis.

### SUMMARY OF THE DISCLOSURE

The current disclosure describes novel Siglec-8 antibodies that bind either the membrane distal V-set domain of Siglec-8, the membrane-distal C2-set domain of Siglec-8, or the membrane proximal C2-set domain of Siglec-8. By providing antibodies that bind the full length Siglec-8 protein and/or truncated forms thereof, in certain examples, treatment of subjects can be tailored based on the Siglec-8 isoform expressed by the subject.

The particular antibodies disclosed herein are referred to as Siglec-8 (S8)-ATX1B4, S8-ATX1B6, S8-ATX1E1, S8-ATX1E2, S8-ATX1E4, S8-ATX1H4, S8-ATX2A3, S8-ATX2A7, S8-ATX2B11, S8-ATX2F10, and S8-ATX2G9. The antibodies can also be referred to with a Sig8-prefix, such as Sig8-1B4, Sig8-1B6, Sig8-1E1, Sig8-1E2, Sig8-1E4, Sig8-1H4, Sig8-2A3, Sig8-2A7, Sig8-2B11, Sig8-2F10, and Sig8-2G9, respectively; or without a prefix such as 1B4, 1B6, 1E1, 1E2, 1E4, 1H4, 2A3, 2A7, 2B11, 2F10, and 2G9, respectively.

In particular embodiments, antibodies that bind the V-set domain of Siglec-8 include 1B6, 1 H4, 1B4, 1E1, 1E4, and 1E2. In particular embodiments, an antibody that binds the membrane-distal C2-set domain of Siglec-8 includes 2A3. In particular embodiments, antibodies that bind the membrane-proximal C2-set domain of Siglec-8 include 2A7 and 2F10. In particular embodiments, antibodies that bind specifically to Siglec-8^{ΔE2} include 2B111 and 2G9.

Binding domains of the antibodies disclosed herein can be used to create numerous engineered formats such as Siglec-8 mAb-based immunotherapeutics and cell-based immunotherapies (e.g., recombinant receptor), among the many other uses described elsewhere herein.

When provided in fully human form, the disclosed antibodies lack human anti-mouse antibody (HAMA) immunization properties and can be given repeatedly to individual patients.

### BRIEF DESCRIPTION OF THE SEVERAL VIEWS OF THE DRAWINGS

Some of the drawings submitted herewith may be better understood in color. Applicant considers the color versions of the drawings as part of the original submission and reserves the right to present color images of the drawings in later proceedings.

FIG. 1. Siglec-8 splice variants and domain-specific monoclonal antibodies (mAbs). Schema of full-length Siglec-8 (Siglec-8^{FL}), a naturally occurring variant of Siglec-8 lacking the exon 2-encoded type 1 Ig-like C2-set domain (Siglec-8^{ΔE2}), and an artificial V-set only construct (Siglec-8^{V-set only}) with depicted binding of mAbs to the natural Ig-like V-set domain (solid line), membrane distal type 2 Ig-like C2-set domain (dashed line) or the membrane proximal type 2 Ig-like C2-set domain (dotted line).

FIG. 2. Immunogens used for the generation of human mAbs against human Siglec-8. Schema of full-length human Siglec-8, mouse CD33 (inset, left) and mouse Siglec-F (inset, right) as well as mouse/human chimeric proteins with extracellular domains including the mouse CD33 V-set domain fused to human Siglec-8 membrane proximal type 2 Ig-like C2-set domain (CD33^{Mu}/Siglec-8^{HU}) or mouse Siglec-F V-set domain fused to human Siglec-8 membrane distal and proximal type 2 Ig-like C2-set domains (Siglec-F^{Mu}/Siglec-8^{Hu}). Transmembrane and intracellular domains were derived from human Siglec-8.

FIG. 3. m3T3 cells expressing human Siglec-8 as an immunogen. m3T3 cells were transduced with lentivirus encoding indicated transgenes and an IRES-EGFP reporter. EGFP⁺ cells were collected via fluorescence activated cell sorting (FACS) to generate m3T3 sublines expressing full-length human Siglec-8 (left top panel, square), mouse/human chimeric proteins including the mouse CD33 V-set domain fused to human Siglec-8 membrane-proximal type 2 Ig-like C2-set domain (CD33^{Mu}/Siglec-8^{Hu}, right top panel, star) or mouse Siglec-F V-set domain fused to human Siglec-8 membrane-proximal type 2 Ig-like C2-set domain (Siglec-F^{Mu}/Siglec-8^{Hu} bottom panel, triangle). m3T3 cells expressing an unrelated protein are included as negative control (bottom panel, circle).

FIG. 4. K562 cells expressing human Siglec-8. Siglec-8 antibody screening tools were generated using K562 cells transduced with lentivirus encoding indicated transgenes and an IRES-EGFP reporter. EGFP⁺ cells were then collected via FACS to generate sublines. K562 cells expressing full-length human Siglec-8 (Siglec-8^{FL}; left panel, third from top), 6-His-tagged human Siglec-8 (6His-Siglec-8^{FL}; left panel, bottom, Siglec-8^{ΔE2} (left panel, second from top), or Siglec-8^{V-set only} (Siglec-8^{V-set}; left panel, top) are shown in the left panel. K562 cells (right panel) were transduced with full-length human Siglec-8 (Siglec-8^{FL}; right panel, top), or CD33^{Mu}/Siglec-8^{Hu} (right panel, bottom). Siglec-8 was detected using a PE-Cy7-conjugated commercially available human Siglec-8 antibody (clone 7C9). Parental K562 cells are shown as negative control (right panel, center). The Siglec-8 mAb clone 7C9 does not bind the membrane proximal domain of Siglec-8.

FIG. 5. Endogenous expression of human Siglec-8. The human mast cell line LUVA expresses endogenous Siglec-8 (right panel) at low levels, detected using a PE-Cy7-conjugated commercially available human Siglec-8 mAb (clone 7C9). K562 cells expressing full-length human Siglec-8 (Siglec-8^{FL}, lower line) are shown as positive control (left panel).

FIG. 6. Selection of Siglec-8 hybridomas for further analysis. Hybridoma supernatants were screened on Siglec-8-negative K562 cells (Parental cells, center lines), K562 cells expressing full-length human Siglec-8 (Siglec-8^{FL}, upper lines), or CD33^{Mu}/Siglec-8^{Hu} (lower lines). Hybridoma supernatant from clone Sig8-2F10 strongly bound both Siglec-8^{FL} and the membrane proximal portion of Siglec-8 in the context of CD33^{Mu}/Siglec-8^{Hu} protein, indicating an antibody that is “pan”-specific for human Siglec-8 protein. Sig8-2A7 Sig8-2B11 and Sig8-2G9 bound to CD33^{Mu}/Siglec-8^{Hu} and, also, to Siglec-8^{FL} to varying degrees. All other antibodies bound to full-length human Siglec-8 (Siglec-8^{FL} but do not bind CD33^{Mu}/Siglec-8^{Hu})

FIG. 7. Production of fully human recombinant Siglec-8 mAbs. Siglec-8 mAb sequences were derived from hybridoma cells utilizing 5′ RACE cloning. HcFv and LcFv regions were cloned into pcDNA3.4 vectors containing either human IgG1, human IgG4, human Ig kappa or human Ig lambda Fc frameworks, sequence verified, and recombinant mAbs expressed using an Expi293 cell-based transient transfection system. mAbs were affinity purified from culture supernatants over HiTrap™ MabSelectSuRe™ columns (Cytiva) using an AKTApurifier chromatography system (Cytiva), and 25 μg of each purified mAb was run on a protein gel under reducing conditions. Protein gels were stained with a Coomassie blue-based dye to visualize proteins. 1E4, 1 H4, 1B6, 2F10, 2A3, 2B111, 1E1, 2G9, 2A7, and 1B4 are shown, 2D5 is a protein loading control mAb.

FIG. 8. Binding specificity of fully human recombinant Siglec-8 mAbs. Sig8-1B6 and Sig8-2F10 were generated as recombinant human mAbs in the human IgG1/kappa framework, and binding of recombinant mAbs tested on K562 cells expressing increasing amounts of target protein with multiplicity of infection (MOI) of transgenes as defined. Sig8-1B6 is a V-set-specific human Siglec-8 mAb, whereas Sig8-2F10 does not bind Siglec-8^{V-set only} but rather binds to Siglec-8^{ΔE2}, indicating that this clone is specific for the membrane proximal C2-set domain of Siglec-8. The Siglec-8 mAb clone 7C9, shown for comparison, displays properties of a V-set-specific human Siglec-8 mAb.

FIGS. 9A-9D. Human Siglec-8 mAbs were tested flow cytometrically against (FIG. 9A) parental human lymphoid RS4; 11 cells (endogenously lacking Siglec-8 expression) and a subline engineered to express Siglec-8^{FL}, (FIG. 9B) parental human mast cell line, HMC1.2, enriched for expression of endogenous Siglec-8; and (FIG. 9C) parental human myeloid K562 (endogenously lacking Siglec-8 expression) and sublines of K562 cells engineered to express either Siglec-8^{FL}, Siglec-8^{ΔE2}, or Siglec-8^{V-set}; and (FIG. 9D) primary human eosinophils, as indicated. A negative control without primary mAbs was included, as was, in some experiments, a negative control with a non-binding primary mAb (13R4). 1B6, 1H4, 1B4, 1E1, 1E4, and 1E2 strongly bound Siglec-8^{FL}, Siglec-8^{ΔE2}, and Siglec-8^{V-set} indicating that these are V-set-specific human Siglec-8 mAbs. 2A7 and 2F10 bound Siglec-8^{FL} and Siglec-8^{ΔE2}, but not Siglec-8^{V-set} consistent with binding to the membrane proximal portion of Siglec-8 and indicating that these antibodies are “pan”-specific for human Siglec-8 protein. 2A3 bound to Siglec-8^{FL} but not to Siglec-8^{ΔE2} or Siglec-8^{V-set} consistent with binding to the membrane distal C2-set domain of Siglec-8. 2B111 and 2G9 only bound to the Siglec-8^{ΔE2} variant Siglec-8 protein.

FIG. 10. Binding of Siglec-8 mAb S8-2F10 to endogenously Siglec-8-expressing LUVA cells. Hybridoma supernatant for S8-2F10 was tested for binding to endogenous Siglec-8 expressed on LUVA cells.

FIG. 11. Internalization of Siglec-8 mAbs. Flow cytometry-based in vitro internalization assays of Siglec-8 mAbs (1B6, 1H4, 2A3, 2A7, 2F10) in parental LUVA cells (“LUVA” panel), parental human HMC1.2 enriched for expression of endogenous Siglec-8 (“HMC1.2” panel), and LUVA cells transduced with human Siglec-8^{FL} (“LUV/Sigle-8^{FL}” panel). Shown are mean±SEM values of 2-3 independent experiments.

FIG. 12. Generation of acute myeloid leukemia (AML) cells expressing human Siglec-8. Siglec-8 mAb screening tools were generated using Siglec-8-negative AML cell lines transduced with lentivirus encoding indicated transgenes and an IRES-EGFP reporter. EGFP⁺ cells were subsequently collected via FACS to generate sublines. EGFP fluorescence (top panels) or Siglec-8 expression (bottom panels) is shown, indicating cells were successfully transduced. Siglec-8 was detected using a PE-Cy7-conjugated commercially available human Siglec-8 mAb (clone 7C9).

FIG. 13. Binding of human Siglec-8 mAbs to TF-1^{Siglec-8} cells. 2A3, 1 H4 and 1B6 mAbs as fully human mAbs in the IgG4 format were screened for binding against TF-1^{Siglec-8} cells.

FIG. 14. Production of fully human recombinant Siglec-8 CD3 IgG-scFv bispecific T cell-directed therapeutics. Siglec-8 mAb heavy chains were combined with light chains that contain a CD3 scFv to generate Siglec-8 CD3 IgG-scFv bispecific T cell-directed therapeutics. Proteins were expressed using an Expi293 cell-based transient transfection system. Proteins were affinity purified from culture supernatants over HiTrap™ MabSelectSuRe™ columns (Cytiva, Marlborough, MA) using an AKTApurifier chromatography system (Cytiva), and 25 μg of each purified protein was run on a protein gel under reducing conditions. Protein gels were stained with a Coomassie blue-based dye to visualize proteins. Sig8-1H4, Sig8-1B6, Sig8-2F10 and Sig8-2A3 are shown.

FIG. 15. Siglec-8 CD3 IgG-scFv bispecific T cell-directed therapeutics retain Siglec-8 binding properties. Binding of Siglec-8/CD3 IgG1-scFv bispecific T cell-directed therapeutics Sig8-2A3/CD3, Sig8-1H4/CD3, Sig8-1B6/CD3 and Sig8-2F10/CD3 were detected with an anti-human secondary mAb.

FIG. 16. T-cell mediated cytotoxicity using Siglec-8 CD3 IgG-scFv bispecific T cell-directed therapeutics. 2F10, 1B6, 1 H4 or 2A3 mAbs were engineered to include a CD3 scFv fused to the light chain. Burgundy dye-labeled CD3+ pan-T cells were mixed with LUVA cells, which express low level of endogenous Siglec-8, at a 10:1 effector:target (E:T) cell ratio. Data is shown using burgundy-negative target cells. Schema for Siglec-8 CD3 IgG-scFv bispecific T cell-directed therapeutics is also shown.

FIG. 17. T cell mediated cytotoxicity using Sig8-1B6 Siglec-8 CD3 IgG-scFv bispecific T cell-directed therapeutic. Burgundy dye-labeled CD3+ pan-T cells were mixed with either Siglec-8-expressing target cells or target antigen-negative parental cell lines at a 3:1 E:T cell ratio. The Sig8-1B6 bispecific antibody (BiAb) was generated using both a human IgG1 (left graph) and a human IgG4 (right graph) framework. Data are shown using burgundy-negative target cells. Schema for Siglec-8 CD3 IgG-scFv bispecific T cell-directed therapeutics is also shown.

FIG. 18. Schema for bispecific antibody construct. SP: signal peptide, V_{L}: variable light chain, V_{H}: variable heavy chain, G₄S: GlySer linker as set forth in SEQ ID NO: 354, (G₄S)₃: GlySer linker as set forth in SEQ ID NO: 352, and His₆: His tag as set forth in SEQ ID NO: 401.

FIGS. 19A-19D. Cytolytic activity of Siglec-8^{V-set}-directed immunotherapies engaging T or NK cells. (FIG. 19A) Parental EOL-1 and HL-60 cells and corresponding sublines transduced with human full-length Siglec-8 (+Siglec-8^{FL}) were incubated with healthy donor T cells at an E:T cell ratio of 1:1 with increasing concentrations of a Siglec-8^{V-set}/CD3 BiAb (11B6). (FIG. 19B) Parental CD19+ RS4; 11 cells and parental human HMC1.2 enriched for expression of endogenous Siglec-8 were incubated with healthy donor T cells at an E:T cell ratio of 3:1 with increasing concentrations of either a CD19/CD3 or a Siglec-8^{V-set}/CD3 BiAb (11B6). (FIG. 19C) Siglec-8 gene edited LUVA cells (Siglec-8^{KO}), parental LUVA cells, and LUVA cells transduced with human full-length Siglec-8 (+Siglec-8^{FL}) were incubated with healthy donor T cells at an E:T cell ratio of 3:1 with increasing concentrations of a Siglec-8^{V-set}/CD3 BiAb (11B6). Dead target cells were enumerated after 48 hours via flow cytometry, and change in dead cells with BiAb treatment compared to cells without BiAb treatment is shown. (FIG. 19D) Parental EOL-1 and RS4; 11 cells and corresponding sublines transduced with human full-length Siglec-8 (+Siglec-8^{FL}) were incubated at an E:T cell ratio of 2:1 with KHYG-1 cells expressing a Siglec-8^{V-set}-directed CAR molecule generated from scFv sequences from 1B6 or 1H4. Dead target cells were enumerated after 48 hours via flow cytometry. For all experiments, mean+SEM values from 3 separate experiments are shown. *p<0.05; **p<0.01; ***p<0.001; ****p<0.0001.

FIGS. 20A-20F. Cytolytic activity of Siglec-8^{C2-set}-directed immunotherapies engaging T or NK cells. (FIG. 20A) Parental EOL-1, ML-1, and RS4; 11 cells and corresponding sublines transduced with human full-length Siglec-8 (+Siglec-8^{FL}) were incubated with healthy donor T cells at an E:T cell ratio of 1:1 with increasing concentrations of a Siglec-8^{C2-set}/CD3 BiAb (2A3). (FIG. 20B) Parental CD19+ RS4; 11 cells and parental human HMC1.2 enriched for expression of endogenous Siglec-8 were incubated with healthy donor T cells at an E:T cell ratio of 3:1 with increasing concentrations of either a CD19/CD3 or a Siglec-8^{C2-set}/CD3 BiAb (2A3). (FIG. 20C) Siglec-8 gene edited LUVA cells (Siglec-8^{KO}), parental LUVA cells, and LUVA cells transduced with human full-length Siglec-8 (+Siglec-8^{FL}) were incubated with healthy donor T cells at an E:T cell ratio of 3:1 with increasing concentrations of a Siglec-8^{C2-set}/CD3 BiAb (2A3). Dead target cells were enumerated after 48 hours via flow cytometry, and change in dead cells with BiAb treatment compared to cells without BiAb treatment is shown. (FIG. 20D) Parental EOL-1 and RS4; 11 cells and corresponding sublines transduced with human full-length Siglec-8 (+Siglec-8^{FL}) were incubated at an E:T cell ratio of 2:1 with KHYG-1 cells expressing a Siglec-8^{C2-set}-directed CAR molecule generated from scFv sequences from 2A3 or 2F10. (FIG. 20E) Parental human HMC1.2 enriched for expression of endogenous Siglec-8 were incubated with KHYG-1 cells expressing either a Siglec-8^{C2-set}-directed CAR molecule (2A3 or 2F10) or a non-binding control CAR molecule (13R4). (FIG. 20F) Parental EOL-1 and RS4; 11 cells and corresponding sublines transduced with human full-length Siglec-8 (+Siglec-8^{FL}) were incubated with healthy donor NK cells at an E:T cell ratio of 3:1 with increasing concentrations of Siglec-8^{C2-set} mAb (2A3) or Siglec-8^{V-set} mAb (lirentelimab) in IgG1 format. Dead target cells were enumerated after 48 hours via flow cytometry. For all experiments, mean+SEM values from 3 separate experiments are shown. *p<0.05; **p<0.01; ***p<0.001.

FIG. 21. Proximity of antibody binding to membrane enhances T cell mediated cytotoxicity using Sig8-1B6 Siglec-8/CD3 bispecific antibody. Quantibrite staining was used to determine Siglec-8 molecules per cell. Percentage of DAPI+ cells relative to no drug is shown using 3 doses of Sig8-1B6 Siglec-8/CD3 bispecific antibody on cells expressing full-length human Siglec-8 (Siglec-8^{FL}), Siglec-8^{ΔE2}, or Siglec-8^{V-set} in TF-1 cells (top panels) or EOL-1 cells (bottom panels). Siglec-8 Burgundy dye-labeled CD3+ pan-T cells were mixed with either target antigen-negative parental cell lines, or Siglec-8-expressing target cells or at a 3:1 E:T cell ratio. Data are shown using burgundy-negative target cells.

FIGS. 22A-22F. Membrane proximity of the target epitope modulates the efficacy of Siglec-8^{V-set}-directed BiAbs and CAR-modified KHYG-1 cells. (FIG. 22A) Parental EOL-1, ML-1, RS4; 11, and TF-1 cells were used to generate sublines expressing either full-length Siglec-8 (Siglec-8^{FL}) or a variant of Siglec-8 containing only the V-set domain in its extracellular portion (Siglec-8^{V-set}) Relative expression of the target proteins was flow cytometrically quantified via QuantiBRITE employing the V-set domain Siglec-8 antibody, 7C9, with representative histograms shown in the inserts. (FIG. 22B) Parental ML-1 and RS4; 11 cells and (FIG. 22C) parental TF-1 cells and corresponding sublines lentivirally forced to express similar levels of either Siglec-8^{FL} and Siglec-8^{V-set} were incubated with healthy donor T cells at an E:T cell ratio of 1:1 with increasing concentrations of a Siglec-8^{V-set}/CD3 BiAb (11B6). Dead target cells were enumerated after 48 hours via flow cytometry, and change in dead cells with BiAb treatment compared to cells without BiAb treatment is shown. (FIG. 22D) Parental EOL-1 and RS4; 11 cells and corresponding sublines overexpressing similar levels of either Siglec-8^{FL} and Siglec-8^{V-set} were incubated at different E:T cell ratios with KHYG-1 cells expressing a Siglec-8^{V-set}-directed CAR molecule (11B6). (FIGS. 22E, 22F) Cells lines as described in FIG. 22D were incubated with healthy donor NK cells at an E:T cell ratio of 3:1 with increasing concentrations of the Siglec-8^{V-set} antibody, (FIG. 22E) lirentelimab, or (FIG. 22F) 1H4 in IgG1 format. Non-viable target cells were enumerated after 48 hours via flow cytometry. For all experiments, mean+SEM values from at least 3 separate experiments are shown. *p<0.05; **p<0.01; ***p<0.001; ****p<0.0001.

FIGS. 23A, 23B. Phenotyping of isolated cells post-enrichment of primary human eosinophils from peripheral blood of patient donors using the Eosinophil Isolation Kit from Miltenyi Biotec. (FIG. 23A) Purified human eosinophils showed low CD16 expression. (FIG. 23B) Primary human eosinophils isolated from peripheral blood of patients were >90% pure, measured by co-expression of CCR3 and Siglec-8. Representative phenotyping of isolated primary human eosinophils from peripheral blood of 1 out of 2 patient donors is shown.

FIGS. 24A-24D. Efficacy of Siglec-8^{C2-set}-directed immunotherapies engaging T or NK cells against primary human eosinophils. (FIG. 24A) Eosinophils isolated from peripheral blood of patients were incubated with increasing concentrations of Siglec-8^{C2-set} mAb (2A3 or 2F10) or non-binding control mAb (13R4), all in IgG1 format. (FIG. 24B) Similar to FIG. 24A but with additional healthy donor NK cells present at an E:T cell ratio of 3:1. (FIG. 24C) Primary human eosinophils were incubated with healthy donor T cells at an E:T cell ratio of 3:1 with increasing concentrations of Siglec-8^{C2-set}/CD3 BiAb using binding sequences from 2A3 or a non-binding control BiAb (13R4). (FIG. 24D) Primary human eosinophils were incubated at various E:T cell ratios with KHYG-1 cells expressing either a Siglec-8^{C2-set}-directed CAR molecule (2A3 or 2F10) or a non-binding control CAR molecule (13R4). For all experiments, live eosinophils were enumerated after 16-18 hours via flow cytometry and change in live eosinophils with treatment compared to without treatment is shown. Shown are mean+SEM values from 2 separate experiments using 2 different patient donors for eosinophils with at least 2 replicates per experiment. *p<0.05; **p<0.01; ***p<0.001.

FIG. 25. Sartorius Octet Red96 biolayer interferometry (BLI) measurements were acquired using 5 μg/ml recombinant human IgG4 versions of Siglec-8 antibodies immobilized to the BLI sensor through standard EDC-catalyzed amide bond formation with amine reactive 2nd generation (AR2G) biosensor and Acrobiosystems Hs_Siglec-8_6His-Avi protein, using PBS, 0.1% BSA, 0.2% Tween-20. ND=no data.

FIGS. 26A-26F. Sequences supporting the disclosure. (26A) signal peptides of light and heavy chains; (26B) Siglec-8 (for cell-based screening tools, cell-based immunogens, cell-based epitope mapping and cell-based cytotoxicity assays), Hs_Siglec-8_V-set+C2-set 2 and Hs_Siglec-8_V-set domain only (for cell-based epitope mapping and cell-based cytotoxicity assays), Mm_CD33/Hs_Siglec-8 and Mm_Siglec-F/Hs_Siglec-8 (for cell-based screening tools and cell-based immunogens), 6His-Siglec-8 (for cell-based screening tools/immunogens), Hs_Siglec-8_ECD-mmFc and Mm_Siglec-F_ECD/Hs_Siglec-8-mmFc Mm_CD33_ECD/Hs_Siglec-8-mmFc (immunogens for antibody generation), and Hs_Siglec-8_6His-Avi (antibody screening tool and for affinity measurements); (26C) fully human recombinant antibody chains S8-ATX2A3_Hs_IgG1_HC, S8-ATX2A3_Hs_IgG4_HC, S8-ATX2A3_Hs_IgL_LC, S8-ATX1H4_Hs_IgG1_HC, S8-ATX1H4_Hs_IgG4_HC, S8-ATX1H4_Hs_IgK_LC, S8-ATX1B6_Hs_IgG1_HC, S8-ATX1B6_Hs_IgG4_HC, S8-ATX1B6_Hs_IgK_LC, S8-ATX2F10_Hs_IgG1_HC, S8-ATX2F10_Hs_IgG4_HC, S8-ATX2F10_Hs_IgK_LC, S8-ATX1B4_Hs_IgG1_HC, S8-ATX1B4_Hs_IgG4_HC, S8-ATX1B4_Hs_IgK_LC, S8-ATX1E1_Hs_IgG1_HC, S8-ATX1E1_Hs_IgG4_HC, S8-ATX1E1_Hs_IgK_LC, S8-ATX1E2_Hs_IgG1_HC, S8-ATX1E2_Hs_IgG4_HC, S8-ATX1E2_Hs_IgK_LC, S8-ATX1E4_Hs_IgG1_HC, S8-ATX1E4_Hs_IgG4_HC, S8-ATX1E4_Hs_IgK_LC, S8-ATX2A7_Hs_IgG1_HC, S8-ATX2A7_Hs_IgG4_HC, S8-ATX2A7_Hs_IgK_LC, S8-ATX2B11_Hs_IgG1_HC, S8-ATX2B11_Hs_IgG4_HC, S8-ATX2B11_Hs_IgK_LC, S8-ATX2G9_Hs_IgG1_HC, S8-ATX2G9_Hs_IgG4_HC, and S8-ATX2G9_Hs_IgK_LC; (26D) fully human Siglec-8 BiAb T-cell engagers S8-ATX1H4_CD3_BiAb, S8-ATX2F10_CD3_BiAb, S8-ATX2A3_CD3_BiAb, and S8-ATX1B6_CD3_BiAb; and (26E) fully human Siglec-8 Ig-BiAb T-cell engagers S8-ATX2F10_CD3_Ig-scFv-BiAb_LC, S8-ATX2A3_CD3_Ig-scFv-BiAb_LC, S8-ATX1B6_CD3_Ig-scFv-BiAb_LC, and S8-ATX1H4_CD3_Ig-scFv-BiAb_LC; (26F) pSLCAR human anti-Siglec-8 chimeric antigen receptors.

### DETAILED DESCRIPTION

In a wide variety of human diseases, many of which are orphan diseases, eosinophils and/or mast cells are thought to play an important pathogenetic role (Kiwamoto et al., Pharmacol Ther. 2012, 135(3):327-336 (“Kiwamoto 2012”)). Beyond asthma, examples of eosinophil-related or mast cell-related diseases include chronic or allergic rhinitis with nasal polyposis, atopic dermatitis, eosinophilic gastrointestinal disorders (e.g., eosinophilic esophagitis, gastritis, and colitis), chronic eosinophilic pneumonia, hypereosinophilic syndromes, anaphylaxis, chronic idiopathic urticaria, eosinophilic cellulitis and fasciitis, Churg-Strauss syndrome, hematologic malignancies involving eosinophils or mast cells (e.g., eosinophilic leukemia), and mastocytosis (Kiwamoto 2012). Some strategies to treat these disorders include targeting eosinophils with monoclonal antibodies (mAbs) blocking interleukin-5 (e.g. mepolizumab or reslizumab), targeting eosinophils and basophils with mAbs blocking the interleukin-5 receptor (e.g. benralizumab), or targeting mast cells and basophils with mAbs that bind free IgE (e.g. omalizumab). Oftentimes, however, these and other existing therapeutics are insufficiently effective, and patients suffer severe, life-threatening, or fatal complications from the underlying disease. Thus there is a need for better treatments. One promising target to improve outcomes in these disorders is sialic acid immunoglobulin (Ig)-like lectin 8 (Siglec-8; also known as SAF2) (Kiwamoto 2012; and Youngblood et al., Cells. 2021, 10(1):19 (“Youngblood 2021”)).

Siglecs are members of the Ig gene family implicated in promoting cell-cell interactions and regulating functions of cells in the innate and adaptive immune systems through glycan recognition (Crocker, et al., Nat Rev Immunol. 2007, 7(4):255-266 (“Crocker 2007”)). They are primarily found on hematopoietic and immune cells, mostly in a highly cell type-restricted pattern. This is also true for Siglec-8, which is only expressed on eosinophils, mast cells and, weakly, basophils (Kiwamoto 2012; Floyd et al., J Biol Chem. 2000, 275(2):861-866 (“Floyd 2000”); Kikly et al., J Allergy Clin Immunol. 2000, 105(6 Pt 1):1093-1100 (“Kikly 2000”); and Liu et al., J Allergy Clin Immunol. 2006, 118(2):496-503 (“Liu 2006”)). Siglec-8 is expressed late in eosinophil and mast cell maturation and is not displayed on hematopoietic stem cells.

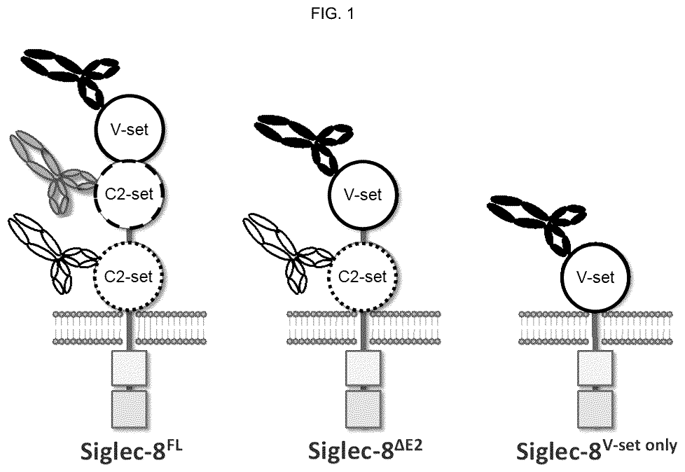

Siglec-8 is characterized by a membrane-distal immunoglobulin (Ig)-like V-set domain, a type 1 Ig-like C2-set domain, and a membrane-proximal type 2 Ig-like C2-set domain in its extracellular portion (FIG. 1) (Kiwamoto 2012; Crocker2007). In addition to full-length Siglec-8 (Siglec-8^{FL}), alternative splicing leads to shorter variants, including one lacking the exon 2-encoded type 1 Ig-like C2-set domain (Siglec-8^{ΔE2}).

Several trials have been initiated with a humanized non-fucosylated IgG1 Siglec-8 mAb (AK002, lirentelimab) after testing in healthy subjects indicated drug tolerability and rapid depletion of eosinophils from the peripheral blood (Youngblood 2021; and Rasmussen et al., J Allergy Clin Immunol. 2018, 141(2 Suppl):AB403). Data from a randomized phase 2 trial in adults with symptomatic eosinophilic gastritis and/or eosinophilic duodenitis, (Dellon et al., N Engl J Med. 2020, 383(17):1624-1634 (“Dellon 2020”)) showed responses in 63% of cases with lirentelimab (vs. 5% with placebo), thereby validating Siglec-8 as a therapeutic drug target. Findings from additional, uncontrolled trials also report beneficial effects of this unconjugated Siglec-8 mAb (lirentelimab) in patients with indolent systemic mastocytosis, chronic urticaria, and severe allergic conjunctivitis (Youngblood 2021).

The current disclosure describes novel Siglec-8 antibodies that bind the V-set domain of Siglec-8, the membrane distal C2-set domain of Siglec-8, or the membrane proximal C2-set domain of Siglec-8. By providing antibodies that bind the full length Siglec-8 protein and/or truncated forms thereof, in certain examples, treatment of subjects can be tailored based on the Siglec-8 isoform expressed by the subject.

In particular embodiments, antibodies that bind the V-set domain of Siglec-8 include 1B6, 1 H4, 1B4, 1E1, 1E4, and 1E2. In particular embodiments, an antibody that binds the membrane-distal C2-set domain of Siglec-8 includes 2A3. In particular embodiments, antibodies that bind the membrane-proximal C2-set domain of Siglec-8 include 2A7 and 2F10. In particular embodiments, antibodies that bind specifically to Siglec-8^{ΔE2} include 2B11 and 2G9. In particular embodiments, the binding distance from the cell membrane correlates with the efficacy of Siglec-8 directed immunotherapies. In particular embodiments, 2A3 internalizes slowly compared to the other binding domains described herein.

When provided in fully human form, the disclosed antibodies lack human anti-mouse antibody (HAMA) immunization properties and can be given repeatedly to individual patients.

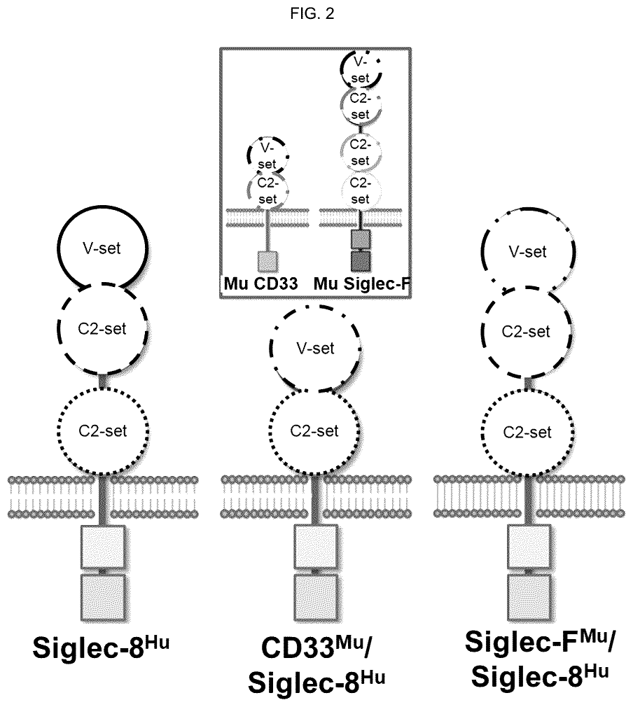

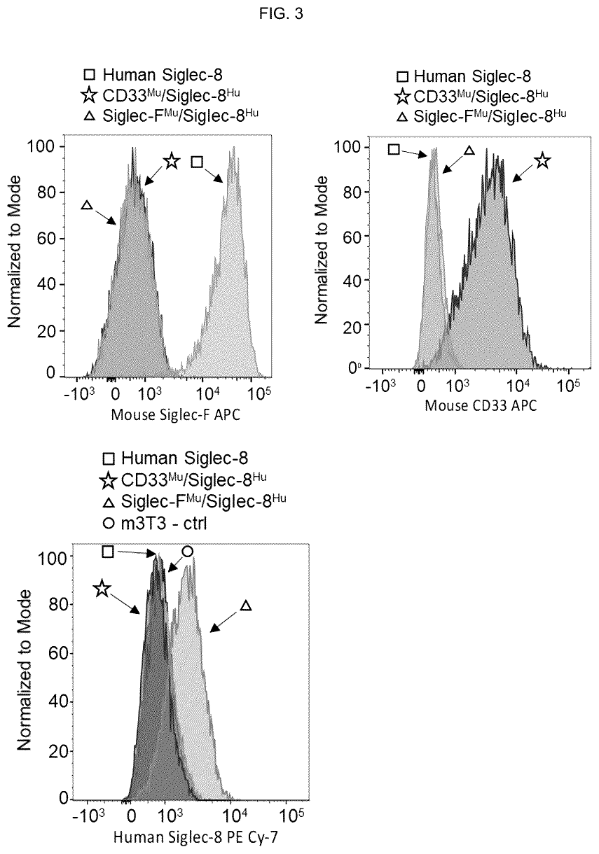

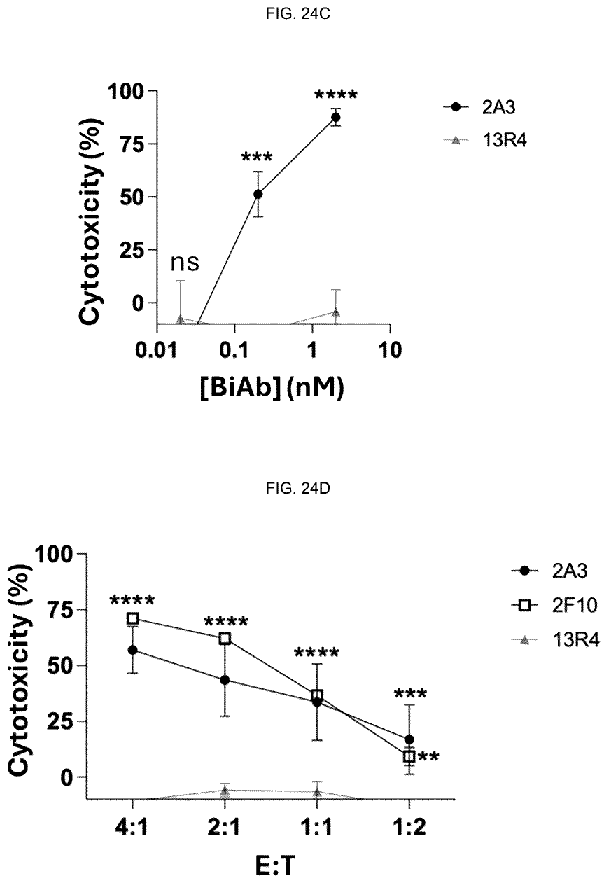

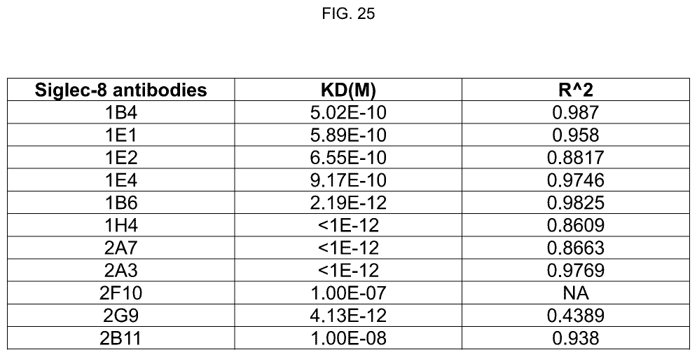

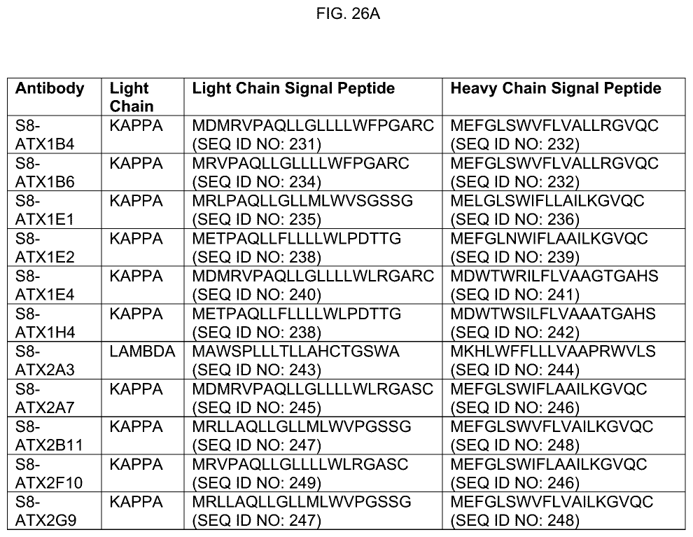

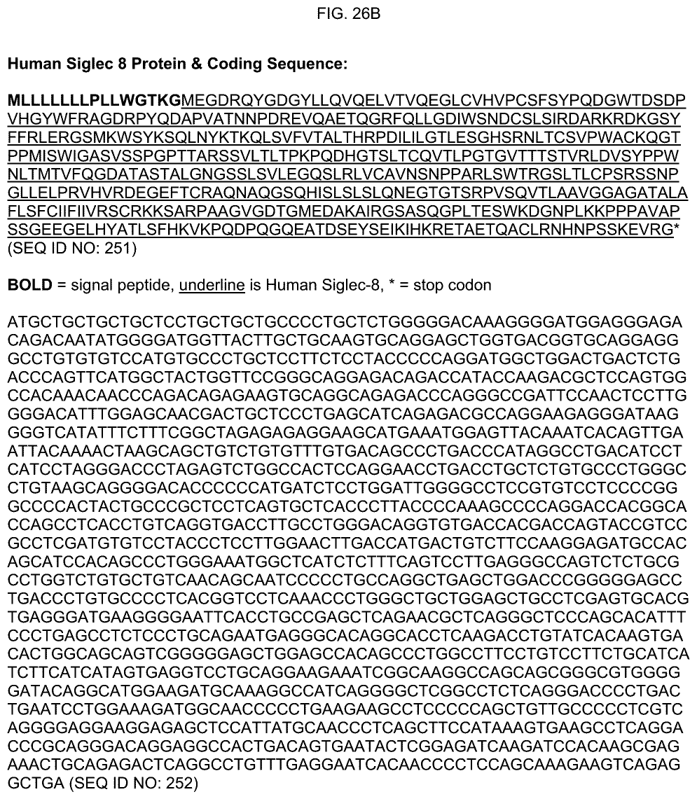

To generate antibodies disclosed herein, the ATX-GX mouse platform (Alloy Therapeutics) was used. ATX-GX mice are transgenic animals developed as in vivo discovery platform and optimized for human antibody sequence developability and diversity, encompassing haplotype diversity and a full human heavy chain repertoire (40V, 23D, and 6J segments), with separate mice for human kappa (19V, 5J segments) and human lambda chain repertoires (22V, 5J segments). The entire sequence of human Siglec-8 was used as one immunogen (“Siglec-8^{Hu}”; FIG. 2). Because recent studies with Siglec-3 (CD33) have shown that membrane-proximal binding of mAbs enhances their immune effector functions (Godwin et al., Leukemia. 2021, 35(9):2496-2507 (“Godwin 2021”)), forcing the diversification of binding epitopes and raising mAbs against the membrane-proximal type 2 Ig-like C2-set domain of Siglec-8 was of interest. Therefore 2 additional chimeric immunogens were generated, one including the membrane-proximal C2-set domain of human Siglec-8 and the V-set domain of murine CD33 (“CD33^{Mu}/Siglec-8^{Hu}”), and the other including the membrane-proximal C2-set domain of human Siglec-8 and the V-set domain of murine Siglec-F (“Siglec-F^{Mu}/Siglec-8^{Hu}”; there is no murine Siglec-8; FIGS. 2 and 24B). These choices were based on an analysis of the phylogenetic tree of Siglec family members indicating mouse CD33 and mouse Siglec-F are at a similar distance away from human Siglec-8. Each of the 3 immunogens were cloned into a lentivirus vector and NIH 3T3 mouse embryo fibroblast cells were transduced. A total of 30 ATX-GX mice were immunized. After 4 rounds of immunogen administrations, electrofusions from spleens of immunized mice were made. Using a 384-well plate approach, hybridoma cells were then plated. Supernatants were screened using K562 cells expressing either Siglec-8^{Hu}, CD33^{Mu}/Siglec-8^{Hu}, or Siglec-F^{Mu}/Siglec-8^{Hu}, and 19 polyclonal populations of hybridoma cells identified that exhibited antibody binding activity at least 5× higher (and up to 14× higher) binding to human Siglec-8-positive K562 target cells compared to parental K562 control cells, and 7 additional polyclonal populations of hybridoma cells that exhibit antibody binding activity at least 5× higher (and up to 20× higher) to mouse CD33/human Siglec-8-positive K562 target cells compared to parental K562 control cells. A total of 15 hybridomas were selected for subcloning. Flow cytometry-based phenotyping studies of supernatants of subcloned hybridoma cells identified not only hybridomas secreting mAbs against the V-set domain (Siglec-8^{dist} mAb) but also several hybridomas secreting mAbs against the membrane-proximal C2-set domain of human Siglec-8 (Siglec-8^{prox} mAb). The heavy and light chain variable regions for the Siglec-8 mAbs have been successfully sequenced in their entireties.

As indicated, the antibodies disclosed herein can be used in the treatment of eosinophil and/or mast cell related disorders. Eosinophils and mast cells are examples of granulocytes, innate immune cells that are polymorphonuclear and characterized by the presence of cytotoxic granules in their cytoplasm. Other types of granulocytes include neutrophils and basophils. Granulocytes generally express CD11b, CD13, CD15, CD16, CD32, and CD33. In particular embodiments, the antibodies disclosed herein can be used in the treatment of Siglec-8-related disorders.

Eosinophils, particularly, can be tissue resident cells and found in blood. In addition to granulocyte expression markers noted above, eosinophils can also express IL-5Ra, CCR3, CD193, Siglec-8 (human) or Siglec-F (mice), and EMR1 (human) or F4/80 (mice). Eosinophils can also be positive for CD9, CD15, CD24, CD35, CD43, CD64, CD116, CD123, CD125, CD126, CD244, and FcεR1 on the cell surface, and can secrete myelin basic protein (MBP), eosinophil-derived neurotoxin (EDN), and/or eosinophil peroxidase (EPX).

Mast cells are inflammatory cells primarily in connective tissue and can express CD117, stem cell factor (SCF) receptor, high-affinity IgE receptor (FcεR1), CD9, CD24, CD35, CD43, CD64, CD116, CD123, CD125, and IL-33R on the cell surface. Like eosinophils, mast cells can also express Siglec-8 (human) or Siglec-F (mice). Mast cells can secrete tryptases, chymases, carboxypeptidases, or a combination thereof; and cathepsins, histamine, tumor necrosis factor (TNF) alpha, IL-3, tumor growth factor (TGF) beta, and nerve growth factor (NGF).

Examples of eosinophil and/or mast cell related disorders include asthma, rhinitis, dermatitis, eosinophilic gastrointestinal disorders, eosinophilic pneumonia, anaphylaxis, urticaria, eosinophilic cellulitis and fasciitis, Churg-Strauss syndrome, hematologic malignancies involving eosinophils or mast cells, and mastocytosis.

In particular embodiments, the antibodies described herein can be used in engineered format for the treatment of subjects in need thereof. In particular embodiments, engineered formats include Siglec-8 mAb-based immune therapeutics or Siglec-8 mAb cell-based immunotherapy. In particular embodiments, Siglec-8 mAb-based immune therapeutics include multi-domain binding molecules and Siglec-8 Antibody Conjugates.

In particular embodiments, the Siglec-8 mAb cell-based immunotherapy includes an anti-Siglec-8 recombinant receptor expressed by a cell. In particular embodiments, the recombinant receptor includes a chimeric antigen receptor (CAR) or an engineered T cell receptor (eTCR). In particular embodiments, the cell includes an immune cell (e.g., T cell, B cell, or natural killer cell). In particular embodiments, the CAR includes an extracellular component linked to an intracellular component through a transmembrane domain. In particular embodiments, the extracellular component includes a binding domain disclosed herein. In particular embodiments, the extracellular component further includes a spacer region. In particular embodiments, the spacer region includes a 60 aa hinge spacer region. In particular embodiments, the transmembrane domain includes a CD28 transmembrane domain. In particular embodiments, the intracellular component includes a CD3ζ intracellular signaling domain and a 4-1BB intracellular signaling domain.

Aspects of the current disclosure are now described with additional details and options as follows: (i) Siglec-8 Antibodies; (ii) Antibody Variants; (iii) Multi-Domain Binding Molecules; (iv) Expression of Recombinant Proteins; (v) Siglec-8 Antibody Conjugates; (vi) Recombinant Receptors; (vii) Compositions and Formulations; (viii) Methods of Use; (ix) Kits; (x) Exemplary Embodiments; (xi) Experimental Example; and (xii) Closing Paragraphs. These headings are provided for organizational purposes only and do not limit the scope or interpretation of the disclosure.

(i) Siglec-8 Antibodies. Unless otherwise indicated, an antibody includes a tetramer structure with two full-length heavy chains and two full-length light chains. The amino-terminal portion of each chain includes a variable region that is responsible for antigen recognition and epitope binding. The variable regions exhibit the same general structure of relatively conserved framework regions (FR) joined by three hyper variable regions, also called complementarity determining regions (CDRs). The CDRs from the two chains of each pair are aligned by the framework regions, which enables binding to a specific epitope. From N-terminal to C-terminal, both light and heavy chain variable regions include the domains FR1, CDR1, FR2, CDR2, FR3, CDR3 and FR4.

The assignment of amino acids to each domain can be in accordance with Kabat numbering (Kabat et al. (1991), “Sequences of Proteins of Immunological Interest,” 5th Ed. Public Health Service, National Institutes of Health, Bethesda, Md. (“Kabat” numbering scheme)); Chothia (Al-Lazikani et al., (1997) JMB 273, 927-948 (“Chothia” numbering scheme)), Martin (Abinandan et al., Mol Immunol. 45:3832-3839 (2008), “Analysis and improvements to Kabat and structurally correct numbering of antibody variable domains”), Gelfand, Contact (MacCallum et al., J. Mol. Biol. 262:732-745 (1996), “Antibody-antigen interactions: Contact analysis and binding site topography,” J. Mol. Biol. 262, 732-745.” (Contact numbering scheme)), IMGT (Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme)), AHo (Honegger A and Plückthun A, “Yet another numbering scheme for immunoglobulin variable domains: an automatic modeling and analysis tool,” J Mol Biol, 2001 Jun. 8; 309(3):657-70, (AHo numbering scheme)), North (North et al., J Mol Biol. 406(2):228-256 (2011), “A new clustering of antibody CDR loop conformations”), or other numbering schemes.

Definitive delineation of a CDR and identification of residues including the binding site of an antibody can be accomplished by solving the structure of the antibody and/or solving the structure of the antibody-epitope complex. In particular embodiments, this can be accomplished by methods such as X-ray crystallography and cryoelectron microscopy. Alternatively, CDRs are determined by comparison to known antibodies (linear sequence) and without resorting to solving a crystal structure. To determine residues involved in binding, a co-crystal structure of the Fab (antibody fragment) bound to the target can optionally be determined. Software programs and bioinformatical tools, such as ABodyBuilder and Paratome can also be used to determine CDR sequences.

The carboxy-terminal portion of each chain defines a constant region, which can be responsible for effector function particularly in the heavy chain (the Fc). Examples of effector functions include: C1q binding and complement dependent cytotoxicity (CDC); antibody-dependent cell-mediated cytotoxicity (ADCC); phagocytosis; down regulation of cell surface receptors (e.g., B-cell receptors); and B-cell activation.

Human light chains are classified as kappa (Igκ) and lambda (Igλ) light chains. In particular embodiments, a human Igκ fragment crystallizable (Fc) region includes the sequence: RTVAAPSVFIFPPSDEQLKSGTASVVCLLNNFYPREAKVQWKVDNALQSGNSQESVTEQDSKD STYSLSSTLTLSKADYEKHKVYACEVTHQGLSSPVTKSFNRGEC (SEQ ID NO: 337). In particular embodiments, a human IgA Fc region includes the sequence:

(SEQ ID NO: 338)
GQPKANPTVTLFPPSSEELQANKATLVCLISDFYPGAVTVAWKADGSPVK
AGVETTKPSKQSNNKYAASSYLSLTPEQWKSHRSYSCQVTHEGSTVEKTV
APTECS.

Heavy chains are classified as mu, delta, gamma, alpha, or epsilon, and define the antibody's isotype as IgM, IgD, IgG, IgA, and IgE, respectively. IgG has several subclasses, including, IgG1, IgG2, IgG3, and IgG4. IgM has subclasses including IgM1 and IgM2. IgA is similarly subdivided into subclasses including IgA1 and IgA2. IgG causes opsonization and cellular cytotoxicity and crosses the placenta, IgA functions on the mucosal surface, IgM is most effective in complement fixation, and IgE mediates degranulation of mast cells and basophils. The function of IgD is still not well understood. Resting B cells, which are immunocompetent but not yet activated, express IgM and IgD. Once activated and committed to secrete antibodies these B cells can express any of the five isotypes. The heavy chain isotypes of IgG, IgA, IgM, IgD and IgE are respectively designated the γ, α, μ, δ, and ε chains.

The constant region of the antibody with multiple binding domains may be of any suitable immunoglobulin subtype. In particular embodiments the subtype of the antibody may be of the class IgG, IgD, IgE, IgA, or IgM. Such an antibody may further belong to any subclass, e.g., IgG1, IgG2a, IgG2b, IgG3 and IgG4. In particular embodiments, a constant region includes a light chain constant region and a heavy chain constant region. A “functional constant heavy chain” or “functional CH” activates an aspect of the immune response.

In particular embodiments, a human IgG1 Fc region includes the sequence:

(SEQ ID NO: 339)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK.

In particular embodiments, a human IgG1 Fc region includes the sequence:

(SEQ ID NO: 340)
THTCPPCPAPEFFGGPSVFFFPPKPKDTFMISRTPEVTCVVVDVSHEDPE
VKFNWYVDGVEVHNAKTKPREEQYNSTYRVVSVETVFHQDWENGKEYKCK
VSNKAFPVPIEKTISKAKGQPREPQVYTLPPSREEMTKNQVSLTCLVKGF
YPSDIAVEWESNGQPENNYKTTPPVLDSDGPFFLYSKLTVDKSRWQQGNV
FSCSVMHEALHNHYTQKSLSLSPGK.

In particular embodiments, a human IgG2 Fc region includes the amino acid sequence:

(SEQ ID NO: 341)
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDISVEWESNGQPENNYKTTPPMLDSDGSFFLYSKLTVDKSRWQQGN
VFSCSVMHEALHNHYTQKSLSLSPGK.

In particular embodiments, a human IgG2 Fc region includes the amino acid sequence:

(SEQ ID NO: 342)
PAPPVAGPSVFLFPPKPKDTLMISRTPEVTCVVVDVSHEDPEVQFNWYVD
GVEVHNAKTKPREEQFNSTFRVVSVLTVVHQDWLNGKEYKCKVSNKGLPA
PIEKTISKTKGQPREPQVYTLPPSREEMTKNQVSLTCLVKGFYPSDIAVE
WESNGQPENNYKTTPPMLDSDGSFFLYSKLTVDKSRWQQGNVFSCSVMHE
ALHNHYTQKSLSLSPGK.

In particular embodiments, a human IgG3 Fc region includes the amino acid sequence:

(SEQ ID NO: 343)
ASTKGPSVFPLAPCSRSTSGGTAALGCLVKDYFPEPVTVSWNSGALTSGV
HTFPAVLQSSGLYSLSSVVTVPSSSLGTQTYTCNVNHKPSNTKVDKRVEL
KTPLGDTTHTCPRCPEPKSCDTPPPCPRCPEPKSCDTPPPCPRCPEPKSC
DTPPPCPRCPAPELLGGPSVFLFPPKPKDTLMISRTPEVTCVVVDVSHED
PEVQFKWYVDGVEVHNAKTKPREEQYNSTFRVVSVLTVLHQDWLNGKEYK
CKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQG
NIFSCSVMHEALHNRFTQKSLSLSPGK.

In particular embodiments, a human IgG3 Fc region includes the amino acid sequence:

(SEQ ID NO: 344)
PAPELLGGPSVFLFPPKPKDTLMISRTPEVTCVVVDVSHEDPEVQFKWYV
DGVEVHNAKTKPREEQFNSTFRVVSVLTVLHQDWLNGKEYKCKVSNKALP
APIEKTISKTKGQPREPQVYTLPPSREEMTKNQVSLTCLVKGFYPSDIAV
EWESSGQPENNYNTTPPMLDSDGSFFLYSKLTVDKSRWQQGNIFSCSVMH
EALHNRFTQKSLSLSPGK.

In particular embodiments, a human IgG4 Fc region includes the amino acid sequence:

(SEQ ID NO: 345)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK.

In particular embodiments, a human IgG4 Fc region includes the amino acid sequence:

(SEQ ID NO: 346)
PAPEFLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLP
SSIEKTISKAKGQPREPQVYTLPPSQEEMTKNQVSLTCLVKGFYPSDIAV
EWESNGQPENNYKTTPPVLDSDGSFFLYSRLTVDKSRWQEGNVFSCSVMH
EALHNHYTQKSLSLSLGK.

In particular embodiments, a human IgA1 constant region includes the amino acid sequence:

(SEQ ID NO: 377)
ASPTSPKVFPLSLCSTQPDGNVVIACLVQGFFPQEPLSVTWSESGQGVTA
RNFPPSQDASGDLYTTSSQLTLPATQCLAGKSVTCHVKHYTNPSQDVTVP
CPVPSTPPTPSPSTPPTPSPSCCHPRLSLHRPALEDLLLGSEANLTCTLT
GLRDASGVTFTWTPSSGKSAVQGPPERDLCGCYSVSSVLPGCAEPWNHGK
TFTCTAAYPESKTPLTATLSKSGNTFRPEVHLLPPPSEELALNELVTLTC
LARGFSPKDVLVRWLQGSQELPREKYLTWASRQEPSQGTTTFAVTSILRV
AAEDWKKGDTFSCMVGHEALPLAFTQKTIDRLAGKPTHVNVSVVMAEVDG
TCY.

In particular embodiments, a human IgA2 constant region includes the amino acid sequence:

(SEQ ID NO: 378)
ASPTSPKVFPLSLDSTPQDGNVVVACLVQGFFPQEPLSVTWSESGQNVTA
RNFPPSQDASGDLYTTSSQLTLPATQCPDGKSVTCHVKHYTNPSQDVTVP
CPVPPPPPCCHPRLSLHRPALEDLLLGSEANLTCTLTGLRDASGATFTWT
PSSGKSAVQGPPERDLCGCYSVSSVLPGCAQPWNHGETFTCTAAHPELKT
PLTANITKSGNTFRPEVHLLPPPSEELALNELVTLTCLARGFSPKDVLVR
WLQGSQELPREKYLTWASRQEPSQGTTTFAVTSILRVAAEDWKKGDTFSC
MVGHEALPLAFTQKTIDRLAGKPTHVNVSVVMAEVDGTCY.

In particular embodiments, a human IgM heavy chain constant region includes the amino acid sequence:

(SEQ ID NO: 382)
VIAELPPKVSVFVPPRDGFFGNPRKSKLICQATGFSPRQIQVSWLREGKQ
VGSGVTTDQVQAEAKESGPTTYKVTSTLTIKESDWLSQSMFTCRVDHRGL
TFQQNASSMCVPDQDTAIRVFAIPPSFASIFLTKSTKLTCLVTDLTTYDS
VTISWTRQNGEAVKTHTNISESHPNATFSAVGEASICEDDWNSGERFTCT
VTHTDLPSPLKQTISRPKGVALHRPDVYLLPPAREQLNLRESATITCLVT
GFSPADVFVQWMQRGQPLSPEKYVTSAPMPEPQAPGRYFAHSILTVSEEE
WNTGETYTCVVAHEALPNRVTERTVDKSTGKPTLYNVSLVMSDTAGTCY.

In particular embodiments, the human IgM heavy chain constant region includes the amino acid sequence: GSASAPTLFPLVSCENSPSDTSSVAVGCLAQDFLPDSITFSWKYKNNSDISSTRGFPSVLRGGK YAATSQVLLPSKDVMQGTDEHVVCKVQHPNGNKEKNVPLPVIAELPPKVSVFVPPRDGFFGNP RKSKLICQATGFSPRQIQVSWLREGKQVGSGVTTDQVQAEAKESGPTTYKVTSTLTIKESDWL SQSMFTCRVDHRGLTFQQNASSMCVPDQDTAIRVFAIPPSFASIFLTKSTKLTCLVTDLTTYDSV TISWTRQNGEAVKTHTNISESHPNATFSAVGEASICEDDWNSGERFTCTVTHTDLPSPLKQTIS RPKGVALHRPDVYLLPPAREQLNLRESATITCLVTGFSPADVFVQWMQRGQPLSPEKYVTSAP MPEPQAPGRYFAHSILTVSEEEWNTGETYTCVVAHEALPNRVTERTVDKSTGKPTLYNVSLVM SDTAGTCY (SEQ ID NO: 385; identical to, e.g., GenBank Accession Nos. pir∥S37768, CAA47708.1, and CAA47714.1). Referring to this SEQ ID NO: 385, the human Cμ1 region ranges from amino acid 5 to amino acid 102; the human Cμ² region ranges from amino acid 114 to amino acid 205, the human Cμ3 region ranges from amino acid 224 to amino acid 319, the Cμ4 region ranges from amino acid 329 to amino acid 430, and the tp ranges from amino acid 431 to amino acid 453.

In particular embodiments, the human IgM heavy chain constant region includes the amino acid sequence: GSASAPTLFPLVSCENSPSDTSSVAVGCLAQDFLPDSITFSWKYKNNSDISSTRGFPSVLRGGK YAATSQVLLPSKDVMQGTDEHVVCKVQHPNGNKEKNVPLPVIAELPPKVSVFVPPRDGFFGNP RKSKLICQATGFSPRQIQVSWLREGKQVGSGVTTDQVQAEAKESGPTTYKVTSTLTIKESDWL GQSMFTCRVDHRGLTFQQNASSMCVPDQDTAIRVFAIPPSFASIFLTKSTKLTCLVTDLTTYDS VTISWTRQNGEAVKTHTNISESHPNATFSAVGEASICEDDWNSGERFTCTVTHTDLPSPLKQTI SRPKGVALHRPDVYLLPPAREQLNLRESATITCLVTGFSPADVFVQWMQRGQPLSPEKYVTSA PMPEPQAPGRYFAHSILTVSEEEWNTGETYTCVVAHEALPNRVTERTVDKSTGKPTLYNVSLV MSDTAGTCY (SEQ ID NO: 386; (UniProt ID P01871)—allele IGHM*04). This sequence differs from SEQ ID NO: 385 by one amino acid at position 191.

Within full-length light and heavy chains, the variable and constant regions are joined by a “J” region of amino acids, with the heavy chain also including a “D” region of amino acids. See, e.g., Fundamental Immunology, Ch. 7 (Paul, W., ed., 2nd ed. Raven Press, N.Y. (1989)).

Antibodies bind epitopes on antigens. The term antigen refers to a molecule or a portion of a molecule capable of being bound by an antibody. An epitope is a region of an antigen that is bound by the variable region of an antibody. Epitope determinants can include chemically active surface groupings of molecules such as amino acids, sugar side chains, phosphoryl or sulfonyl groups, and can have specific three-dimensional structural characteristics, and/or specific charge characteristics. When the antigen is a protein or peptide, the epitope includes specific amino acids within that protein or peptide that contact the variable region of an antibody.

Unless otherwise indicated, the term “antibody” includes (in addition to antibodies having two full-length heavy chains and two full-length light chains as described above) variants, derivatives, and fragments thereof, examples of which are described below. Furthermore, unless explicitly excluded, antibodies can include monoclonal antibodies, polyclonal antibodies, human antibodies, humanized antibodies, bispecific antibodies, trispecific antibodies, tetraspecific antibodies, multi-specific antibodies, linear antibodies, minibodies, domain antibodies, synthetic antibodies, single-chain variable fragments (scFv), chimeric antibodies, antibody fusions, and fragments thereof, respectively. In particular embodiments, antibodies can include oligomers or multiplexed versions of antibodies.

A monoclonal antibody refers to an antibody obtained from a population of substantially homogeneous antibodies, i.e., the individual antibodies including the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which include different antibodies directed against different epitopes, each monoclonal antibody of a monoclonal antibody preparation is directed against a single epitope on an antigen. Thus, the modifier “monoclonal” indicates the character of the antibody as being obtained from a substantially homogeneous population of antibodies and is not to be construed as requiring production of the antibody by any particular method. For example, monoclonal antibodies can be made by a variety of techniques, including the hybridoma method, recombinant DNA methods, phage-display methods, and methods utilizing transgenic animals containing all or part of the human immunoglobulin loci.

A “human antibody” is one which includes an amino acid sequence which corresponds to that of an antibody produced by a human or a human cell or derived from a non-human source that utilizes human antibody repertoires or other human antibody-encoding sequences.

A “human consensus framework” is a framework that represents the most commonly occurring amino acid residues in a selection of human immunoglobulin V_{L} or V_{H} framework sequences. Generally, the selection of human immunoglobulin variable light chain (V_{L}) or variable heavy chain (V_{H}) sequences is from a subgroup of variable domain sequences. The subgroup of sequences can be a subgroup as in Kabat et al., Sequences of Proteins of Immunological Interest, Fifth Edition, NIH Publication 91-3242, Bethesda Md. (1991), vols. 1-3. In particular embodiments, for the V_{L}, the subgroup is subgroup kappa I as in Kabat et al. (supra). In particular embodiments, for the V_{H}, the subgroup is subgroup III as in Kabat et al. (supra).

The choice of constant region can depend, in part, whether antibody-dependent cell-mediated cytotoxicity, antibody dependent cellular phagocytosis and/or complement dependent cytotoxicity are desired. For example, human isotopes IgG1 and IgG3 have strong complement-dependent cytotoxicity, human isotype IgG2 has weak complement-dependent cytotoxicity and human IgG4 lacks complement-dependent cytotoxicity. Human IgG1 and IgG3 also induce stronger cell mediated effector functions than human IgG2 and IgG4.

In particular embodiments, a light chain constant region includes a human Igκ Fc region or a human Igλ Fc region.

In particular embodiments, a heavy chain constant region includes a human IgG1 Fc region or a human IgG4 Fc region. In particular embodiments, a human IgG4 Fc region includes an IgG4 (S228P) Fc region.

In particular embodiments, IgG4 (S228P) Fc region includes the sequence:

(SEQ ID NO: 84)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK.

Human constant regions show allotypic variation and isoallotypic variation between different individuals, that is, the constant regions can differ in different individuals at one or more polymorphic positions. Isoallotypes differ from allotypes in that sera recognizing an isoallotype bind to a non-polymorphic region of one or more other isotypes.

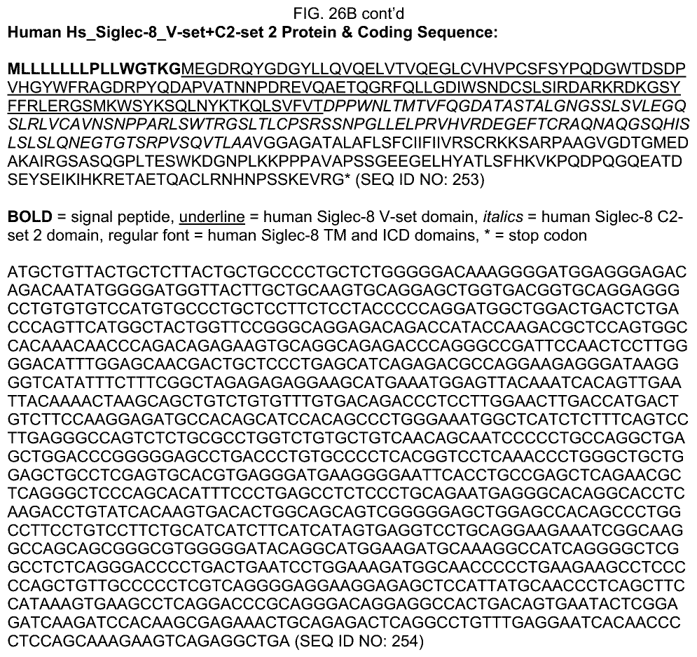

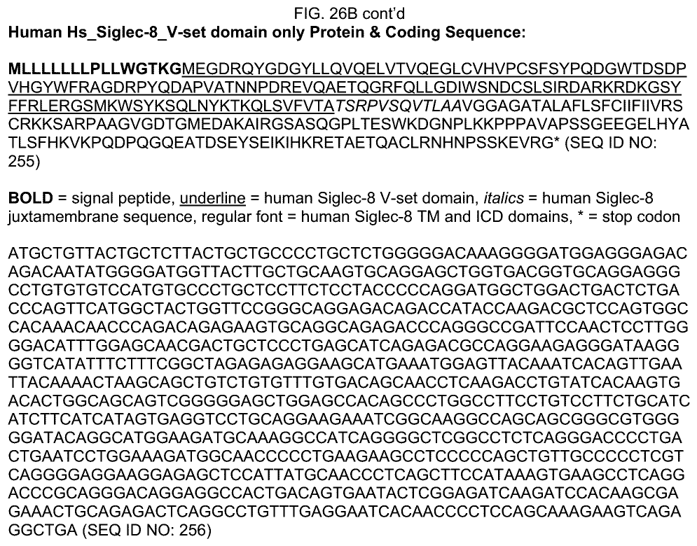

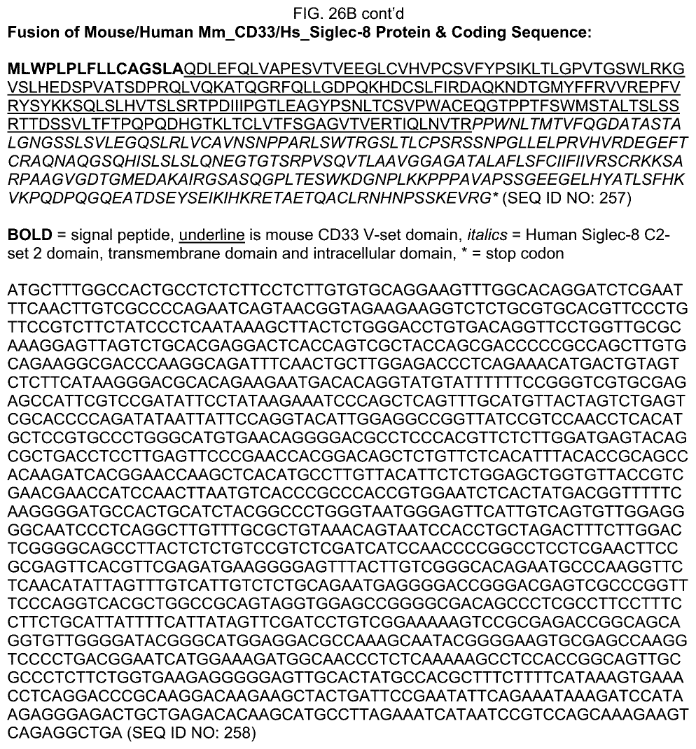

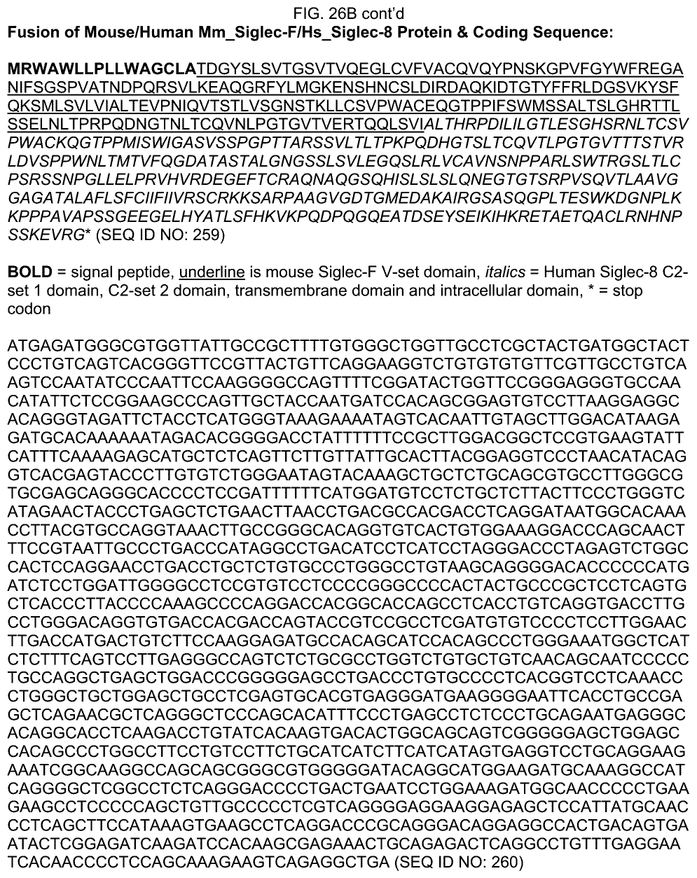

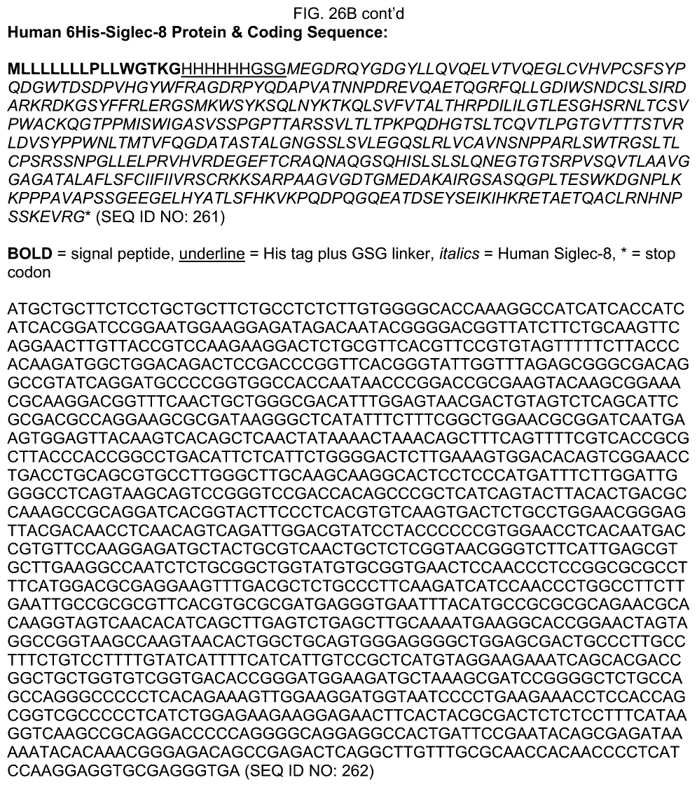

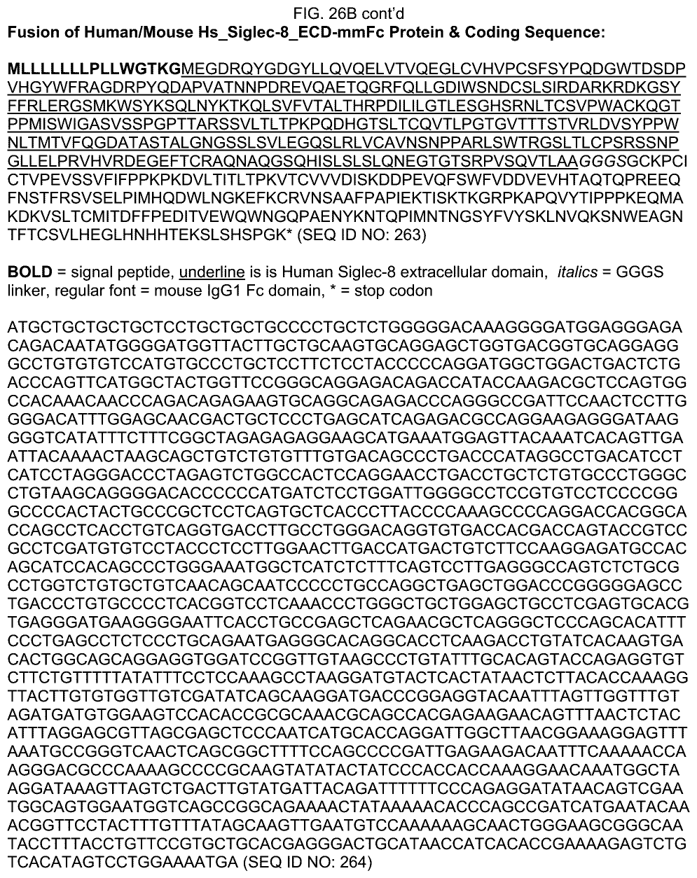

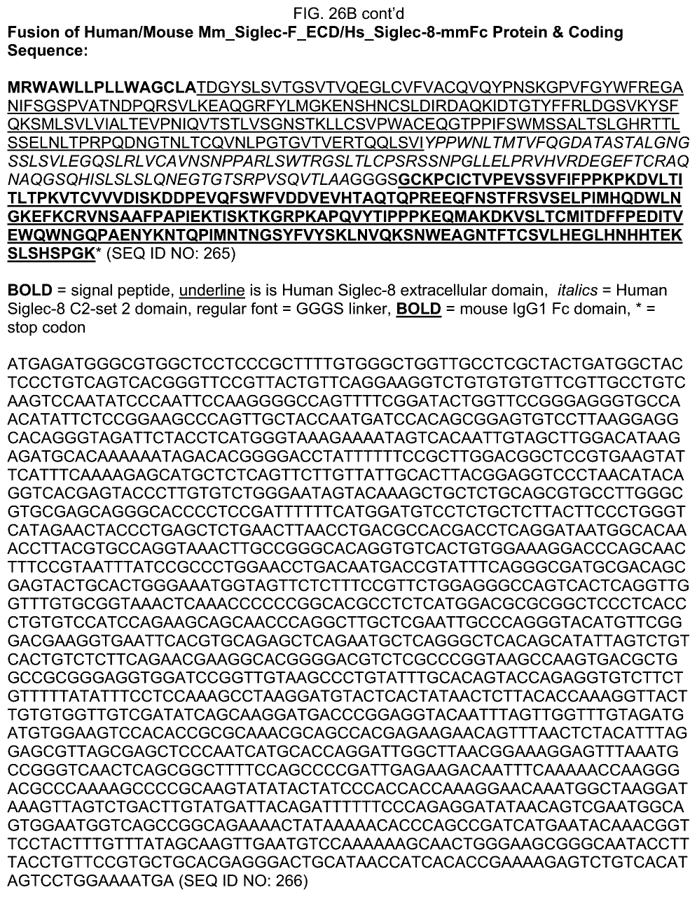

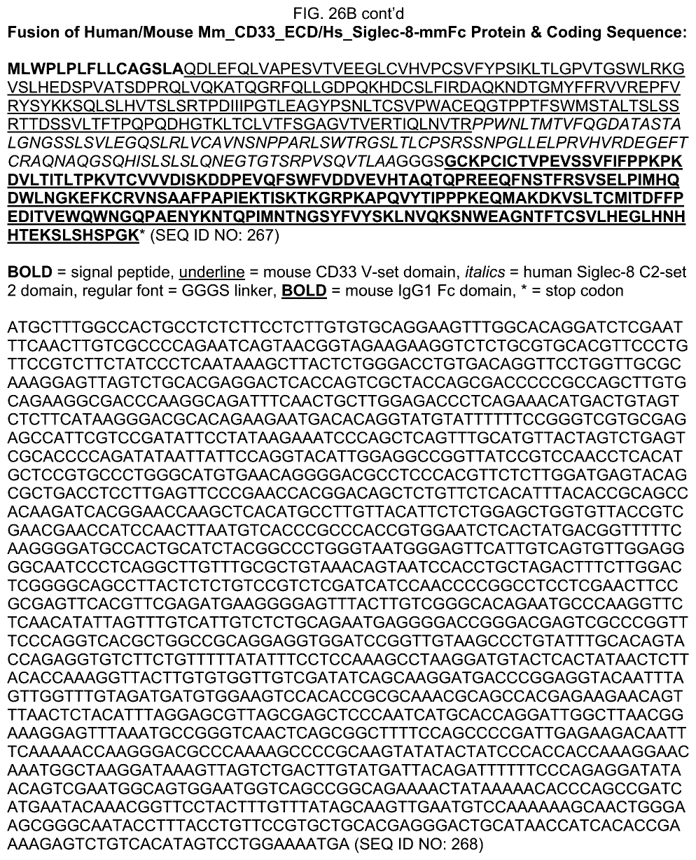

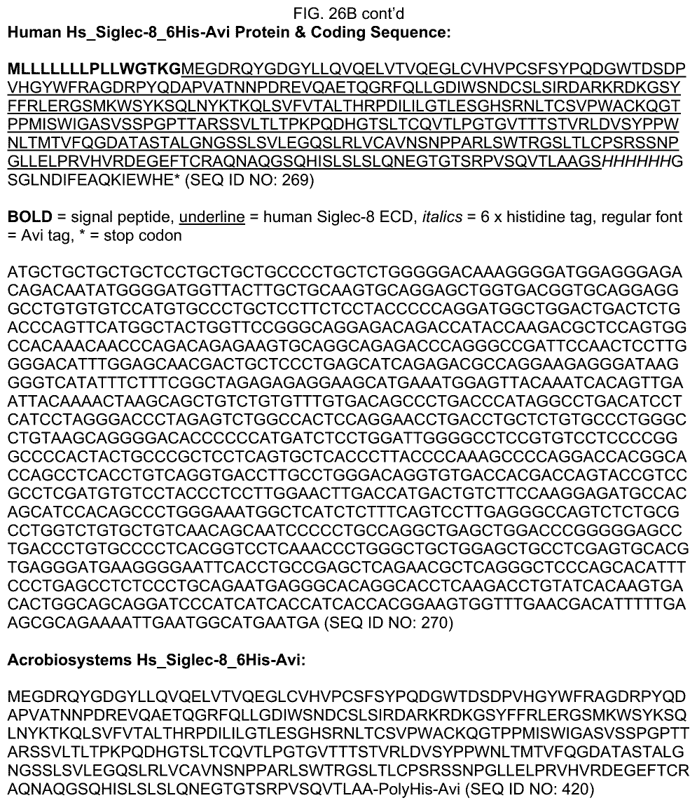

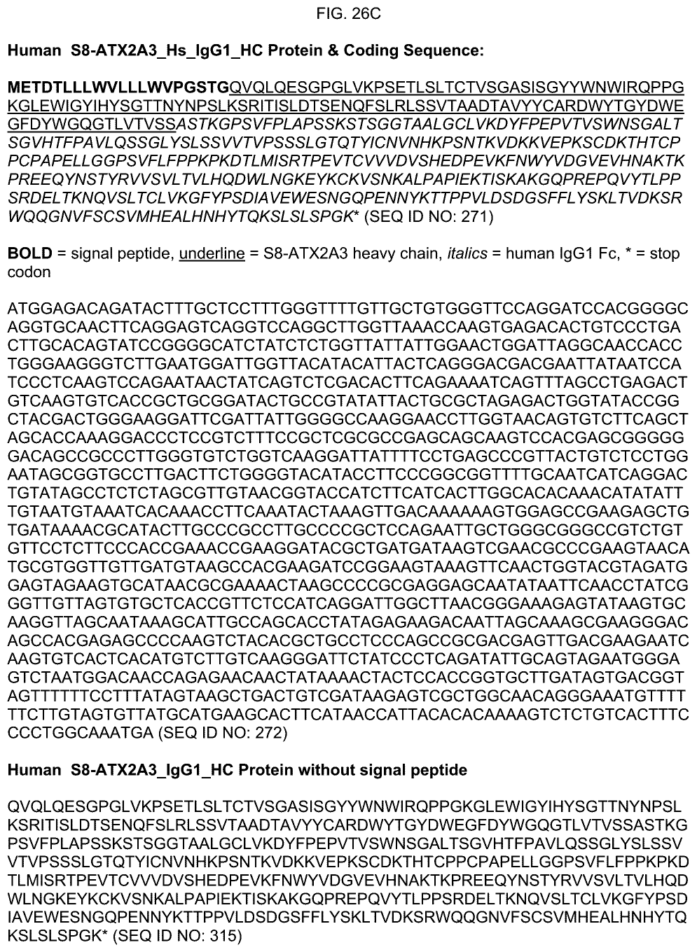

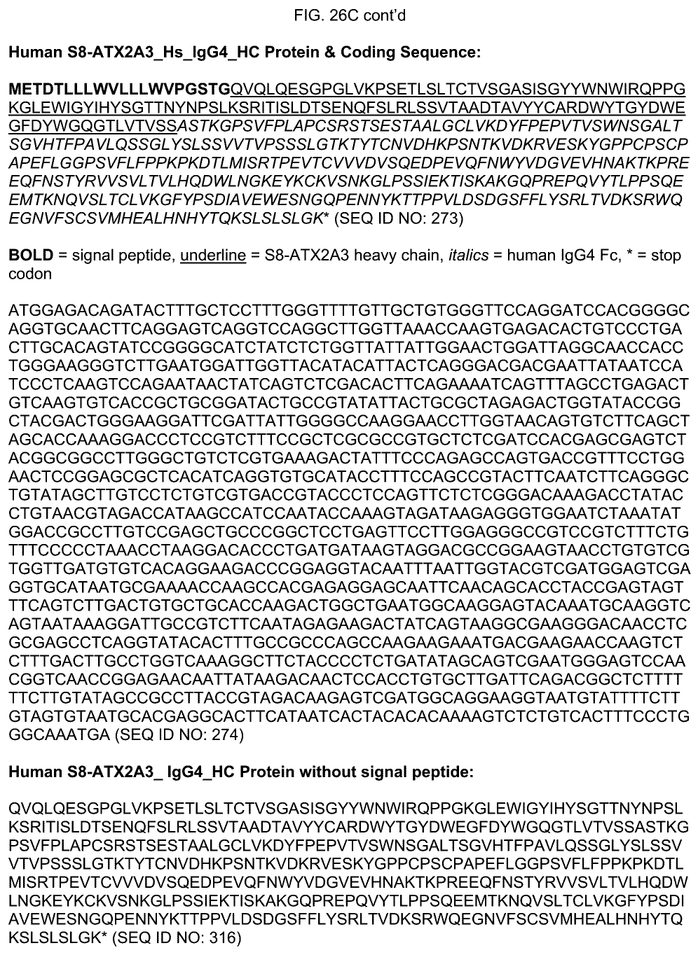

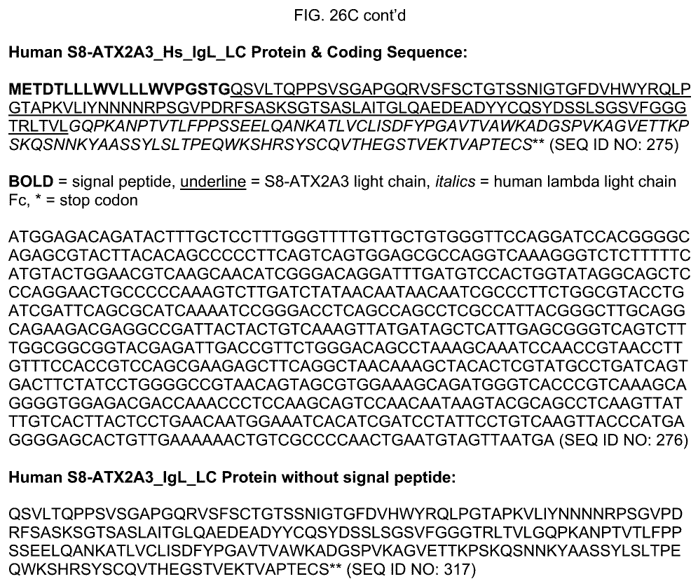

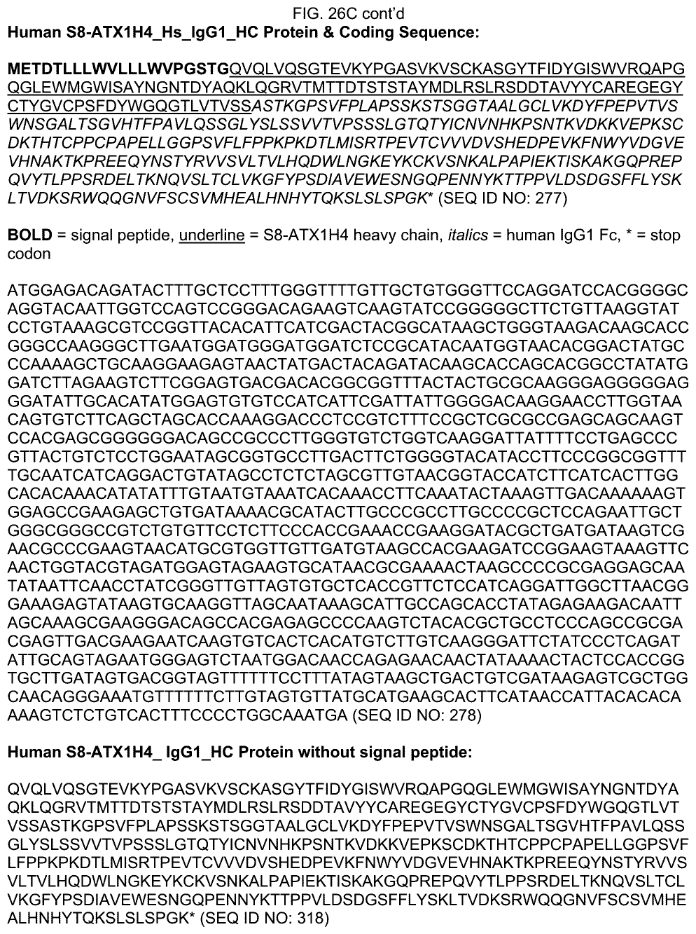

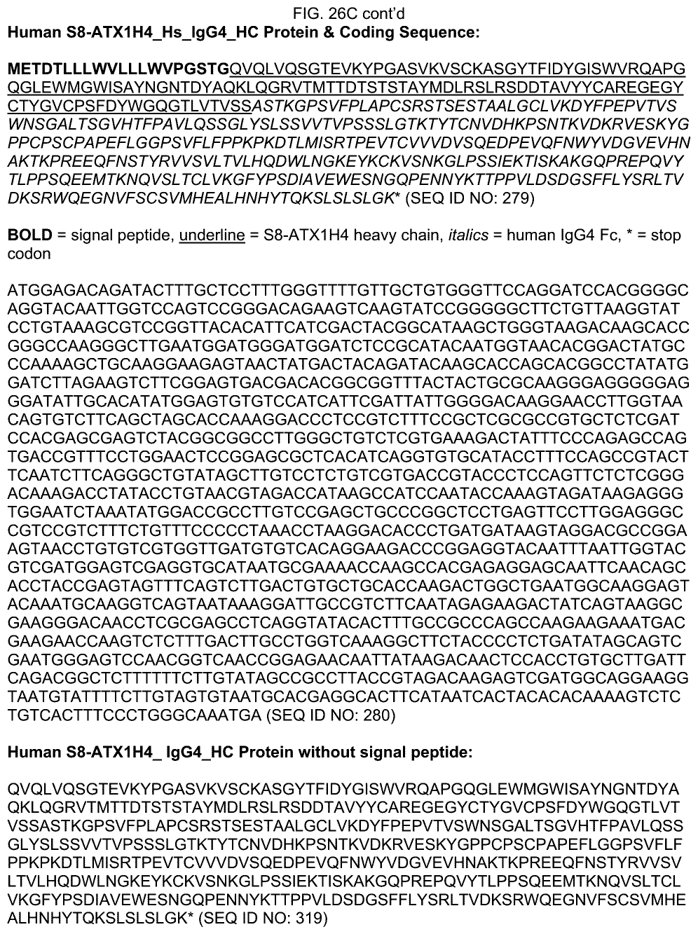

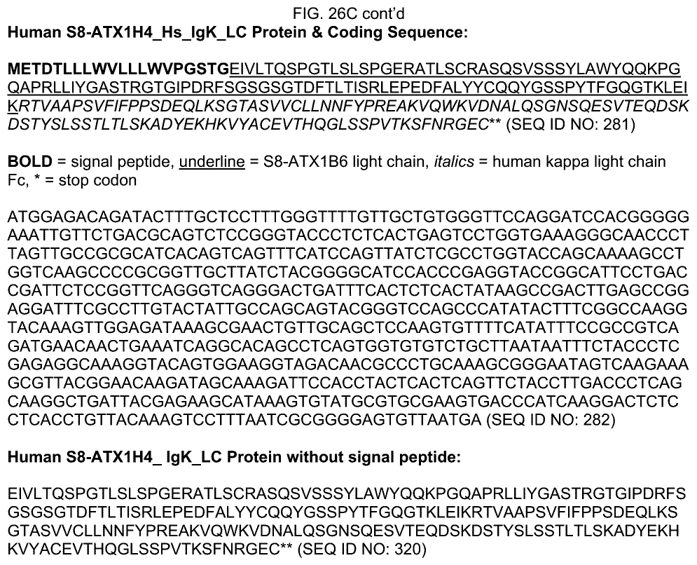

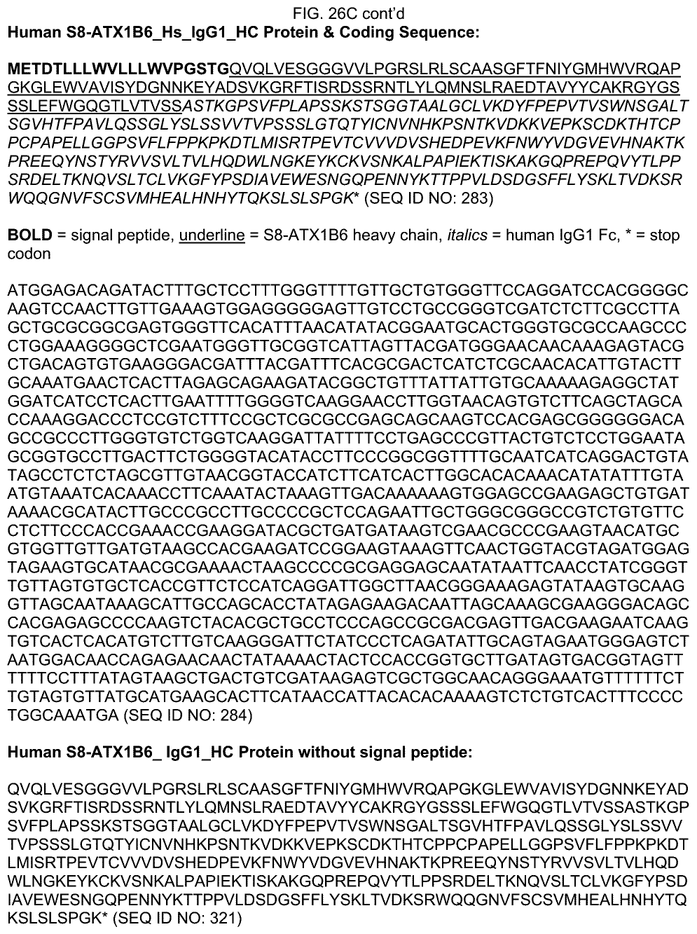

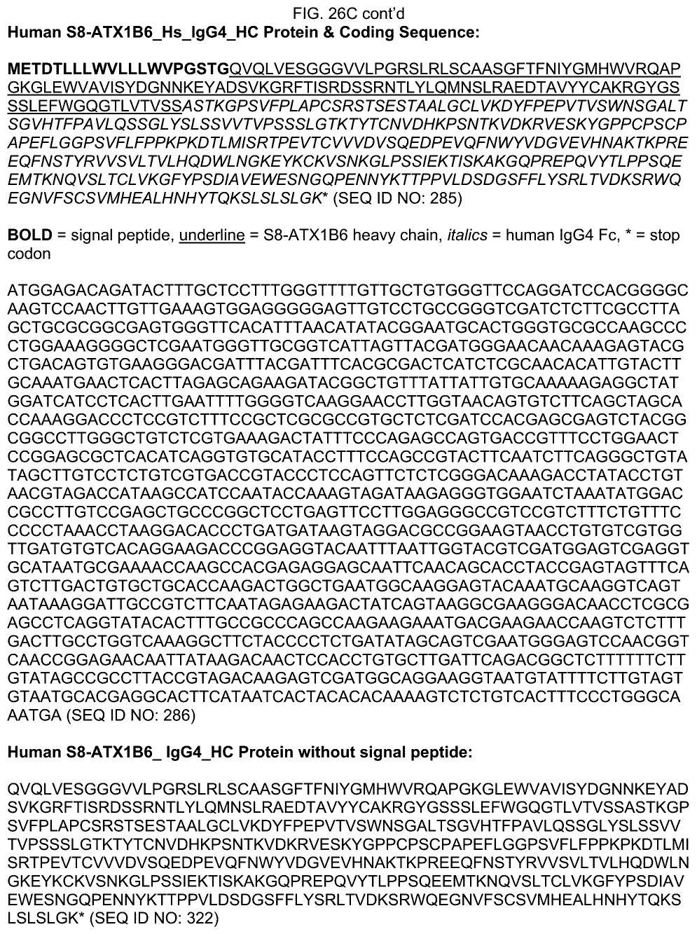

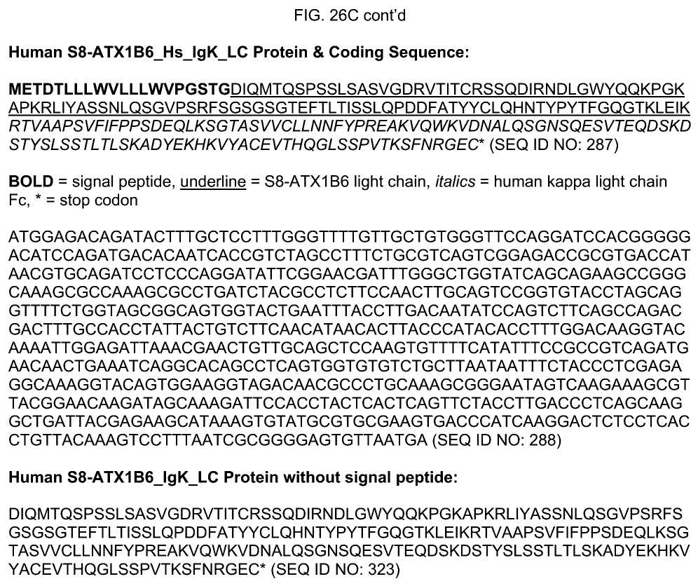

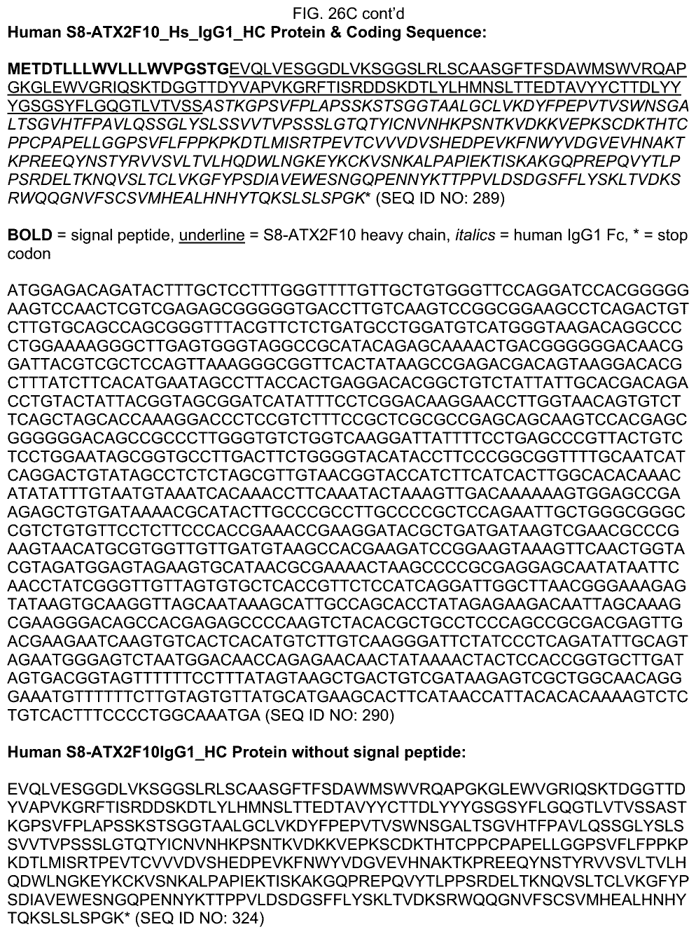

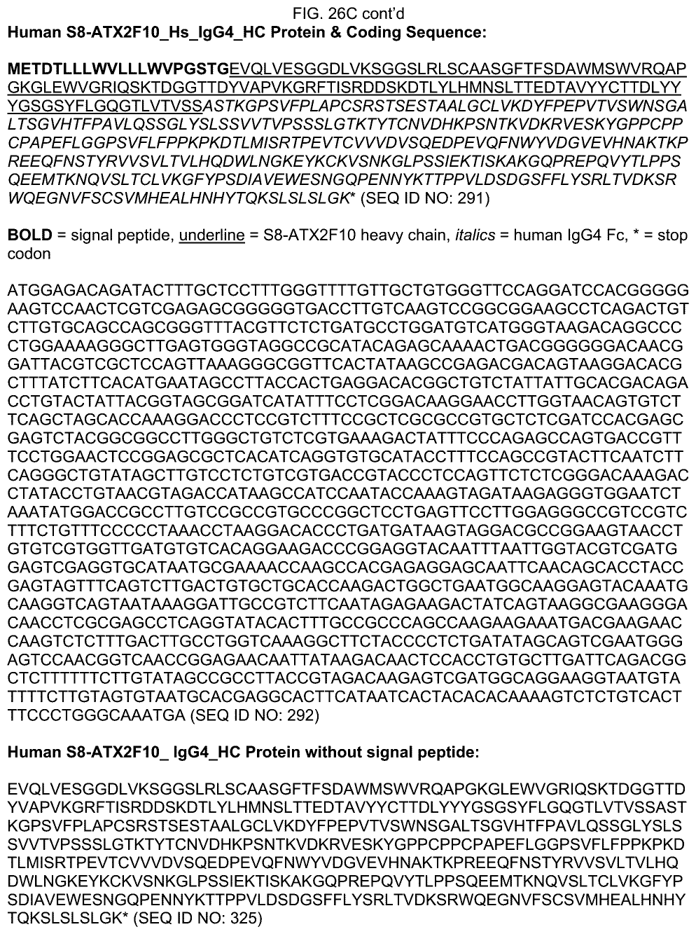

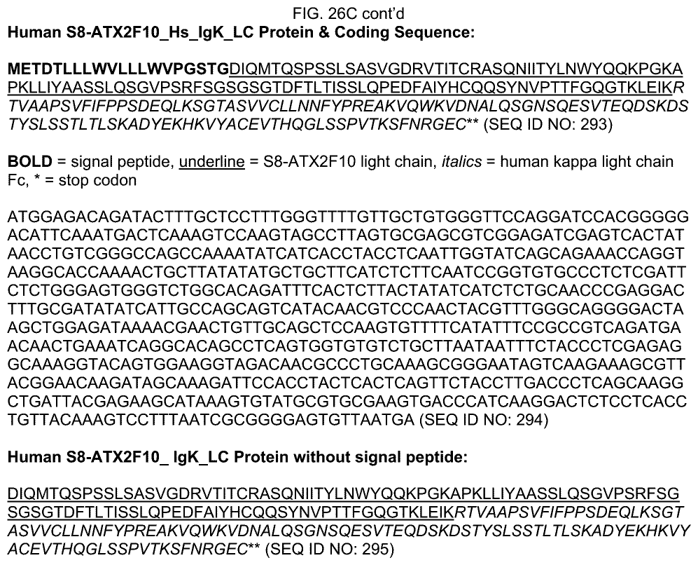

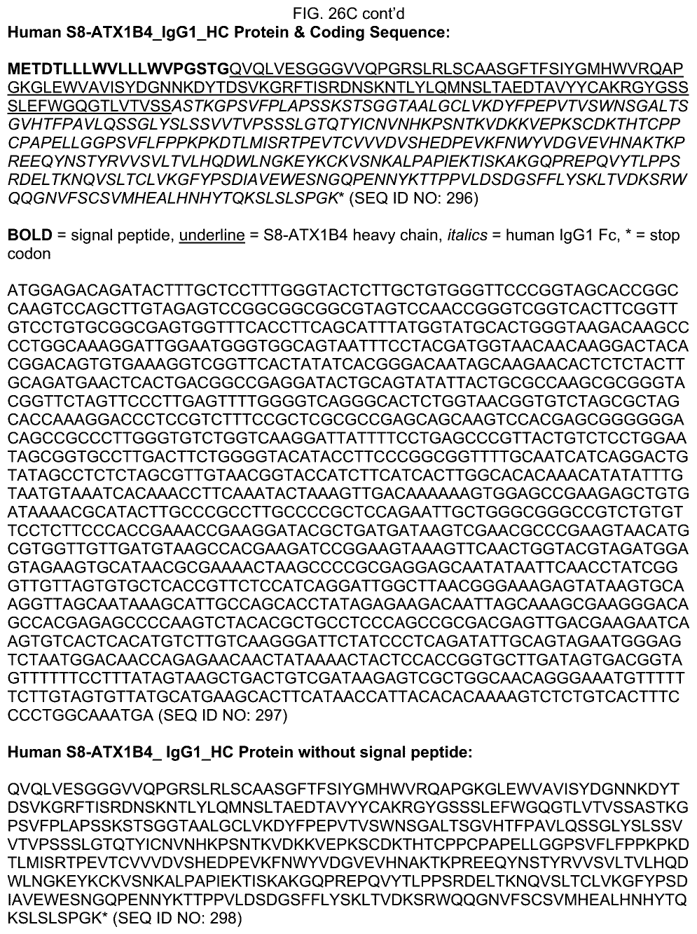

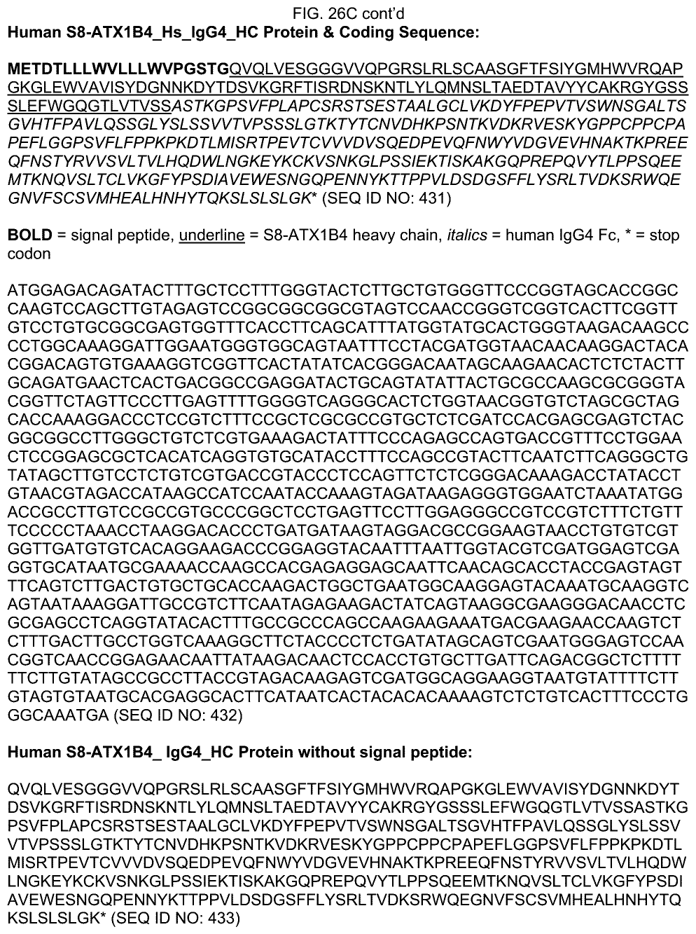

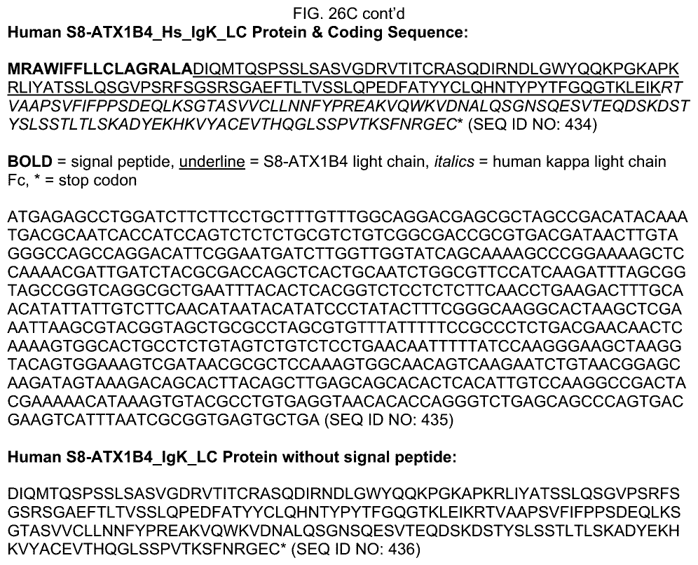

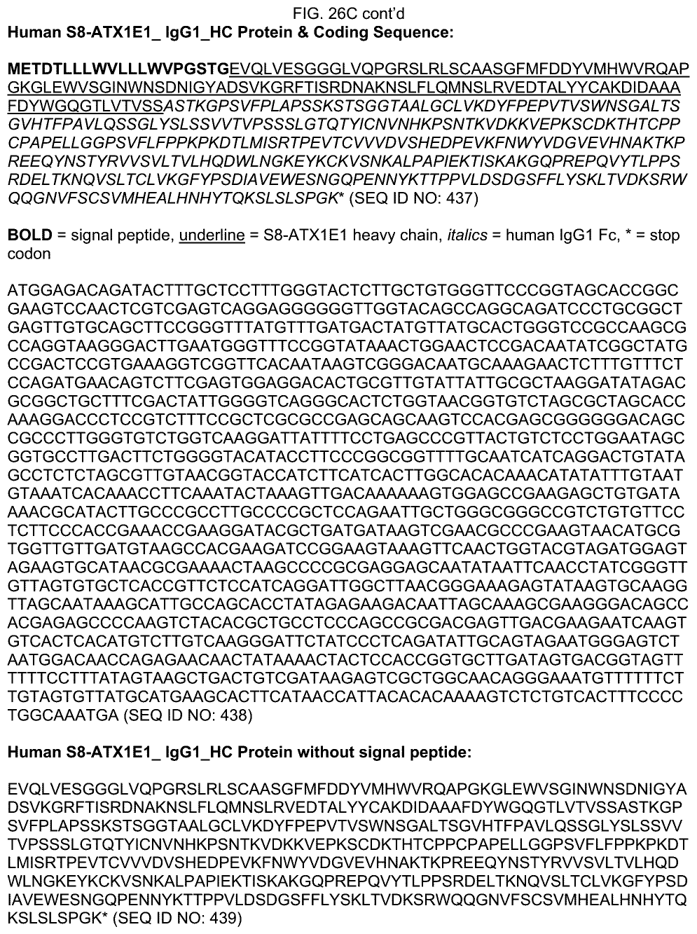

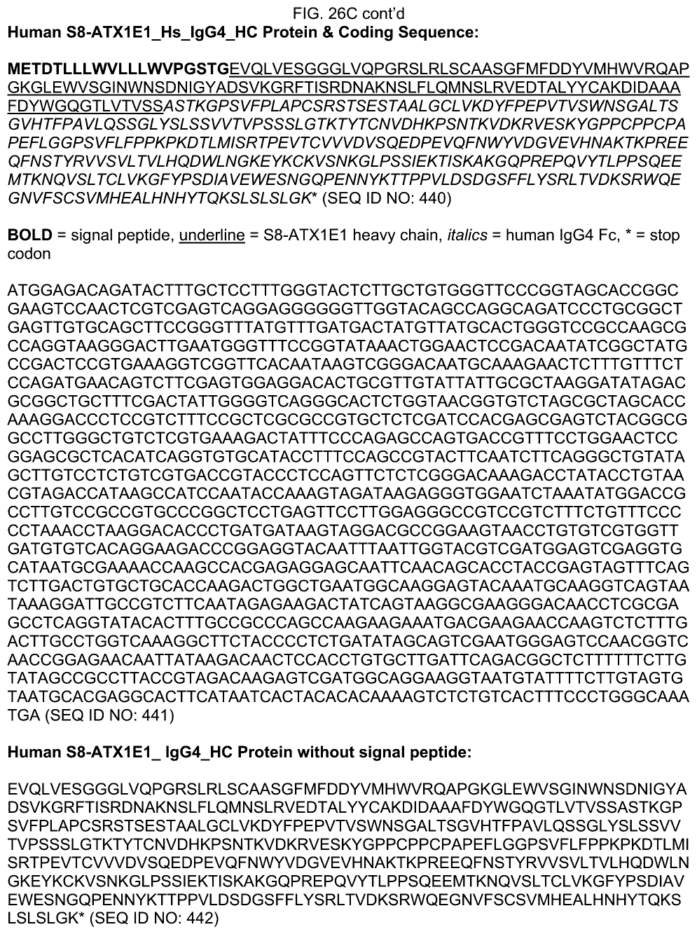

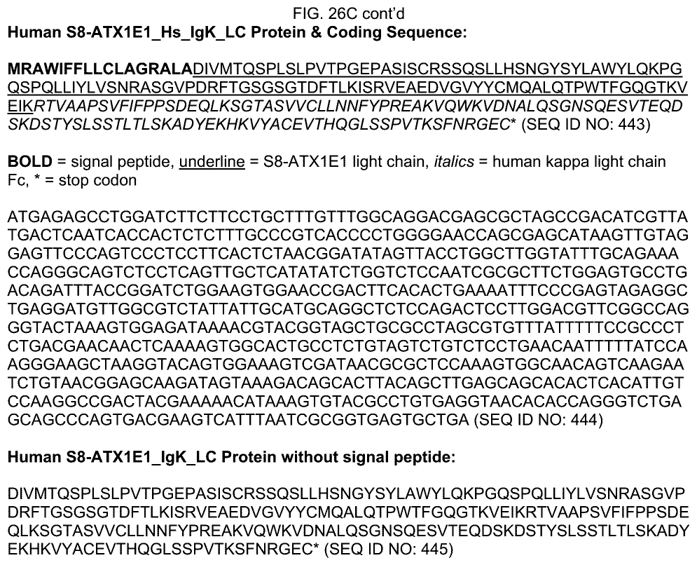

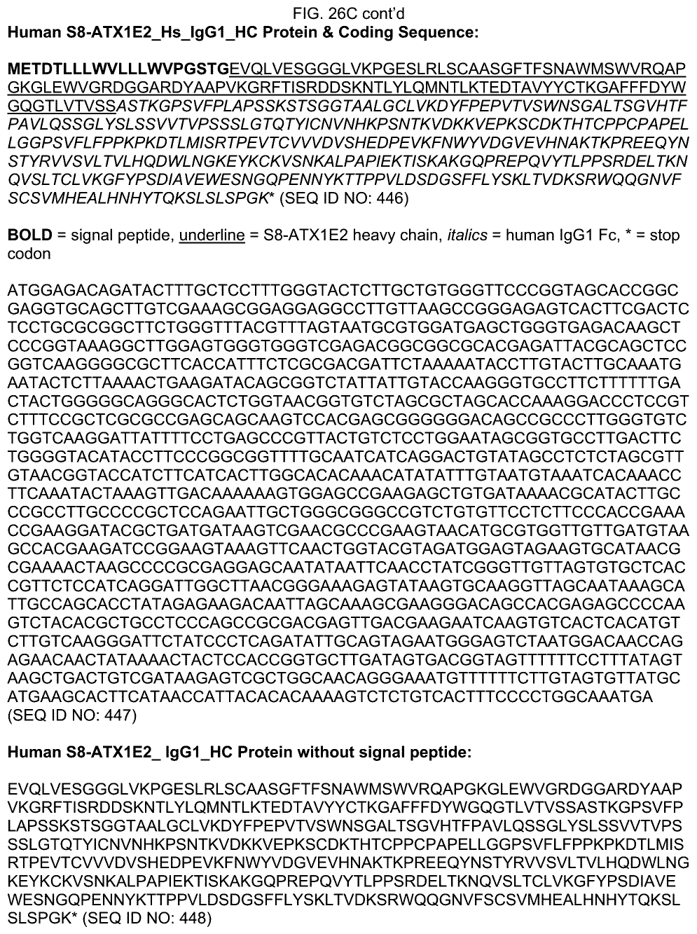

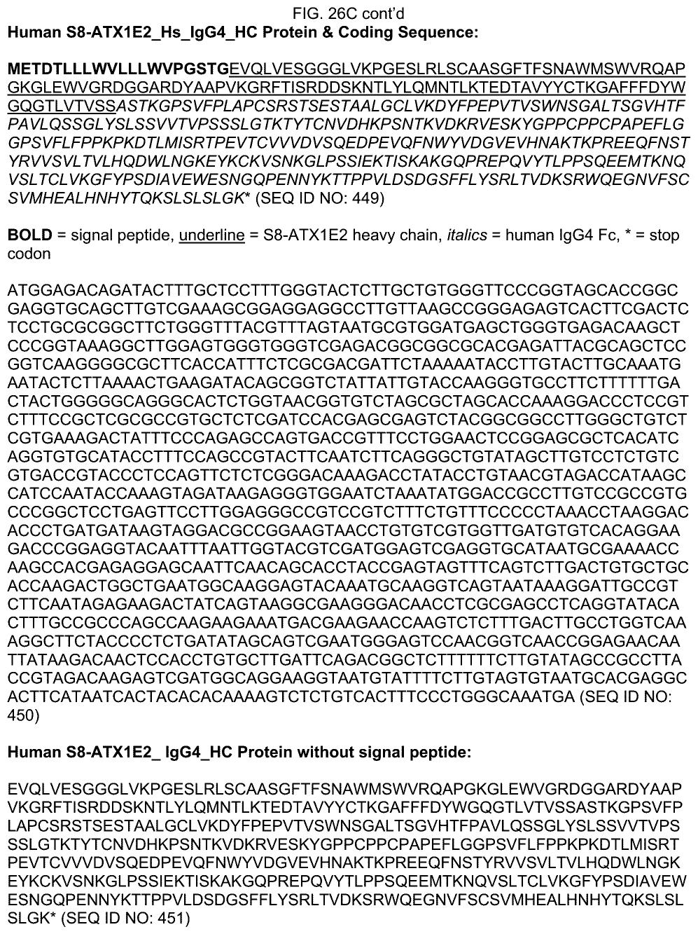

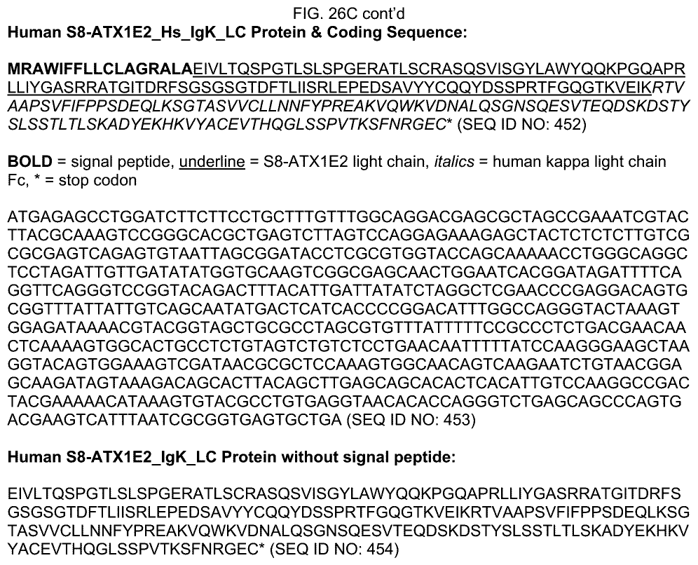

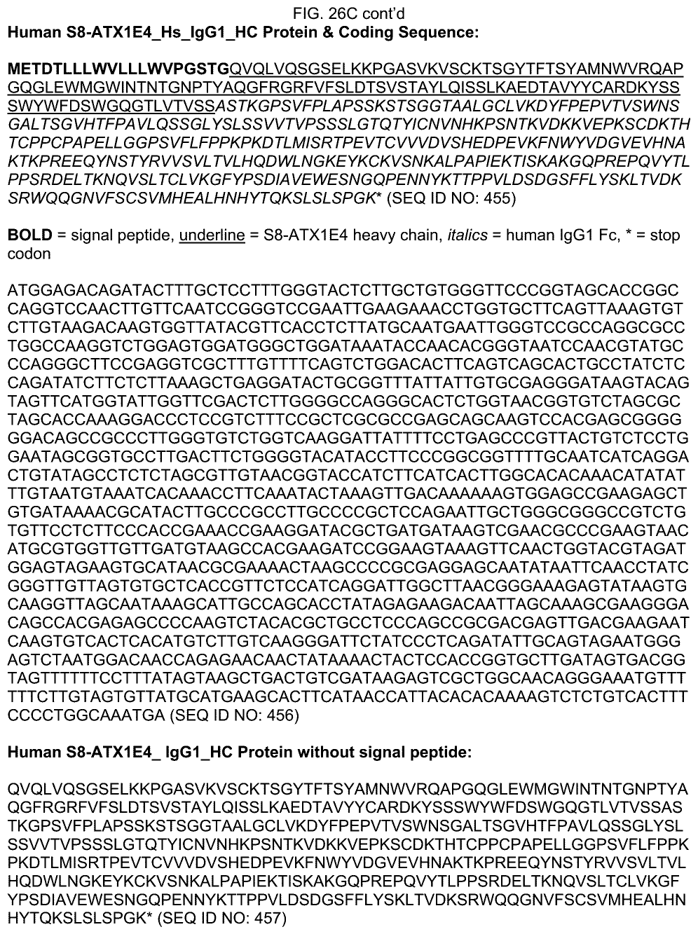

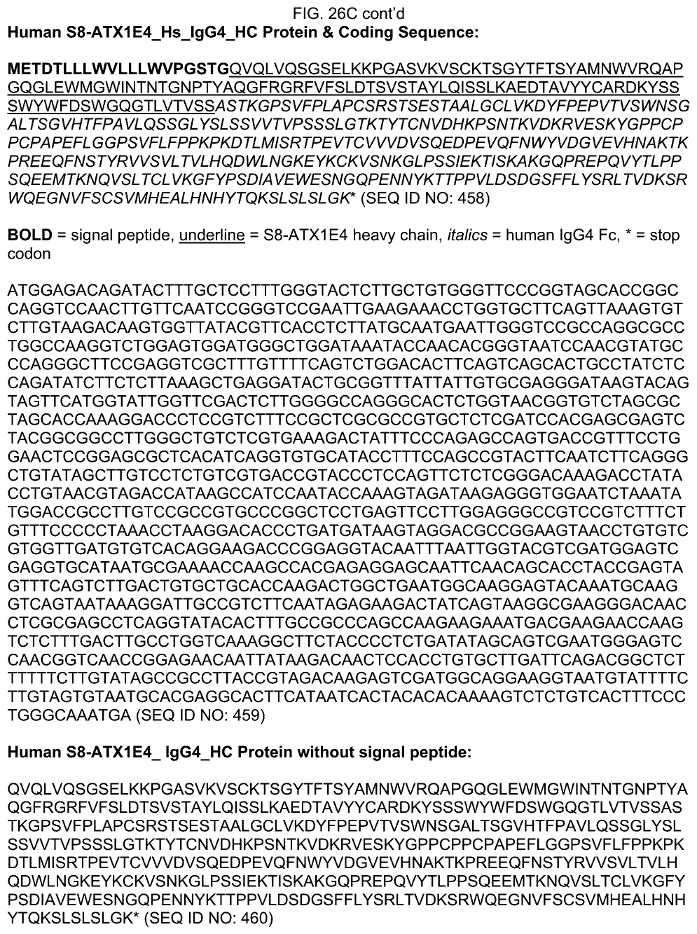

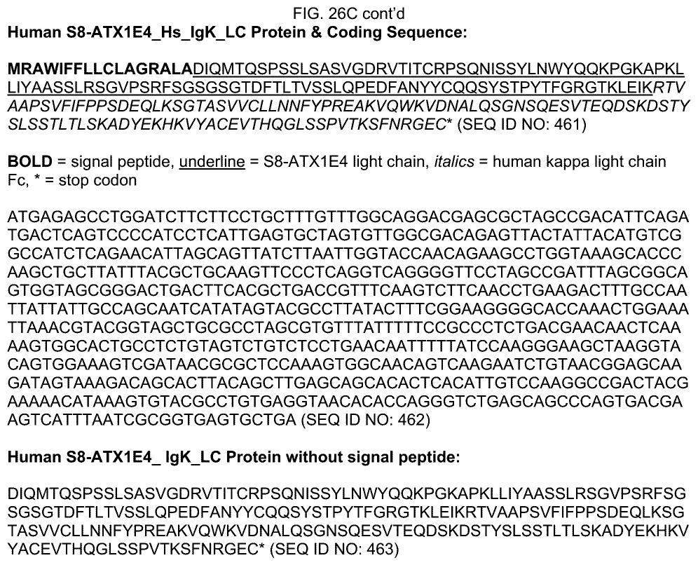

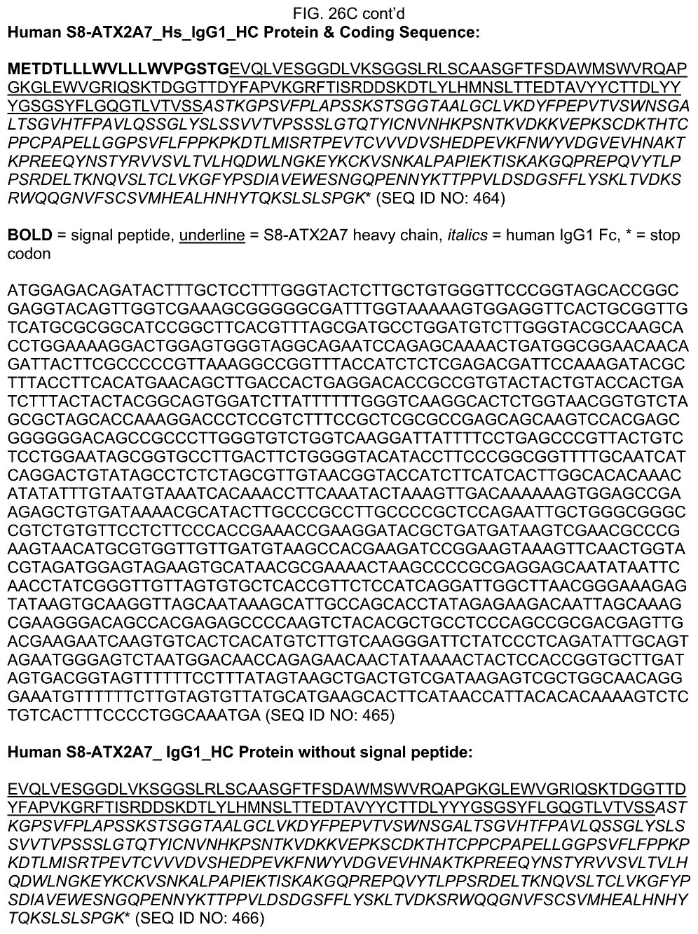

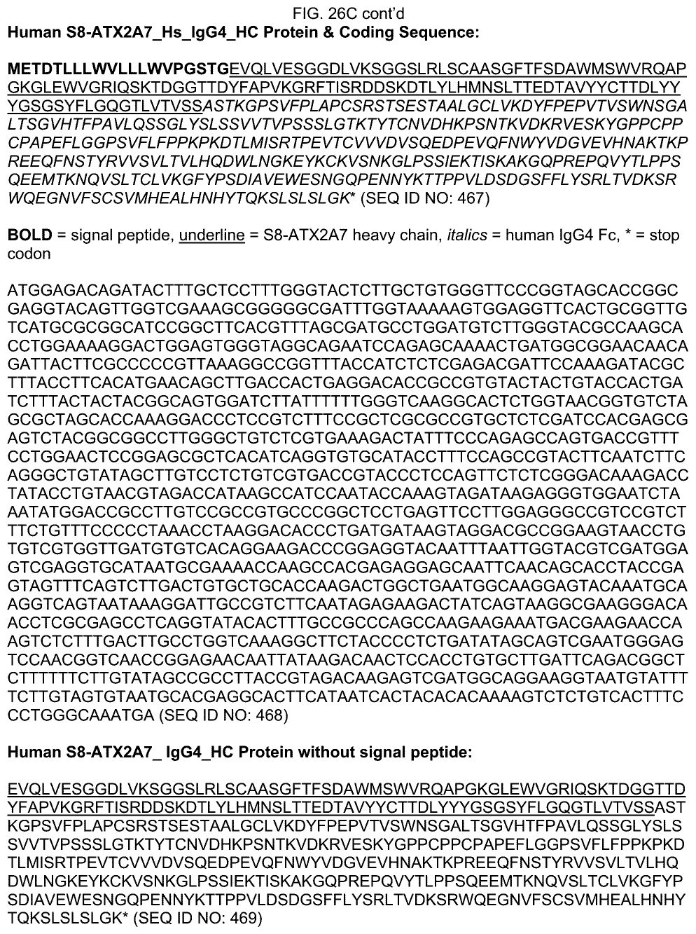

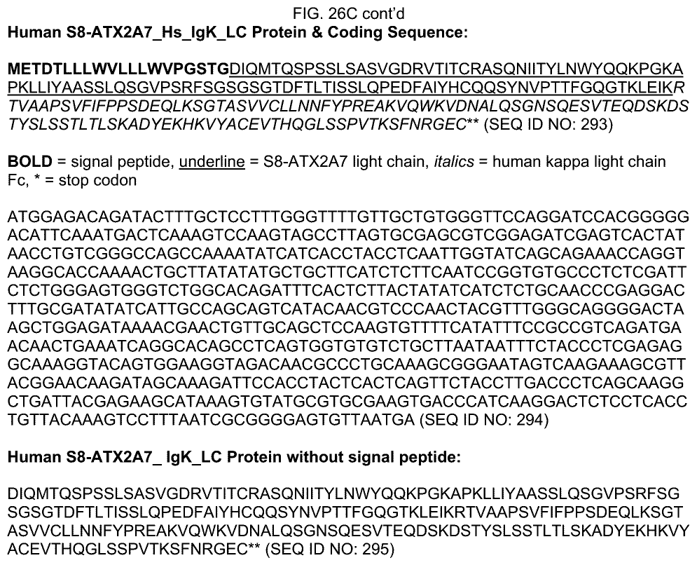

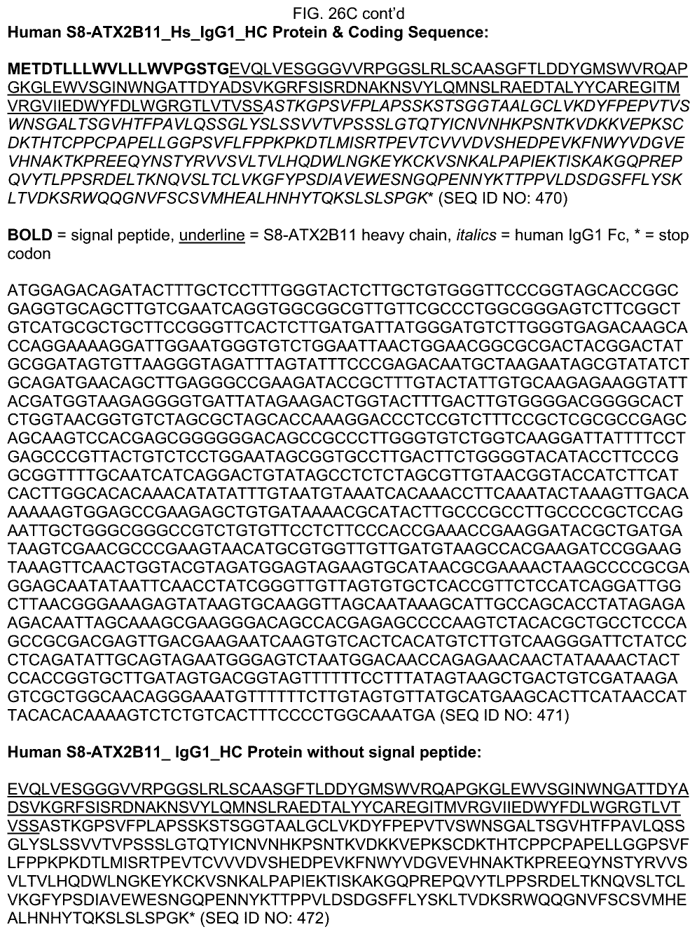

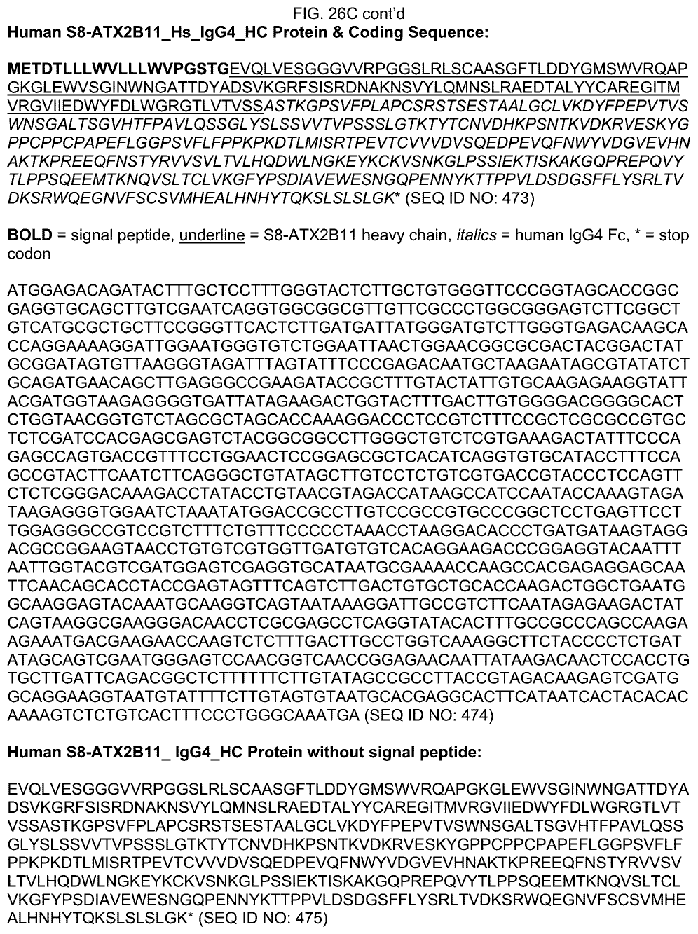

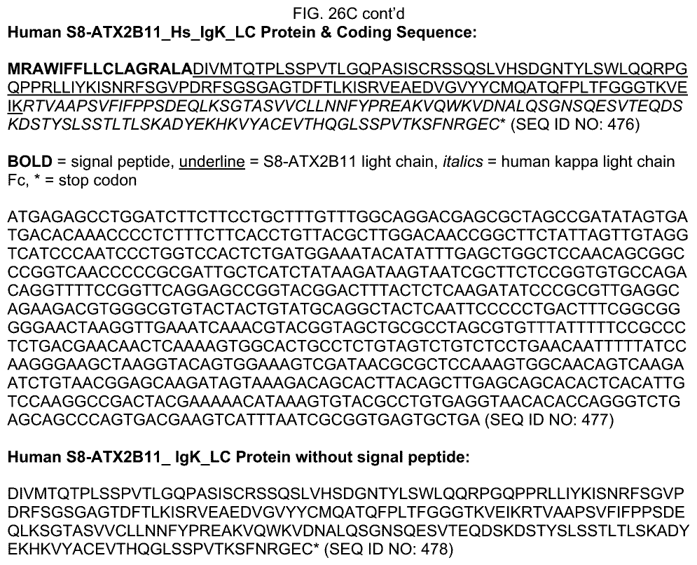

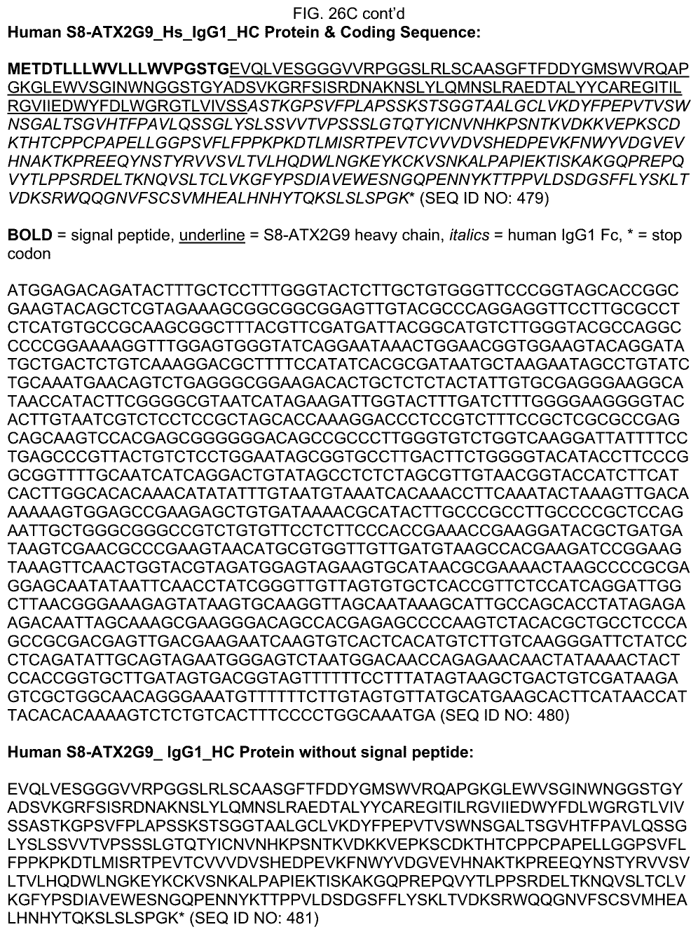

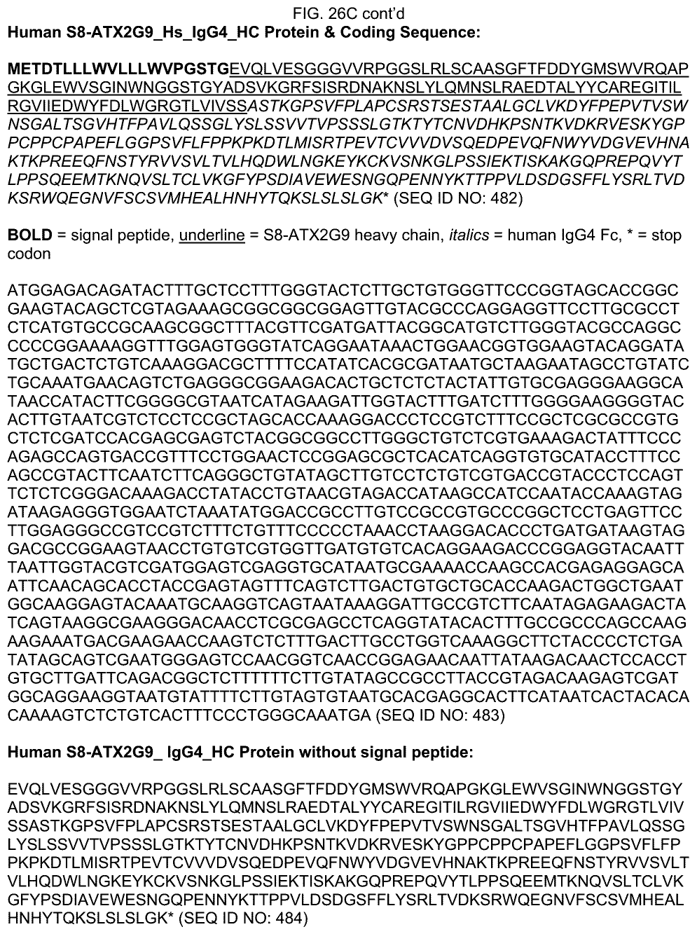

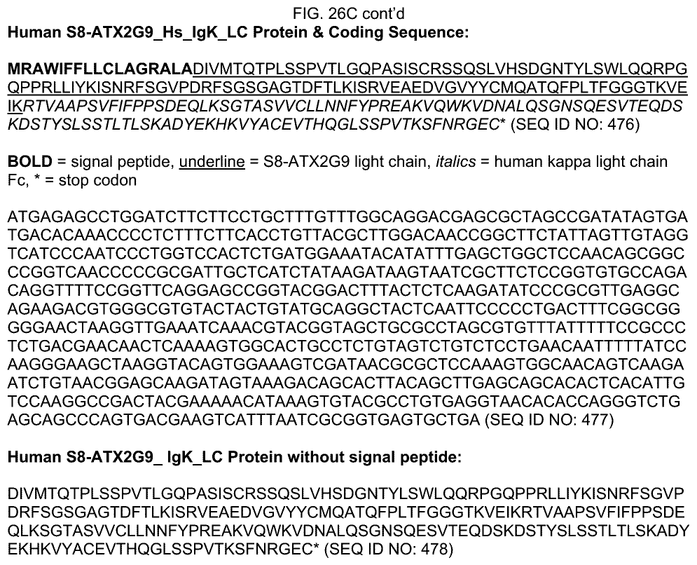

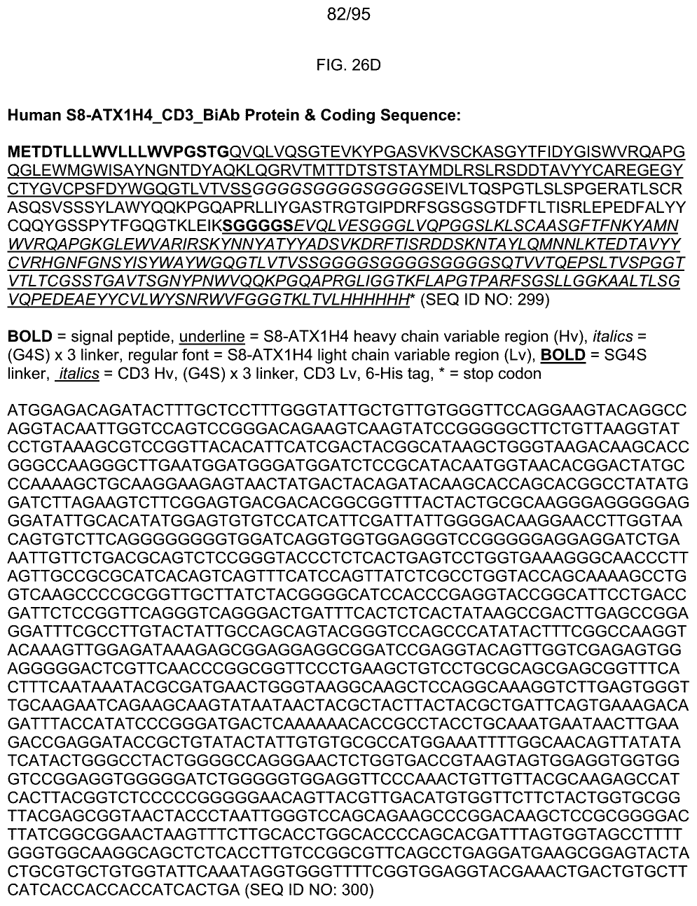

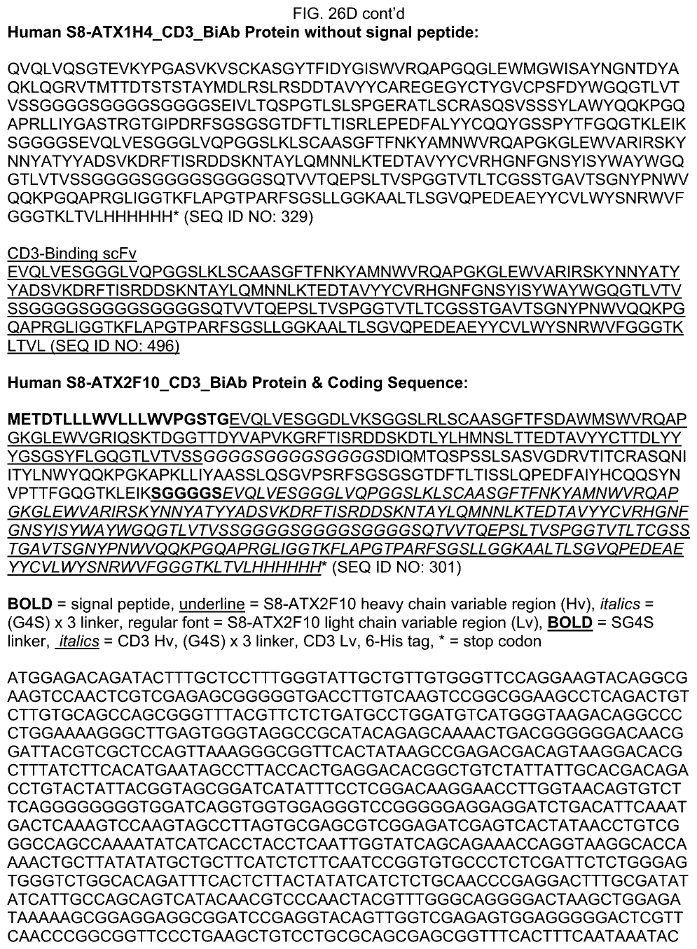

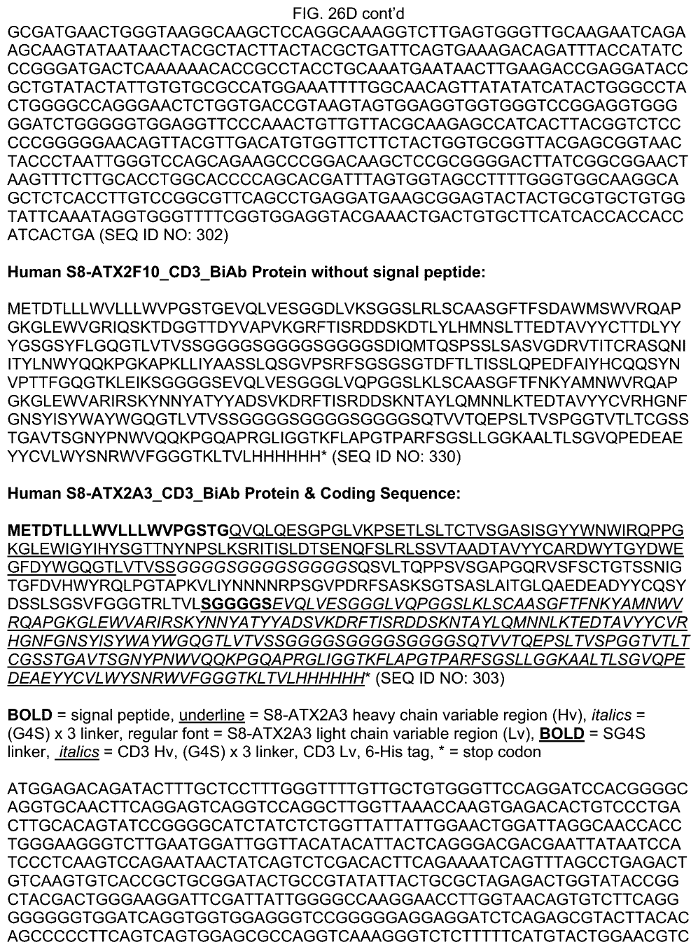

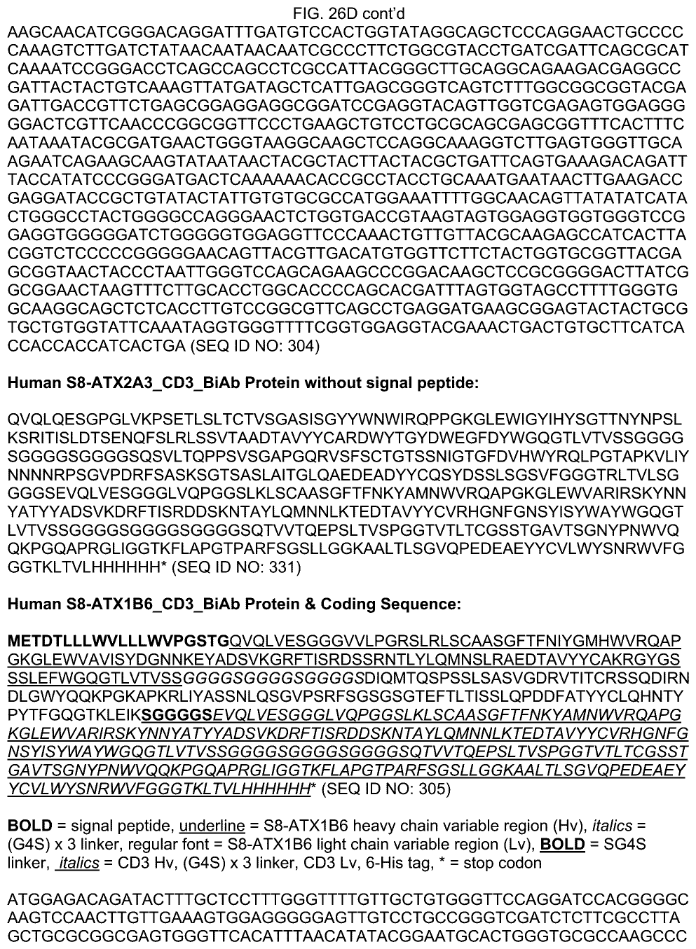

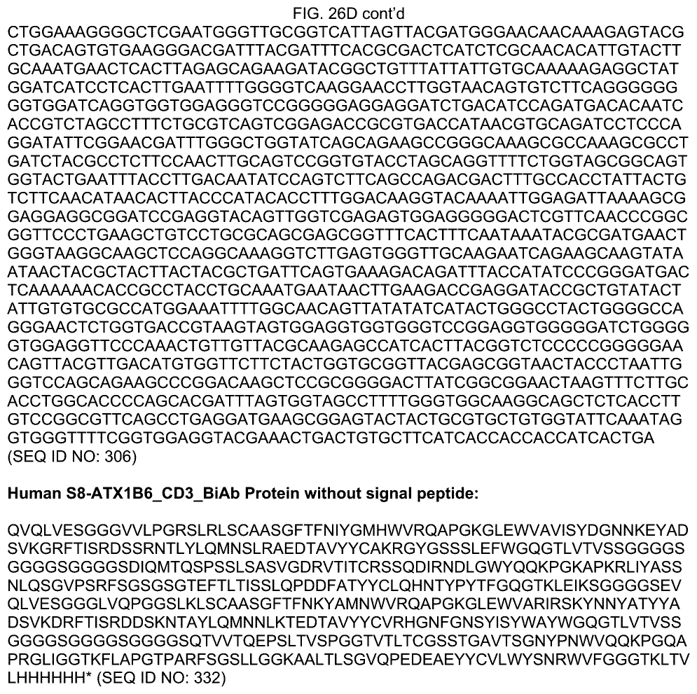

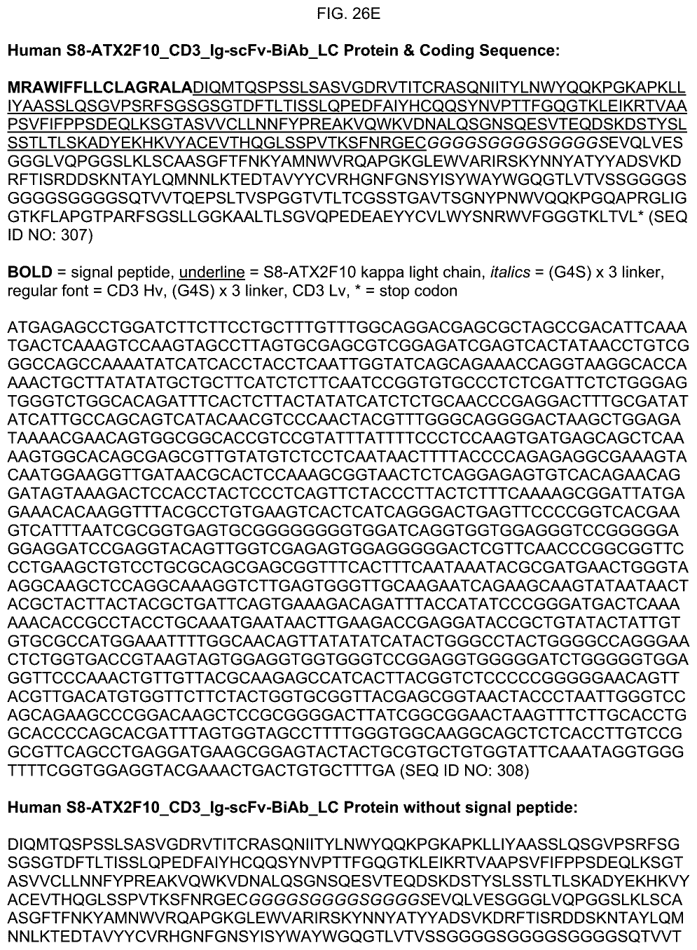

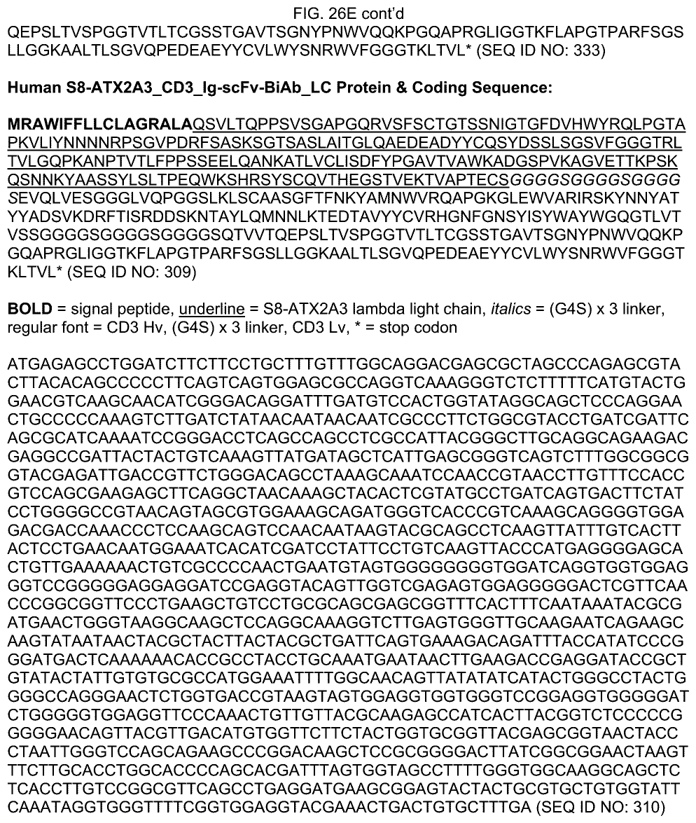

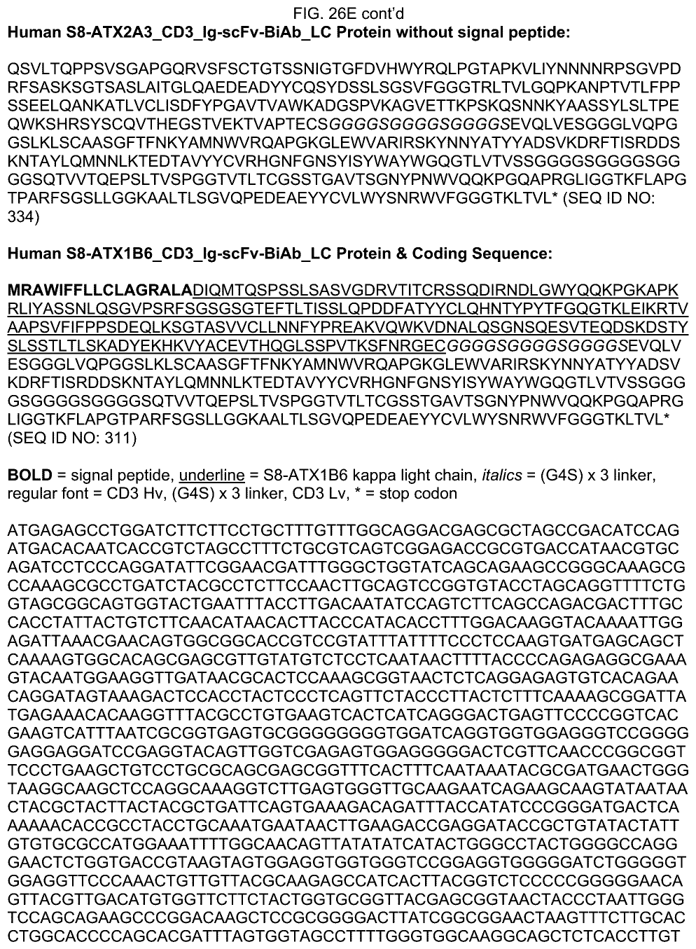

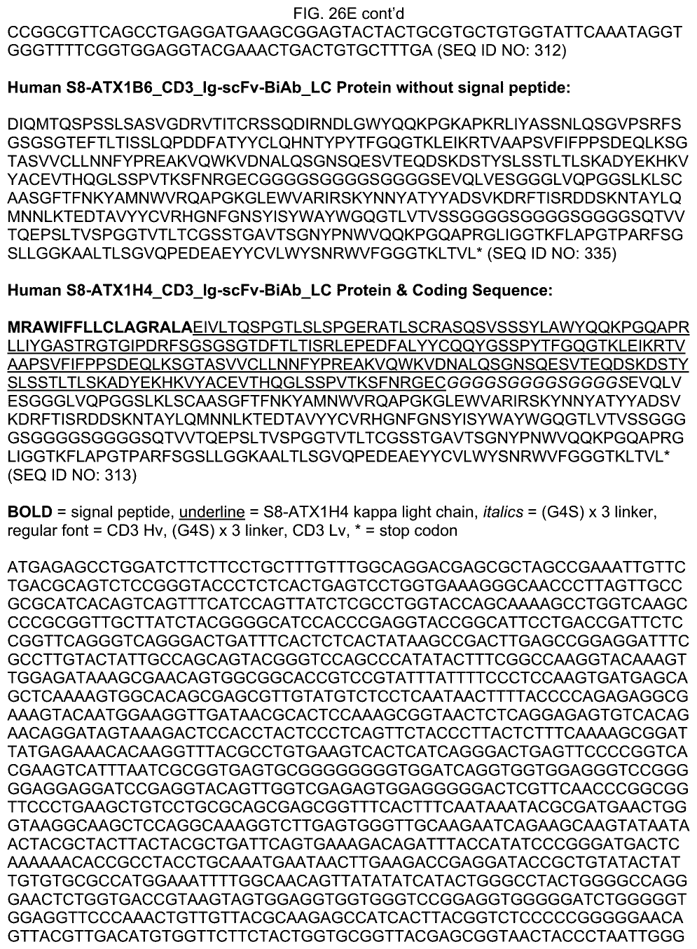

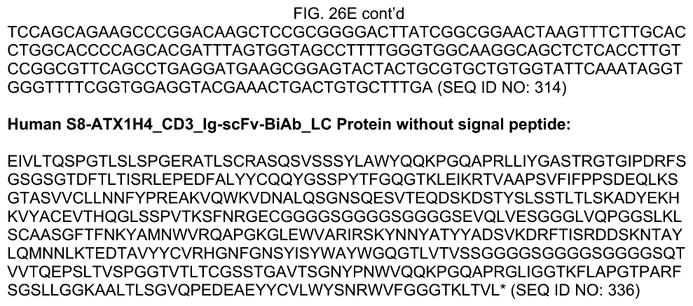

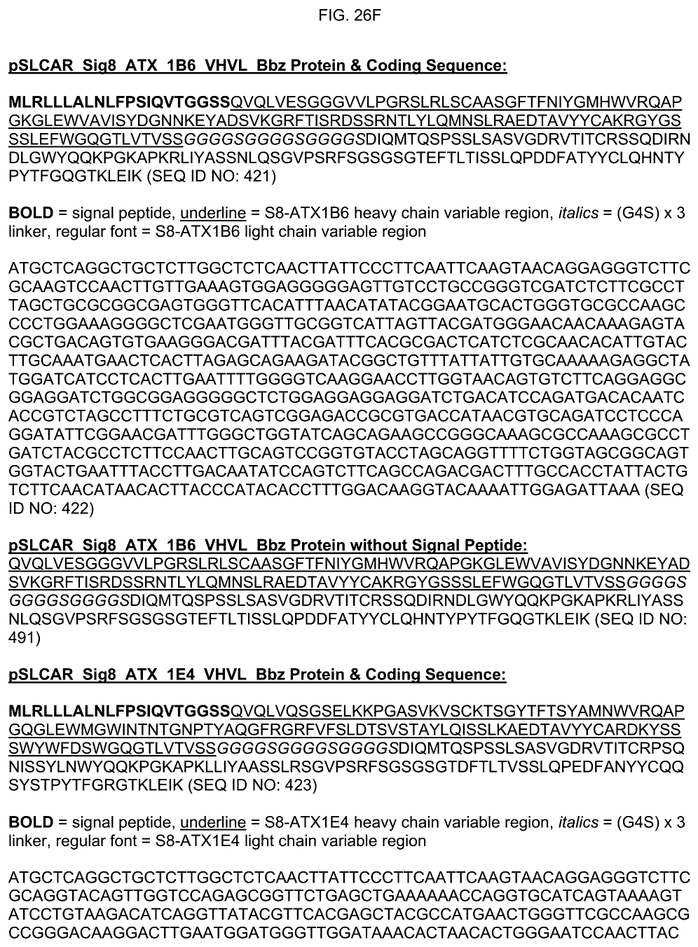

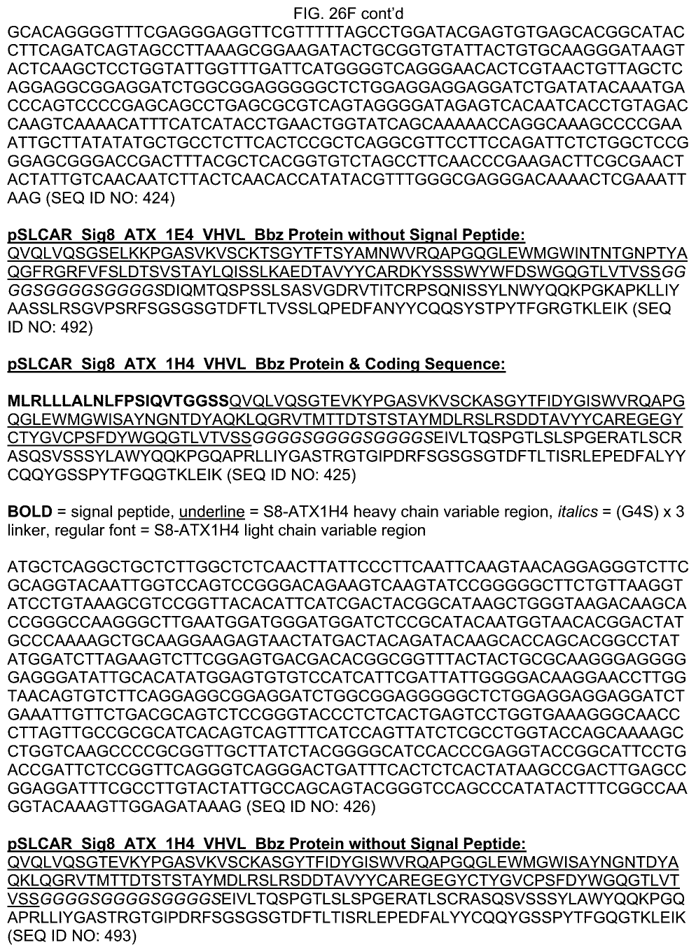

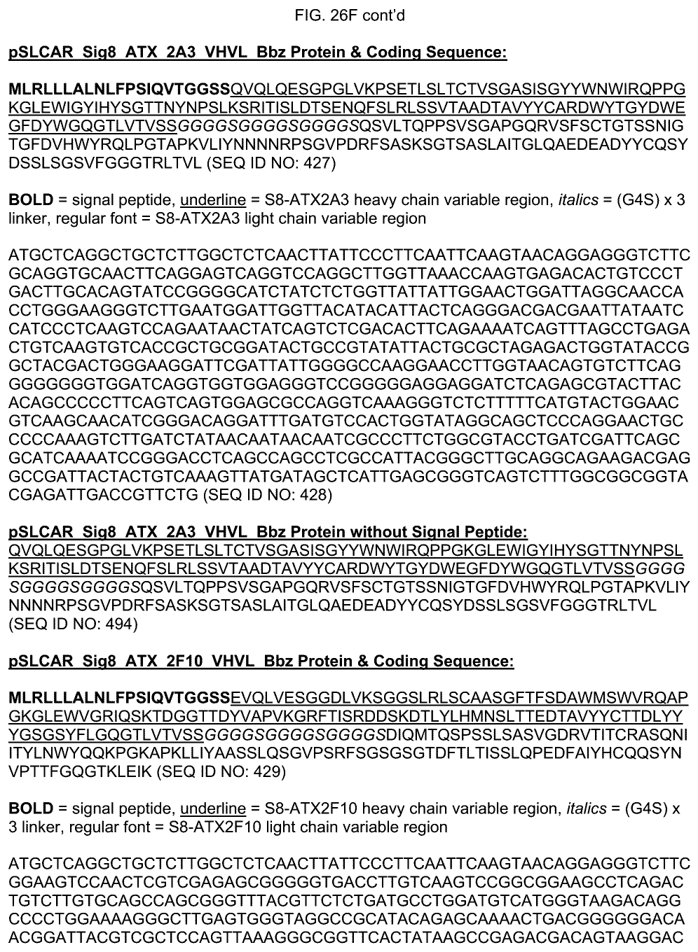

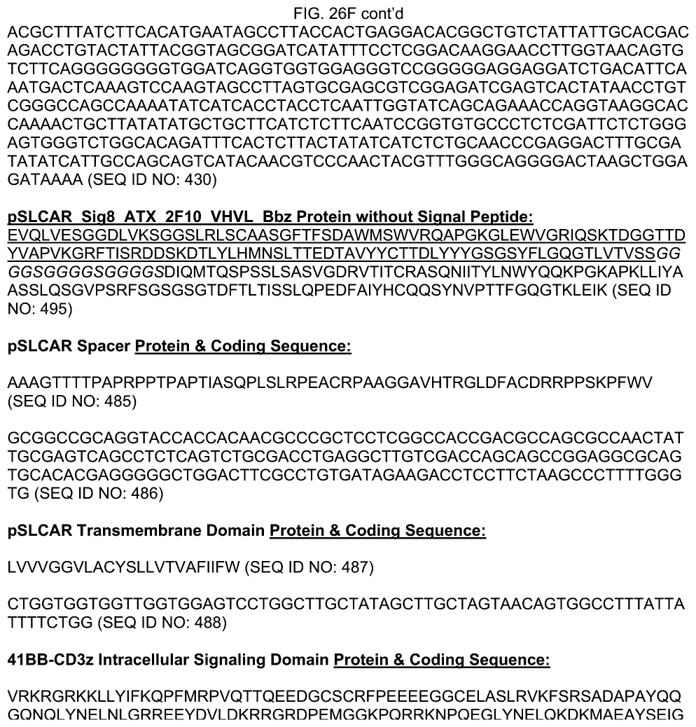

In particular embodiments, antibodies provided herein bind Siglec-8 and truncations and variations thereof. In particular embodiments, Siglec-8 and truncations and variations thereof include or are encoded by the sequences provided in FIG. 26B. Referring to the antibodies provided herein, the following CDR sets are provided. A CDR set refers to 3 light chain (LO) CDRs and 3 heavy chain (HO) CDRs that together result in binding to Siglec-8.

TABLE 1
CDR Sets according to North:

| Antibody | LC_CDR1 | LC_CDR2 | LC_CDR3 | HC_CDR1 | HC_CDR2 | HC_CDR3 |
|---|---|---|---|---|---|---|
| 1B4 | RASQDIRN | YATSSLQ | LQHNTYP | AASGFTF | VISYDGNNK | AKRGYGS |
| | DLG | S | YT | SIYGMH | D | SSLEF |
| | (SEQ ID NO: | (SEQ ID | (SEQ ID | (SEQ ID | (SEQ ID NO: | (SEQ ID |
| | 26) | NO: 27) | NO: 28) | NO: 29) | 30) | NO: 31) |
| 1B6 | RSSQDIRN | YASSNLQ | LQHNTYP | AASGFTF | VISYDGNNK | AKRGYGS |
| | DLG | S | YT | NIYGMH | E | SSLEF |
| | (SEQ ID NO: | (SEQ ID | (SEQ ID | (SEQ ID | (SEQ ID NO: | (SEQ ID |
| | 34) | NO: 35) | NO: 28) | NO: 36) | 37) | NO: 31) |
| 1E1 | RSSQSLLH | YLVSNRA | MQALQT | AASGFMF | GINWNSDNI | AKDIDAAA |
| | SNGYSYLA | S | PWT | DDYVMH | G | FDY |
| | (SEQ ID NO: | (SEQ ID | (SEQ ID | (SEQ ID | (SEQ ID NO: | (SEQ ID |
| | 38) | NO: 39) | NO: 40) | NO: 41) | 42) | NO: 43) |
| 1E2 | RASQSVIS | YGASRRA | QQYDSS | AASGFTF | RDGGARD | TKGAFFF |
| | GYLA | T | PRT | SNAWMS | (SEQ ID NO: | DY |
| | (SEQ ID NO: | (SEQ ID | (SEQ ID | (SEQ ID | 49) | (SEQ ID |
| | 45) | NO: 46) | NO: 47) | NO: 48) | | NO: 50) |
| 1E4 | RPSQNISSY | YAASSLR | QQSYST | KTSGYTF | WINTNTGN | ARDKYSS |
| | LN | S | PYT | TSYAMN | PT | SWYWFD |
| | (SEQ ID NO: | (SEQ ID | (SEQ ID | (SEQ ID | (SEQ ID NO: | S |
| | 51) | NO: 52) | NO: 53) | NO: 54) | 55) | (SEQ ID |
| | | | | | | NO: 56) |
| 1H4 | RASQSVSS | YGASTRG | QQYGSS | KASGYTFI | WISAYNGN | AREGEGY |
| | SYLA (SEQ | T | PYT | DYGIS | TD | CTYGVCP |
| | ID NO: 57) | (SEQ ID | (SEQ ID | (SEQ ID | (SEQ ID NO: | SFDY |
| | | NO: 58) | NO: 59) | NO: 60) | 61) | (SEQ ID |
| | | | | | | NO: 62) |
| 2A3 | TGTSSNIGT | YNNNNRP | QSYDSSL | TVSGASIS | YIHYSGTTN | ARDWYTG |
| | GFDVH | S | SGSV | GYYWN | (SEQ ID NO: | YDWEGFD |
| | (SEQ ID NO: | (SEQ ID | (SEQ ID | (SEQ ID | 67) | Y |
| | 63) | NO: 64) | NO: 65) | NO: 66) | | (SEQ ID |
| | | | | | | NO: 68) |
| 2A7 | RASQNIITY | YAASSLQ | QQSYNV | AASGFTF | RIQSKTDG | TTDLYYY |
| 2F10 | LN | S | PTT | SDAWMS | GTTD | GSGSYF |
| | (SEQ ID NO: | (SEQ ID | (SEQ ID | (SEQ ID | (SEQ ID NO: | (SEQ ID |
| | 69) | NO: 70) | NO: 71) | NO: 72) | 73) | NO: 74) |
| 2B11 | RSSQSLVH | YKISNRFS | MQATQF | AASGFTL | GINWNGAT | AREGITM |
| | SDGNTYLS | (SEQ ID | PLT | DDYGMS | TD | VRGVIIED |
| | (SEQ ID NO: | NO: 76) | (SEQ ID | (SEQ ID | (SEQ ID NO: | WYFDL |
| | 75) | | NO: 77) | NO: 78) | 79) | (SEQ ID |
| | | | | | | NO: 80) |
| 2G9 | RSSQSLVH | YKISNRFS | MQATQF | AASGFTF | GINWNGGS | AREGITIL |
| | SDGNTYLS | (SEQ ID | PLT | DDYGMS | TG | RGVIIEDW |
| | (SEQ ID NO: | NO: 76) | (SEQ ID | (SEQ ID | (SEQ ID NO: | YFDL |
| | 75) | | NO: 77) | NO: 81) | 82) | (SEQ ID |
| | | | | | | NO: 83) |

TABLE 2
CDR Sets according to IMGT:

| Antibody | LC_CDR1 | LC_CDR2 | LC_CDR3 | HC_CDR1 | HC_CDR2 | HC_CDR3 |
|---|---|---|---|---|---|---|
| 1B4 | QDIRND | ATS | LQHNTYP | GFTFSIYG | ISYDGNNK | AKRGYGS |
| | (SEQ | | YT | (SEQ ID | (SEQ ID NO: | SSLEF |
| | ID | | (SEQ ID | NO: 87) | 88) | (SEQ ID |
| | NO: | | NO: 28) | | | NO: 31) |
| | 86) | | | | | |
| 1B6 | QDIRND | ASS | LQHNTYP | GFTFNIYG | ISYDGNNK | AKRGYGS |
| | (SEQ ID | | YT | (SEQ ID | (SEQ ID NO: | SSLEF |
| | NO: 86) | | (SEQ ID | NO: 90) | 88) | (SEQ ID |
| | | | NO: 28) | | | NO: 31) |
| 1E1 | QSLLHSN | LVS | MQALQT | GFMFDDY | INWNSDNI | AKDIDAAA |
| | GYSY | | PWT | V | (SEQ ID NO: | FDY |
| | (SEQ ID | | (SEQ ID | (SEQ ID | 93) | (SEQ ID |
| | NO: 91) | | NO: 40) | NO: 92) | | NO: 43) |
| 1E2 | QSVISGY | GAS | QQYDSS | GFTFSNA | RDGGAR | TKGAFFF |
| | (SEQ ID | | PRT | W | (SEQ ID NO: | DY |
| | NO: 95) | | (SEQ ID | (SEQ ID | 97) | (SEQ ID |
| | | | NO: 47) | NO: 96) | | NO: 50) |
| 1E4 | QNISSY | AAS | QQSYST | GYTFTSY | INTNTGNP | ARDKYSS |
| | (SEQ ID | | PYT | A | (SEQ ID NO: | SWYWFD |
| | NO: 98) | | (SEQ ID | (SEQ ID | 100) | S |
| | | | NO: 53) | NO: 99) | | (SEQ ID |
| | | | | | | NO: 56) |
| 1H4 | QSVSSSY | GAS | QQYGSS | GYTFIDYG | ISAYNGNT | AREGEGY |
| | (SEQ ID | | PYT | (SEQ ID | (SEQ ID NO: | CTYGVCP |
| | NO: 101) | | (SEQ ID | NO: 102) | 103) | SFDY |
| | | | NO: 59) | | | (SEQ ID |
| | | | | | | NO: 62) |
| 2A3 | SSNIGTG | NNN | QSYDSSL | GASISGY | IHYSGTT | ARDWYTG |
| | FD | | SGSV | Y | (SEQ ID NO: | YDWEGFD |
| | (SEQ ID | | (SEQ ID | (SEQ ID | 106) | Y |
| | NO: 104) | | NO: 65) | NO: 105) | | (SEQ ID |
| | | | | | | NO: 68) |
| 2A7 | QNIITY | AAS | QQSYNV | GFTFSDA | IQSKTDGGT | TTDLYYY |
| | (SEQ ID | | PTT | W | T | GSGSYF |
| | NO: 107) | | (SEQ ID | (SEQ ID | (SEQ ID NO: | (SEQ ID |
| | | | NO: 71) | NO: 108) | 109) | NO: 74) |
| 2B11 | QSLVHSD | KIS | MQATQF | GFTLDDY | INWNGATT | AREGITM |
| | GNTY | | PLT | G | (SEQ ID NO: | VRGVIIED |
| | (SEQ ID | | (SEQ ID | (SEQ ID | 112) | WYFDL |
| | NO: 110) | | NO: 77) | NO: 111) | | (SEQ ID |
| | | | | | | NO: 80) |
| 2F10 | QNIITY | AAS | QQSYNV | GFTFSDA | IQSKTDGGT | TTDLYYY |
| | (SEQ ID | | PTT | W | T | GSGSYF |
| | NO: 107) | | (SEQ ID | (SEQ ID | (SEQ ID NO: | (SEQ ID |
| | | | NO: 71) | NO: 108) | 109) | NO: 74) |
| 2G9 | QSLVHSD | KIS | MQATQF | GFTFDDY | INWNGGST | AREGITIL |
| | GNTY | | PLT | G | (SEQ ID NO: | RGVIIEDW |
| | (SEQ ID | | (SEQ ID | (SEQ ID | 114) | YFDL |
| | NO: 110) | | NO: 77) | NO: 113) | | (SEQ ID |
| | | | | | | NO: 83) |

TABLE 3
CDR Sets according to Kabat:

| Antibody | LC_CDR1 | LC_CDR2 | LC_CDR3 | HC_CDR1 | HC_CDR2 | HC_CDR3 |
|---|---|---|---|---|---|---|
| 1B4 | RASQDIRN | ATSSLQS | LQHNTYP | IYGMH | VISYDGNNK | RGYGSSS |
| | DLG | (SEQ ID | YT | (SEQ ID | DYTDSVKG | LEF |
| | (SEQ ID | NO: 116) | (SEQ ID | NO: 117) | (SEQ ID NO: | (SEQ ID |
| | NO: 26) | | NO: 28) | | 118) | NO: 119) |
| 1B6 | RSSQDIRN | ASSNLQS | LQHNTYP | IYGMH | VISYDGNNK | RGYGSSS |
| | DLG | (SEQ ID | YT | (SEQ ID | EYADSVKG | LEF |
| | (SEQ ID | NO: 121) | (SEQ ID | NO: 117) | (SEQ ID NO: | (SEQ ID |
| | NO: 34) | | NO: 28) | | 122) | NO: 119) |
| 1E1 | RSSQSLLH | LVSNRAS | MQALQT | DYVMH | GINWNSDNI | DIDAAAFD |
| | SNGYSYLA | (SEQ ID | PWT | (SEQ ID | GYADSVKG | Y |
| | (SEQ ID | NO: 123) | (SEQ ID | NO: 124) | (SEQ ID NO: | (SEQ ID |
| | NO: 38) | | NO: 40) | | 125) | NO: 126) |
| 1E2 | RASQSVIS | GASRRAT | QQYDSS | NAWMS | RDGGARDY | GAFFFDY |
| | GYLA | (SEQ ID | PRT | (SEQ ID | AAPVKG | (SEQ ID |
| | (SEQ ID | NO: 127) | (SEQ ID | NO: 128) | (SEQ ID NO: | NO: 130) |
| | NO: 45) | | NO: 47) | | 129) | |
| 1E4 | RPSQNISS | AASSLRS | QQSYST | SYAMN | WINTNTGN | DKYSSSW |
| | YLN | (SEQ ID | PYT | (SEQ ID | PTYAQGFR | YWFDS |
| | (SEQ ID | NO: 131) | (SEQ ID | NO: 132) | G | (SEQ ID |
| | NO: 51) | | NO: 53) | | (SEQ ID NO: | NO: 134) |
| | | | | | 133) | |
| 1H4 | RASQSVSS | GASTRGT | QQYGSS | DYGIS | WISAYNGN | EGEGYCT |
| | SYLA | (SEQ ID | PYT | (SEQ ID | TDYAQKLQ | YGVCPSF |
| | (SEQ ID | NO: 135) | (SEQ ID | NO: 136) | G | DY |
| | NO: 57) | | NO: 59) | | (SEQ ID NO: | (SEQ ID |
| | | | | | 137) | NO: 138) |
| 2A3 | TGTSSNIG | NNNNRPS | QSYDSSL | SGYYWN | YIHYSGTTN | DWYTGYD |
| | TGFDVH | (SEQ ID | SGSV | (SEQ ID | YNPSLKS | WEGFDY |
| | (SEQ ID | NO: 139) | (SEQ ID | NO: 140) | (SEQ ID NO: | (SEQ ID |
| | NO: 63) | | NO: 65) | | 141) | NO: 142) |
| 2A7 | RASQNIITY | AASSLQS | QQSYNV | DAWMS | RIQSKTDG | DLYYYGS |
| | LN | (SEQ ID | PTT | (SEQ ID | GTTDYFAP | GSYF |
| | (SEQ ID | NO: 143) | (SEQ ID | NO: 144) | VKG | (SEQ ID |
| | NO: 69) | | NO: 71) | | (SEQ ID NO: | NO: 146) |
| | | | | | 145) | |
| 2B11 | RSSQSLVH | KISNRFS | MQATQF | DYGMS | GINWNGAT | EGITMVR |
| | SDGNTYLS | (SEQ ID | PLT | (SEQ ID | TDYADSVK | GVIIEDWY |
| | (SEQ ID | NO: 147) | (SEQ ID | NO: 148) | G | FDL |
| | NO: 75) | | NO: 77) | | (SEQ ID NO: | (SEQ ID |
| | | | | | 149) | NO: 150) |
| 2F10 | RASQNIITY | AASSLQS | QQSYNV | DAWMS | RIQSKTDG | DLYYYGS |
| | LN | (SEQ ID | PTT | (SEQ ID | GTTDYVAP | GSYF |
| | (SEQ ID | NO: 143) | (SEQ ID | NO: 144) | VKG | (SEQ ID |
| | NO: 69) | | NO: 71) | | (SEQ ID NO: | NO: 146) |
| | | | | | 151) | |
| 2G9 | RSSQSLVH | KISNRFS | MQATQF | DYGMS | GINWNGGS | EGITILRG |
| | SDGNTYLS | (SEQ ID | PLT | (SEQ ID | TGYADSVK | VIIEDWYF |
| | (SEQ ID | NO: 147) | (SEQ ID | NO: 148) | G | DL |
| | NO: 75) | | NO: 77) | | (SEQ ID NO: | (SEQ ID |
| | | | | | 152) | NO: 153) |

TABLE 4
CDR Sets according to Chothia:

| Antibody | LC_CDR1 | LC_CDR2 | LC_CDR3 | HC_CDR1 | HC_CDR2 | HC_CDR3 |
|---|---|---|---|---|---|---|
| 1B4 | RASQDIRN | ATSSLQS | LQHNTYP | GFTFSIY | SYDGNN | RGYGSSSL |
| | DLG | (SEQ ID | YT | (SEQ ID | (SEQ ID | EF |
| | (SEQ ID NO: | NO: 116) | (SEQ ID | NO: 156) | NO: 157) | (SEQ ID |
| | 26) | | NO: 28) | | | NO: 119) |
| 1B6 | RSSQDIRN | ASSNLQS | LQHNTYP | GFTFNIY | SYDGNN | RGYGSSSL |
| | DLG | (SEQ ID | YT | (SEQ ID | (SEQ ID | EF |
| | (SEQ ID NO: | NO: 121) | (SEQ ID | NO: 158) | NO: 157) | (SEQ ID |
| | 34) | | NO: 28) | | | NO: 119) |
| 1E1 | RSSQSLLH | LVSNRAS | MQALQT | GFMFDDY | NWNSDN | DIDAAAFD |
| | SNGYSYLA | (SEQ ID | PWT | (SEQ ID | (SEQ ID | Y |
| | (SEQ ID NO: | NO: 123) | (SEQ ID | NO: 159) | NO: 160) | (SEQ ID |
| | 38) | | NO: 40) | | | NO: 126) |
| 1E2 | RASQSVISG | GASRRAT | QQYDSS | GFTFSNA | DGGA | GAFFFDY |
| | YLA | (SEQ ID | PRT | (SEQ ID | (SEQ ID | (SEQ ID |
| | (SEQ ID NO: | NO: 127) | (SEQ ID | NO: 161) | NO: 162) | NO: 130) |
| | 45) | | NO: 47) | | | |
| 1E4 | RPSQNISSY | AASSLRS | QQSYST | GYTFTSY | NTNTGN | DKYSSSW |
| | LN | (SEQ ID | PYT | (SEQ ID | (SEQ ID | YWFDS |
| | (SEQ ID NO: | NO: 131) | (SEQ ID | NO: 163) | NO: 164) | (SEQ ID |
| | 51) | | NO: 53) | | | NO: 134) |
| 1H4 | RASQSVSS | GASTRGT | QQYGSS | GYTFIDY | SAYNGN | EGEGYCTY |
| | SYLA | (SEQ ID | PYT | (SEQ ID | (SEQ ID | GVCPSFDY |
| | (SEQ ID NO: | NO: 135) | (SEQ ID | NO: 165) | NO: 166) | (SEQ ID |
| | 57) | | NO: 59) | | | NO: 138) |
| 2A3 | TGTSSNIGT | NNNNRPS | QSYDSSL | GASISGY | HYSGT | DWYTGYD |
| | GFDVH | (SEQ ID | SGSV | (SEQ ID | (SEQ ID | WEGFDY |
| | (SEQ ID NO: | NO: 139) | (SEQ ID | NO: 167) | NO: 168) | (SEQ ID |
| | 63) | | NO: 65) | | | NO: 142) |
| 2A7 | RASQNIITYL | AASSLQS | QQSYNV | GFTFSDA | QSKTDGG | DLYYYGSG |
| | N | (SEQ ID | PTT | (SEQ ID | T | SYF |
| | (SEQ ID NO: | NO: 143) | (SEQ ID | NO: 169) | (SEQ ID | (SEQ ID |
| | 69) | | NO: 71) | | NO: 170) | NO: 146) |
| 2B11 | RSSQSLVH | KISNRFS | MQATQF | GFTLDDY | NWNGAT | EGITMVRG |
| | SDGNTYLS | (SEQ ID | PLT | (SEQ ID | (SEQ ID | VIIEDWYFD |
| | (SEQ ID NO: | NO: 147) | (SEQ ID | NO: 171) | NO: 172) | L |
| | 75) | | NO: 77) | | | (SEQ ID |
| | | | | | | NO: 150) |
| 2F10 | RASQNIITYL | AASSLQS | QQSYNV | GFTFSDA | QSKTDGG | DLYYYGSG |
| | N | (SEQ ID | PTT | (SEQ ID | T | SYF |
| | (SEQ ID NO: | NO: 143) | (SEQ ID | NO: 169) | (SEQ ID | (SEQ ID |
| | 69) | | NO: 71) | | NO: 170) | NO: 146) |
| 2G9 | RSSQSLVH | KISNRFS | MQATQF | GFTFDDY | NWNGGS | EGITILRGV |
| | SDGNTYLS | (SEQ ID | PLT | (SEQ ID | (SEQ ID | IIEDWYFDL |
| | (SEQ ID NO: | NO: 147) | (SEQ ID | NO: 173) | NO: 174) | (SEQ ID |
| | 75) | | NO: 77) | | | NO: 153) |

TABLE 5
CDR Sets according to Contact:

| Antibody | LC_CDR1 | LC_CDR2 | LC_CDR3 | HC_CDR1 | HC_CDR2 | HC_CDR3 |
|---|---|---|---|---|---|---|
| 1B4 | RNDLGWY | RLIYATSS | LQHNTYP | SIYGMH | WVAVISYD | AKRGYGS |
| | (SEQ ID NO: | LQ | Y | (SEQ ID | GNNKD | SSLE |
| | 175) | (SEQ ID | (SEQ ID | NO: 178) | (SEQ ID NO: | (SEQ ID |
| | | NO: 176) | NO: 177) | | 179) | NO: 180) |
| 1B6 | RNDLGWY | RLIYASSN | LQHNTYP | NIYGMH | WVAVISYD | AKRGYGS |
| | (SEQ ID NO: | LQ | Y | (SEQ ID | GNNKE | SSLE |
| | 175) | (SEQ ID | (SEQ ID | NO: 184) | (SEQ ID NO: | (SEQ ID |
| | | NO: 183) | NO: 177) | | 185) | NO: 180) |
| 1E1 | YSYLAWY | LLIYLVSN | MQALQT | DDYVMH | WVSGINWN | AKDIDAAA |
| | (SEQ ID NO: | RA | PW | (SEQ ID | SDNIG | FD |
| | 186) | (SEQ ID | (SEQ ID | NO: 189) | (SEQ ID NO: | (SEQ ID |
| | | NO: 187) | NO: 188) | | 190) | NO: 191) |
| 1E2 | SGYLAWY | LLIYGASR | QQYDSS | SNAWMS | WVGRDGG | TKGAFFFD |
| | (SEQ ID NO: | RA | PR | (SEQ ID | ARD | (SEQ ID |
| | 192) | (SEQ ID | (SEQ ID | NO: 195) | (SEQ ID NO: | NO: 197) |
| | | NO: 193) | NO: 194) | | 196) | |
| 1E4 | SSYLNWY | LLIYAASS | QQSYST | TSYAMN | WMGWINTN | ARDKYSSS |
| | (SEQ ID NO: | LR | PY | (SEQ ID | TGNPT | WYWFD |
| | 198) | (SEQ ID | (SEQ ID | NO: 201) | (SEQ ID NO: | (SEQ ID |
| | | NO: 199) | NO: 200) | | 202) | NO: 203) |
| 1H4 | SSYLAWY | LLIYGAST | QQYGSS | IDYGIS | WMGWISAY | AREGEGY |
| | (SEQ ID NO: | RG | PY | (SEQ ID | NGNTD | CTYGVCPS |
| | 204) | (SEQ ID | (SEQ ID | NO: 207) | (SEQ ID NO: | FD |
| | | NO: 205) | NO: 206) | | 208) | (SEQ ID |
| | | | | | | NO: 209) |
| 2A3 | GFDVHWY | VLIYNNNN | QSYDSSL | SGYYWN | WIGYIHYSG | ARDWYTG |
| | (SEQ ID NO: | RP | SGS | (SEQ ID | TTN | YDWEGFD |
| | 210) | (SEQ ID | (SEQ ID | NO: 140) | (SEQ ID NO: | (SEQ ID |
| | | NO: 211) | NO: 212) | | 213) | NO: 214) |
| 2A7 | ITYLNWY | LLIYAASS | QQSYNV | SDAWMS | WVGRIQSK | TTDLYYYG |
| | (SEQ ID NO: | LQ | PT | (SEQ ID | TDGGTTD | SGSY |
| | 215) | (SEQ ID | (SEQ ID | NO: 218) | (SEQ ID NO: | (SEQ ID |
| | | NO: 216) | NO: 217) | | 219) | NO: 220) |
| 2B11 | NTYLSWL | LLIYKISNR | MQATQF | DDYGMS | WVSGINWN | AREGITMV |
| | (SEQ ID NO: | F | PL | (SEQ ID | GATTD | RGVIIEDW |
| | 221) | (SEQ ID | (SEQ ID | NO: 224) | (SEQ ID NO: | YFD |
| | | NO: 222) | NO: 223) | | 225) | (SEQ ID |
| | | | | | | NO: 226) |
| 2F10 | ITYLNWY | LLIYAASS | QQSYNV | SDAWMS | WVGRIQSK | TTDLYYYG |
| | (SEQ ID NO: | LQ | PT | (SEQ ID | TDGGTTD | SGSY |
| | 215) | (SEQ ID | (SEQ ID | NO: 218) | (SEQ ID NO: | (SEQ ID |
| | | NO: 216) | NO: 217) | | 219) | NO: 220) |
| 2G9 | NTYLSWL | LLIYKISNR | MQATQF | DDYGMS | WVSGINWN | AREGITILR |
| | (SEQ ID NO: | F | PL | (SEQ ID | GGSTG | GVIIEDWY |
| | 221) | (SEQ ID | (SEQ ID | NO: 224) | (SEQ ID NO: | FD |
| | | NO: 222) | NO: 223) | | 227) | (SEQ ID |
| | | | | | | NO: 228) |

In particular embodiments, a Siglec-8 antibody includes a variable light chain (V_{L}) and a variable heavy chain (V_{H}) including:

TABLE 6
Variable Regions.

| Antibody | V_{L} | V_{H} |
|---|---|---|
| 1B4 | DIQMTQSPSSLSASVGDRVTI | QVQLVESGGGVVQPGRSLRLSCAASGF |
| | TCRASQDIRNDLGWYQQKP | TFSIYGMHWVRQAPGKGLEWVAVISYD |
| | GKAPKRLIYATSSLQSGVPS | GNNKDYTDSVKGRFTISRDNSKNTLYLQ |
| | RFSGSRSGAEFTLTVSSLQP | MNSLTAEDTAVYYCAKRGYGSSSLEFW |
| | EDFATYYCLQHNTYPYTFGQ | GQGTLVTVSS (SEQ ID NO: 2) |
| | GTKLEIK (SEQ ID NO: 1) | |
| 1B6 | DIQMTQSPSSLSASVGDRVTI | QVQLVESGGGVVLPGRSLRLSCAASGFT |
| | TCRSSQDIRNDLGWYQQKP | FNIYGMHWVRQAPGKGLEWVAVISYDG |
| | GKAPKRLIYASSNLQSGVPS | NNKEYADSVKGRFTISRDSSRNTLYLQM |
| | RFSGSGSGTEFTLTISSLQPD | NSLRAEDTAVYYCAKRGYGSSSLEFWG |
| | DFATYYCLQHNTYPYTFGQG | QGTLVTVSS (SEQ ID NO: 5) |
| | TKLEIK (SEQ ID NO: 4) | |
| 1E1 | DIVMTQSPLSLPVTPGEPASI | EVQLVESGGGLVQPGRSLRLSCAASGF |
| | SCRSSQSLLHSNGYSYLAWY | MFDDYVMHWVRQAPGKGLEWVSGINW |
| | LQKPGQSPQLLIYLVSNRAS | NSDNIGYADSVKGRFTISRDNAKNSLFLQ |
| | GVPDRFTGSGSGTDFTLKIS | MNSLRVEDTALYYCAKDIDAAAFDYWGQ |
| | RVEAEDVGVYYCMQALQTP | GTLVTVSS (SEQ ID NO: 7) |
| | WTFGQGTKVEIK (SEQ ID | |
| | NO: 6) | |
| 1E2 | EIVLTQSPGTLSLSPGERATL | EVQLVESGGGLVKPGESLRLSCAASGFT |
| | SCRASQSVISGYLAWYQQKP | FSNAWMSWVRQAPGKGLEWVGRDGGA |
| | GQAPRLLIYGASRRATGITDR | RDYAAPVKGRFTISRDDSKNTLYLQMNT |
| | FSGSGSGTDFTLIISRLEPED | LKTEDTAVYYCTKGAFFFDYWGQGTLVT |
| | SAVYYCQQYDSSPRTFGQG | VSS (SEQ ID NO: 10) |
| | TKVEIK (SEQ ID NO: 9) | |
| 1E4 | DIQMTQSPSSLSASVGDRVTI | QVQLVQSGSELKKPGASVKVSCKTSGYT |
| | TCRPSQNISSYLNWYQQKPG | FTSYAMNWVRQAPGQGLEWMGWINTN |
| | KAPKLLIYAASSLRSGVPSRF | TGNPTYAQGFRGRFVFSLDTSVSTAYLQ |
| | SGSGSGTDFTLTVSSLQPED | ISSLKAEDTAVYYCARDKYSSSWYWFDS |
| | FANYYCQQSYSTPYTFGRGT | WGQGTLVTVSS (SEQ ID NO: 14) |
| | KLEIK (SEQ ID NO: 13) | |
| 1H4 | EIVLTQSPGTLSLSPGERATL | QVQLVQSGTEVKYPGASVKVSCKASGY |
| | SCRASQSVSSSYLAWYQQK | TFIDYGISWVRQAPGQGLEWMGWISAY |
| | PGQAPRLLIYGASTRGTGIPD | NGNTDYAQKLQGRVTMTTDTSTSTAYM |
| | RFSGSGSGTDFTLTISRLEPE | DLRSLRSDDTAVYYCAREGEGYCTYGV |
| | DFALYYCQQYGSSPYTFGQ | CPSFDYWGQGTLVTVSS (SEQ ID NO: |
| | GTKLEIK (SEQ ID NO: 15) | 16) |
| 2A3 | QSVLTQPPSVSGAPGQRVSF | QVQLQESGPGLVKPSETLSLTCTVSGASI |
| | SCTGTSSNIGTGFDVHWYRQ | SGYYWNWIRQPPGKGLEWIGYIHYSGTT |
| | LPGTAPKVLIYNNNNRPSGV | NYNPSLKSRITISLDTSENQFSLRLSSVTA |
| | PDRFSASKSGTSASLAITGLQ | ADTAVYYCARDWYTGYDWEGFDYWGQ |
| | AEDEADYYCQSYDSSLSGSV | GTLVTVSS (SEQ ID NO: 18) |
| | FGGGTRLTVL (SEQ ID NO: | |
| | 17) | |
| 2A7 | DIQMTQSPSSLSASVGDRVTI | EVQLVESGGDLVKSGGSLRLSCAASGFT |
| | TCRASQNIITYLNWYQQKPG | FSDAWMSWVRQAPGKGLEWVGRIQSK |
| | KAPKLLIYAASSLQSGVPSRF | TDGGTTDYFAPVKGRFTISRDDSKDTLYL |
| | SGSGSGTDFTLTISSLQPEDF | HMNSLTTEDTAVYYCTTDLYYYGSGSYF |
| | AIYHCQQSYNVPTTFGQGTK | LGQGTLVTVSS (SEQ ID NO: 20) |
| | LEIK (SEQ ID NO: 19) | |
| 2B11 | DIVMTQTPLSSPVTLGQPASI | EVQLVESGGGVVRPGGSLRLSCAASGF |
| | SCRSSQSLVHSDGNTYLSWL | TLDDYGMSWVRQAPGKGLEWVSGINW |
| | QQRPGQPPRLLIYKISNRFSG | NGATTDYADSVKGRFSISRDNAKNSVYL |
| | VPDRFSGSGAGTDFTLKISR | QMNSLRAEDTALYYCAREGITMVRGVIIE |
| | VEAEDVGVYYCMQATQFPLT | DWYFDLWGRGTLVTVSS (SEQ ID NO: |
| | FGGGTKVEIK (SEQ ID NO: | 22) |
| | 21) | |
| 2F10 | DIQMTQSPSSLSASVGDRVTI | EVQLVESGGDLVKSGGSLRLSCAASGFT |
| | TCRASQNIITYLNWYQQKPG | FSDAWMSWVRQAPGKGLEWVGRIQSK |
| | KAPKLLIYAASSLQSGVPSRF | TDGGTTDYVAPVKGRFTISRDDSKDTLY |
| | SGSGSGTDFTLTISSLQPEDF | LHMNSLTTEDTAVYYCTTDLYYYGSGSY |
| | AIYHCQQSYNVPTTFGQGTK | FLGQGTLVTVSS (SEQ ID NO: 23) |
| | LEIK (SEQ ID NO: 19) | |
| 2G9 | DIVMTQTPLSSPVTLGQPASI | EVQLVESGGGVVRPGGSLRLSCAASGF |
| | SCRSSQSLVHSDGNTYLSWL | TFDDYGMSWVRQAPGKGLEWVSGINW |
| | QQRPGQPPRLLIYKISNRFSG | NGGSTGYADSVKGRFSISRDNAKNSLYL |
| | VPDRFSGSGAGTDFTLKISR | QMNSLRAEDTALYYCAREGITILRGVIIED |
| | VEAEDVGVYYCMQATQFPLT | WYFDLWGRGTLVIVSS (SEQ ID NO: 24) |
| | FGGGTKVEIK (SEQ ID NO: | |
| | 21) | |

In particular embodiments, an scFv includes a V_{L} and a V_{H} provided herein. In particular embodiments, the order of the V_{L} and V_{H} includes V_{L}-linker-V_{H} or V_{H} linker-V_{L}. In particular embodiments, the linker can be a Gly-Ser linker (examples described elsewhere herein). In particular embodiments, the linker can be the Whitlow linker (GSTSGSGKPGSGEGSTKG (SEQ ID NO: 85)).

(ii) Antibody Variants. Antibodies disclosed herein can be utilized to prepare various forms of relevant binding domain molecules. For example, particular embodiments can include binding fragments of an antibody, e.g., Fv, Fab, Fab′, F(ab′)₂, and single chain Fv fragments (scFvs) or any biologically effective fragments of an immunoglobulin that bind specifically to an epitope described herein.

In particular embodiments, an antibody fragment is used. An “antibody fragment” denotes a portion of a full-length antibody that retains the ability to bind to an epitope. Antibody fragments can be made by various techniques, including proteolytic digestion of an intact antibody as well as production by recombinant host-cells (e.g., mammalian suspension cell lines, E. coli or phage), as described herein. Antibody fragments can be screened for their binding properties in the same manner as intact antibodies. Examples of antibody fragments include Fv, scFv, Fab, Fab′, Fab′-SH, F(ab′)₂; diabodies; and linear antibodies.

A single chain variable fragment (scFv) is a fusion protein of the variable regions of the heavy and light chains of immunoglobulins connected with a short linker peptide. Fv fragments include the V_{L} and V_{H} domains of a single arm of an antibody but lack the constant regions. Although the two domains of the Fv fragment, V_{L} and V_{H}, are coded by separate genes, they can be joined, using, for example, recombinant methods, by a synthetic linker that enables them to be made as a single protein chain in which the V_{L} and V_{H} regions pair to form monovalent molecules (single chain Fv (scFv)). A single scFv amino acid sequence can be in 2 different orientations, HcFv-LcFv, or LcFv-HcFv. A bispecific antibody in the scFv-scFv format can therefore be generated in 4 different orientations, 1. HcFv-LcFv-HcFv-LcFv, 2. HcFv-LcFv-LcFv-HcFv, 3. LcFv-HcFv, HcFv-LcFv, or 4. LcFv-HcFv-LcFv-HcFv. Each of these scFv orientations can be used in, for example, a chimeric antigen receptor. For additional information regarding Fv and scFv, see e.g., Bird, et al., Science 242:423-426, 1988; Huston, et al., Proc. Natl. Acad. Sci. USA 85:5879-5883, 1988; Plueckthun, in The Pharmacology of Monoclonal Antibodies, vol. 113, Rosenburg and Moore (eds.), Springer-Verlag, New York), (1994) 269-315; WO 1993/16185; U.S. Pat. Nos. 5,571,894; and 5,587,458.

Linker sequences that are used to connect the VL and VH of an scFv are generally five to 35 amino acids in length. In particular embodiments, a VL-VH linker includes from five to 35, ten to 30 amino acids or from 15 to 25 amino acids. Variation in the linker length may retain or enhance activity, giving rise to superior efficacy in activity studies. Linker sequences of scFv are commonly Gly-Ser linkers, described in more detail elsewhere herein.

Additional examples of antibody-based binding domain formats include scFv-based grababodies and soluble VH domain antibodies. These antibodies form binding regions using only variable heavy chain regions. See, for example, Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008.

A Fab fragment is a monovalent antibody fragment including V_{L}, V_{H}, CL and CH1 domains. A F(ab′)₂ fragment is a bivalent fragment including two Fab fragments linked by a disulfide bridge at the hinge region. For discussion of Fab and F(ab′)₂ fragments having increased in vivo half-life, see U.S. Pat. No. 5,869,046. Diabodies include two epitope-binding sites that may be bivalent. See, for example, EP 0404097; WO1993/01161; and Holliger, et al., Proc. Natl. Acad. Sci. USA 90:6444-6448, 1993. Dual affinity retargeting antibodies (DART™; based on the diabody format but featuring a C-terminal disulfide bridge for additional stabilization (Moore et al., Blood 117:4542-51, 2011)) can also be used. Antibody fragments can also include isolated CDRs. For a review of antibody fragments, see Hudson, et al., Nat. Med. 9:129-134, 2003.

In particular embodiments, one or more amino acid modifications may be introduced into the Fc region of an antibody, thereby generating an Fc region variant. The Fc region variant may include a human Fc region sequence (e.g., a human IgG1, IgG2, IgG3 or IgG4 Fc region) including an amino acid modification (e.g., a substitution) at one or more amino acid positions. Numerous Fc modifications are known in the art, and a representative sampling of such possible modifications are described herein.

In particular embodiments, variants (including Fc variants) have been modified from a reference sequence to produce an administration benefit. Exemplary administration benefits can include (1) reduced susceptibility to proteolysis, (2) reduced susceptibility to oxidation, (3) altered binding affinity for forming protein complexes, (4) altered binding affinities, (5) reduced immunogenicity; and/or (6) extended half-life. While the disclosure below describes these modifications in terms of their application to antibodies, when applicable to another particular Siglec-8 binding domain format (e.g., bispecific antibodies), the modifications can also be applied to these other formats.

In particular embodiments the antibodies can be mutated to increase their affinity for Fc receptors. Exemplary mutations that increase the affinity for Fc receptors include: G236A/S239D/A330L/I332E (GASDALIE). Smith et al., Proceedings of the National Academy of Sciences of the United States of America, 109(16), 6181-6186, 2012. In particular embodiments, an antibody variant includes an Fc region with one or more amino acid substitutions which improve ADCC, e.g., substitutions at positions 298, 333, and/or 334 of the Fc region (EU numbering of residues). In particular embodiments, alterations are made in the Fc region that result in altered C1q binding and/or Complement Dependent Cytotoxicity (CDC), e.g., as described in U.S. Pat. No. 6,194,551, WO 99/51642, and Idusogie et al., J. Immunol. 164: 4178-4184, 2000.

In particular embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further below. In particular embodiments, residue 5400 (EU numbering) of the heavy chain Fc region is selected. Cysteine engineered antibodies may be generated as described, e.g., in U.S. Pat. No. 7,521,541.

Antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at position 297 in the Fc region (Eu numbering of Fc region residues); however, Asn297 may also be located ±3 amino acids upstream or downstream of position 297, i.e., between positions 294 and 300, due to minor sequence variations in antibodies. Such fucosylation variants may have improved ADCC function. See, e.g., WO2000/61739; WO 2001/29246; WO2002/031140; US2002/0164328; WO2003/085119; WO2003/084570; US2003/0115614; US2003/0157108; US2004/0093621; US2004/0110704; US2004/0132140; US2004/0110282; US2004/0109865; WO2005/035586; WO2005/035778; WO2005/053742; Okazaki et al. J. Mol. Biol. 336:1239-1249 (2004); and Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545, 1986, and knockout cell lines, such as alpha-1,6-fucosyltransferase gene, FUT8, knockout CHO cells (see, e.g., Yamane-Ohnuki et al., Biotech. Bioeng. 87: 614, 2004; Kanda et al., Biotechnol. Bioeng., 94(4):680-688, 2006; and WO2003/085107).

In particular embodiments, modified antibodies include those wherein one or more amino acids have been replaced with a non-amino acid component, or where the amino acid has been conjugated to a functional group or a functional group has been otherwise associated with an amino acid. The modified amino acid may be, e.g., a glycosylated amino acid, a PEGylated amino acid, a farnesylated amino acid, an acetylated amino acid, a biotinylated amino acid, an amino acid conjugated to a lipid moiety, or an amino acid conjugated to an organic derivatizing agent. Amino acid(s) can be modified, for example, co-translationally or post-translationally during recombinant production (e.g., N-linked glycosylation at N-X-S/T motifs during expression in mammalian cells) or modified by synthetic means. The modified amino acid can be within the sequence or at the terminal end of a sequence. Modifications also include nitrited constructs.

In particular embodiments, variants include glycosylation variants wherein the number and/or type of glycosylation site has been altered compared to the amino acid sequences of a reference sequence. In particular embodiments, glycosylation variants include a greater or a lesser number of N-linked glycosylation sites than the reference sequence. An N-linked glycosylation site is characterized by the sequence: Asn-X-Ser or Asn-X-Thr, wherein the amino acid residue designated as X can be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. Also provided is a rearrangement of N-linked carbohydrate chains wherein one or more N-linked glycosylation sites (e.g., those that are naturally occurring) are eliminated and one or more new N-linked sites are created. Additional antibody variants include cysteine variants wherein one or more cysteine residues are deleted from or substituted for another amino acid (e.g., serine) as compared to the reference sequence. These cysteine variants can be useful when antibodies must be refolded into a biologically active conformation such as after the isolation of insoluble inclusion bodies. These cysteine variants generally have fewer cysteine residues than the reference sequence, and typically have an even number to minimize interactions resulting from unpaired cysteines.

PEGylation particularly is a process by which polyethylene glycol (PEG) polymer chains are covalently conjugated to other molecules such as proteins. Several methods of PEGylating proteins have been reported in the literature. For example, N-hydroxy succinimide (NHS)-PEG was used to PEGylate the free amine groups of lysine residues and N-terminus of proteins; PEGs bearing aldehyde groups have been used to PEGylate the amino-termini of proteins in the presence of a reducing reagent; PEGs with maleimide functional groups have been used for selectively PEGylating the free thiol groups of cysteine residues in proteins; and site-specific PEGylation of acetyl-phenylalanine residues can be performed.

Covalent attachment of proteins to PEG has proven to be a useful method to increase the half-lives of proteins in the body (Abuchowski, A. et al., Cancer Biochem. Biophys., 1984, 7:175-186; Hershfield, M. S. et al., N. Engl. J. Medicine, 1987, 316:589-596; and Meyers, F. J. et al., Clin. Pharmacol. Ther., 49:307-313, 1991). The attachment of PEG to proteins not only protects the molecules against enzymatic degradation, but also reduces their clearance rate from the body. The size of PEG attached to a protein has significant impact on the half-life of the protein. The ability of PEGylation to decrease clearance is generally not a function of how many PEG groups are attached to the protein, but the overall molecular weight of the altered protein. Usually the larger the PEG is, the longer the in vivo half-life of the attached protein. In addition, PEGylation can also decrease protein aggregation (Suzuki et al., Biochem. Bioph. Acta 788:248, 1984), alter protein immunogenicity (Abuchowski et al., J. Biol. Chem. 252: 3582, 1977), and increase protein solubility as described, for example, in PCT Publication No. WO 92/16221).

Several sizes of PEGs are commercially available (Nektar Advanced PEGylation Catalog 2005-2006; and NOF DDS Catalogue Ver 7.1), which are suitable for producing proteins with targeted circulating half-lives. A variety of active PEGs have been used including mPEG succinimidyl succinate, mPEG succinimidyl carbonate, and PEG aldehydes, such as mPEG-propionaldehyde.

In particular embodiments, the antibody can be fused or coupled to an Fc polypeptide that includes amino acid alterations that extend the in vivo half-life of an antibody that contains the altered Fc polypeptide as compared to the half-life of a similar antibody containing the same Fc polypeptide without the amino acid alterations. In particular embodiments, Fc polypeptide amino acid alterations can include M252Y, S254T, T256E, M428L, and/or N434S and can be used together, separately or in any combination. For example, M428L/N434S is a pair of mutations that increase the half-life of antibodies in serum, as described in Zalevsky et al., Nature Biotechnology 28, 157-159, 2010. Other alterations that can be helpful are described in U.S. Pat. Nos. 7,083,784, 7,670,600, US Publication No. 2010/0234575, PCT/US2012/070146, and Zwolak, Scientific Reports 7: 15521, 2017. In particular embodiments, any substitution at one of the following amino acid positions in an Fc polypeptide can be considered an Fc alteration that extends half-life: 250, 251, 252, 259, 307, 308, 332, 378, 380, 428, 430, 434, 436. Each of these alterations or combinations of these alterations can be used to extend the half-life of a bispecific antibody as described herein.

In particular embodiments, Fc modifications include hulgG4 ProAlaAla, hulgG2m4, and/or hulgG2sigma mutations. In particular embodiments, one or several amino acids at the amino or carboxy terminus of the light and/or heavy chain, such as the C-terminal lysine of the heavy chain, may be missing or derivatized in a proportion or all of the molecules. Substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004). For additional information regarding Fc mutations that create administration benefits, see Saunders, Conceptual Approaches to Modulating Antibody Effector Functions and Circulation Half-Life, Frontiers in Immunology (2019) Vol. 10, Article 1296.

(iii) Multi-Domain Binding Molecules. Multi-domain binding molecules include at least two binding domains, wherein at least one binding domain includes a Siglec-8 binding domain disclosed herein. In particular embodiments, a multi-domain binding molecule includes at least one, at least two, at least, three, at least four binding domains that bind an epitope on Siglec-8. In particular embodiments, all of the binding domains of a multi-domain binding molecule bind Siglec-8. In particular embodiments, a multi-domain binding molecule includes a bispecific antibody, a trispecific antibody, and so on.

Multi-domain binding molecules include bispecific antibodies which bind at least two epitopes wherein at least one of the epitopes is located on Siglec-8. Multi-domain binding molecules include trispecific antibodies which binds at least 3 epitopes, wherein at least one of the epitopes is located on Siglec-8, and so on.

Bispecific antibodies can be prepared as full length antibodies or antibody fragments (for example, F(ab′)₂ bispecific antibodies). For example, WO 1996/016673 describes a bispecific anti-ErbB2/anti-Fc gamma RIII antibody; U.S. Pat. No. 5,837,234 describes a bispecific anti-ErbB2/anti-Fc gamma RI antibody; WO 1998/002463 describes a bispecific anti-ErbB2/anti-Fc alpha antibody; and U.S. Pat. No. 5,821,337 describes a bispecific anti-ErbB2/anti-CD3 antibody.

Some additional exemplary bispecific antibodies have two heavy chains (each having three heavy chain CDRs, followed by (N-terminal to C-terminal) a CH1 domain, a hinge, a CH2 domain, and a CH3 domain), and two immunoglobulin light chains that confer antigen-binding specificity through association with each heavy chain. However, as indicated, additional architectures are envisioned, including bi-specific antibodies in which the light chain(s) associate with each heavy chain but do not (or minimally) contribute to antigen-binding specificity, or that can bind one or more of the epitopes bound by the heavy chain antigen-binding regions, or that can associate with each heavy chain and enable binding of one or both of the heavy chains to one or both epitopes.

Two antibodies or fragments thereof can be linked through a linker to form a bispecific antibody. In particular embodiments, the two antibodies or fragments thereof can bind the same epitope or different epitopes. Examples of linkers can be found in Chen et al., Adv Drug Deliv Rev. 65(10): 1357-1369, 2013. Linkers can be flexible, rigid, or semi-rigid, depending on the desired functional domain presentation to a target.

Commonly used flexible linkers include linker sequences with the amino acids glycine and serine (Gly-Ser linkers). In particular embodiments, the linker sequence includes sets of glycine and serine repeats such as from one to ten repeats of (GlyₓSer_{y})ₙ, wherein x and y are independently an integer from 0 to 10 provided that x and y are not both 0 and wherein n is an integer of 1, 2, 3, 4, 5, 6, 7, 8, 9 or 10). Particular examples include (Gly₄Ser)ₙ (SEQ ID NO: 347), (Gly₃Ser)ₙ(Gly₄Ser)ₙ (SEQ ID NO: 348), (Gly₃Ser)ₙ(Gly₂Ser)ₙ (SEQ ID NO: 349), and (Gly₃Ser)ₙ(Gly₄Ser)₁ (SEQ ID NO: 350). In particular embodiments, the linker is (Gly₄Ser)₄ (SEQ ID NO: 351), (Gly₄Ser)₃ (SEQ ID NO: 352), (Gly₄Ser)₂ (SEQ ID NO: 353), (Gly₄Ser)₁ (SEQ ID NO: 354), (Gly₃Ser)₂ (SEQ ID NO: 355), (Gly₃Ser)₁ (SEQ ID NO: 356), (Gly₂Ser)₂ (SEQ ID NO: 357) or (Gly₂Ser)₁, GGSGGGSGGSG (SEQ ID NO: 358), GGSGGGSGSG (SEQ ID NO: 359), or GGSGGGSG (SEQ ID NO: 360).

In particular embodiments, the linker includes the Whitlow linker: GSTSGSGKPGSGEGSTKG (SEQ ID NO: 85; Whitlow et al., Protein Eng 6(8):989-95, 1993). In particular embodiments, the linker used in an scFv includes the Whitlow linker.

Linkers that include one or more antibody hinge regions and/or immunoglobulin heavy chain constant regions, such as CH3 alone or a CH2CH3 sequence can also be used. Additional examples of linkers can be found in Chen et al., (supra). Linkers can be flexible, rigid, or semi-rigid, depending on the desired functional domain presentation to a target.

In some situations, flexible linkers may be incapable of maintaining a distance or positioning of binding domains needed for a particular use. In these instances, rigid or semi-rigid linkers may be useful. Examples of rigid or semi-rigid linkers include proline-rich linkers. In particular embodiments, a proline-rich linker is a peptide sequence having more proline residues than would be expected based on chance alone. In particular embodiments, a proline-rich linker is one having at least 30%, at least 35%, at least 36%, at least 39%, at least 40%, at least 48%, at least 50%, or at least 51% proline residues. Particular examples of proline-rich linkers include fragments of proline-rich salivary proteins (PRPs).

In particular embodiments, binding domains disclosed herein can be used to create bi-, tri, (or more) specific immune cell engaging molecules. Immune cell engaging molecules have at least one binding domain that binds a receptor on an immune cell and alters the activation state of the immune cell. Examples of multi-domain immune cell engaging molecules include those which bind both an immune cell (e.g., T-cell or NK-cells) activating epitope and Siglec-8, with the goal of bringing immune cells to Siglec-8-expressing cells to destroy them. See, for example, US 2008/0145362. Such molecules are referred to herein as immune-activating multi-specifics or I-AMS). BiTEs® (Amgen, Thousand Oaks, CA) are one form of I-AMS. Immune cells that can be targeted for localized activation by I-AMS within the current disclosure include, for example, B-cells, T-cells, natural killer (NK) cells, and macrophages which are discussed in more detail herein.

I-AMS disclosed herein can target any T-cell activating epitope that upon binding induces T-cell activation. Examples of such T-cell activating epitopes are on T-cell markers including CD2, CD3, CD7, CD27, CD28, CD30, CD40, CD83, 4-1BB (CD137), OX40, lymphocyte function-associated antigen-1 (LFA-1), LIGHT, NKG2C, and B7-H3. Binding domains that bind T-cell markers are known in the art. B cell activation can be initiated by binding of an antigen to the B cell receptor (BCR) (e.g., IgM or IgD). Exemplary immune cell activating factors for NK cells include IL-15 and CD137.

In particular embodiments, the CD3 binding domain (e.g., scFv) is derived from the OKT3 antibody (the same as the one utilized in blinatumomab), otelixizumab, teplizumab, visilizumab, 20G6-F3, 4B4-D7, 4E7-C9, 18F5-H10, or TR66. The OKT3 antibody is described in detail in U.S. Pat. No. 5,929,212.

In particular embodiments, a binding domain is “derived from” a reference antibody when the binding domain includes the CDRs of the reference antibody, according to a known numbering scheme (e.g., Kabat, Chothia, Martin, or others).

Other forms of bispecific binding molecules include the single chain “Janusins” described in Traunecker et al. (Embo Journal, 10, 3655-3659, 1991).

Bispecific binding molecules with extended half-lives are described in, for example, U.S. Pat. No. 8,921,528 and US Patent Publication No. 2014/0308285.

In particular embodiments, multi-domain binding molecules are multimers of an antibody disclosed herein. Multimerization strategies include formation of a fusion protein using protein linkers or use of IgA or IgM constant regions as a multimerization scaffold. In certain aspects, multimerization is achieved by linking antibodies or binding domains of antibodies in a fusion protein with protein linkers. Fusion proteins include different protein domains linked to each other directly or through intervening linker segments such that the function of each included domain is retained.

Certain examples include fusion protein with two or three copies of an antibody or binding domain disclosed herein, each linked with the Gly-Ser linker (Gly₄Ser)ₙ (SEQ ID NO: 347) wherein n is an integer of 1, 2, 3, 4, 5, 6, 7, 8, 9 or 10. In particular embodiments, n is 3.

In particular embodiments, a bispecific antibody includes 2 scFvs connected by a linker. In particular embodiments, the scFv-scFv format includes different orientations including: HcFv-LcFv-HcFv-LcFv; HcFv-LcFv-LcFv-HcFv; LcFv-HcFv, HcFv-LcFv; and LcFv-HcFv-LcFv-HcFv. —Particular examples of multi-domain binding molecules and associated coding sequences are provided in FIG. 26E.

A “multimerization domain” is a domain that causes two or more proteins (monomers) to interact with each other through covalent and/or non-covalent association(s). Multimerization domains are highly conserved protein sequences that can include different types of sequence motifs such as leucine zipper, helix loop-helix, ankyrin and PAS (Feuerstein et al, Proc. Natl. Acad. Sci. USA, 91:10655-10659, 1994). Multimerization domains present in proteins can bind to form dimers, trimers, tetramers, pentamers, hexamers, heptamers, etc., depending on the number of units/monomers incorporated into the multimer, and/or homomultimers or heteromultimers, depending on whether the binding monomers are the same type or a different type (U.S. patent Ser. No. 10/030,065).

Dimerization domains can include protein sequence motifs such as coiled coils, acid patches, zinc fingers, calcium hands, a CH1-CL pair, an “interface” with an engineered “knob” and/or “protruberance” (U.S. Pat. No. 5,821,333), leucine zippers (U.S. Pat. No. 5,932,448), SH2 and SH3 (Vidal et al., Biochemistry, 43:7336-44, 2004), PTB (Zhou et al., Nature, 378:584-592, 1995), W W (Sudol Prog Biochys MoL Bio, 65:113-132, 1996), PDZ (Kim et al., Nature, 378: 85-88, 1995; Komau et al., Science, 269:1737-1740, 1995) and WD40 (Hu et al., J Biol Chem., 273:33489-33494, 1998). Additional examples of molecules that contain dimerization domains/motifs are receptor dimer pairs such as the interleukin-8 receptor (IL-8R), integrin heterodimers such as LFA-I and GPIIIb/IIIa, dimeric ligand polypeptides such as nerve growth factor (NGF), neurotrophin-3 (NT-3), interleukin-8 (IL-8), vascular endothelial growth factor (VEGF), VEGF-C, VEGF-D, PDGF members, and brain-derived neurotrophic factor (BDNF) (Arakawa et al., J Biol. Chem., 269:27833-27839, 1994; Radziejewski et al., Biochem, 32: 1350, 1993) and variants of some of these domains with modified affinities (PCT Publication No. WO 2012/001647).

In particular embodiments, the sequence corresponding to a dimerization motif/domain includes the leucine zipper domain of Jun (U.S. Pat. No. 5,932,448; RIARLEEKVKTLKAQNSELASTANMLREQVAQLKQKVMN (SEQ ID NO: 362)), the dimerization domain of Fos (U.S. Pat. No. 5,932,448; LTDTLQAETDQLEDKKSALQTEIANLLKEKEKLEFILAA (SEQ ID NO: 363)), a consensus sequence for a WW motif (PCT Publication No. WO 1997/037223), the dimerization domain of the SH2B adapter protein from GenBank Accession no. AAF73912.1 (Nishi et al., Mol Cell Biol, 25: 2607-2621, 2005; WREFCESHARAAALDFARRFRLYLASHPQYAGPGAEAAFSRRFAELFLQHFEAEVARAS (SEQ ID NO: 364)), the SH3 domain of IB1 from GenBank Accession no. AAD22543.1 (Kristensen et al., EMBO J., 25: 785-797, 2006; THRAIFRFVPRHEDELELEVDDPLLVELQAEDYWYEAYNMRTGARGVFPAYYAIE (SEQ ID. NO: 365)), the PTB domain of human DOK-7 from GenBank Accession no. NP_005535.1 (Wagner et al., Cold Spring Harb Perspect Biol. 5: a008987, 2013; LGEVHRFHVTVAPGTKLESGPATLHLCNDVLVLARDIPPAVTGQWKLSDLRRYGAVPSGFIFEG GTRCGYWAGVFFLSSAEGEQISFLFDCIVRGISPTKG (SEQ ID NO: 366)), the PDZ-like domain of SATB1 from UniProt Accession No. Q01826 (Galande et al., Mol Cell Biol. August; 21: 5591-5604, 2001; DCKEEHAEFVLVRKDMLFNQLIEMALLSLGYSHSSAAQAKGLIQVGKWNPVPLSYVTDAPDAT VADMLQDVYHVVTLKIQLHSCPKLEDLPPEQWSHTTVRNALKDLLKDMNQSS (SEQ ID NO: 367)), the WD40 repeats of APAF from UniProt Accession No. 014727 (Jorgensen et al., 2009. PLOS One. 4(12):e8463; CAPWPMVEKLIKQCLKENPQERPTSAQVFDILNSAELVCLTRRILLPKNVIVECMVATHHNSRN ASIWLGCGHTDRGQLSFLDLNTEGYTSEEVADSRILCLALVHLPVEKESWIVSGTQSGTLLVINT EDGKKRHTLEKMTDSVTCLYCNSFSKQSKQKNFLLVGTADGKLAIFEDKTVKLKGAAPLKILNIG NVSTPLMCLSESTNSTERNVMWGGCGSQLFSYAAFSDSNIITVVVDTALYIAKQNSPVVEVWD KKTEKLCGLIDCVHFLREVMVKETKIFSFSNDFTIQKLIETRTNKESKHKMSYSGRVKTLCLQKN TALWIGTGGGHILLLDLSTRRLIRVIYNFCNSVRVMMTAQLGSLKNVMLVLGYNRKNTEGTQKQ KEIQSCLTVWDINLPHEVQNLEKHIEVRKELAEKMRRTSVE (SEQ ID NO: 368)), the PAS motif of the dioxin receptor from UniProt Accession No. I6L9E7 (Pongratz et al., Mol Cell Biol, 18:4079-4088, 1998; DQELKHLILEAADGFLFIVSCETGRVVYVSDSVTPVLNQQQSEWFGSTLYDQVHPDDVDKLRE QLSTSENALTGR (SEQ ID NO: 369)) and the EF hand motif of parvalbumin from UniProt Accession No. P20472 (Jamalian et al., Int J Proteomics, 2014: 153712, 2014; LSAKETKMLMAAGDKDGDGKIGVDEFSTLVAES (SEQ ID NO: 370)).

In particular embodiments, the dimerization domain can be a dimerization and docking domain (DDD) on one antibody and an anchoring domain (AD) on another antibody to facilitate a stably tethered structure. In particular embodiments, the DDD (DDD1 and DDD2) are derived from the regulatory subunits of a cAMP-dependent protein kinase (PKA), and the AD (AD1 and AD2) are derived from a specific region found in various A-kinase anchoring proteins (AKAPs) that mediates association with the R subunits of PKA. In particular embodiments, DDD1 includes the amino acid sequence: SHIQIPPGLTELLQGYTVEVLRQQPPDLVEFAVEYFTRLREARA (SEQ ID NO: 371). In particular embodiments, DDD2 includes the amino acid sequence: CGHIQIPPGLTELLQGYTVEVLRQQPPDLVEFAVEYFTRLREARA (SEQ ID NO: 372). In particular embodiments, AD1 includes the amino acid sequence: QIEYLAKQIVDNAIQQA (SEQ ID NO: 373). In particular embodiments, AD2 includes the amino acid sequence: CGQIEYLAKQIVDNAIQQAGC (SEQ ID NO: 374). However, one skilled in the art will realize that other DDDs and ADs are known and can be used such as: the 4-helix bundle type DDD domains may be obtained from p53, DCoH (pterin 4 alpha carbinolamine dehydratase/dimerization cofactor of hepatocyte nuclear factor 1 alpha (TCF1)) and HNF-1 (hepatocyte nuclear factor 1). Other AD sequences of potential use may be found in Patent Publication No. US2003/0232420A1.

The X-type four-helix bundle dimerization motif that is a structural characteristic of the DDD (Newlon, et al. EMBO J. 2001; 20: 1651-1662; Newlon, et al. Nature Struct Biol. 1999; 3: 222-227) is found in other classes of proteins, such as the S100 proteins (for example, S100B and calcyclin), and the hepatocyte nuclear factor (HNF) family of transcriptional factors (for example, HNF-1a and HNF-1β). Over 300 proteins that are involved in either signal transduction or transcriptional activation also contain a module of 65-70 amino acids termed the sterile a motif (SAM) domain, which has a variation of the X-type four-helix bundle present on its dimerization interface. For S100B, this X-type four-helix bundle enables the binding of each dimer to two p53 peptides derived from the c-terminal regulatory domain (residues 367-388) with micromolar affinity (Rustandi, et al. Biochemistry. 1998; 37: 1951-1960). Similarly, the N-terminal dimerization domain of HNF-1α (HNF-p1) was shown to associate with a dimer of DCoH (dimerization cofactor for HNF-1) via a dimer of HNF-p1 (Rose, et al. Nature Struct Biol. 2000; 7: 744-748). In alternative embodiments, these naturally occurring systems can also be used to provide stable multimeric structures with multiple functions or binding specificities. Other binding events such as those between an enzyme and its substrate/inhibitor, for example, cutinase and phosphonates (Hodneland, et al. Proc Natl Acad Sci USA. 2002; 99: 5048-5052), may also be utilized to generate the two associating components (the “docking” step), which are subsequently stabilized covalently (the “lock” step).

In particular embodiments, dimerization of antibodies can be induced by a chemical inducer. This method of dimerization requires one antibody to contain a chemical inducer of dimerization binding domain 1 (CBD1) and the second antibody to contain the second chemical inducer of dimerization binding domain (CBD2), wherein CBD1 and CBD2 are capable of simultaneously binding to a chemical inducer of dimerization (CID). If the CID is rapamycin, CBD1 and CBD2 can be the rapamycin binding domain of FK-binding protein 12 (FKBP12) and the FKBP12-Rapamycin Binding (FRB) domain of mTOR. In particular embodiments, FKBP12 includes the sequence:

(SEQ ID NO: 375)
MGVQVETISPGDGRTFPKRGQTCWHYTGMLEDGKKFDSSRDRNPFKFMLG
KQEVIRGWEEGVAQMSVGQRAKLTISPDYAYGATGHPGIIPPHATLVFDV
ELLKLE.

In particular embodiments, FRB includes the sequence: MASRILWHEMWHEGLEEASRLYFGERNVKGMFEVLEPLHAMMERGPQTLKETSFNQAYGRD LMEAQEWCRKYMKSGNVKDLTQAWDLYYHVFRRISKLES (SEQ ID NO: 376). If the CID is FK506/cyclosporin fusion protein or a derivative thereof, CBD1 and CBD2 can be the FK506 (Tacrolimus) binding domain of FK-binding protein 12 (FKBP12) and the cyclosporin binding domain of cyclophilin A. If the CID is estrone/biotin fusion protein or a derivative thereof, CBD1 and CBD2 can be an oestrogen-binding domain (EBD) and a streptavidin binding domain. If the CID is dexamethasone/methotrexate fusion molecule or a derivative thereof, CBD1 and CBD2 can be a glucocorticoid-binding domain (GBD) and a dihydrofolate reductase (DHFR) binding domain. If the CID is O⁶-benzylguanine derivative/methotrexate fusion molecule or a derivative thereof, CBD1 and CBD2 can be an O⁶-alkylguanine-DNA alkyltransferase (AGT) binding domain and a dihydrofolate reductase (DHFR) binding domain. If the CID is RSL1 or a derivative thereof, CBD1 and CBD2 can be a retinoic acid receptor domain and an ecodysone receptor domain. If the CID is AP1903 or a derivative thereof, CBD1 and CBD2 can be the FK506 binding protein (FKBP12) binding domains including a F36V mutation. Use of the CID binding domains can also be used to alter the affinity to the CID. For instance, altering amino acids at positions 2095, 2098, and 2101 of FRB can alter binding to Rapamycin: KTW has high, KHF intermediate and PLW is low (Bayle et al, Chemistry & Biology 13, 99-107, January 2006).

In particular embodiments, antibodies can multimerize using a transmembrane polypeptide derived from a FcεRI chain. In particular embodiments, an antibody can include a part of a FcεRI alpha chain and another antibody can include a part of an FcεRI beta chain or variant thereof such that said FcεRI chains spontaneously dimerize together to form a dimeric antibody. In particular embodiments, antibodies can include a part of a FcεRI alpha chain and a part of a FcεRI gamma chain or variant thereof such that said FcεRI chains spontaneously trimerize together to form a trimeric antibody, and in another embodiment the multi-chain antibody can include a part of FcεRI alpha chain, a part of FcεRI beta chain and a part of FcεRI gamma chain or variants thereof such that said FcεRI chains spontaneously tetramerize together to form a tetrameric antibody.

In particular embodiments, additional methods of causing dimerization can be utilized. Additional modifications to generate a dimerization domain in antibody could include: replacing the C-terminus domain with murine counterparts; generating a second interchain disulfide bond in the C-terminus domain by introducing a second cysteine residue into both antibodies; swapping interacting residues in each of the antibodies in the C-terminus domains (“knob-in-hole”); and fusing the variable domains of the antibodies directly to CD3 (CD3 fusion) (Schmitt et al., Hum. Gene Ther. 2009. 20:1240-1248).

Particular embodiments can utilize multimerization domains, such as C4b multimerization domains or ferritin multimerization domains. Full-length native C4b includes seven α-chains linked together by a multimerization (i.e., heptamerization) domain at the C-terminus of the α-chains. Blom et al., (2004) Mol Immunol 40: 1333-1346. Ferritin is an iron storage protein found in almost all living organisms, and has been extensively studied and engineered for a number of biochemical/biomedical purposes (US 20090233377; Meldrum, et al. Science 257, 522-523 (1992); U.S. 20110038025; Yamashita, Biochim Biophys Acta 1800, 846-857 (2010), including as a multimerizing vaccine platform for displaying peptide epitopes (US 20060251679 (2006); Li, et al. Industrial Biotechnol 2, 143-147 (2006)).

Multimerization with encapsulin and lumazine synthase can also be performed. Both can be linked to antibodies to create self-assembling 60mer particles (Jardine et al., 2013, Science 340, 711-716 and Kanekiyo et al., 2015, Cell 162, 1090-1100).

Multimerized antibodies and antibody-like molecules such as IgA and IgM antibodies have emerged as promising drug candidates in the fields of, e.g., immuno-oncology and infectious diseases allowing for improved specificity, improved avidity, and the ability to bind to multiple binding targets. See, e.g., U.S. Pat. Nos. 9,951,134, 10,400,038, and 9,938,347, U.S. Patent Application Publication Nos. US20190100597A1, US20180118814A1, US20180118816A1, US20190185570A1, and US20180265596A1, and PCT Publication Nos. WO 2018/017888, WO 2018/017763, WO 2018/017889, WO 2018/017761, and WO 2019/165340.

Particular embodiments include using IgA and IgM constant region domains to allow the binding portion of molecules provided herein to readily multimerize into dimers, pentamers or hexamers. Basic immunoglobulin structures in vertebrate systems are described above and are well understood. (See, e.g., Harlow et al., Antibodies: A Laboratory Manual, Cold Spring Harbor Laboratory Press, 2nd ed. 1988).

Immunoglobulin A (IgA), as the major class of antibody present in the mucosal secretions of most mammals, represents a key first line of defense against invasion by inhaled and ingested pathogens. IgA is also found at significant concentrations in the serum of many species, where it functions as a second line of defense mediating elimination of pathogens that have breached the mucosal surface. Receptors specific for the Fc region of IgA, FcaR, are key mediators of IgA effector function. Native IgA is a tetrameric protein including two identical light chains (κ or λ) and two identical heavy chains. IgA, similarly to IgG, contains three constant domains (CA1-CA3), with a hinge region between the CA1 and CA2 domains. The main difference between IgA1 and IgA2 resides in the hinge region that lies between the two Fab arms and the Fc region. IgA1 has an extended hinge region due to the insertion of a duplicated stretch of amino acids, which is absent in IgA2. Both forms of IgA have the capacity to form dimers, in which two monomer units, are arranged in an end-to-end configuration stabilized by disulfide bridges and incorporation of a J-chain. J-chains are also part of IgM pentamers and are discussed in more detail below. In particular embodiments, binding domains disclosed herein can be expressed as an IgA antibody. In particular embodiments, binding domains disclosed herein can be expressed as an IgM antibody. In particular embodiments, binding domains disclosed herein can be expressed as an IgG antibody.

Both IgA and IgM (discussed further below in relation to pentamers and hexamers) possess an 18-amino acid extension in the C terminus called the “tail-piece” (tp). The IgA and IgM tp is highly conserved among various animal species. The conserved penultimate cysteine residue in the IgA and IgM tp has been demonstrated to be involved in multimerization by forming a disulfide bond between heavy chains to permit formation of a multimer. Both tp contain an N-linked carbohydrate addition site, the presence of which is required for dimer formation in IgA and J-chain incorporation and pentamer formation in IgM. However, the structure and composition of the N-linked carbohydrates in the tp differ, suggesting differences in the accessibility of the glycans to processing by glycosyltransferases. Particularly, the IgA (atp) and IgM (μtp) tp differ at seven amino acid positions.

The human IgA1 constant region typically includes the amino acid sequence represented in SEQ ID NO: 377. Referring to this SEQ ID NO: 377, the human CA1 domain extends from amino acid 6 to amino acid 98; the human IgA1 hinge region extends from amino acid 102 to amino acid 124, the human CA2 domain extends from amino acid 125 to amino acid 219, the human CA3 domain extends from amino acid 228 to amino acid 330, and the tp extends from amino acid 331 to amino acid 352.

The human IgA2 constant region typically includes the amino acid sequence represented in SEQ ID NO: 378. Referring to this SEQ ID NO: 378, the human CA1 domain extends from amino acid 6 to amino acid 98, the human IgA2 hinge region extends from amino acid 102 to amino acid 111, the human CA2 domain extends from amino acid 113 to amino acid 206, the human CA3 domain extends from amino acid 215 to amino acid 317, and the tp extends from amino acid 318 to amino acid 340.

As indicated, two IgA binding units can form a complex with two additional polypeptide chains, the J chain (e.g., SEQ ID NO: 379, the mature human J chain) and the secretory component to form a bivalent secretory IgA (sIgA)-derived binding molecule. An exemplary precursor secretory component includes the sequence MLLFVLTCLLAVFPAISTKSPIFGPEEVNSVEGNSVSITCYYPPTSVNRHTRKYWCRQGARGGC ITLISSEGYVSSKYAGRANLTNFPENGTFVVNIAQLSQDDSGRYKCGLGINSRGLSFDVSLEVS QGPGLLNDTKVYTVDLGRTVTINCPFKTENAQKRKSLYKQIGLYPVLVIDSSGYVNPNYTGRIRL DIQGTGQLLFSVVINQLRLSDAGQYLCQAGDDSNSNKKNADLQVLKPEPELVYEDLRGSVTFH CALGPEVANVAKFLCRQSSGENCDVVVNTLGKRAPAFEGRILLNPQDKDGSFSVVITGLRKED AGRYLCGAHSDGQLQEGSPIQAWQLFVNEESTIPRSPTVVKGVAGGSVAVLCPYNRKESKSIK YWCLWEGAQNGRCPLLVDSEGWVKAQYEGRLSLLEEPGNGTFTVILNQLTSRDAGFYWCLTN GDTLWRTTVEIKIIEGEPNLKVPGNVTAVLGETLKVPCHFPCKFSSYEKYWCKWNNTGCQALP SQDEGPSKAFVNCDENSRLVSLTLNLVTRADEGWYWCGVKQGHFYGETAAVYVAVEERKAA GSRDVSLAKADAAPDEKVLDSGFREIENKAIQDPRLFAEEKAVADTRDQADGSRASVDSGSSE EQGGSSRALVSTLVPLGLVLAVGAVAVGVARARHRKNVDRVSIRSYRTDISMSDFENSREFGA NDNMGASSITQETSLGGKEEFVATTESTTETKEPKKAKRSSKEEAEMAYKDFLLQSSTVAAEA QDGPQEA (SEQ ID NO: 380). An exemplary mature secretory component includes KSPIFGPEEVNSVEGNSVSITCYYPPTSVNRHTRKYWCRQGARGGCITLISSEGYVSSKYAGR ANLTNFPENGTFVVNIAQLSQDDSGRYKCGLGINSRGLSFDVSLEVSQGPGLLNDTKVYTVDL GRTVTINCPFKTENAQKRKSLYKQIGLYPVLVIDSSGYVNPNYTGRIRLDIQGTGQLLFSVVINQL RLSDAGQYLCQAGDDSNSNKKNADLQVLKPEPELVYEDLRGSVTFHCALGPEVANVAKFLCR QSSGENCDVVVNTLGKRAPAFEGRILLNPQDKDGSFSVVITGLRKEDAGRYLCGAHSDGQLQE GSPIQAWQLFVNEESTIPRSPTVVKGVAGGSVAVLCPYNRKESKSIKYWCLWEGAQNGRCPLL VDSEGWVKAQYEGRLSLLEEPGNGTFTVILNQLTSRDAGFYWCLTNGDTLWRTTVEIKIIEGEP NLKVPGNVTAVLGETLKVPCHFPCKFSSYEKYWCKWNNTGCQALPSQDEGPSKAFVNCDEN SRLVSLTLNLVTRADEGWYWCGVKQGHFYGETAAVYVAVEERKAAGSRDVSLAKADAAPDEK VLDSGFREIENKAIQDPR (SEQ ID NO: 381). While not wishing to be bound by theory, and as indicated above, the assembly of two IgA binding units into a dimeric IgA-derived binding molecule is thought to involve the CA3 and tp domains. See, e.g., Braathen, R., et al., J. Biol. Chem. 277:42755-42762 (2002). Accordingly, a multimerizing dimeric IgA-derived binding molecule provided in this disclosure typically includes IgA constant regions that include at least the CA3 and tp domains.

An engineered IgA heavy chain constant region can additionally include a CA2 domain or a fragment thereof, an IgA hinge region or fragment thereof, a CA1 domain or a fragment thereof, and/or other IgA (or other immunoglobulin, e.g., IgG) heavy chain domains, including, e.g., an IgG hinge region. In certain embodiments, a binding molecule as provided herein can include a complete IgA heavy chain constant region (e.g., SEQ ID NO: 377 or SEQ ID NO: 378), or a variant, derivative, or analog thereof.

In particular embodiments, the IgA heavy chain constant regions can include amino acids 125 to 353 of SEQ ID NO: 377 or amino acids 113 to 340 of SEQ ID NO: 378. In particular embodiments, the IgA heavy chain constant regions can each further include an IgA or IgG hinge region situated N-terminal to the IgA CA2 domains. For example, the IgA heavy chain constant regions can include amino acids 102 to 353 of SEQ ID NO: 377 or amino acids 102 to 340 of SEQ ID NO: 378. In particular embodiments, the IgA heavy chain constant regions can each further include an IgA CA1 domain situated N-terminal to the IgA hinge region.

Each of the strategies discussed above can be used to create IgA antibody-based dimers.

Particular embodiments include IgM immunoglobulin constant region domains that allow the binding portion of molecules provided herein to readily multimerize into pentamers or hexamers.

Particular embodiments include IgM constant regions (or variants thereof). These embodiments have the ability to form hexamers, or in association with a J-chain, form pentamers. Embodiments with an IgM constant region typically include at least the Cμ4-tp domains of the IgM constant region but can include heavy chain constant region domains from other antibody isotypes, e.g., IgG, from the same species or from a different species. In particular embodiments, one or more constant region domains can be deleted so long as the IgM antibody is capable of forming hexamers and/or pentamers. Thus, an IgM antibody can be, e.g., a hybrid IgM/IgG antibody or can be a “multimerizing fragment” of an IgM-derived binding molecule.

The assembly of five or six IgM binding units into a pentameric or hexameric IgM antibody is thought to involve the Cμ4 and tp domains. See, e.g., Braathen, R., et al., J Biol. Chem. 277:42755-42762 (2002). Accordingly, a pentameric or hexameric IgM antibody described in this disclosure typically includes at least the Cμ4 and/or tp domains (also referred to herein collectively as Cμ4-tp). A “multimerizing fragment” of an IgM heavy chain constant region thus includes at least the Cμ4-tp domains. An IgM heavy chain constant region can additionally include a Cμ3 domain or a fragment thereof, a Cμ2 domain or a fragment thereof, a Cμ1 domain or a fragment thereof, and/or other IgM heavy chain domains.

Five IgM monomers form a complex with a J-chain to form a native IgM molecule. The J-chain is considered to facilitate polymerization of μ chains before IgM is secreted from antibody-producing cells. Sequences for the human IGJ gene are known in the art, for example, (IGMT Accession: J00256, X86355, M25625, AJ879487). The J chain establishes the disulfide bridges between IgM antibodies to form multimeric structures such as pentamers. See, for example, Sorensen et al. International Immunology, (2000), pages 19-27. While crystallization of IgM has proved to be notoriously challenging, Czajkowsky and Shao (PNAS 106(35): 14960-14965, 2009) published a homology-based structural model of IgM, based on the structure of the IgE Fc domain and the known disulfide pairings. The authors report that the human IgM pentamer is a mushroom-shaped molecule with a flexural bias. The IgM heavy (μ) chain contains five N-linked glycosylation sites: Asn-171, Asn-332, Asn-395, Asn-402 and Asn-563. In an IgM antibody where each binding unit is bivalent, the binding molecule itself can have 10 or 12 valencies.

The Kabat numbering system for the human IgM constant domain can be found in Kabat, et. al. “Tabulation and Analysis of Amino acid and nucleic acid Sequences of Precursors, V-Regions, C-Regions, J-Chain, T-Cell Receptors for Antigen, T-Cell Surface Antigens, b-2 Microglobulins, Major Histocompatibility Antigens, Thy-I, Complement, C-Reactive Protein, Thymopoietin, Integrins, Post-gamma Globulin, a-2 Macroglobulins, and Other Related Proteins,” U.S. Dept of Health and Human Services (1991). IgM constant regions can be numbered sequentially (i.e., amino acid #1 starting with the first amino acid of the constant region) or by using the Kabat numbering scheme.

A “full length IgM antibody heavy chain” is a polypeptide that includes, in N-terminal to C-terminal direction, an antibody heavy chain variable domain (VH), an antibody heavy chain constant domain 1 (CM1 or Cμ1), an antibody heavy chain constant domain 2 (CM2 or Cμ2), an antibody heavy chain constant domain 3 (CM3 or Cμ3), and an antibody heavy chain constant domain 4 (CM4 or Cμ4) that can include a tp, as indicated above.

In particular embodiments, each binding unit of a multimeric binding molecule as provided herein includes two IgM heavy chain constant regions or multimerizing fragments or variants thereof, each including at least an IgM Cμ4 domain and an IgM tp domain. In certain embodiments the IgM heavy chain constant regions can each further include an IgM Cμ3 domain situated N-terminal to the IgM Cμ4 and IgM tp domains.

In particular embodiments, the IgM heavy chain constant regions can each further include an IgM Cμ2 domain situated N-terminal to the IgM Cμ3 domain. Exemplary multimeric binding molecules provided herein include human IgM constant regions that include the wild-type human Cμ2, Cμ3, and Cμ4-tp domains which are represented in SEQ ID NO: 382.

In certain IgM-derived multimeric binding molecules as provided herein each IgM constant region can include, instead of, or in addition to an IgM Cμ2 domain, an IgG hinge region or functional variant thereof situated N-terminal to the IgM Cμ3 domain. An exemplary variant human IgG1 hinge region amino acid sequence in which the cysteine at position 6 is substituted with serine is VEPKSSDKTHTCPPCPAP (SEQ ID NO: 383). An exemplary IgM constant region of this type includes the variant human IgG1 hinge region fused to a multimerizing fragment of the human IgM constant region including the Cμ3, Cμ4, and tp domains, and includes the amino acid sequence:

(SEQ ID NO: 384)
VEPKSSDKTHTCPPCPAPDQDTAIRVFAIPPSFASIFLTKSTKLTCLVTD
LTTYDSVTISWTRQNGEAVKTHTNISESHPNATFSAVGEASICEDDWNSG
ERFTCTVTHTDLPSPLKQTISRPKGVALHRPDVYLLPPAREQLNLRESAT
ITCLVTGFSPADVFVQWMQRGQPLSPEKYVTSAPMPEPQAPGRYFAHSIL
TVSEEEWNTGETYTCVVAHEALPNRVTERTVDKSTGKPTLYNVSLVMSDT
AGTCY.

Human IgM constant regions, and also certain non-human primate IgM constant regions, as provided herein typically include five (5) naturally-occurring asparagine (N)-linked glycosylation motifs or sites. As used herein “an N-linked glycosylation motif” includes the amino acid sequence N-X1-S/T, wherein N is asparagine, X1 is any amino acid except proline (P), and S/T is serine (S) or threonine (T). The glycan is attached to the nitrogen atom of the asparagine residue. See, e.g., Drickamer K, Taylor M E (2006), Introduction to Glycobiology (2nd ed.). Oxford University Press, USA. N-linked glycosylation motifs occur in the human IgM heavy chain constant regions of SEQ ID NO: 385 or SEQ ID NO: 386 starting at positions 46 (“N1”), 209 (“N2”), 272 (“N3”), 279 (“N4”), and 440 (“N5”). These five motifs are conserved in non-human primate IgM heavy chain constant regions, and four of the five are conserved in the mouse IgM heavy chain constant region. Each of these sites in the human IgM heavy chain constant region, except for N4, can be mutated to prevent glycosylation at that site, while still allowing IgM expression and assembly into a hexamer or pentamer.

The human IgM heavy chain constant region typically includes the amino acid sequence represented in SEQ ID NOs: 385 and 386.

Other forms of the human IgM constant region with minor sequence variations exist, including GenBank Accession Nos. P01871.4, CAB37838.1, and pir∥MHHU. The amino acid substitutions, insertions, and/or deletions at positions corresponding to SEQ ID NO: 385 described herein can likewise be incorporated into alternate human IgM sequences, as well as into IgM constant region amino acid sequences of other species, e.g., those shown in FIG. 1 of PCT/US2019/020374.

In certain aspects, a variant human IgM constant region includes an amino acid substitution corresponding to the wild-type human IgM constant region at position P311, P313, R344, E345, S401, E402, and/or E403 of SEQ ID NO: 385. These positions correspond to the Kabat numbering system as follows: S401 of SEQ ID NO: 385 corresponds to S524 of Kabat; E402 of SEQ ID NO: 385 corresponds to E525 of Kabat; E403 of SEQ ID NO: 385 corresponds to E526 of Kabat; R344 of SEQ ID NO: 385 corresponds to R467 of Kabat; and E345 of SEQ ID NO: 385 corresponds to E468 of Kabat.

In particular embodiments, “corresponds to” means the designated position of SEQ ID NO: 385 and the amino acid in the sequence of the IgM constant region of any species which is homologous to the specified position. See FIG. 1 of PCT/US2019/020374.

In particular embodiments, P311 of SEQ ID NO: 385 can be substituted, e.g., with alanine (P311A), serine (P311S), or glycine (P311G) and/or P313 of SEQ ID NO: 385 can be substituted, e.g., with alanine (P313A), serine (P313S), or glycine (P313G). P311 and P313 of SEQ ID NO: 385 can be substituted with alanine (P311A) and serine (P313S), respectively as shown in the following sequence: (mutations underlined)

(SEQ ID NO: 387)
GSASAPTLFPLVSCENSPSDTSSVAVGCLAQDFLPDSITFSWKYKNNSDI
SSTRGFPSVLRGGKYAATSQVLLPSKDVMQGTDEHVVCKVQHPNGNKEKN
VPLPVIAELPPKVSVFVPPRDGFFGNPRKSKLICQATGFSPRQIQVSWLR
EGKQVGSGVTTDQVQAEAKESGPTTYKVTSTLTIKESDWLSQSMFTCRVD
HRGLTFQQNASSMCVPDQDTAIRVFAIPPSFASIFLTKSTKLTCLVTDLT
TYDSVTISWTRQNGEAVKTHTNISESHPNATFSAVGEASICEDDWNSGER
FTCTVTHTDLASSLKQTISRPKGVALHRPDVYLLPPAREQLNLRESATIT
CLVTGFSPADVFVQWMQRGQPLSPEKYVTSAPMPEPQAPGRYFAHSILTV
SEEEWNTGETYTCVVAHEALPNRVTERTVDKSTGKPTLYNVSLVMSDTAG
TCY.

In certain aspects, S401 of SEQ ID NO: 385 can be substituted with any amino acid. In certain aspects, S401 of SEQ ID NO: 385 can be substituted with alanine (A) as follows (alanine substitution underlined):

(SEQ ID NO: 388)
GSASAPTLFPLVSCENSPSDTSSVAVGCLAQDFLPDSITFSWKYKNNSDI
SSTRGFPSVLRGGKYAATSQVLLPSKDVMQGTDEHVVCKVQHPNGNKEKN
VPLPVIAELPPKVSVFVPPRDGFFGNPRKSKLICQATGFSPRQIQVSWLR
EGKQVGSGVTTDQVQAEAKESGPTTYKVTSTLTIKESDWLSQSMFTCRVD
HRGLTFQQNASSMCVPDQDTAIRVFAIPPSFASIFLTKSTKLTCLVTDLT
TYDSVTISWTRQNGEAVKTHTNISESHPNATFSAVGEASICEDDWNSGER
FTCTVTHTDLPSPLKQTISRPKGVALHRPDVYLLPPAREQLNLRESATIT
CLVTGFSPADVFVQWMQRGQPLSPEKYVTSAPMPEPQAPGRYFAHSILTV
AEEEWNTGETYTCVVAHEALPNRVTERTVDKSTGKPTLYNVSLVMSDTAG
TCY.

In certain aspects, E402 of SEQ ID NO: 385 can be substituted with any amino acid. In certain aspects, E402 of SEQ ID NO: 385 can be substituted with alanine (A) as follows (alanine substitution underlined):

(SEQ ID NO: 389)
GSASAPTLFPLVSCENSPSDTSSVAVGCLAQDFLPDSITFSWKYKNNSDI
SSTRGFPSVLRGGKYAATSQVLLPSKDVMQGTDEHVVCKVQHPNGNKEKN
VPLPVIAELPPKVSVFVPPRDGFFGNPRKSKLICQATGFSPRQIQVSWLR
EGKQVGSGVTTDQVQAEAKESGPTTYKVTSTLTIKESDWLSQSMFTCRVD
HRGLTFQQNASSMCVPDQDTAIRVFAIPPSFASIFLTKSTKLTCLVTDLT
TYDSVTISWTRQNGEAVKTHTNISESHPNATFSAVGEASICEDDWNSGER
FTCTVTHTDLPSPLKQTISRPKGVALHRPDVYLLPPAREQLNLRESATIT
CLVTGFSPADVFVQWMQRGQPLSPEKYVTSAPMPEPQAPGRYFAHSILTV
SAEEWNTGETYTCVVAHEALPNRVTERTVDKSTGKPTLYNVSLVMSDTAG
TCY.

In certain aspects, E403 of SEQ ID NO: 385 can be substituted with any amino acid. In certain aspects, E403 of SEQ ID NO: 385 can be substituted with alanine (A) as follows (alanine substitution underlined):

(SEQ ID NO: 390)
GSASAPTLFPLVSCENSPSDTSSVAVGCLAQDFLPDSITFSWKYKNNSDI
SSTRGFPSVLRGGKYAATSQVLLPSKDVMQGTDEHVVCKVQHPNGNKEKN
VPLPVIAELPPKVSVFVPPRDGFFGNPRKSKLICQATGFSPRQIQVSWLR
EGKQVGSGVTTDQVQAEAKESGPTTYKVTSTLTIKESDWLSQSMFTCRVD
HRGLTFQQNASSMCVPDQDTAIRVFAIPPSFASIFLTKSTKLTCLVTDLT
TYDSVTISWTRQNGEAVKTHTNISESHPNATFSAVGEASICEDDWNSGER
FTCTVTHTDLPSPLKQTISRPKGVALHRPDVYLLPPAREQLNLRESATIT
CLVTGFSPADVFVQWMQRGQPLSPEKYVTSAPMPEPQAPGRYFAHSILTV
SEAEWNTGETYTCVVAHEALPNRVTERTVDKSTGKPTLYNVSLVMSDTAG
TCY.

In certain aspects, R344 of SEQ ID NO: 385 can be substituted with any amino acid. In certain aspects, R344 of SEQ ID NO: 385 can be substituted with alanine (A) as follows (alanine substitution underlined):

(SEQ ID NO: 391)
GSASAPTLFPLVSCENSPSDTSSVAVGCLAQDFLPDSITFSWKYKNNSDI
SSTRGFPSVLRGGKYAATSQVLLPSKDVMQGTDEHVVCKVQHPNGNKEKN
VPLPVIAELPPKVSVFVPPRDGFFGNPRKSKLICQATGFSPRQIQVSWLR
EGKQVGSGVTTDQVQAEAKESGPTTYKVTSTLTIKESDWLSQSMFTCRVD
HRGLTFQQNASSMCVPDQDTAIRVFAIPPSFASIFLTKSTKLTCLVTDLT
TYDSVTISWTRQNGEAVKTHTNISESHPNATFSAVGEASICEDDWNSGER
FTCTVTHTDLPSPLKQTISRPKGVALHRPDVYLLPPAREQLNLAESATIT
CLVTGFSPADVFVQWMQRGQPLSPEKYVTSAPMPEPQAPGRYFAHSILTV
SEEEWNTGETYTCVVAHEALPNRVTERTVDKSTGKPTLYNVSLVMSDTAG
TCY.

In certain aspects, E345 of SEQ ID NO: 385 can be substituted with any amino acid. In certain aspects, E345 of SEQ ID NO: 385 can be substituted with alanine (A) as follows (alanine substitution underlined):

(SEQ ID NO: 392)
GSASAPTLFPLVSCENSPSDTSSVAVGCLAQDFLPDSITFSWKYKNNSDI
SSTRGFPSVLRGGKYAATSQVLLPSKDVMQGTDEHVVCKVQHPNGNKEKN
VPLPVIAELPPKVSVFVPPRDGFFGNPRKSKLICQATGFSPRQIQVSWLR
EGKQVGSGVTTDQVQAEAKESGPTTYKVTSTLTIKESDWLSQSMFTCRVD
HRGLTFQQNASSMCVPDQDTAIRVFAIPPSFASIFLTKSTKLTCLVTDLT
TYDSVTISWTRQNGEAVKTHTNISESHPNATFSAVGEASICEDDWNSGER
FTCTVTHTDLPSPLKQTISRPKGVALHRPDVYLLPPAREQLNLRASATIT
CLVTGFSPADVFVQWMQRGQPLSPEKYVTSAPMPEPQAPGRYFAHSILTV
SEEEWNTGETYTCVVAHEALPNRVTERTVDKSTGKPTLYNVSLVMSDTAG
TCY.

As indicated, five IgM binding units can form a complex with a J-chain to form a pentameric IgM antibody. The precursor form of the human J-chain includes: MKNHLLFWGVLAVFIKAVHVKAQEDERIVLVDNKCKCARITSRIIRSSEDPNEDIVERNIRIIVPLN NRENISDPTSPLRTRFVYHLSDLCKKCDPTEVELDNQIVTATQSNICDEDSATETCYTYDRNKC YTAVVPLVYGGETKMVETALTPDACYPD (SEQ ID NO: 393). The signal peptide extends from amino acid 1 to amino acid 22 of SEQ ID NO: 393 and the mature human J-chain extends from amino acid 23 to amino acid 159 of SEQ ID NO: 393.

The mature human J-chain includes the amino acid sequence

(SEQ ID NO: 379)
QEDERIVLVDNKCKCARITSRIIRSSEDPNEDIVERNIRIIVPLNNRENI
SDPTSPLRTRFVYHLSDLCKKCDPTEVELDNQIVTATQSNICDEDSATET
CYTYDRNKCYTAVVPLVYGGETKMVETALTPDACYPD.

The term “J-chain” as used herein refers to the J-chain of native sequence IgM or IgA antibodies of any animal species. When specified, it can also refer to any functional fragment thereof, derivative thereof, and/or variant thereof, including a mature human J-chain amino acid sequence provided herein as SEQ ID NO: 379. A functional fragment, derivative, and/or variant of a J-chain has at least 90% sequence identity to the reference J-chain and retains the multimerizing function of the reference J-chain.

In certain aspects, the J-chain of the IgM antibody as provided herein includes an amino acid substitution at the amino acid position corresponding to amino acid Y102, T103, N49 or S51 of SEQ ID NO: 379.

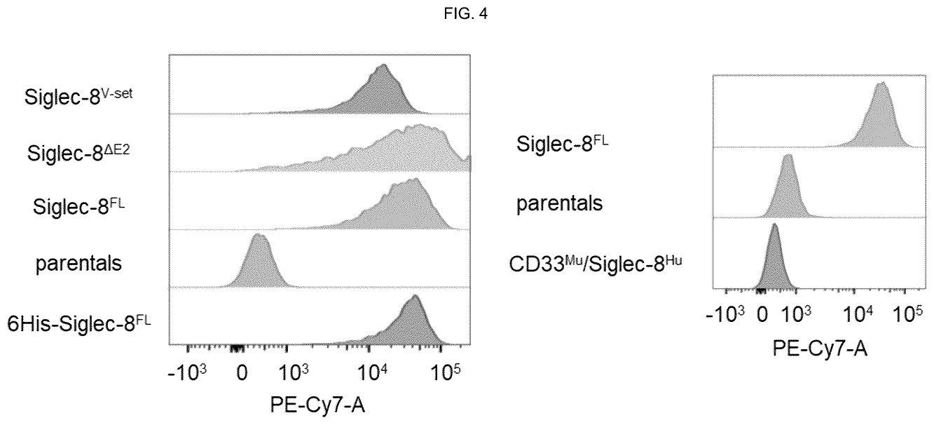

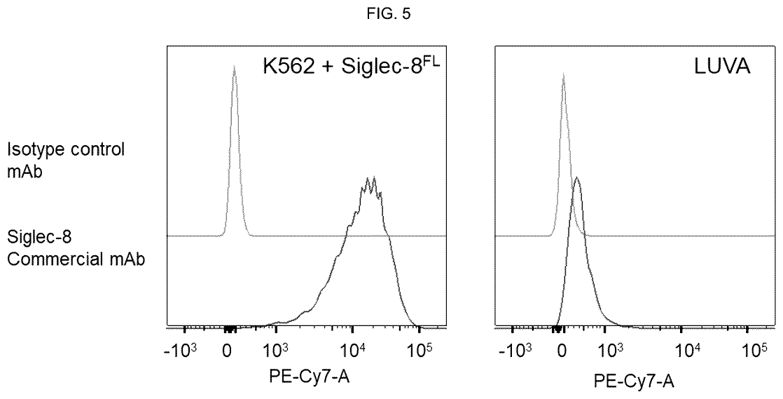

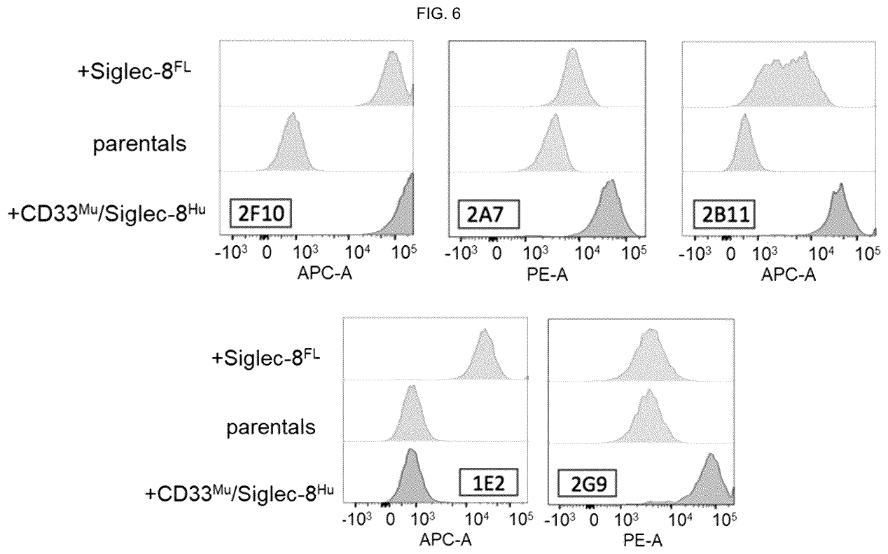

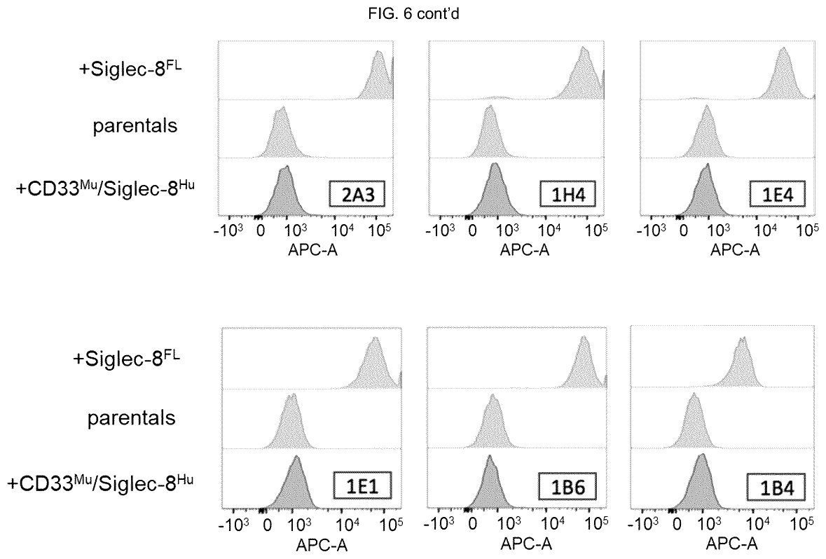

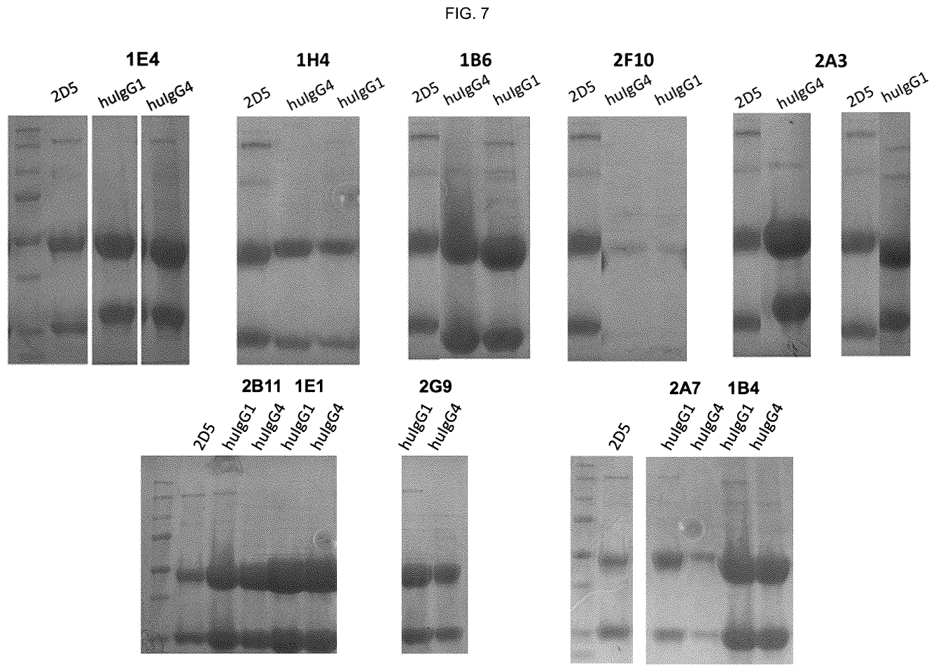

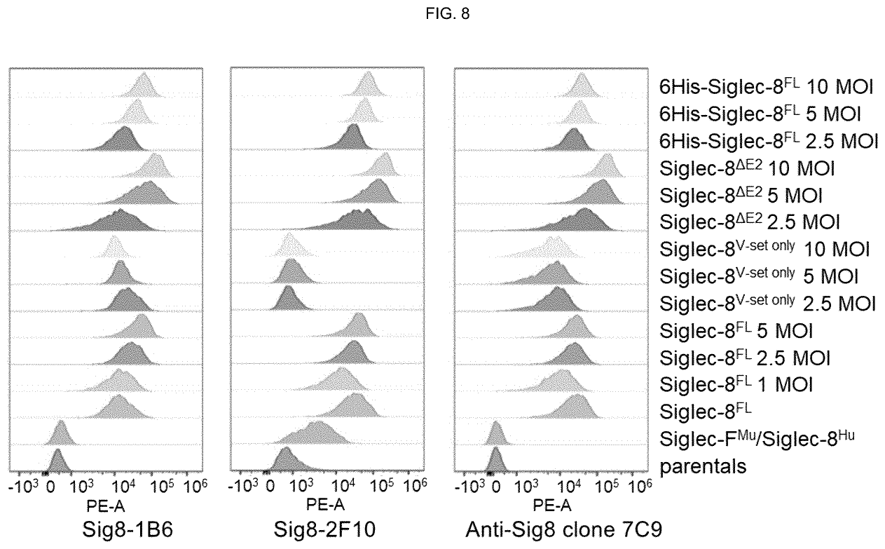

By “an amino acid corresponding to” a position of SEQ ID NO: 379 is meant the amino acid in the sequence of the J-chain of any species which is homologous to the referenced residue in the human J-chain. For example, the position corresponding to Y102 in SEQ ID NO: 379 is conserved in the J-chain amino acid sequences of at least 43 other species. The position corresponding to T103 in SEQ ID NO: 379 is conserved in the J-chain amino acid sequences of at least 37 other species. The positions corresponding to N49 and S51 in SEQ ID NO: 379 are conserved in the J-chain amino acid sequences of at least 43 other species. See FIG. 4 of U.S. Pat. No. 9,951,134 and FIG. 2 of PCT/US2019/020374.

In certain aspects, the amino acid corresponding to Y102 of SEQ ID NO: 379 can be substituted with any amino acid. In certain aspects, the amino acid corresponding to Y102 of SEQ ID NO: 379 can be substituted with alanine (alanine substitution underlined):

(SEQ ID NO: 394)
QEDERIVLVDNKCKCARITSRIIRSSEDPNEDIVERNIRIIVPLNNRENI
SDPTSPLRTRFVYHLSDLCKKCDPTEVELDNQIVTATQSNICDEDSATET
CATYDRNKCYTAVVPLVYGGETKMVETALTPDACYPD,

With serine (serine substitution underlined):

(SEQ ID NO: 395)
QEDERIVLVDNKCKCARITSRIIRSSEDPNEDIVERNIRIIVPLNNRENI
SDPTSPLRTRFVYHLSDLCKKCDPTEVELDNQIVTATQSNICDEDSATET
CSTYDRNKCYTAVVPLVYGGETKMVETALTPDACYPD,

Or with arginine (arginine substitution underlined):

(SEQ ID NO: 396)
QEDERIVLVDNKCKCARITSRIIRSSEDPNEDIVERNIRIIVPLNNRENI
SDPTSPLRTRFVYHLSDLCKKCDPTEVELDNQIVTATQSNICDEDSATET
CRTYDRNKCYTAVVPLVYGGETKMVETALTPDACYPD.

In certain aspects, the amino acid corresponding to T103 of SEQ ID NO: 379 can be substituted with any amino acid. In a particular aspect, the amino acid corresponding to T103 of SEQ ID NO: 379 can be substituted with alanine as follows (alanine substitution underlined):

(SEQ ID NO: 397)
QEDERIVLVDNKCKCARITSRIIRSSEDPNEDIVERNIRIIVPLNNRENI
SDPTSPLRTRFVYHLSDLCKKCDPTEVELDNQIVTATQSNICDEDSATET
CYAYDRNKCYTAVVPLVYGGETKMVETALTPDACYPD.

In certain aspects, the variant J-chain or functional fragment thereof of the IgM antibody as provided herein includes an amino acid substitution at the amino acid position corresponding to amino acid N49 or amino acid S51 of SEQ ID NO: 379, provided that S51 is not substituted with threonine (T), or wherein the J-chain includes amino acid substitutions at the amino acid positions corresponding to both amino acids N49 and S51 of SEQ ID NO: 379.

The amino acids corresponding to N49 and S51 of SEQ ID NO: 379 along with the amino acid corresponding to 150 of SEQ ID NO: 379 include an N-linked glycosylation motif in the J-chain. Accordingly, mutations at N49 and/or S51 (with the exception of a single threonine substitution at S51) can prevent glycosylation at this motif. In certain aspects, the asparagine at the position corresponding to N49 of SEQ ID NO: 379 can be substituted with any amino acid. In certain aspects, the asparagine at the position corresponding to N49 of SEQ ID NO: 379 can be substituted with alanine (A), glycine (G), threonine (T), serine (S) or aspartic acid (D). In a particular aspect the position corresponding to N49 of SEQ ID NO: 379 can be substituted with alanine (A). In a particular aspect the J-chain is a variant human J-chain and includes the amino acid sequence:

(SEQ ID NO: 398)
QEDERIVLVDNKCKCARITSRIIRSSEDPNEDIVERNIRIIVPLNNREAI
SDPTSPLRTRFVYHLSDLCKKCDPTEVELDNQIVTATQSNICDEDSATET
CYTYDRNKCYTAVVPLVYGGETKMVETALTPDACYPD.

In certain aspects, the serine at the position corresponding to S51 of SEQ ID NO: 379 can be substituted with any amino acid except threonine. In certain aspects, the serine at the position corresponding to S51 of SEQ ID NO: 379 can be substituted with alanine (A) or glycine (G). In a particular aspect the position corresponding to S51 of SEQ ID NO: 379 can be substituted with alanine (A). In a particular aspect the variant J-chain or functional fragment thereof is a variant human J-chain and includes the amino acid sequence:

(SEQ ID NO: 399)
EDERIVLVDNKCKCARITSRIIRSSEDPNEDIVERNIRIIVPLNNRENIA
DPTSPLRTRFVYHLSDLCKKCDPTEVELDNQIVTATQSNICDEDSATETC
YTYDRNKCYTAVVPLVYGGETKMVETALTPDACYPD.

Particular embodiments include a heterologous polypeptide (e.g., a single-domain antibody binding domain) fused to the J-chain or functional fragment thereof via a peptide linker, e.g., a peptide linker including at least 5 amino acids, but no more than 25 amino acids. In certain aspects, the peptide linker includes (GGGGS)n (SEQ ID NO: 347) wherein n is 1-5.

A single-domain antibody binding domain can be introduced into the J-chain at any location that allows the binding of the binding domain to its binding target without interfering with J-chain function or the function of an associated IgA, IgM, or hybrid IgG antibody. Insertion locations include at or near the C-terminus, at or near the N-terminus or at an internal location that, based on the three-dimensional structure of the J-chain, is accessible. In certain aspects, the antigen-binding domain can be introduced into the mature human J-chain of SEQ ID NO: 379 between cysteine residues 92 and 101 of SEQ ID NO: 379. In a further aspect, the antigen-binding domain can be introduced into the human J-chain of SEQ ID NO: 379 at or near a glycosylation site. In a further aspect, the antigen-binding domain can be introduced into the human J-chain of SEQ ID NO: 379 within 10 amino acid residues from the C-terminus, or within 10 amino acids from the N-terminus.

In particular embodiments, the single-domain antibody is introduced into the native human J-chain sequence of SEQ ID NO: 379 by chemical or chemo-enzymatic derivatization. In particular embodiments, the single-domain antibody is introduced into the native human J-chain sequence of SEQ ID NO: 379 by a chemical linker. In some embodiments, the chemical linker is a cleavable or non-cleavable linker. In particular embodiments, the cleavable linker is a chemically labile linker or an enzyme-labile linker. In some embodiments, the linker is selected from the group including N-succinimidyl-3-(2-pyridyldithio) propionate (SPDP), succinimidyl-4-(N-maleimidomethyl) cyclohexane-1-carboxylate (SMCC), N-succinimidyl-4-(2-pyridylthio) pentanoate (SPP), iminothiolane (IT), afunctional derivatives of imidoesters, active esters, aldehydes, bis-azido compounds, bis-diazonium derivatives, diisocyanates, and bis-active fluorine compounds. In particular embodiments, the modified J-chain is modified by insertion of an enzyme recognition site, and by post-translationally attaching a binding moiety at the enzyme recognition site through a peptide or non-peptide linker.

In certain aspects the modified J-chain can include the formula X[Lₙ]J or J[Lₙ]X, where J includes a mature native J-chain or functional fragment thereof, X includes a heterologous binding domain, and [Lₙ] is a linker sequence including n amino acids, where n is a positive integer from 1 to 100, 1 to 50, or 1 to 25. In certain aspects N is 5, 10, 15, or 20.

J-chains from the following species can also be used in certain embodiments: Pan troglodytes, Pongo abelii, Callithrix jacchus, Macaca mulatta, Papio Anubis, Saimiri boliviensis, Tupaia chinensis, Tursiops truncatus, Orcinus orca, Loxodonta Africana, Leptonychotes weddellii, Ceratotherium simum, Felis catus, Canis familiaris, Ailuropoda melanoleuca, Mustela furo, Equus caballus, Cavia porcellus, Camelus ferus, Capra hircus, Chinchilla lanigera, Mesocricetus auratus, Ovis aries, Myotis lucifugus, Pantholops hodgsonii, Bos taurus, Mus musculus, Rattus norvegicus, Echinops telfairi, Oryctolagus cuniculus, Monodelphis domestica, Alligator mississippiensis, Chrysemys picta, Sarcophilus harrisii, Ornithorhynchus anatinus, Melopsittacus undulatus, Anas platyrhynchos, Gallus gallus, Meleagris gallopavo, Falco peregrinus, Zonotrichia albicollis, and Pteropus alecto.

In particular embodiments, alternative multimerization domains can be used. In particular embodiments, a multimerization domain includes a dimerization and docking domain (DDD) which can be derived from the cAMP-dependent protein kinase (PKA) regulatory subunits and can be paired with an anchoring domain (AD). The AD can be derived from a specific region found in various A-kinase anchoring proteins (AKAPs) that mediates association with the R subunits of PKA. Additional DDDs and ADs include: the 4-helix bundle type DDD (Newlon, et al. 1999; 3: 222-227) domains obtained from p53, DCoH (pterin 4 α carbinolamine dehydratase/dimerization cofactor of hepatocyte nuclear factor 1 α (TCF1)) and HNF-1 (hepatocyte nuclear factor 1) (Rose, et al. Other AD sequences of potential use may be found in US 2003/0232420A1.

In particular embodiments, the multimerization domain includes complementary binding domains that can dimerize. In particular embodiments, the binding domain is a transmembrane polypeptide derived from a FcεRI chain. In particular embodiments, an antibody or fragment thereof can include a part of a FcεRI α chain and another antibody or fragment thereof can include a part of an FcεRI β chain such that said FcεRI chains spontaneously dimerize together to form a dimeric antibody (e.g., bispecific antibody). In particular embodiments, an antibody or fragment thereof can include a part of a FcεRI α chain and another antibody or fragment thereof part of a FcεRI γ chain such that said FcεRI chains spontaneously trimerize together to form a trimeric antibody, and in another embodiment the multi-domain binding molecule can include a part of FcεRI α chain, a part of FcεRI β chain and a part of FcεRI γ chain such that said FcεRI chains spontaneously tetramerize together to form a tetrameric multi-domain binding molecule.

Leucine zippers are described in U.S. Pat. No. 5,932,448; SH2 and SH3 are described in Vidal et al., Biochemistry, 43:7336-44, 2004); PTB is described in Zhou et al., Nature, 378:584-592, 1995); WW is described in Sudol Prog Biochys MoL Bio, 65:113-132, 1996; PDZ is described in Kim et al., Nature, 378: 85-88, 1995 and Komau et al., Science, 269:1737-1740, 1995; and WD40 is described in.

Additional multimerization domains and systems are described in, for example, Hodneland, et al. 2002; 99: 5048-5052; Arakawa et al., Biochem, 32: 1350, 1993; WO2012001647A2; U.S. Pat. Nos. 5,821,333; and 5,932,448. In particular embodiments, a multimerization domain includes the dimerization domain of the SH2B adapter protein from GenBank Accession no. AAF73912.1 (Nishi et al., Mol Cell Biol, 25: 2607-2621, 2005), the SH3 domain of IB1 from GenBank Accession no. AAD22543.1 (Kristensen el al., EMBO J., 25: 785-797, 2006), the PTB domain of human DOK-7 from GenBank Accession no. NP_005535.1 (Wagner et al., Cold Spring Harb Perspect Biol. 5: a008987, 2013), the PDZ-like domain of SATB1 from UniProt Accession No. 001826 (Galande et al., Mol Cell Biol. August; 21: 5591-5604, 2001), the WD40 repeats of APAF from UniProt Accession No. 014727 (Jorgensen et al., 2009. PLOS One. 4(12):e8463), the PAS motif of the dioxin receptor from UniProt Accession No. 16L9E7 (Pongratz et al., Mol Cell Biol, 18:4079-4088, 1998) and the EF hand motif of parvalbumin from UniProt Accession No. P20472 (Jamalian et al., Int J Proteomics, 2014: 153712, 2014). C4b, dextrameric, and ferritin-based multimerization can be used.

In particular embodiments, complementary binding domains can be induced using a third molecule or chemical inducer. This method of dimerization requires that one antibody or fragment thereof include a chemical inducer of dimerization binding domain 1 (CBD1) and the second antibody or fragment thereof include the second chemical inducer of dimerization binding domain (CBD2), wherein CBD1 and CBD2 are capable of simultaneously binding to a chemical inducer of dimerization (CID). CBD1 may include a rapamycin binding domain of FK-binding protein 12 (FKBP12) and CBD2 may include a FKBP12-Rapamycin Binding (FRB) domain of mTOR.

In particular embodiments, a multimerized antibody can include binding domains that bind similar antigens, binding domains that bind different antigens, or combinations thereof. In particular embodiments, a multimerized antibody can include binding domains that bind similar epitopes, binding domains that bind different epitopes, or combinations thereof.

(iv) Expression of Recombinant Proteins. Siglec-8 antibodies and multidomain binding molecules can be produced by recombinant expression. Recombinant polynucleotide constructs typically include an expression control sequence operably linked to the coding sequences of antibody chains, including naturally-associated or heterologous promoter regions. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the cross-reacting antibodies.

In particular embodiments, mammalian cells are used as a host for expressing nucleotide segments encoding immunoglobulins or fragments thereof. See Winnacker, From Genes to Clones, (VCH Publishers, NY, 1987). A number of suitable host cell lines capable of secreting intact heterologous proteins have been developed in the art, and include CHO cell lines (e.g., DG44), various COS cell lines, HeLa cells, HEK293 cells, L cells, and non-antibody-producing myelomas including Sp2/0 and NS0. Preferably, the cells are nonhuman. Expression vectors for these cells can include expression control sequences, such as an origin of replication, a promoter, an enhancer (Queen et al., Immunol. Rev. 89:49 (1986)), and necessary processing information sites, such as ribosome binding sites, RNA splice sites, polyadenylation sites, and transcriptional terminator sequences. In particular embodiments, expression control sequences are promoters derived from endogenous genes, cytomegalovirus, SV40, adenovirus, and bovine papillomavirus (see Co et al., J. Immunol. 1992, 148:1149).

Once expressed, antibodies can be purified according to standard procedures of the art, including high-performance liquid chromatography (HPLC) purification, column chromatography, gel electrophoresis and the like (see generally, Scopes, Protein Purification (Springer-Verlag, NY, 1982)).

In particular embodiments, antibodies are formed using the Daedalus expression system as described in Pechman et al. (Am J Physiol 294: R1234-R1239, 2008). The Daedalus system utilizes inclusion of minimized ubiquitous chromatin opening elements in transduction vectors to reduce or prevent genomic silencing and to help maintain the stability of decigram levels of expression. This system can bypass tedious and time-consuming steps of other protein production methods by employing the secretion pathway of serum-free adapted human suspension cell lines, such as 293 Freestyle. Using optimized lentiviral vectors, yields of 20-100 mg/l of correctly folded and post-translationally modified, endotoxin-free protein of up to 70 kDa in size, can be achieved in conventional, small-scale (100 ml) culture. At these yields, most proteins can be purified using a single size-exclusion chromatography step, immediately appropriate for use in structural, biophysical or therapeutic applications. Bandaranayake et al., Nucleic Acids Res., 39(21) 2011. In some instances, purification by chromatography may not be needed due to the purity of manufacture according to the methods described herein.

(v) Siglec-8 Antibody Conjugates. Siglec-8 antibody conjugates include a Siglec-8 antibody disclosed herein linked to another molecule, other than an additional binding domain. Examples of antibody conjugates include antibody radioisotope conjugates, antibody immunotoxins, antibody-drug conjugates (ADCs), antibody-detectable label conjugates, and antibody-particle conjugates.

Antibody-radioisotope conjugates include a Siglec-8 antibody linked to a radioisotope for use in nuclear medicine. Nuclear medicine refers to the diagnosis and/or treatment of disorders by administering radioactive isotopes (radioisotopes or radionuclides) to a subject. Therapeutic nuclear medicine is often referred to as radiation therapy or radioimmunotherapy (RIT).

Examples of radioactive isotopes that can be conjugated to Siglec-8 antibodies of the present disclosure include iodine-131 yttrium-90, arsenic-72, arsenic-74, iodine-131, indium-111, and lutetium-177, as well as alpha-emitting radionuclides such as astatine-211, actinium-225, bismuth-212 or bismuth-213. Methods for preparing radioimmunoconjugates are established in the art. Examples of radioimmunoconjugates are commercially available, including Zevalin™ (DEC Pharmaceuticals), and similar methods can be used to prepare radioimmunoconjugates using the antibodies of the disclosure.

Examples of radionuclides that are useful for radiation therapy include ²²⁵Ac and ²²⁷Th. ²²⁵Ac is a radionuclide with the half-life of ten days. As ²²⁵Ac decays the daughter isotopes ²²¹Fr, ²¹³Bi, and ²⁰⁹Pb are formed. ²²⁷Th has a half-life of 19 days and forms the daughter isotope ²²³Ra.

Additional examples of useful radioisotopes include ²²⁸Ac, ¹¹¹Ag, ¹²⁴Am, ⁷⁴As, ²¹¹At, ²⁰⁹At, ¹⁹⁴Au, ¹²⁸Ba, ⁷Be, ²⁰⁶Bi, ²⁴⁵Bk, ²⁴⁶Bk, ⁷⁶Br, ¹¹C, ¹⁴C, ⁴⁷Ca, ²⁵⁴Cf, ²⁴²Cm, ⁵¹Cr, ⁶⁷Cu, ¹⁵³Dy, ¹⁵⁷Dy, ¹⁵⁹Dy, ¹⁶⁵Dy, ¹⁶⁶Dy, ¹⁷¹ Er, ²⁵⁰Es, ²⁵⁴Es, ¹⁴⁷Eu, ¹⁵⁷Eu, ⁵²Fe, ⁵⁹Fe, ²⁵¹ Fm, ²⁵²Fm, ²⁵³Fm, ⁶⁶Ga, ⁷²Ga, ¹⁴⁶Gd, ¹⁵³Gd, ⁶⁸Ge, ³H, ¹⁷⁰Hf, 171Hf, ¹⁹³Hg, ¹⁹³mHg, ¹⁶⁰mHo, ¹³⁰I, ¹³¹I, ¹³⁵I, ¹¹⁴mIn, ¹⁸⁵Ir, ⁴²K, ⁴³K, ⁷⁶Kr, ⁷⁹Kr, ⁸¹mKr, ¹³²La, ²⁶²Lr, ¹⁶⁹Lu, ¹⁷⁴mLu, ¹⁷⁶mLu, ²⁵⁷Md, ²⁶⁰Md, ²⁸Mg, ⁵²Mn, ⁹⁰Mo, ²⁴Na, ⁹⁵Nb, ¹³⁸Nd, ⁵⁷Ni, ⁶⁶Ni, ²³⁴Np, ¹⁵O, ¹⁸Os, ¹⁸⁹mOs, ¹⁹¹Os, ³²P, ²⁰¹Pb, ¹⁰¹Pd, ¹⁴³Pr, ¹⁹¹Pt, ²⁴³Pu, ²²⁵Ra, ⁸¹Rb, ¹⁸⁸Re, ¹⁰⁵Rh, ²¹¹Rn, ¹⁰³Ru, ³⁵S, ⁴⁴Sc, ⁷²Se, ¹⁵³Sm, ¹²⁵Sn, ⁹¹Sr, ¹⁷³Ta, ¹⁵⁴Tb, ¹²⁷Te, ²³⁴Th, ⁴⁵Ti, ¹⁶⁶Tm, ²³⁰U, ²³⁷U, ²⁴⁰U, ⁴⁸V, ¹⁷⁸W, ¹⁸¹W, ¹⁸⁸W, ¹²⁵Xe, ¹²⁷Xe, ¹³³Xe, ¹³³mXe, ¹³⁵Xe, ⁸⁵mY, ⁸⁶Y, ⁹⁰Y, ⁹³Y, ¹⁶⁹Yb, ¹⁷⁵Yb, ⁶⁵Zn, ⁷¹mZn, ⁸⁶Zr, ⁹⁵Zr, and/or ⁹⁷Zr. Radioisotopes can be used as a type of detectable label called a radiolabel. In particular embodiments, a radioisotope includes ¹³¹I, ⁹⁰Y, and/or ²¹¹At. In particular embodiments, a radioisotope is selected that includes a half-life (t_{1/2}) that enables high-yield radiolabeling and drug delivery. In particular embodiments, a radioisotope is selected that includes a half-life (t_{1/2}) of 7.2 hours. In particular embodiments, a radioisotope is selected that does not emit daughter radionuclides that cause organ toxicity.

In particular embodiments, the Siglec-8 antibody can be formed as an antibody immunotoxin. Antibody immunotoxins include a Siglec-8 antibody disclosed herein conjugated to one or more cytotoxins (e.g., protein toxins, enzymatically active toxins of bacterial, fungal, plant, or animal origin, or fragments thereof). A toxin can be any agent that is detrimental to cells. Frequently used plant toxins are divided into two classes: (1) holotoxins (or class II ribosome inactivating proteins), such as ricin, abrin, mistletoe lectin, and modeccin, and (2) hemitoxins (class I ribosome inactivating proteins), such as pokeweed antiviral protein (PAP), saporin, Bryodin 1, bouganin, and gelonin. Commonly used bacterial toxins include diphtheria toxin (DT) and Pseudomonas exotoxin (PE). Kreitman, Current Pharmaceutical Biotechnology 2:313-325 (2001). The toxin may be obtained from essentially any source and can be a synthetic or a natural product.

Immunotoxins with multiple (e.g., four) cytotoxins per binding domain can be prepared by partial reduction of the binding domain with an excess of a reducing reagent such as dithiothreitol (DTT) or tris(2-carboxyethyl)phosphine (TCEP) at 37° C. for 30 min, then the buffer can be exchanged by elution through SEPHADEX G-25 resin with 1 mM DTPA (diethylene triamine penta-acetic acid) in Dulbecco's phosphate-buffered saline (DPBS). The eluent can be diluted with further DPBS, and the thiol concentration of the binding domain can be measured using 5,5′-dithiobis(2-nitrobenzoic acid) [Ellman's reagent]. An excess, for example 5-fold, of the linker-cytotoxin conjugate can be added at 4° C. for 1 hr, and the conjugation reaction can be quenched by addition of a substantial excess, for example 20-fold, of cysteine. The resulting immunotoxin mixture can be purified on SEPHADEX G-25 equilibrated in PBS to remove unreacted linker-cytotoxin conjugate, desalted if desired, and purified by size-exclusion chromatography. The resulting immunotoxin can then be sterile filtered, for example, through a 0.2 μm filter, and can be lyophilized if desired for storage.

Antibody-drug conjugates allow for the targeted delivery of a drug moiety to a Siglec-8 expressing cell, in particular embodiments intracellular accumulation therein, where systemic administration of unconjugated drugs may result in unacceptable levels of toxicity to normal cells (Polakis (2005) Current Opinion in Pharmacology 5:382-387).

In particular embodiments, antibody-drug conjugates refer to targeted molecules which combine properties of both antibodies and cytotoxic drugs (e.g., chemotherapeutic drugs) by targeting potent cytotoxic drugs to antigen-expressing cells (Teicher (2009) Current Cancer Drug Targets 9:982-1004), thereby enhancing the therapeutic index by maximizing efficacy and minimizing off-target toxicity (Carter and Senter (2008) The Cancer Jour. 14(3):154-169; Chari, (2008) Acc. Chem. Res. 41:98-107). See also Kamath & Iyer (Pharm Res. 32(11): 3470-3479, 2015), which describes considerations for the development of antibody-drug conjugates.

The drug moiety (D) of an antibody-drug conjugate may include any compound, moiety or group that has a cytotoxic or cytostatic effect. Drug moieties may impart their cytotoxic and cytostatic effects by mechanisms including tubulin binding, DNA binding or intercalation, and inhibition of RNA polymerase, protein synthesis, and/or topoisomerase. Exemplary drugs include actinomycin D, anthracycline, auristatin, calicheamicin, camptothecin, CC1065, colchicin, cytochalasin B, daunorubicin, 1-dehydrotestosterone, dihydroxy anthracinedione, dolastatin, doxorubicin, duocarmycin, elinafide, emetine, ethidium bromide, etoposide, gramicidin D, glucocorticoids, lidocaine, maytansinoid (including monomethyl auristatin E [MMAE]; vedotin), mithramycin, mitomycin, mitoxantrone, nemorubicin, PNU-159682, procaine, propranolol, puromycin, pyrrolobenzodiazepine (PBD), taxane, taxol, tenoposide, tetracaine, trichothecene, vinblastine, vinca alkaloid, vincristine, and stereoisomers, isosteres, analogs, and derivatives thereof that have cytotoxic activity.

The drug may be obtained from essentially any source; it may be synthetic or a natural product isolated from a selected source, e.g., a plant, bacterial, insect, mammalian or fungal source. The drug may also be a synthetically modified natural product or an analogue of a natural product.

In particular embodiments, the antibody-drug conjugates include an antibody conjugated, i.e., covalently attached, to the drug moiety. In particular embodiments, the Siglec-8 antibody is covalently attached to the drug moiety through a linker. A linker can include any chemical moiety that is capable of linking an antibody, antibody fragment (e.g., antigen binding fragments) or functional equivalent to another moiety, such as a drug moiety. Linkers can be susceptible to cleavage (cleavable linker), such as, acid-induced cleavage, photo-induced cleavage, peptidase-induced cleavage, esterase-induced cleavage, and disulfide bond cleavage, at conditions under which the compound or the antibody remains active. Alternatively, linkers can be substantially resistant to cleavage (e.g., stable linker or noncleavable linker). In some aspects, the linker is a procharged linker, a hydrophilic linker, or a dicarboxylic acid-based linker. The antibody-drug conjugate selectively delivers an effective dose of a drug to cells (e.g., cancer cells) whereby greater selectivity, i.e., a lower efficacious dose, may be achieved while increasing the therapeutic index (“therapeutic window”).

To prepare antibody-drug conjugates, linker-cytotoxin conjugates can be made by conventional methods analogous to those described by Doronina et al. (Bioconjugate Chem. 17: 114-124, 2006). Antibody-drug conjugates with multiple (e.g., four) drugs per antibody can be prepared by partial reduction of the antibody with an excess of a reducing reagent such as dithiothreitol (DTT) or tris(2-carboxyethyl)phosphine (TCEP) at 37° C. for 30 min, then the buffer can be exchanged by elution through SEPHADEX G-25 resin with 1 mM DTPA in Dulbecco's phosphate-buffered saline (DPBS). The eluent can be diluted with further DPBS, and the thiol concentration of the antibody can be measured using 5,5′-dithiobis(2-nitrobenzoic acid) [Ellman's reagent]. An excess, for example 5-fold, of the linker-cytotoxin conjugate can be added at 4° C. for 1 hr, and the conjugation reaction can be quenched by addition of a substantial excess, for example 20-fold, of cysteine. The resulting ADC mixture can be purified on SEPHADEX G-25 equilibrated in PBS to remove unreacted linker-cytotoxin conjugate, desalted if desired, and purified by size-exclusion chromatography. The resulting ADC can then be sterile filtered, for example, through a 0.2 μm filter, and can be lyophilized if desired for storage. Methods used to produce immunotoxins can similarly be used to prepare antibody-drug conjugates.

Antibody-detectable label conjugates include a Siglec-8 antibody linked to a detectable label. Detectable labels can include any suitable label or detectable group detectable by, for example, optical, spectroscopic, photochemical, biochemical, immunochemical, electrical, optical or chemical means. In particular embodiments, detectable labels can include fluorescent labels, chemiluminescent labels, spectral colorimetric labels, enzymatic labels, and affinity tags.

Fluorescent labels can be particularly useful in cell staining, identification, imaging, and isolation uses. Exemplary fluorescent labels include blue fluorescent proteins (e.g. eBFP, eBFP2, Azurite, mKalama1, GFPuv, Sapphire, T-sapphire); cyan fluorescent proteins (e.g. eCFP, Cerulean, CyPet, AmCyanl, Midoriishi-Cyan, mTurquoise); green fluorescent proteins (e.g. GFP, GFP-2, tagGFP, turboGFP, EGFP, Emerald, Azami Green, Monomeric Azami Green (mAzamigreen)), CopGFP, AceGFP, avGFP, ZsGreenl, Oregon Green™ (Thermo Fisher Scientific)); Luciferase; orange fluorescent proteins (mOrange, mKO, Kusabira-Orange, Monomeric Kusabira-Orange, mTangerine, tdTomato); red fluorescent proteins (mKate, mKate2, mPlum, DsRed monomer, mCherry, mRuby, mRFP1, DsRed-Express, DsRed2, DsRed-Monomer, HcRed-Tandem, HcRedl, AsRed2, eqFP611, mRaspberry, mStrawberry, Jred, Texas Red™ (Thermo Fisher Scientific)); far red fluorescent proteins (e.g., mPlum and mNeptune); yellow fluorescent proteins (e.g., YFP, eYFP, Citrine, SYFP2, Venus, YPet, PhiYFP, ZsYellowl); and tandem conjugates.

Chemiluminescent labels can include lucigenin, luminol, luciferin, isoluminol, theromatic acridinium ester, imidazole, acridinium salt, or oxalate ester.

Spectral colorimetric labels can include colloidal gold or colored glass or plastic (e.g., polystyrene, polypropylene, and latex) beads.

Enzymatic labels can produce, for example, a chemiluminescent signal, a color signal, or a fluorescent signal. Enzymes can include malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase.

Affinity tags can include, for example, His tag (HHHHHH (SEQ ID NO: 401)), Flag tag (DYKDDDD (SEQ ID NO: 402), Xpress tag (DLYDDDDK (SEQ ID NO: 403)), Avi tag (GLNDIFEAQKIEWHE (SEQ ID NO: 404)), Calmodulin binding peptide (CBP) tag (KRRWKKNFIAVSAANRFKKISSSGAL (SEQ ID NO: 405)), Polyglutamate tag (EEEEEE (SEQ ID NO: 406)), HA tag (YPYDVPDYA (SEQ ID NO: 407)), Myc tag (EQKLISEEDL (SEQ ID NO: 408)), Strep tag (WRHPQFGG (SEQ ID NO: 409)), STREP® tag II (WSHPQFEK (SEQ ID NO: 410); IBA Institut fur Bioanalytik, Germany; see, e.g., U.S. Pat. No. 7,981,632), Softag 1 (SLAELLNAGLGGS (SEQ ID NO: 411)), Softag 3 (TQDPSRVG (SEQ ID NO: 412)), and V5 tag (GKPIPNPLLGLDST (SEQ ID NO: 413)).

Antibody-particle conjugates include an antibody linked to a particle. In particular embodiments, particles include microparticles, nanoparticles, nanoshells, nanobeads, microbeads, or nanodots. Particles can include, for example, latex beads, polystyrene beads, fluorescent beads, and/or colored beads, and can be made from organic matter and/or inorganic matter. They can be made of any suitable materials that allow for the conjugation of capture proteins, such as Siglec-8 antibodies disclosed herein, to their surface. Examples of suitable materials include: ceramics, glass, polymers, and magnetic materials. Suitable polymers include polystyrene, poly-(methyl methacrylate), poly-(lactic acid), (poly-(lactic-co-glycolic acid)), polyesters, polyethers, polyolefins, polyalkylene oxides, polyamides, polyurethanes, polysaccharides, celluloses, polyisoprenes, methylstyrene, acrylic polymers, thoria sol, latex, nylon, Teflon cross-linked dextrans (e.g., Sepharose), chitosan, agarose, and cross-linked micelles. Additional examples include carbon graphited, titanium dioxide, and paramagnetic materials. See, e.g., “Microsphere Detection Guide” from Bangs Laboratories, Fishers Ind. In particular embodiments, microparticles can be made of one or more materials. In particular embodiments, microparticles are paramagnetic microparticles. Particular embodiments utilize carboxy-modified polystyrene latex (CML) flow cytometry beads and/or magnetic MagPlex® (Luminex, Austin, TX) flow cytometry beads. In particular embodiments, particles can carry a payload.

In particular embodiments, an antibody as disclosed herein can be linked to a conjugate by any method known in the art. In particular embodiments, the constant region can be modified to allow for site specific conjugation. Such techniques include the use of naturally occurring or engineered cysteine residues, disulfide bridges, poly-histidine sequences, glycoengineering tags, and transglutaminase recognition sequences. Antibody fragments can also be modified for site-specific conjugation, see for example, Kim et al., Mol Cancer Ther 7(8), 2008.

(vi) Recombinant Receptors. Siglec-8 antibodies disclosed herein can be utilized within recombinant receptors such as chimeric antigen receptors (CAR) and/or engineered T cell receptors (eTCR).

CAR include several distinct subcomponents that allow genetically modified cells (e.g., regulatory T cells) to recognize and kill cells expressing an antigen (e.g., Siglec-8). The subcomponents include at least an extracellular component and an intracellular component. The extracellular component includes a binding domain that binds a Siglec-8 epitope that is preferentially present on the surface of cells or in the area thereof. When the binding domain binds such epitopes, the intracellular component activates the cell to destroy the bound cell. CAR additionally include a transmembrane domain that directly or indirectly links the extracellular component to the intracellular component, and other subcomponents that can increase the CAR's function. For example, the inclusion of a spacer region and/or one or more linker sequences can allow the CAR to have additional conformational flexibility, often increasing the binding domain's ability to bind the targeted epitope.

eTCR disclosed herein include a Siglec-8 antibody disclosed herein linked to the C_{α} and/or C_{β} chains of a TCR. A TCR is a heterodimeric fusion protein that typically includes an α and β chain. Each chain includes a variable region (V_{α} and V_{β}) and a constant region (C_{α} and C_{β}). In particular embodiments, an eTCR does not include the native TCR variable region but does include the native TCR constant region. In particular embodiments, the eTCR includes a Siglec-8 antibody as the variable region of the α and β chain. In particular embodiments, eTCR include a C_{α} and/or C_{β} chain sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a known or identified TCR C_{α} or C_{β}.

In particular embodiments, the extracellular component of a recombinant receptor includes a binding domain that binds Siglec-8. Particular embodiments of binding domains include a Siglec-8 antibody and/or the CDRs or variable chains thereof as disclosed herein, such as those provided in Tables 1-6.

Recombinant receptors can additionally include spacer regions, transmembrane domains, intracellular effector domains, transduction markers, and tags.

Spacer regions are used to create appropriate distances and/or flexibility between sub-components of a protein. Spacer regions typically include 10 to 250 amino acids, 10 to 200 amino acids, 10 to 150 amino acids, 10 to 100 amino acids, 10 to 50 amino acids, or 10 to 25 amino acids. Exemplary spacer regions include all or a portion of an immunoglobulin hinge region. In particular embodiments, the spacer includes a 60 aa hinge domain spacer region. In particular embodiments the 60 aa hinge domain spacer region includes the sequence as set forth in SEQ ID NO: 485.

Transmembrane domains typically have a three-dimensional structure that is thermodynamically stable in a cell membrane, and generally ranges in length from 15 to 30 amino acids. The structure of a transmembrane domain can include an α helix, a β barrel, a β sheet, a β helix, or any combination thereof. Transmembrane domains can include at least the transmembrane region(s) of the α, β or ζ chain of a T-cell receptor, CD28, CD27, CD3, CD45, CD4, CD5, CD8, CD9, CD16, CD22; CD33, CD37, CD64, CD80, CD86, CD134, CD137 and CD154.

A transmembrane domain can include one or more additional amino acids adjacent to the transmembrane region, e.g., one or more amino acid within the extracellular region of the expressed protein (e.g., up to 15 amino acids of the extracellular region) and/or one or more additional amino acids within the intracellular region of the expressed protein (e.g., up to 15 amino acids of the intracellular components).

Intracellular effector domains activate the expressing cell when the binding domain binds antigen (Siglec-8). The term “effector domain” is thus meant to include any portion of the intracellular domain sufficient to transduce an activation signal.

An effector domain can include one, two, three or more intracellular signaling components (e.g., receptor signaling domains, cytoplasmic signaling sequences), co-stimulatory domains, or combinations thereof. Exemplary effector domains include signaling and stimulatory domains selected from: 4-1BB (CD137), CD3γ, CD3δ, CD3ε, CD3ζ, CD27, CD28, DAP10, ICOS, LAG3, NKG2D, NOTCH1, OX40, ROR2, SLAMF1, TCRα, TCRβ, TRIM, Wnt, Zap70, or any combination thereof. In particular embodiments, exemplary effector domains include signaling and co-stimulatory domains selected from: CD86, FcγRIIa, DAP12, CD30, CD40, PD-1, lymphocyte function-associated antigen-1 (LFA-1), LIGHT, NKG2C, B7-H3, a ligand that binds CD83, CDS, ICAM-1, GITR, BAFFR, SLAMF7, NKp80 (KLRF1), CD127, CD19, CD4, CD8α, CD8β, IL2Rβ, IL2Rγ, IL7Rα, ITGA4, VLA1, CD49a, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (CD150, IPO-3), BLAME (SLAMF8), SELPLG (CD162), LTBR, GADS, PAG/Cbp, NKp44, NKp30, or NKp46.

Intracellular signaling component sequences that act in a stimulatory manner may include iTAMs. Examples of iTAMs including primary cytoplasmic signaling sequences include those derived from CD3γ, CD3δ, CD3ε, CD3ζ, CD5, CD22, CD66d, CD79a, CD79b, and common FcRγ (FCER1G), FcγRIIa, FcRβ (Fcε Rib), DAP10, and DAP12. In particular embodiments, variants of CD3ζ retain at least one, two, three, or all ITAM regions.

A co-stimulatory domain is a domain whose activation can be required for an efficient lymphocyte response to cellular marker binding. Some molecules are interchangeable as intracellular signaling components or co-stimulatory domains. Examples of costimulatory domains include CD27, CD28, 4-1BB (CD137), OX40, PD-1, ICOS, lymphocyte function-associated antigen-1 (LFA-1), NKG2C, and a ligand that binds CD83.

Transduction markers may be selected from, for example, at least one of a truncated CD19 (tCD19; see Budde et al., Blood 122: 1660, 2013); a truncated human EGFR (tEGFR; see Wang et al., Blood 118: 1255, 2011); an extracellular domain of human CD34; and/or RQR8 which combines target epitopes from CD34 (see Fehse et al, Mol. Therapy 1(5 Pt 1); 448-456, 2000) and CD20 antigens (see Philip et al, Blood 124: 1277-1278). Methods to genetically modify cells to express recombinant receptors are well-known in the art.

Recombinant receptors can additionally include tags, such as the tags described as affinity tags elsewhere herein.

(vii) Compositions and Formulations. Any of the antibodies described herein (e.g., Siglec-8 antibodies, multi-domain binding molecules, antibody conjugates, therapeutics) in any exemplary format can be formulated alone or in combination into compositions for administration to subjects. Additionally, nucleic acids encoding the antibodies can also be formulated into compositions for administration (e.g., nucleic acids encapsulated within nanoparticles (e.g., liposomes or polymer-based nanoparticles) and/or as part of a vector delivery system (e.g., a viral vector or plasmid). Antibodies (e.g., Siglec-8 antibodies, multi-domain binding molecules, antibody conjugates) and/or nucleic acids encoding antibodies are collectively referred to herein as “active ingredients”.

Salts and/or pro-drugs of the active ingredients can also be used.

A pharmaceutically acceptable salt includes any salt that retains the activity of the active ingredient and is acceptable for pharmaceutical use. A pharmaceutically acceptable salt also refers to any salt which may form in vivo as a result of administration of an acid, another salt, or a prodrug which is converted into an acid or salt.

Suitable pharmaceutically acceptable acid addition salts can be prepared from an inorganic acid or an organic acid. Examples of such inorganic acids are hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids.

Suitable pharmaceutically acceptable base addition salts include metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine.

A prodrug includes an active ingredient which is converted to a therapeutically active compound after administration, such as by cleavage or by hydrolysis of a biologically labile group.

In particular embodiments, the compositions include active ingredients of at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA (ethylene-diamine-tetra-acetic acid).

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the antibodies or helps to prevent denaturation or adherence to the container wall. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, α-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

The compositions disclosed herein can be formulated for administration by, for example, injection, inhalation, infusion, perfusion, lavage, or ingestion. The compositions disclosed herein can further be formulated for intravenous, intradermal, intraarterial, intranodal, intralymphatic, intraperitoneal, intralesional, intraprostatic, intravaginal, intrarectal, topical, intrathecal, intratumoral, intramuscular, intravesicular, oral, sublingual, and/or subcutaneous administration.

For injection, compositions can be formulated as aqueous solutions, such as in buffers including Hanks' solution, Ringer's solution, or physiological saline. The aqueous solutions can include formulatory agents such as suspending, stabilizing, and/or dispersing agents. Alternatively, the composition can be in lyophilized and/or powder form for constitution with a suitable vehicle, e.g., sterile pyrogen-free water, before use.

For oral administration, the compositions can be formulated as tablets, pills, dragees, capsules, liquids, gels, syrups, slurries, suspensions and the like. For oral solid compositions such as powders, capsules and tablets, suitable excipients include binders (gum tragacanth, acacia, cornstarch, gelatin), fillers such as sugars, e.g., lactose, sucrose, mannitol and sorbitol; dicalcium phosphate, starch, magnesium stearate, sodium saccharine, cellulose, magnesium carbonate; cellulose preparations such as maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP); granulating agents; and binding agents. If desired, disintegrating agents can be added, such as corn starch, potato starch, alginic acid, cross-linked polyvinylpyrrolidone, agar, or alginic acid or a salt thereof such as sodium alginate. If desired, solid dosage forms can be sugar-coated or enteric-coated using standard techniques. Flavoring agents, such as peppermint, oil of wintergreen, cherry flavoring, orange flavoring, etc. can also be used.

Compositions can be formulated as an aerosol. In particular embodiments, the aerosol is provided as part of an anhydrous, liquid or dry powder inhaler. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas. In the case of a pressurized aerosol, a dosage unit may be determined by providing a valve to deliver a metered amount. Capsules and cartridges of gelatin for use in an inhaler or insufflator may also be formulated including a powder mix of the composition and a suitable powder base such as lactose or starch.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salt.

Additionally, compositions can be formulated as sustained-release systems utilizing semipermeable matrices of solid polymers including at least one type of antibody. Various sustained-release materials have been established and are well known by those of ordinary skill in the art. Sustained-release systems may, depending on their chemical nature, release one or more antibodies following administration for a few weeks up to over 100 days. Depot preparations can be administered by injection; parenteral injection; instillation; or implantation into soft tissues, a body cavity, or occasionally into a blood vessel with injection through fine needles.

Depot compositions can include a variety of bioerodible polymers including poly(lactide), poly(glycolide), poly(caprolactone) and poly(lactide)-co(glycolide) (PLG) of desirable lactide:glycolide ratios, average molecular weights, polydispersities, and terminal group chemistries. Blending different polymer types in different ratios using various grades can result in characteristics that borrow from each of the contributing polymers.

The use of different solvents (for example, dichloromethane, chloroform, ethyl acetate, triacetin, N-methyl pyrrolidone, tetrahydrofuran, phenol, or combinations thereof) can alter microparticle size and structure in order to modulate release characteristics. Other useful solvents include water, ethanol, dimethyl sulfoxide (DMSO), N-methyl-2-pyrrolidone (NMP), acetone, methanol, isopropyl alcohol (IPA), ethyl benzoate, and benzyl benzoate.

Exemplary release modifiers can include surfactants, detergents, internal phase viscosity enhancers, complexing agents, surface active molecules, co-solvents, chelators, stabilizers, derivatives of cellulose, (hydroxypropyl)methyl cellulose (HPMC), HPMC acetate, cellulose acetate, pluronics (e.g., F68/F127), polysorbates, Span® (Croda Americas, Wilmington, Delaware), poly(vinyl alcohol) (PVA), Brij® (Croda Americas, Wilmington, Delaware), sucrose acetate isobutyrate (SAIB), salts, and buffers.

Excipients that partition into the external phase boundary of nanoparticles such as surfactants including polysorbates, dioctylsulfosuccinates, poloxamers, PVA, can also alter properties including particle stability and erosion rates, hydration and channel structure, interfacial transport, and kinetics in a favorable manner.

Additional processing of the disclosed sustained release depot compositions can utilize stabilizing excipients including mannitol, sucrose, trehalose, and glycine with other components such as polysorbates, PVAs, and dioctylsulfosuccinates in buffers such as Tris, citrate, or histidine. A freeze-dry cycle can also be used to produce very low moisture powders that reconstitute to similar size and performance characteristics of the original suspension.

In particular embodiments, the compositions include active ingredients of at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

In certain examples, cells are genetically modified to express an antibody disclosed herein including a disclosed binding domain (as part of, for example, a CAR or eTCR). In these embodiments, genetically modified cells can be prepared as formulations for delivery in buffers such as Hanks' solution, Ringer's solution, or physiological saline. Cells can be genetically modified using methods known in the art. Exemplary targeted genetic engineering approaches include the use of CRISPR/Cas nuclease systems, zinc finger nucleases (ZFNs), and/or transcription activator like effector nucleases (TALENs). In particular embodiments, the cells are B cells genetically modified to express an antibody. Methods to genetically modify a B cell to express an antibody are described in PCT/US2018/056789.

Therapeutically effective amounts of cells within formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells.

In particular embodiments, cells are in a formulation volume of a liter or less, 500 ml or less, 250 ml or less, or 100 ml or less. Hence, the density of administered cells is typically greater than 10⁴ cells/ml, 10⁵ cells/ml, 10⁶ cells/ml, 10⁷ cells/ml, or 10⁸ cells/ml.

Any composition or formulation disclosed herein can advantageously include any other pharmaceutically acceptable carriers which include those that do not produce significantly adverse, allergic, or other untoward reactions that outweigh the benefit of administration. Exemplary pharmaceutically acceptable carriers are disclosed in Remington's Pharmaceutical Sciences, 18th Ed. Mack Printing Company, 1990. Moreover, compositions and formulations can be prepared to meet sterility, pyrogenicity, general safety, and purity standards as required by U.S. FDA Office of Biological Standards and/or other relevant foreign regulatory agencies.

(viii) Methods of Use. Methods disclosed herein include treating subjects. Subjects include, e.g., humans, veterinary animals (dogs, cats, reptiles, birds) livestock (e.g., horses, cattle, goats, pigs, chickens) and research animals (e.g., monkeys, rats, mice, fish). Treating subjects includes delivering therapeutically effective amounts. Therapeutically effective amounts include those that provide effective amounts, prophylactic treatments and/or therapeutic treatments.

An “effective amount” is the amount of a composition or formulation necessary to result in a desired physiological change in the subject. Effective amounts are often administered for research purposes. Effective amounts disclosed herein can cause a statistically significant effect in an animal model or in vitro assay relevant to the assessment of a condition's development, progression, and/or resolution.

A “prophylactic treatment” includes a treatment administered to a subject who does not display signs or symptoms of a condition or displays only early signs or symptoms of a condition such that treatment is administered for the purpose of diminishing or decreasing the risk of developing the condition further. Thus, a prophylactic treatment functions as a preventative treatment against a condition. In particular embodiments, prophylactic treatments reduce, delay, or prevent the worsening of a condition.

A “therapeutic treatment” includes a treatment administered to a subject who displays symptoms or signs of a condition and is administered to the subject for the purpose of diminishing or eliminating those signs or symptoms of the condition. The therapeutic treatment can reduce, control, or eliminate the presence or activity of the condition and/or reduce control or eliminate side effects of the condition.

Function as an effective amount, prophylactic treatment, or therapeutic treatment are not mutually exclusive, and in particular embodiments, administered dosages may accomplish more than one treatment type.

In certain examples, the condition to be researched, diagnosed, or treated includes a Siglec-8-related disorder. Siglec-8 related disorders include disorders associated with aberrant eosinophil or mast cell activity. Disorders associated with aberrant eosinophil or mast cell activity include asthma, chronic or allergic rhinitis with nasal polyposis, atopic dermatitis, eosinophilic gastrointestinal disorders (e.g., eosinophilic esophagitis, gastritis, and colitis), chronic eosinophilic pneumonia, hypereosinophilic syndromes, anaphylaxis, chronic idiopathic urticaria, eosinophilic cellulitis and fasciitis, Churg-Strauss syndrome, hematologic malignancies involving eosinophils or mast cells (e.g., eosinophilic leukemia) and mastocytosis.

Asthma, also known as reversible obstructive airway disease, is characterized by hyperresponsiveness of the tracheobronchial tree to respiratory irritants and bronchoconstrictor chemicals, producing attacks of wheezing, dyspnea, chest tightness, and cough that are reversible spontaneously or with treatment. It is a chronic disease involving the entire airway but varies in severity from occasional mild transient episodes to severe, chronic, life-threatening bronchial obstruction. Physical signs of an asthma attack include tachypnea, audible wheezing, and use of the accessory muscles of respiration. Rapid pulse and elevated blood pressure are also typically present, as are elevated levels of eosinophils in the peripheral blood and nasal secretions. In addition, asthma involves chronic inflammation of the airways, acute exacerbations varying in frequency between different patients and in response to environmental triggers. In severe cases, chronic remodeling of the airways may occur.

Allergic rhinitis, also known as allergic rhinoconjunctivitis or hay fever, is a chronic disease, which may first appear at any age, but the onset is usually during childhood or adolescence. A typical attack includes profuse watery rhinorrhea, paroxysmal sneezing, nasal obstruction and itching of the nose and palate. Postnasal mucus drainage also causes sore throat, throat clearing and cough. There can also be symptoms of allergic blepharoconjunctivitis, with intense itching of the conjunctivae and eyelids, redness, tearing, and photophobia. Severe attacks are often accompanied by systemic malaise, weakness, fatigue, and sometimes, muscle soreness after intense periods of sneezing.

Nasal polyposis is a chronic inflammatory disease of the upper respiratory tract characterized by an outgrowth of inflamed tissue into the nasal cavity. Nasal polyposis causes nasal obstruction, hyposmia, and recurrent infections with a significantly higher impact to quality of life than perennial allergic rhinitis (Li et al., Characterizing T-Cell Phenotypes in Nasal Polyposis in Chinese Patients, J Investig Allergol Clin Immunol 19(4):276-282, 2009).

Atopic dermatitis, also known as eczema, neurodermatitis, atopic eczema or Besnier's prurigo, is common chronic skin disorder specific to a subset of patients with the familial and immunologic features of atopy. The essential feature is a pruritic dermal inflammatory response, which induces a characteristic symmetrically distributed skin eruption with predilection for certain sites. Onset of the disease can occur at any age, and lesions begin acutely with erythematous edematous papule or plaque with scaling. Itching leads to weeping and crusting, then to chronic lichenification. Chronic lesions feature epidermal hyperplasia, hyperkeratosis and parakeratosis, and the dermis is infiltrated with mononuclear cells, Langerhans' cells and mast cells.

Urticaria and angioedema refer to the physical swelling, erythema and itching in superficial cutaneous blood vessels, and is the hallmark cutaneous feature of systemic anaphylaxis. Systemic anaphylaxis is the occurrence of an IgE-mediated reaction simultaneously in multiple organs. Vascular collapse, acute airway obstruction, cutaneous vasodilation and edema, and gastrointestinal and genitourinary muscle spasm occur almost simultaneously, although not always to the same degree. The pathology of anaphylaxis includes angioedema and hyperinflated lungs, with mucous plugging of airways and focal atelectasis. On a cellular level, the lungs appear similarly as during an acute asthma attack, with hypersecretion of bronchial submucosal glands, mucosal and submucosal edema, peribronchial vascular congestion and eosinophilia in the bronchial walls. Pulmonary edema and hemorrhage may be present. Bronchial muscle spasm, hyperinflation, and even rupture of alveoli may also be present. Important features of human anaphylaxis include edema, vascular congestion, and eosinophilia in the lamina propria of the larynx, trachea, epiglottis and hypopharynx.

Eosinophilic gastritis (EG) and/or eosinophilic gastroenteritis (EGE), particularly, represent rare types of eosinophilic gastrointestinal disorder and are characterized by chronic inflammation due to patchy or diffuse infiltration of eosinophils into layers of the stomach (EG) or stomach and small intestine (EGE) (Prussin (2014) Gastroenterol. Clin. North. Am. 43:317-327; Reed et al. (2015) Dig. Liver Dis. 47:197-201; Zhang and Li (2017). Gastroenterol. Hepatol. 32:64-72). Diagnosis is made based on clinical presentation (gastrointestinal symptoms) combined with increased tissue eosinophils in biopsy specimens from the stomach and duodenum without any other cause for the eosinophilia. Symptoms commonly include nausea, vomiting, abdominal pain, diarrhea, bloating, early satiety, and weight loss.

Thus, symptoms of various eosinophil or mast cell disorders include one or more of allergic reactions, difficulty in breathing, inflammation of the airways, reduced airflow during breathing, sinus pain, hyposmia, nasal obstruction, fever, fatigue, nausea, vomiting, abdominal pain, dysphagia, diarrhea, weight loss, skin reactions (e.g., hives and rashes), edema, joint pain, and hypotension.

Therapeutically effective amounts can reduce one or more symptoms associated with eosinophil or mast cell disorders.

In certain examples, therapeutically effective amounts reduce eosinophil or mast cell number or activity. For example, therapeutically effective amounts can result in a decrease in the number of eosinophils or mast cells in serum, blood, tissue, or urine. Therapeutically effective amounts can decrease the number of eosinophils observed in gastrointestinal (GI) biopsies, as compared to a reference level or previous measure.

In relation to hematologic malignancies involving eosinophils or mast cells, therapeutically effective amounts can provide an anti-cancer effect. Anti-cancer effects can include a decrease in the number of cancer cells, an increase in life expectancy, prolonged subject life, induced chemo- or radiosensitivity in cancer cells, inhibited cancer cell proliferation, reduced cancer-associated pain, and/or reduced relapse or re-occurrence of cancer following treatment.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of condition, stage of condition, previous or concurrent therapeutic interventions, idiopathy of the subject and route of administration.

Useful doses can range from 0.1 to 5 μg/kg or from 0.5 to 1 μg/kg. In other examples, a dose can include 1 μg/kg, 15 μg/kg, 30 μg/kg, 50 μg/kg, 55 μg/kg, 70 μg/kg, 90 μg/kg, 150 μg/kg, 350 μg/kg, 500 μg/kg, 750 μg/kg, 1000 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Useful doses can range from 0.1 to 5 μCi/kg or from 0.5 to 1 μCi/kg. In other examples, a dose can include 1 μCi/kg, 15 μCi/kg, 30 μCi/kg, 50 μCi/kg, 55 μCi/kg, 70 μCi/kg, 90 μCi/kg, 150 μCi/kg, 350 μCi/kg, 500 μCi/kg, 750 μCi/kg, or 1000 μCi/kg. In particular embodiments, a dose includes up to 500 μCi/kg.

Exemplary doses of cell-based compositions can include 10⁴ to 10⁹ cells/kg body weight, or 10³ to 10¹¹ cells/kg body weight. Therapeutically effective amounts to administer can include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly). In particular embodiments, the treatment protocol may be dictated by a clinical trial protocol or an FDA-approved treatment protocol.

The compositions described herein can be administered by, for example, injection, inhalation, infusion, perfusion, lavage, or ingestion. Routes of administration can include intravenous, intradermal, intraarterial, intranodal, intravesicular, intrathecal, intraperitoneal, intraparenteral, intranasal, intralesional, intramuscular, oral, subcutaneous, and/or sublingual administration. Formulations are generally be administered by injection.

In particular embodiments, the antibody used in the treatment will depend on the Siglec-8 disorder or isoform of Siglec-8 that the subject has. Furthermore, the format by which the antibody is presented (e.g., scFv, bispecific immune cell engager, multi-domain binding molecule, recombinant receptor, etc.) will be depend on the needs of the patient.

In particular embodiments, the antibodies that bind the V-set domain of Siglec-8 include 11B6, 1 H4, 11B4, 1E1, 1E4, and 1E2. In particular embodiments, an antibody that binds the membrane-distal C2-set domain of Siglec-8 include 2A3. In particular embodiments, antibodies that bind the membrane-proximal C2-set domain of Siglec-8 include 2A7 and 2F10. In particular embodiments, antibodies that bind specifically to Siglec-8^{ΔE2} include 2B111 and 2G9.

(ix) Kits. Also provided herein are kits including at least one antibody or sequences encoding at least one antibody disclosed herein. Kits may be formed with components to practice, for example, the methods described herein. In particular embodiments, the kit includes a Siglec-8 antibody, a multidomain binding molecule, an antibody conjugate, or a multimerized antibody as described herein, or sequences encoding a human Siglec-8 antibody, a multidomain binding molecule, an antibody conjugate, or a multimerized antibody as described herein. In particular embodiments, the kit includes cells expressing a recombinant receptor or compositions to modify cells to express a recombinant receptor. The kit may include material(s), which may be desirable from a user standpoint, such as a buffer(s), a diluent(s), a standard(s), and/or other material useful in sample processing, washing, or conducting any other step of the method described herein.

In particular embodiments, a kit includes an antibody conjugate or sequence encoding an antibody conjugate and any other materials needed for treatment of Siglec-8-related conditions.

The kit according to the present disclosure may also include instructions for carrying out the method. Instructions included in the kit of the present disclosure may be affixed to packaging material or may be included as a package insert. While instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site which provides instructions.

Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (x) Exemplary Embodiments

1. An antibody or binding fragment thereof including a set of complementarity determining regions (CDRs):
according to North including:
a light chain (LC) CDR1 having the sequence as set forth in SEQ ID NO: 26, an LC CDR2 having the sequence as set forth in SEQ ID NO: 27, an LC CDR3 having the sequence as set forth in SEQ ID NO: 28, a heavy chain (HC) CDR1 having the sequence as set forth in SEQ ID NO: 29, an HC CDR2 having the sequence as set forth in SEQ ID NO: 30, and an HC CDR3 having the sequence as set forth in SEQ ID NO: 31;
an LC CDR1 having the sequence as set forth in SEQ ID NO: 34, an LC CDR2 having the sequence as set forth in SEQ ID NO: 35, an LC CDR3 having the sequence as set forth in SEQ ID NO: 28, an HC CDR1 having the sequence as set forth in SEQ ID NO: 36, an HC CDR2 having the sequence as set forth in SEQ ID NO: 37, and an HC CDR3 having the sequence as set forth in SEQ ID NO: 31;
an LC CDR1 having the sequence as set forth in SEQ ID NO: 38, an LC CDR2 having the sequence as set forth in SEQ ID NO: 39, an LC CDR3 having the sequence as set forth in SEQ ID NO: 40 an HC CDR1 having the sequence as set forth in SEQ ID NO:41, an HC CDR2 having the sequence as set forth in SEQ ID NO: 42, and an HC CDR3 having the sequence as set forth in SEQ ID NO: 43;
an LC CDR1 having the sequence as set forth in SEQ ID NO: 45, an LC CDR2 having the sequence as set forth in SEQ ID NO: 46, an LC CDR3 having the sequence as set forth in SEQ ID NO: 47, an HC CDR1 having the sequence as set forth in SEQ ID NO:48 an HC CDR2 having the sequence as set forth in SEQ ID NO: 49, and an HC CDR3 having the sequence as set forth in SEQ ID NO: 50;
an LC CDR1 having the sequence as set forth in SEQ ID NO: 51, an LC CDR2 having the sequence as set forth in SEQ ID NO: 52, an LC CDR3 having the sequence as set forth in SEQ ID NO: 53, an HC CDR1 having the sequence as set forth in SEQ ID NO:54, an HC CDR2 having the sequence as set forth in SEQ ID NO: 55, and an HC CDR3 having the sequence as set forth in SEQ ID NO: 56;
an LC CDR1 having the sequence as set forth in SEQ ID NO: 57, an LC CDR2 having the sequence as set forth in SEQ ID NO: 58, an LC CDR3 having the sequence as set forth in SEQ ID NO: 59, an HC CDR1 having the sequence as set forth in SEQ ID NO:60, an HC CDR2 having the sequence as set forth in SEQ ID NO: 61, and an HC CDR3 having the sequence as set forth in SEQ ID NO: 62;
an LC CDR1 having the sequence as set forth in SEQ ID NO: 63, an LC CDR2 having the sequence as set forth in SEQ ID NO: 64, an LC CDR3 having the sequence as set forth in SEQ ID NO: 65, an HC CDR1 having the sequence as set forth in SEQ ID NO:66, an HC CDR2 having the sequence as set forth in SEQ ID NO: 67, and an HC CDR3 having the sequence as set forth in SEQ ID NO: 68;
an LC CDR1 having the sequence as set forth in SEQ ID NO: 69, an LC CDR2 having the sequence as set forth in SEQ ID NO: 70, an LC CDR3 having the sequence as set forth in SEQ ID NO: 71, an HC CDR1 having the sequence as set forth in SEQ ID NO:72, an HC CDR2 having the sequence as set forth in SEQ ID NO: 73, and an HC CDR3 having the sequence as set forth in SEQ ID NO: 74;
an LC CDR1 having the sequence as set forth in SEQ ID NO: 75, an LC CDR2 having the sequence as set forth in SEQ ID NO: 76, an LC CDR3 having the sequence as set forth in SEQ ID NO: 77, an HC CDR1 having the sequence as set forth in SEQ ID NO:78, an HC CDR2 having the sequence as set forth in SEQ ID NO: 79, and an HC CDR3 having the sequence as set forth in SEQ ID NO: 80;
an LC CDR1 having the sequence as set forth in SEQ ID NO: 69, an LC CDR2 having the sequence as set forth in SEQ ID NO: 70, an LC CDR3 having the sequence as set forth in SEQ ID NO: 71, an HC CDR1 having the sequence as set forth in SEQ ID NO:72, an HC CDR2 having the sequence as set forth in SEQ ID NO: 73, and an HC CDR3 having the sequence as set forth in SEQ ID NO: 74; or
an LC CDR1 having the sequence as set forth in SEQ ID NO: 75, an LC CDR2 having the sequence as set forth in SEQ ID NO: 76, an LC CDR3 having the sequence as set forth in SEQ ID NO: 77, an HC CDR1 having the sequence as set forth in SEQ ID NO:81, an HC CDR2 having the sequence as set forth in SEQ ID NO: 82, and an HC CDR3 having the sequence as set forth in SEQ ID NO: 83;
according to IMGT including:
an LC CDR1 having the sequence as set forth in SEQ ID NO: 86, an LC CDR2 having the sequence ATS, an LC CDR3 having the sequence as set forth in SEQ ID NO: 28, an HC CDR1 having the sequence as set forth in SEQ ID NO:87, an HC CDR2 having the sequence as set forth in SEQ ID NO: 88, and an HC CDR3 having the sequence as set forth in SEQ ID NO: 31;
an LC CDR1 having the sequence as set forth in SEQ ID NO: 86, an LC CDR2 having the sequence ASS, an LC CDR3 having the sequence as set forth in SEQ ID NO: 28, an HC CDR1 having the sequence as set forth in SEQ ID NO:90, an HC CDR2 having the sequence as set forth in SEQ ID NO: 88, and an HC CDR3 having the sequence as set forth in SEQ ID NO: 31;
an LC CDR1 having the sequence as set forth in SEQ ID NO: 91, an LC CDR2 having the sequence LVS, an LC CDR3 having the sequence as set forth in SEQ ID NO: 40, an HC CDR1 having the sequence as set forth in SEQ ID NO:92, an HC CDR2 having the sequence as set forth in SEQ ID NO: 93, and an HC CDR3 having the sequence as set forth in SEQ ID NO: 43;
an LC CDR1 having the sequence as set forth in SEQ ID NO: 95, an LC CDR2 having the sequence GAS, an LC CDR3 having the sequence as set forth in SEQ ID NO: 47, an HC CDR1 having the sequence as set forth in SEQ ID NO:96, an HC CDR2 having the sequence as set forth in SEQ ID NO: 97, and an HC CDR3 having the sequence as set forth in SEQ ID NO: 50;
an LC CDR1 having the sequence as set forth in SEQ ID NO: 98, an LC CDR2 having the sequence AAS, an LC CDR3 having the sequence as set forth in SEQ ID NO: 53, an HC CDR1 having the sequence as set forth in SEQ ID NO:99, an HC CDR2 having the sequence as set forth in SEQ ID NO: 100, and an HC CDR3 having the sequence as set forth in SEQ ID NO: 56;
an LC CDR1 having the sequence as set forth in SEQ ID NO: 101, an LC CDR2 having the sequence GAS, an LC CDR3 having the sequence as set forth in SEQ ID NO: 59, an HC CDR1 having the sequence as set forth in SEQ ID NO:102, an HC CDR2 having the sequence as set forth in SEQ ID NO: 103 and an HC CDR3 having the sequence as set forth in SEQ ID NO: 62;
an LC CDR1 having the sequence as set forth in SEQ ID NO: 104, an LC CDR2 having the sequence NNN, an LC CDR3 having the sequence as set forth in SEQ ID NO: 65, an HC CDR1 having the sequence as set forth in SEQ ID NO:105, an HC CDR2 having the sequence as set forth in SEQ ID NO: 106, and an HC CDR3 having the sequence as set forth in SEQ ID NO: 68;
an LC CDR1 having the sequence as set forth in SEQ ID NO: 107, an LC CDR2 having the sequence AAS, an LC CDR3 having the sequence as set forth in SEQ ID NO: 71, an HC CDR1 having the sequence as set forth in SEQ ID NO:108, an HC CDR2 having the sequence as set forth in SEQ ID NO: 109, and an HC CDR3 having the sequence as set forth in SEQ ID NO: 74;
an LC CDR1 having the sequence as set forth in SEQ ID NO: 110, an LC CDR2 having the sequence KIS, an LC CDR3 having the sequence as set forth in SEQ ID NO: 77, an HC CDR1 having the sequence as set forth in SEQ ID NO:111, an HC CDR2 having the sequence as set forth in SEQ ID NO: 112, and an HC CDR3 having the sequence as set forth in SEQ ID NO: 80;
an LC CDR1 having the sequence as set forth in SEQ ID NO: 107, an LC CDR2 having the sequence AAS, an LC CDR3 having the sequence as set forth in SEQ ID NO: 71, an HC CDR1 having the sequence as set forth in SEQ ID NO:108, an HC CDR2 having the sequence as set forth in SEQ ID NO: 109, and an HC CDR3 having the sequence as set forth in SEQ ID NO: 74; or
an LC CDR1 having the sequence as set forth in SEQ ID NO: 110, an LC CDR2 having the sequence KIS, an LC CDR3 having the sequence as set forth in SEQ ID NO: 77, an HC CDR1 having the sequence as set forth in SEQ ID NO:113, an HC CDR2 having the sequence as set forth in SEQ ID NO: 114, and an HC CDR3 having the sequence as set forth in SEQ ID NO: 83;
   according to Kabat including:
an LC CDR1 having the sequence as set forth in SEQ ID NO: 26, an LC CDR2 having the sequence as set forth in SEQ ID NO: 116, an LC CDR3 having the sequence as set forth in SEQ ID NO: 28, an HC CDR1 having the sequence as set forth in SEQ ID NO: 117, an HC CDR2 having the sequence as set forth in SEQ ID NO:118, and an HC CDR3 having the sequence as set forth in SEQ ID NO: 119;
an LC CDR1 having the sequence as set forth in SEQ ID NO: 34, an LC CDR2 having the sequence as set forth in SEQ ID NO: 121, an LC CDR3 having the sequence as set forth in SEQ ID NO: 28, an HC CDR1 having the sequence as set forth in SEQ ID NO: 117, an HC CDR2 having the sequence as set forth in SEQ ID NO:122, and an HC CDR3 having the sequence as set forth in SEQ ID NO: 119;
an LC CDR1 having the sequence as set forth in SEQ ID NO: 38, an LC CDR2 having the sequence as set forth in SEQ ID NO: 123, an LC CDR3 having the sequence as set forth in SEQ ID NO: 40, an HC CDR1 having the sequence as set forth in SEQ ID NO: 124, an HC CDR2 having the sequence as set forth in SEQ ID NO:125, and an HC CDR3 having the sequence as set forth in SEQ ID NO: 126;
an LC CDR1 having the sequence as set forth in SEQ ID NO: 45, an LC CDR2 having the sequence as set forth in SEQ ID NO: 127, an LC CDR3 having the sequence as set forth in SEQ ID NO: 47, an HC CDR1 having the sequence as set forth in SEQ ID NO: 128, an HC CDR2 having the sequence as set forth in SEQ ID NO:129, and an HC CDR3 having the sequence as set forth in SEQ ID NO: 130;
an LC CDR1 having the sequence as set forth in SEQ ID NO: 51, an LC CDR2 having the sequence as set forth in SEQ ID NO: 131, an LC CDR3 having the sequence as set forth in SEQ ID NO: 53, an HC CDR1 having the sequence as set forth in SEQ ID NO: 132, an HC CDR2 having the sequence as set forth in SEQ ID NO:133, and an HC CDR3 having the sequence as set forth in SEQ ID NO: 134;
an LC CDR1 having the sequence as set forth in SEQ ID NO: 57, an LC CDR2 having the sequence as set forth in SEQ ID NO: 135, an LC CDR3 having the sequence as set forth in SEQ ID NO: 59, an HC CDR1 having the sequence as set forth in SEQ ID NO: 136, an HC CDR2 having the sequence as set forth in SEQ ID NO:137, and an HC CDR3 having the sequence as set forth in SEQ ID NO: 138;
an LC CDR1 having the sequence as set forth in SEQ ID NO: 63, an LC CDR2 having the sequence as set forth in SEQ ID NO: 139, an LC CDR3 having the sequence as set forth in SEQ ID NO: 65, an HC CDR1 having the sequence as set forth in SEQ ID NO: 140, an HC CDR2 having the sequence as set forth in SEQ ID NO:141, and an HC CDR3 having the sequence as set forth in SEQ ID NO: 142;
an LC CDR1 having the sequence as set forth in SEQ ID NO: 69, an LC CDR2 having the sequence as set forth in SEQ ID NO: 143, an LC CDR3 having the sequence as set forth in SEQ ID NO: 71, an HC CDR1 having the sequence as set forth in SEQ ID NO: 144, an HC CDR2 having the sequence as set forth in SEQ ID NO:145, and an HC CDR3 having the sequence as set forth in SEQ ID NO: 146;
an LC CDR1 having the sequence as set forth in SEQ ID NO: 75, an LC CDR2 having the sequence as set forth in SEQ ID NO: 147, an LC CDR3 having the sequence as set forth in SEQ ID NO: 77, an HC CDR1 having the sequence as set forth in SEQ ID NO: 148, an HC CDR2 having the sequence as set forth in SEQ ID NO:149, and an HC CDR3 having the sequence as set forth in SEQ ID NO: 150;
an LC CDR1 having the sequence as set forth in SEQ ID NO: 69, an LC CDR2 having the sequence as set forth in SEQ ID NO: 143, an LC CDR3 having the sequence as set forth in SEQ ID NO: 71, an HC CDR1 having the sequence as set forth in SEQ ID NO: 144, an HC CDR2 having the sequence as set forth in SEQ ID NO:151, and an HC CDR3 having the sequence as set forth in SEQ ID NO: 146; or
an LC CDR1 having the sequence as set forth in SEQ ID NO: 75, an LC CDR2 having the sequence as set forth in SEQ ID NO: 147, an LC CDR3 having the sequence as set forth in SEQ ID NO: 77, an HC CDR1 having the sequence as set forth in SEQ ID NO: 148, an HC CDR2 having the sequence as set forth in SEQ ID NO:152, and an HC CDR3 having the sequence as set forth in SEQ ID NO: 153;
   according to Chothia including:
an LC CDR1 the sequence as set forth in SEQ ID NO: 26, an LC CDR2 the sequence as set forth in SEQ ID NO: 116, an LC CDR3 having the sequence as set forth in SEQ ID NO: 28, an HC CDR1 having the sequence as set forth in SEQ ID NO: 156, an HC CDR2 having the sequence as set forth in SEQ ID NO:157, and an HC CDR3 having the sequence as set forth in SEQ ID NO: 119;
an LC CDR1 having the sequence as set forth in SEQ ID NO: 34, an LC CDR2 having the sequence as set forth in SEQ ID NO: 121, an LC CDR3 having the sequence as set forth in SEQ ID NO: 28, an HC CDR1 having the sequence as set forth in SEQ ID NO: 158, an HC CDR2 having the sequence as set forth in SEQ ID NO:157, and an HC CDR3 having the sequence as set forth in SEQ ID NO: 119;
an LC CDR1 having the sequence as set forth in SEQ ID NO: 38, an LC CDR2 having the sequence as set forth in SEQ ID NO: 123, an LC CDR3 having the sequence as set forth in SEQ ID NO: 40, an HC CDR1 having the sequence as set forth in SEQ ID NO: 159, an HC CDR2 having the sequence as set forth in SEQ ID NO:160, and an HC CDR3 having the sequence as set forth in SEQ ID NO: 126;
an LC CDR1 having the sequence as set forth in SEQ ID NO: 45, an LC CDR2 having the sequence as set forth in SEQ ID NO: 127, an LC CDR3 having the sequence as set forth in SEQ ID NO: 47, an HC CDR1 having the sequence as set forth in SEQ ID NO: 161, an HC CDR2 having the sequence as set forth in SEQ ID NO:162, and an HC CDR3 having the sequence as set forth in SEQ ID NO: 130;
an LC CDR1 having the sequence as set forth in SEQ ID NO: 51, an LC CDR2 having the sequence as set forth in SEQ ID NO: 131, an LC CDR3 having the sequence as set forth in SEQ ID NO: 53, an HC CDR1 having the sequence as set forth in SEQ ID NO: 163, an HC CDR2 having the sequence as set forth in SEQ ID NO:164, and an HC CDR3 having the sequence as set forth in SEQ ID NO: 134;
an LC CDR1 having the sequence as set forth in SEQ ID NO: 57, an LC CDR2 having the sequence as set forth in SEQ ID NO: 135, an LC CDR3 having the sequence as set forth in SEQ ID NO: 59, an HC CDR1 having the sequence as set forth in SEQ ID NO: 165, an HC CDR2 having the sequence as set forth in SEQ ID NO:166, and an HC CDR3 having the sequence as set forth in SEQ ID NO: 138;
an LC CDR1 having the sequence as set forth in SEQ ID NO: 63, an LC CDR2 having the sequence as set forth in SEQ ID NO: 139, an LC CDR3 having the sequence as set forth in SEQ ID NO: 65, an HC CDR1 having the sequence as set forth in SEQ ID NO: 167, an HC CDR2 having the sequence as set forth in SEQ ID NO:168, and an HC CDR3 having the sequence as set forth in SEQ ID NO: 142;
an LC CDR1 having the sequence as set forth in SEQ ID NO: 69, an LC CDR2 having the sequence as set forth in SEQ ID NO: 143, an LC CDR3 having the sequence as set forth in SEQ ID NO: 71, an HC CDR1 having the sequence as set forth in SEQ ID NO: 169, an HC CDR2 having the sequence as set forth in SEQ ID NO:170, and an HC CDR3 having the sequence as set forth in SEQ ID NO: 146;
an LC CDR1 having the sequence as set forth in SEQ ID NO: 75, an LC CDR2 having the sequence as set forth in SEQ ID NO: 147, an LC CDR3 having the sequence as set forth in SEQ ID NO: 77, an HC CDR1 having the sequence as set forth in SEQ ID NO: 171, an HC CDR2 having the sequence as set forth in SEQ ID NO:172, and an HC CDR3 having the sequence as set forth in SEQ ID NO: 150;
an LC CDR1 having the sequence as set forth in SEQ ID NO: 69, an LC CDR2 having the sequence as set forth in SEQ ID NO: 143, an LC CDR3 having the sequence as set forth in SEQ ID NO: 71, an HC CDR1 having the sequence as set forth in SEQ ID NO: 169, an HC CDR2 having the sequence as set forth in SEQ ID NO:170, and an HC CDR3 having the sequence as set forth in SEQ ID NO: 146; or
an LC CDR1 having the sequence as set forth in SEQ ID NO: 75, an LC CDR2 having the sequence as set forth in SEQ ID NO: 147, an LC CDR3 having the sequence as set forth in SEQ ID NO: 77, an HC CDR1 having the sequence as set forth in SEQ ID NO: 173, an HC CDR2 having the sequence as set forth in SEQ ID NO:174, and an HC CDR3 having the sequence as set forth in SEQ ID NO: 153;
   according to Contact including:
an LC CDR1 having the sequence as set forth in SEQ ID NO: 175, an LC CDR2 having the sequence as set forth in SEQ ID NO: 176, an LC CDR3 having the sequence as set forth in SEQ ID NO: 177, an HC CDR1 having the sequence as set forth in SEQ ID NO: 178, an HC CDR2 having the sequence as set forth in SEQ ID NO:179, and an HC CDR3 having the sequence as set forth in SEQ ID NO: 180;
an LC CDR1 having the sequence as set forth in SEQ ID NO: 175, an LC CDR2 having the sequence as set forth in SEQ ID NO: 183, an LC CDR3 having the sequence as set forth in SEQ ID NO: 177, an HC CDR1 having the sequence as set forth in SEQ ID NO: 184, an HC CDR2 having the sequence as set forth in SEQ ID NO:185, and an HC CDR3 having the sequence as set forth in SEQ ID NO: 180;
an LC CDR1 having the sequence as set forth in SEQ ID NO: 186, an LC CDR2 having the sequence as set forth in SEQ ID NO: 187, an LC CDR3 having the sequence as set forth in SEQ ID NO: 188, an HC CDR1 having the sequence as set forth in SEQ ID NO: 189, an HC CDR2 having the sequence as set forth in SEQ ID NO:190, and an HC CDR3 having the sequence as set forth in SEQ ID NO: 191;
an LC CDR1 having the sequence as set forth in SEQ ID NO: 192, an LC CDR2 having the sequence as set forth in SEQ ID NO: 193, an LC CDR3 having the sequence as set forth in SEQ ID NO: 194, an HC CDR1 having the sequence as set forth in SEQ ID NO: 195, an HC CDR2 having the sequence as set forth in SEQ ID NO:196, and an HC CDR3 having the sequence as set forth in SEQ ID NO: 197;
an LC CDR1 having the sequence as set forth in SEQ ID NO: 198, an LC CDR2 having the sequence as set forth in SEQ ID NO: 199, an LC CDR3 having the sequence as set forth in SEQ ID NO: 200, an HC CDR1 having the sequence as set forth in SEQ ID NO: 201, an HC CDR2 having the sequence as set forth in SEQ ID NO:202, and an HC CDR3 having the sequence as set forth in SEQ ID NO: 203;
an LC CDR1 having the sequence as set forth in SEQ ID NO: 204, an LC CDR2 having the sequence as set forth in SEQ ID NO: 205, an LC CDR3 having the sequence as set forth in SEQ ID NO: 206, an HC CDR1 having the sequence as set forth in SEQ ID NO: 207, an HC CDR2 having the sequence as set forth in SEQ ID NO:208, and an HC CDR3 having the sequence as set forth in SEQ ID NO: 209;
an LC CDR1 having the sequence as set forth in SEQ ID NO: 210, an LC CDR2 having the sequence as set forth in SEQ ID NO: 211, an LC CDR3 having the sequence as set forth in SEQ ID NO: 212, an HC CDR1 having the sequence as set forth in SEQ ID NO: 140, an HC CDR2 having the sequence as set forth in SEQ ID NO:213, and an HC CDR3 having the sequence as set forth in SEQ ID NO: 214;
an LC CDR1 having the sequence as set forth in SEQ ID NO: 215, an LC CDR2 having the sequence as set forth in SEQ ID NO: 216, an LC CDR3 having the sequence as set forth in SEQ ID NO: 217, an HC CDR1 having the sequence as set forth in SEQ ID NO: 218, an HC CDR2 having the sequence as set forth in SEQ ID NO:219, and an HC CDR3 having the sequence as set forth in SEQ ID NO: 220;
an LC CDR1 having the sequence as set forth in SEQ ID NO: 221, an LC CDR2 having the sequence as set forth in SEQ ID NO: 222, an LC CDR3 having the sequence as set forth in SEQ ID NO: 223, an HC CDR1 having the sequence as set forth in SEQ ID NO: 224, an HC CDR2 having the sequence as set forth in SEQ ID NO:225, and an HC CDR3 having the sequence as set forth in SEQ ID NO: 226;
an LC CDR1 having the sequence as set forth in SEQ ID NO: 215, an LC CDR2 having the sequence as set forth in SEQ ID NO: 216, an LC CDR3 having the sequence as set forth in SEQ ID NO: 217, an HC CDR1 having the sequence as set forth in SEQ ID NO: 218, an HC CDR2 having the sequence as set forth in SEQ ID NO:219, and an HC CDR3 having the sequence as set forth in SEQ ID NO: 220; or
an LC CDR1 having the sequence as set forth in SEQ ID NO: 221, an LC CDR2 having the sequence as set forth in SEQ ID NO: 222, an LC CDR3 having the sequence as set forth in SEQ ID NO: 223, an HC CDR1 having the sequence as set forth in SEQ ID NO: 224, an HC CDR2 having the sequence as set forth in SEQ ID NO:227, and an HC CDR3 having the sequence as set forth in SEQ ID NO: 228.

2. An antibody or binding fragment thereof of embodiment 1 including:
a variable light chain having the sequence as set forth in SEQ ID NO: 4 and a variable heavy chain having the sequence as set forth in SEQ ID NO: 5 or a variable light chain having a sequence having at least 90% sequence identity to the sequence as set forth in SEQ ID NO: 4 and a variable heavy chain having a sequence having at least 90% sequence identity to the sequence as set forth in SEQ ID NO: 5;
a variable light chain having the sequence as set forth in SEQ ID NO: 13 and a variable heavy chain having the sequence as set forth in SEQ ID NO: 14 or a variable light chain having a sequence having at least 90% sequence identity to the sequence as set forth in SEQ ID NO: 13 and a variable heavy chain having a sequence having at least 90% sequence identity to the sequence as set forth in SEQ ID NO: 14;
a variable light chain having the sequence as set forth in SEQ ID NO: 15 and a variable heavy chain having the sequence as set forth in SEQ ID NO: 16 or a variable light chain having a sequence having at least 90% sequence identity to the sequence as set forth in SEQ ID NO: 15 and a variable heavy chain having a sequence having at least 90% sequence identity to the sequence as set forth in SEQ ID NO: 16;
a variable light chain having the sequence as set forth in SEQ ID NO: 17 and a variable heavy chain having the sequence as set forth in SEQ ID NO: 18 or a variable light chain having a sequence having at least 90% sequence identity to the sequence as set forth in SEQ ID NO: 17 and a variable heavy chain having a sequence having at least 90% sequence identity to the sequence as set forth in SEQ ID NO: 18;
a variable light chain having the sequence as set forth in SEQ ID NO: 9 and a variable heavy chain having the sequence as set forth in SEQ ID NO: 10 or a variable light chain having a sequence having at least 90% sequence identity to the sequence as set forth in SEQ ID NO: 9 and a variable heavy chain having a sequence having at least 90% sequence identity to the sequence as set forth in SEQ ID NO: 10;
a variable light chain having the sequence as set forth in SEQ ID NO: 1 and a variable heavy chain having the sequence as set forth in SEQ ID NO: 2 or a variable light chain having a sequence having at least 90% sequence identity to the sequence as set forth in SEQ ID NO: 1 and a variable heavy chain having a sequence having at least 90% sequence identity to the sequence as set forth in SEQ ID NO: 2;
a variable light chain having the sequence as set forth in SEQ ID NO: 6 and a variable heavy chain having the sequence as set forth in SEQ ID NO: 7 or a variable light chain having a sequence having at least 90% sequence identity to the sequence as set forth in SEQ ID NO: 6 and a variable heavy chain having a sequence having at least 90% sequence identity to the sequence as set forth in SEQ ID NO: 7;
a variable light chain having the sequence as set forth in SEQ ID NO: 19 and a variable heavy chain having the sequence as set forth in SEQ ID NO: 20 or a variable light chain having a sequence having at least 90% sequence identity to the sequence as set forth in SEQ ID NO: 19 and a variable heavy chain having a sequence having at least 90% sequence identity to the sequence as set forth in SEQ ID NO: 20;
a variable light chain having the sequence as set forth in SEQ ID NO: 19 and a variable heavy chain having the sequence as set forth in SEQ ID NO: 23 or a variable light chain having a sequence having at least 90% sequence identity to the sequence as set forth in SEQ ID NO: 19 and a variable heavy chain having a sequence having at least 90% sequence identity to the sequence as set forth in SEQ ID NO: 23;
a variable light chain having the sequence as set forth in SEQ ID NO: 21 and a variable heavy chain having the sequence as set forth in SEQ ID NO: 22 or a variable light chain having a sequence having at least 90% sequence identity to the sequence as set forth in SEQ ID NO: 21 and a variable heavy chain having a sequence having at least 90% sequence identity to the sequence as set forth in SEQ ID NO: 22; or
a variable light chain having the sequence as set forth in SEQ ID NO: 21 and a variable heavy chain having the sequence as set forth in SEQ ID NO: 24 or a variable light chain having a sequence having at least 90% sequence identity to the sequence as set forth in SEQ ID NO: 21 and a variable heavy chain having a sequence having at least 90% sequence identity to the sequence as set forth in SEQ ID NO: 24.

3. The antibody or binding fragment thereof of embodiments 1 or 2, wherein the antibody or binding fragment thereof further includes a human constant region.

4. The antibody or binding fragment thereof of embodiment 3, wherein the human constant region includes a human light chain constant region and/or a human heavy chain constant region.

5. The antibody or binding fragment thereof of embodiment 4, wherein the human light chain constant region includes a human Igκ light chain constant region or a human IgA light chain constant region.

6. The antibody or binding fragment thereof of embodiments 4 or 5, wherein the human light chain constant region includes a human Igκ light chain constant region.

7. The antibody or binding fragment thereof of embodiments 5 or 6, wherein the human Igκ light chain constant region includes the sequence as set forth in SEQ ID NO: 337.

8. The antibody or binding fragment thereof of any of embodiments 1-7, including a variable light chain and a human Igκ light chain constant region including the sequence as set forth in SEQ ID NOs: 281, 287, 293, 434, 443, 452, 461, 476, 295, 320, 323, 436, 445, 454, 463, or 478 or a sequence having at least 95% sequence identity to SEQ ID NOs: 281, 287, 293, 434, 443, 453, 461, 476, 295, 320, 323, 436, 445, 454, 463, or 478.

9. The antibody or binding fragment thereof of any of embodiments 1-8, including a variable light chain and a human Igκ light chain constant region encoded by the sequence as set forth in SEQ ID NOs: 282, 288, 294, 435, 444, 453, 462 or 477 or a sequence having at least 95% sequence identity to SEQ ID NO: 282, 288, 294, 435, 444, 453, 462 or 477.

10. The antibody or binding fragment thereof of embodiments 4 or 5, wherein the human light chain constant region includes a human IgA light chain constant region.

11. The antibody or binding fragment thereof of embodiment 10, wherein the human IgA light chain constant region includes the sequence as set forth in SEQ ID NO: 338.

12. The antibody or binding fragment thereof of any of embodiments 1-11, including a variable light chain and a human IgA light chain constant region including the sequence as set forth in SEQ ID NO: 275 or SEQ ID NO: 317 or a sequence having at least 95% sequence identity to SEQ ID NO: 275 or SEQ ID NO: 317.

13. The antibody or binding fragment thereof of any of embodiments 1-12, including a variable light chain and a human IgA light chain constant region encoded by the sequence as set forth in SEQ ID NO: 276 or a sequence having at least 95% sequence identity to SEQ ID NO: 276.

14. The antibody or binding fragment thereof of any of embodiments 3-13, wherein the human constant region includes a human heavy chain constant region.

15. The antibody or binding fragment thereof of embodiment 14, wherein the human heavy chain constant region includes an IgG heavy chain constant region, an IgA heavy chain constant region, an IgM heavy chain constant region, an IgD heavy chain constant region, or an IgE heavy chain constant region.

16. The antibody or binding fragment thereof of embodiment 15, wherein the IgG heavy chain constant region includes an IgG1 heavy chain constant region, an IgG2 heavy chain constant region, an IgG3 heavy chain constant region, or an IgG4 heavy chain constant region.

17. The antibody or binding fragment thereof of embodiments 15 or 16, wherein the IgG heavy chain constant region includes an IgG1 heavy chain constant region.

18. The antibody or binding fragment thereof of embodiment 17, including a variable heavy chain and an IgG1 heavy chain constant region including the sequence as set forth in SEQ ID NOs: 271, 277, 283, 289, 296, 437, 446, 455, 464, 470, 479, 315, 318, 321, 324, 298, 439, 448, 457, 466, 472, or 481 or a sequence having at least 95% sequence identity to SEQ ID NOs: 271, 277, 283, 289, 296, 437, 446, 455, 464, 470, 479, 315, 318, 321, 324, 298, 439, 448, 457, 466, 472, or 481.

19. The antibody or binding fragment thereof of embodiments 17 or 18, including a variable heavy chain and an IgG1 heavy chain constant region encoded by the sequence as set forth in SEQ ID NOs: 272, 278, 284, 290, 297, 438, 447, 456, 465, 471, or 480 or a sequence having at least 95% sequence identity to SEQ ID NOs: 272, 278, 284, 290, 297, 438, 447, 456, 465, 471, or 480.

20. The antibody or binding fragment thereof of embodiments 15 or 16, wherein the IgG heavy chain constant region includes IgG4 heavy chain constant region.

21. The antibody or binding fragment thereof of embodiment 20, including a variable heavy chain and an IgG4 heavy chain constant region including the sequence as set forth in SEQ ID NOs: 273, 279, 285, 291, 431, 440, 449, 458, 467, 473, 482, 316, 319, 322, 325, 433, 442, 451, 460, 469, 475, or 484 or a sequence having at least 95% sequence identity to SEQ ID NOs: 273, 279, 285, 291, 431, 440, 449, 458, 467, 473, 482, 316, 319, 322, 325, 433, 442, 451, 460, 469, 475, or 484.

22. The antibody or binding fragment thereof of embodiments 20 or 21, including a variable heavy chain and an IgG4 heavy chain constant region encoded by the sequence as set forth in SEQ ID NOs: 274, 280, 286, 292, 432, 441, 450, 459, 468, 474, or 483 or a sequence having at least 95% sequence identity to SEQ ID NOs: 274, 280, 286, 292, 432, 441, 450, 459, 468, 474, or 483.

23. The antibody or binding fragment thereof of any of embodiments 1-22, wherein the antibody or fragment thereof further includes a signal peptide.

24. The antibody or binding fragment thereof of embodiment 23, wherein the signal peptide includes the sequence as set forth in SEQ ID NOs: 231, 232, 234, 235, 236, 238, 239, 240, 241, 242, 243, 244, 245, 246, 247, 248, or 249.

25. A single chain variable fragment (scFv) including a variable light chain and a variable heavy chain having an antibody or binding fragment thereof of any of embodiments 1-24, wherein the variable light chain and the variable heavy chain are linked by a flexible linker.

26. The scFv of embodiment 25, wherein the flexible linker includes a Gly-Ser linker.

27. The scFv of embodiment 26, wherein the Gly-Ser linker includes the sequence as set forth in any of SEQ ID NO: 347-360.

28. The scFv of any of embodiments 25-27, wherein the flexible linker includes a Whitlow linker.

29. The scFv of any of embodiments 25-28, wherein the scFv has the sequence as set forth in SEQ ID NOs: 421, 423, 425, 427, 429, 491, 492, 493, 494, or 495 or has a sequence with at least 90% sequence identity to a sequence as set forth in SEQ ID NOs: 421, 423, 425, 427, 429, 491, 492, 493, 494, or 495.

30. The scFv of any of embodiments 25-29, wherein the scFv is encoded by the sequence set forth in SEQ ID NO: 422, 424, 426, 428, or 430, or a sequence having at least 90% sequence identity to SEQ ID NO: 422, 424, 426, 428, or 430.

31. A multi-domain binding molecule including at least two binding domains wherein at least one binding domain includes the antibody or binding fragment thereof of any of embodiments 1-24.

32. The multi-domain binding molecule of embodiment 31, wherein the multi-domain binding molecule includes an immune cell engaging molecule.

33. The multi-domain binding molecule of embodiment 32, wherein the immune cell engaging molecule activates a B cell, T cell, natural killer (NK) cell, or macrophage.

34. The multi-domain binding molecule of embodiment 33, wherein the T cell is a CD3 T cell, a CD4 T cell, a CD8 T cell, a central memory T cell, an effector memory T cell, and/or a naïve T cell.

35. The multi-domain binding molecule of any of embodiments 32-34, wherein a binding domain of the immune cell engaging molecule binds CD3, CD28, CD8, NKG2D, CD8, CD16, KIR2DL4, KIR2DS1, KIR2DS2, KIR3DS1, NKG2C, NKG2E, NKG2D, NKp30, NKp44, NKp46, NKp80, DNAM-1, CD11b, CD11c, CD64, CD68, CD119, CD163, CD206, CD209, F4/80, IFGR2, Toll-like receptors 1-9, IL-4Rα, or MARCO.

36. The multi-domain binding molecule of any of embodiments 32-35, wherein a binding domain of the immune cell engaging molecule binds CD3.

37. The multi-domain binding molecule of any of embodiments 31-36, wherein the multi-domain binding molecule includes a single chain variable fragment (scFv).

38. The multi-domain binding molecule of embodiment 37, wherein the scFv includes a variable light chain and a variable heavy chain having a CDR set of any of embodiments 1-24, wherein the variable light chain and the variable heavy chain are linked by a flexible linker.

39. The multi-domain binding molecule of embodiment 38, wherein the flexible linker includes a Gly-Ser linker.

40. The multi-domain binding molecule of embodiment 39, wherein the Gly-Ser linker includes the sequence as set forth in any of SEQ ID NO: 347-360.

41. The multi-domain binding molecule of any of embodiments 37-40, wherein the scFv includes the sequence as set forth in SEQ ID NOs: 421, 423, 425, 427, 429, 491, 492, 493, 494, or 495 or has a sequence with at least 90% sequence identity to a sequence as set forth in SEQ ID NOs: 421, 423, 425, 427, 429, 491, 492, 493, 494, or 495.

42. The multi-domain binding molecule of any of embodiments 37-41, wherein the scFv is encoded by the sequence set forth in SEQ ID NOs: 422, 424, 426, 428, or 430, or a sequence having at least 90% sequence identity to SEQ ID NOs: 422, 424, 426, 428, or 430.

43. The multi-domain binding molecule of any of embodiments 31-42, wherein the at least two binding domains are joined by a protein linker.

44. The multi-domain binding molecule of embodiment 43, wherein the protein linker is a Gly-Ser linker.

45. The multi-domain binding molecule of embodiment 44, wherein the Gly-Ser linker is (GlyₓSer_{y})ₙ wherein x and y are independently an integer from 0 to 10 provided that x and y are not both 0 and wherein n is an integer of 1, 2, 3, 4, 5, 6, 7, 8, 9 or 10.

46. The multi-domain binding molecule of any of embodiments 31-45, including 2, 3, 4, 5, 6, 7, 8, 9, or 10 copies of the antibody or binding fragment thereof of any of embodiments 1-24.

47. The multi-domain binding molecule of any of embodiments 31-46, wherein the multi-domain binding molecule is a dimer, trimer, tetramer, pentamer, hexamer, or heptamer.

48. The multi-domain binding molecule of embodiments 46 or 47, wherein at least two copies of the antibody or binding fragment thereof are linked to an Fc region of an antibody.

49. The multi-domain binding molecule of embodiment 48, wherein the Fc region is an IgA Fc region or an IgM Fc region.

50. The multi-domain binding molecule of embodiments 48 or 49, wherein the Fc region includes a multimerizing fragment of an IgA Fc region or a multimerizing fragment of an IgM Fc region.

51. The multi-domain binding molecule of embodiment 50, wherein the multimerizing fragment of the IgA Fc region includes an IgA tailpiece.

52. The multi-domain binding molecule of embodiments 50 or 51, wherein the multimerizing fragment of the IgA Fc region includes an IgA CA3 domain and an IgA tailpiece.

53. The multi-domain binding molecule of any of embodiments 50-52, wherein the multimerizing fragment of the IgA Fc region includes an IgA CA2 domain, an IgA CA3 domain, and an IgA tailpiece.

54. The multi-domain binding molecule of any of embodiments 50-53, wherein the multimerizing fragment of the IgA Fc region includes an IgA CA1 domain, an IgA CA2 domain, an IgA CA3 domain, and an IgA tailpiece.

55. The multi-domain binding molecule of any of embodiments 50-54, wherein the multimerizing fragment of the IgM Fc region includes an IgM tailpiece.

56. The multi-domain binding molecule of any of embodiments 50-55, wherein the multimerizing fragment of the IgM Fc region includes a Cμ4 domain and an IgM tailpiece.

57. The multi-domain binding molecule of any of embodiments 50-56, wherein the multimerizing fragment of the IgM Fc region includes a Cμ3 domain, a Cμ4 domain, and an IgM tailpiece.

58. The multi-domain binding molecule of any of embodiments 50-57, wherein the multimerizing fragment of the IgM Fc region includes a Cμ2 domain, a Cμ3 domain, a Cμ4 domain, and an IgM tailpiece.

59. The multi-domain binding molecule of any of embodiments 50-58, wherein the multimerizing fragment of the IgM Fc region includes a Cμ1 domain, a Cμ2 domain, a Cμ3 domain, a Cμ4 domain, and an IgM tailpiece.

60. The multi-domain binding molecule of any of embodiments 31-59, wherein the multi-domain binding molecule has the sequence as set forth in SEQ ID NOs: 299, 301, 303, 305, 329, 330, 331, or 332, or a sequence having at least 95% sequence identity to the sequence as set forth in SEQ ID NOs: 299, 301, 303, 305, 329, 330, 331, or 332.

61. The multi-domain binding molecule of any of embodiments 31-60, wherein the multi-domain binding molecule is encoded by the sequence as set forth in SEQ ID NOs: 300, 302, 304, or 306, or a sequence having at least 95% sequence identity to the sequence as set forth in SEQ ID NOs: 300, 302, 304, or 306.

62. A conjugate including the antibody or binding fragment thereof of any of embodiments 1-24 linked to a radioactive isotope, an immunotoxin, a drug, a detectable label, or a particle. 63. The conjugate of embodiment 62, wherein the radioactive isotope includes ²²⁸Ac, ¹¹¹Ag, ¹²⁴Am, ⁷⁴As, ²¹¹ At, ²⁰⁹At, ¹⁹⁴Au, ¹²⁸Ba, ⁷Be, ²⁰⁶Bi, ²⁴⁵Bk, ²⁴⁶Bk, ⁷⁶Br, ¹¹C, ¹⁴C, ⁴⁷Ca, ²⁵⁴Cf, ²⁴²Cm, ⁵¹Cr, ⁶⁷CU, ¹⁵³Dy, ¹⁵⁷Dy, ¹⁵⁹Dy, ¹⁶⁵Dy, ¹⁶⁶Dy, ¹⁷¹Er, ²⁵⁰Es, ²⁵⁴Es, ¹⁴⁷Eu, ¹⁵⁷Eu, ⁵²Fe, ⁵⁹Fe, ²⁵¹Fm, ²⁵²Fm, ²⁵³Fm, ⁶⁶Ga, ⁷²Ga, ¹⁴⁶Gd, ¹⁵³Gd, ⁶⁸Ge, ³H, ¹⁷⁰Hf, ¹⁷¹Hf, ¹⁹³Hg, ¹⁹³mHg, ¹⁶⁰mHo, ¹³⁰I, ¹³¹I, ¹³⁵I, ¹¹⁴mIn, ¹⁸⁵Ir, ⁴²K, ⁴³K, ⁷⁶Kr, ⁷⁹Kr, ⁸¹mKr, ¹³²La, ²⁶²Lr, ¹⁶⁹Lu, ¹⁷⁴mLu, ¹⁷⁶mLu, ²⁵⁷Md, ²⁶⁰Md, ²⁸Mg, ⁵²Mn, ⁹⁰Mo, ²⁴Na, ⁹⁵Nb, ¹³⁸Nd, ⁵⁷Ni, ⁶⁶Ni, ²³⁴Np, ¹⁵O, ¹⁸²Os, ¹⁸⁹mOs, ¹⁹¹Os, ³²P, ²⁰¹Pb, ¹⁰¹Pd, ¹⁴³Pr, ¹⁹¹Pt, ²⁴³Pu, ²²⁵Ra, ⁸¹Rb, ¹⁸⁸Re, ¹⁰⁵Rh, ²¹¹ Rn, ¹⁰³Ru, ³⁵S, ⁴⁴Sc, ⁷²Se, ¹⁵³Sm, ¹²⁵Sn, ⁹¹Sr, ¹⁷³Ta, ¹⁵⁴Tb, ¹²⁷Te, ²³⁴Th, ⁴⁵Ti, ¹⁶⁶Tm, ²³⁰U, ²³⁷U, ²⁴⁰U, ⁴⁸V, ¹⁷⁸W, ¹⁸¹W, ¹⁸⁸W, ¹²⁵Xe, ¹²⁷Xe, ¹³³Xe, ¹³³mXe, ¹³⁵Xe, ⁸⁵mY, ⁸⁶Y, ⁹⁰Y, ⁹³Y, ¹⁶⁹Yb, ¹⁷⁵Yb, ⁶⁵Zn, ⁷¹ mZn, ⁸⁶Zr, ⁹⁵Zr, or ⁹⁷Zr.

64. The conjugate of embodiments 62 or 63, wherein the radioactive isotope includes ¹³¹I, ⁹⁰Y, or ²¹¹At.

65. The conjugate of embodiment 62, wherein the immunotoxin includes a plant toxin or bacterial toxin.

66. The conjugate of embodiment 65, wherein the plant toxin includes ricin, abrin, mistletoe lectin, modeccin, pokeweed antiviral protein, saporin, Bryodin 1, bouganin, or gelonin.

67. The conjugate of embodiment 65, wherein the bacterial toxin includes diphtheria toxin or Pseudomonas exotoxin.

68. The conjugate of embodiment 62, wherein the drug includes a cytotoxic drug.

69. The conjugate of embodiment 68, wherein the cytotoxic drug includes actinomycin D, anthracycline, auristatin, calicheamicin, camptothecin, CC1065, colchicin, cytochalasin B, daunorubicin, 1-dehydrotestosterone, dihydroxy anthracinedione, dolastatin, doxorubicin, duocarmycin, elinafide, emetine, ethidium bromide, etoposide, gramicidin D, glucocorticoids, lidocaine, maytansinoid, mithramycin, mitomycin, mitoxantrone, nemorubicin, PNU-159682, procaine, propranolol, puromycin, pyrrolobenzodiazepine, taxane, taxol, tenoposide, tetracaine, trichothecene, vinblastine, vinca alkaloid, or vincristine.

70. The conjugate of embodiment 62, wherein the detectable label includes a fluorescent label, a chemiluminescent label, a spectral colorimetric label, an enzymatic label, or an affinity tag.

71. The conjugate of embodiment 70, wherein the fluorescent label includes blue fluorescent protein, cyan fluorescent protein, green fluorescent protein, luciferase, orange fluorescent protein, red fluorescent protein, far red fluorescent protein, or yellow fluorescent protein.

72. The conjugate of embodiment 70, wherein the chemiluminescent label includes lucigenin, luminol, luciferin, isoluminol, theromatic acridinium ester, imidazole, acridinium salt, or oxalate ester.

73. The conjugate of embodiment 70, wherein the spectral colorimetric label includes colloidal gold.

74. The conjugate of embodiment 70, wherein the enzymatic label includes malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase, or acetylcholinesterase.

75. The conjugate of embodiment 70, wherein the affinity tag includes the sequence as set forth in any of SEQ ID NOs: 401-413.

76. A chimeric antigen receptor (CAR) that, when expressed by a cell, includes an extracellular component linked to an intracellular component by a transmembrane domain, wherein the extracellular component includes an antibody or binding fragment thereof of any of embodiments 1-24.

77. The CAR of embodiment 76, wherein the antibody or binding fragment thereof includes a single chain variable fragment (scFv) including a variable light chain connected to a variable heavy chain by a flexible linker.

78. The CAR of embodiment 77, wherein the flexible linker includes a Gly-Ser linker.

79. The CAR of embodiment 78, wherein the Gly-Ser linker includes the sequence as set forth in any of SEQ ID NO: 347-360.

80. The CAR of any of embodiments 77-79, wherein the flexible linker includes a Whitlow linker.

81. The CAR of any of embodiments 77-80, wherein the scFv has the sequence as set forth in SEQ ID NOs: 421, 423, 425, 427, 429, 491, 492, 493, 494, or 495 or a sequence with at least 90% sequence identity to a sequence as set forth in SEQ ID NOs: 421, 423, 425, 427, 429, 491, 492, 493, 494, or 495.

82. The CAR of any of embodiments 77-81, wherein the scFv is encoded by the sequence set forth in SEQ ID NOs: 422, 424, 426, 428, or 430, or a sequence having at least 90% sequence identity to SEQ ID NOs: 422, 424, 426, 428, or 430.

83. The CAR of any of embodiments 77-82, wherein the intracellular component includes an effector domain including: 4-1BB (CD137), CD3γ, CD3δ, CD3ε, CD3ζ, CD27, CD28, DAP10, ICOS, LAG3, NKG2D, NOTCH1, OX40, ROR2, SLAMF1, TCRα, TCRβ, TRIM, Wnt, Zap70, or a combination thereof.

84. The CAR of any of embodiments 76-83, wherein the intracellular component includes an effector domain including a 4-1 BB intracellular signaling domain and a CD3ζ intracellular signaling domain.

85. The CAR of embodiments 83 or 84, wherein the effector domain has a sequence as set forth in SEQ ID NO: 489 or a sequence having at least 90% sequence identity to SEQ ID NO: 489.

86. The CAR of any of embodiments 83-85, wherein the effector domain is encoded by a sequence as set forth in SEQ ID NO: 490 or a sequence having at least 90% sequence identity to SEQ ID NO: 490.

87. The CAR of any of embodiments 76-86, wherein the transmembrane domain includes a transmembrane region of: the α, β or ζ chain of a T-cell receptor; CD28; CD27; CD3; CD45; CD4; CD5; CD8; CD9; CD16; CD22; CD33; CD37; CD64; CD80; CD86; CD134; CD137; CD154; or a combination thereof.

88. The CAR of any of embodiments 76-87, wherein the transmembrane domain includes a CD28 transmembrane domain.

89. The CAR of embodiment 88, wherein the CD28 transmembrane domain has a sequence as set forth in SEQ ID NO: 487 or a sequence having at least 90% sequence identity to SEQ ID NO: 487.

90. The CAR of embodiments 88 or 89, wherein the CD28 transmembrane domain is encoded by a sequence as set forth in SEQ ID NO: 488 or a sequence having at least 90% sequence identity to SEQ ID NO: 488.

91. The CAR of any of embodiments 76-90, wherein the CAR further includes a spacer region.

92. The CAR of embodiment 91, wherein the spacer region includes a 60 aa hinge domain spacer region.

93. The CAR of embodiment 92, wherein the 60 aa hinge domain spacer region has a sequence as set forth in SEQ ID NO: 485 or a sequence having at least 90% sequence identity to SEQ ID NO: 485.

94. The CAR of embodiment 93, wherein the 60 aa hinge domain spacer region is encoded by a sequence as set forth in SEQ ID NO: 486 or a sequence having at least 90% sequence identity to SEQ ID NO: 486.

95. The CAR of any of embodiments 76-94, wherein the transmembrane domain includes a CD28 transmembrane domain, and the intracellular component includes a CD3ζ intracellular signaling domain and a 4-1 BB intracellular signaling domain.

96. The CAR of embodiment 95, wherein the CAR further includes a 60 aa hinge domain spacer region.

97. The CAR of embodiment 96, wherein the CAR further includes a self-cleaving peptide and a reporter.

98. An engineered T cell receptor (eTCR) including a constant alpha domain (Cα), a constant beta domain (Cβ), and the scFv of any of embodiments 25-30 linked to the Ca domain and/or the Cβ domain.

99. The eTCR of embodiment 98, wherein the scFv is linked to the Cα domain.

100. The eTCR of embodiments 98 or 99, wherein the scFv is linked to the Cβ domain.

101. The eTCR of any of embodiments 98-100, wherein one scFv is linked to the Cα domain and one scFv of any of embodiments 25-30 is linked to the Cβ domain.

102. A multi-domain binding molecule including a CD3-binding single chain variable fragment (scFv) linked to an antibody or binding fragment thereof of any of embodiments 1-24.

103. The multi-domain binding molecule of embodiment 102, wherein the CD3-binding scFv has the sequence as set forth in SEQ ID NO: 496 or a sequence having at least 90% sequence identity to SEQ ID NO: 496.

104. The multi-domain binding molecule of embodiments 102 or 103, including the sequence as set forth in any one of SEQ ID NOs: 307, 309, 311, 313, 333, 334, 335, or 336, or a sequence having at least 95% sequence identity to the sequence as set forth in SEQ ID NO: 307, 309, 311, 313, 333, 334, 335, or 336.

105. The multi-domain binding molecule of any of embodiments 102-104, wherein the multi-domain binding molecule is encoded by the sequence as set forth in any of SEQ ID NOs: 308, 310, 312, or 314, or a sequence having at least 95% sequence identity to the sequence as set forth in any of SEQ ID NOs: 308, 310, 312, or 314.

106. A nucleic acid encoding an antibody or binding fragment thereof of any of embodiments 1-24, the scFv of any of embodiments 25-30, the multi-domain binding molecule of any of embodiments 31-61 or 102-105, the CAR of any of embodiments 76-97, or the eTCR of any of embodiments 98-101.

107. The nucleic acid of embodiment 106, wherein the nucleic acid includes the sequence as set forth in SEQ ID NOs: 272, 274, 276, 278, 280, 282, 284, 286, 288, 290, 292, 294, 297, 300, 302, 304, 306, 308, 310, 312, 314, 422, 424, 426, 428, 430, 432, 435, 438, 441, 444, 447, 450, 453, 456, 459, 462, 465, 468, 471, 474, 477, 480, 483, 486, 488, or 490, or a sequence having at least 95% sequence identity to the sequence as set forth in SEQ ID NOs: 272, 274, 276, 278, 280, 282, 284, 286, 288, 290, 292, 294, 296, 297, 302, 304, 306, 308, 310, 312, 314, 422, 424, 426, 428, 430, 432, 435, 438, 441, 444, 447, 450, 453, 456, 459, 462, 465, 468, 471, 474, 477, 480, 483, 486, 488, or 490.

108. A cell genetically modified to express the antibody or binding fragment thereof of any of embodiments 1-24, the scFv of any of embodiments 25-30, the multi-domain binding molecule of any of embodiments 31-61 or 102-105, the CAR of any of embodiments 76-97, or the eTCR of any of embodiments 98-101.

109. The cell of embodiment 108, wherein the cell is an immune cell.

110. The cell of embodiment 109, wherein the immune cell is a T cell, B cell, natural killer cell, or macrophage.

111. The cell of embodiments 109 or 110, wherein the immune cell is a natural killer cell.

112. The cell of embodiments 109 or 110, wherein the immune cell is a T cell.

113. A composition including the antibody or binding fragment thereof of any of embodiments 1-24, the scFv of any of embodiments 25-30, the multi-domain binding molecule of any of embodiments 31-61 or 102-105, the conjugate of any of embodiments 62-75, or the nucleic acid of embodiment 106; and a pharmaceutically acceptable carrier.

114. A formulation including the cell of any of embodiments 108-112 and a pharmaceutically acceptable carrier.

115. A kit including the antibody or binding fragment thereof of any of embodiments 1-24, the scFv of any of embodiments 25-30, the multi-domain binding molecule of any of embodiments 31-61 or 102-105, the CAR of any of embodiments 76-97, the eTCR of any of embodiments 98-101, the conjugate of any of embodiments 62-75, the nucleic acid of embodiment 106, the cell of any of embodiments 108-112, the composition of embodiment 113, or the formulation of embodiment 114.

116. A method of treating a subject in need thereof including administering a therapeutically effective amount of the composition of embodiment 113 and/or the formulation of embodiment 114 thereby treating the subject in need thereof.

117. The method of embodiment 116, wherein the therapeutically effective amount provides a prophylactic or a therapeutic treatment against a mast-cell or eosinophil-related disorder.

118. The method of embodiment 117, wherein the mast-cell or eosinophil-related disorder includes asthma, rhinitis, dermatitis, an eosinophilic gastrointestinal disorder, eosinophilic pneumonia, anaphylaxis, urticaria, eosinophilic cellulitis and fasciitis, Churg-Strauss syndrome, a hematologic malignancy involving eosinophils or mast cells, or mastocytosis.

119. The method of embodiment 118, wherein the rhinitis includes chronic rhinitis.

120. The method of embodiments 118 or 119, wherein the rhinitis includes allergic rhinitis.

121. The method of any of embodiments 118-120, wherein the dermatitis is atopic dermatitis.

122. The method of any of embodiments 118-121, wherein the eosinophilic gastrointestinal disorder includes eosinophilic esophagitis, gastritis, or colitis.

123. The method of any of embodiments 118-122, wherein the urticaria includes chronic idiopathic urticaria.

124. The method of any of embodiments 118-123, wherein the hematologic malignancy involving eosinophils or mast cells includes eosinophilic leukemia.

125. The method of any of embodiments 116-124, wherein the administering is through intravenous, intradermal, intraarterial, intranodal, intravesicular, intrathecal, intraperitoneal, intraparenteral, intranasal, intralesional, intramuscular, oral, intrapulmonary, subcutaneous, or sublingual administering.

### (xi) Experimental Example. Siglec-8-Directed Immunotherapy for Eosinophilic and Mast Cell Disorders

Abstract. Current treatment strategies for diseases in which eosinophils and/or mast cells play an important pathogenic role are often insufficiently effective. One promising target to improve outcomes in these disorders is sialic acid immunoglobulin (Ig)-like lectin 8 (Siglec-8). However, there are few monoclonal antibodies (mAbs) against Siglec-8 available, and, so far, Siglec-8-directed treatments have largely focused on unconjugated mAbs, which may have inadequate efficacy. Here, transgenic mice were used to raise a diverse panel of fully human mAbs that either recognize the V-set domain, membrane-distal C2-set domain, or membrane-proximal C2-set domain of full-length Siglec-8 as a basis for novel therapeutics. These mAbs were efficiently internalized into Siglec-8+ cells, demonstrating their ability to deliver cytotoxic payloads. T cell-engaging bispecific antibodies (BiAbs) and chimeric antigen receptor (CAR)-modified natural killer (NK) cells using single chain variable fragments (scFv) from Siglec-8 mAbs showed highly potent cytolytic activity against Siglec-8 positive cells even in cases of very low target antigen expression, whereas they elicited no cytolytic activity against Siglec-8 negative cells. Siglec-8^{V-set}-directed T cell-engaging BiAbs and Siglec-8^{V-set}-directed CAR-modified NK cells induced substantially greater cytotoxicity against cells expressing an artificial smaller Siglec-8 variant containing only the V-set domain than cells expressing full-length Siglec-8, consistent with the notion that targeting membrane-proximal epitopes enhances effector functions of Siglec-8 antibody-based therapeutics. Indeed, Siglec-8^{C2-set}-directed T cell-engaging BiAbs and Siglec-8^{C2-set}-directed CAR-modified NK cells showed high antigen-specific cytolytic activity. Together, these data demonstrate Siglec-8-directed immunotherapies as highly potent, and useful in treating patients with eosinophilic and mast cell disorders.

Introduction. In a wide variety of human diseases, eosinophils and/or mast cells play an important pathogenetic role (Kiwamoto 2012; Kuang & Bochner, Semin Immunopathol. 2021, 43(3):459-475 (“Kuang & Bochner 2021”); Pitlick et al., World Allergy Organ J. 2022, 15(8):100676 (“Pitlick 2022”); Kolkhir et al., Nat Rev Immunol. 2022, 22(5):294-308 (“Kolkhir 2022”); Kaufmann & Simon, Annu Rev Pharmacol Toxicol. 2023, 63:231-247 (“Kaufmann & Simon 2023”)). Current strategies to treat eosinophil-related and/or mast cell-related disorders include targeting eosinophils with monoclonal antibodies (mAbs) blocking interleukin-5 (e.g. mepolizumab or reslizumab) or the interleukin-5 receptor (e.g. benralizumab) or blocking mast cells with mAbs that bind free IgE (e.g. omalizumab). Oftentimes, however, these and other existing therapeutics are insufficiently effective, and patients suffer severe, life-threatening, or fatal complications. Thus, there is a need for better treatments. One promising target to improve outcomes in these disorders is sialic acid immunoglobulin (Ig)-like lectin 8 (Siglec-8; also known as SAF2) (Kiwamoto 2012; Kuang & Bochner 2021; Pitlick 2022; Kolkhir 2022; Kaufmann & Simon 2023; Youngblood et al., Cells. 2020, 10(1); Dellon & Spergel, Ann Allergy Asthma Immunol. 2023, 130(1):21-27).

In a randomized clinical trial, the humanized non-fucosylated IgG1 Siglec-8 mAb (lirentelimab [AK002]) reduced eosinophil counts and alleviated symptoms in patients with eosinophilic gastritis and duodenitis (Dellon 2020), validating Siglec-8 as a drug target. However, there is a paucity of anti-Siglec-8 mAbs, and how Siglec-8 is best targeted therapeutically was unknown. In vitro findings showing Siglec-8 mAbs inhibit mast cell function but do not induce mast cell apoptosis (Kolkhir 2022; Youngblood et al., Int Arch Allergy Immunol. 2019, 180(2):91-102 (“Youngblood 2019”); Kerr et al., Clin Exp Allergy. 2020, 50(8):904-914 (“Kerr 2020”); Schanin et al., Mucosal Immunol. 2021, 14(2):366-376 (“Schanin 2021”)) indicating that unconjugated mAbs may have insufficient activity in some Siglec-8-positive disorders. This is reminiscent of the experience with Siglec-3 (CD33)—currently the most widely targeted Siglec family member—for which unconjugated mAbs have been largely ineffective in the clinic (Cowan et al. Front Biosci (Landmark Ed). 2013, 18:1311-1334; Laszlo et al. Blood Rev. 2014, 28(4):143-153). More potent treatment modalities, e.g. T cell- or natural killer (NK) cell-directed bispecific antibodies (BiAbs) or chimeric antigen receptor (CAR) modified immune effector cells, may be required for success of therapies targeting Siglec-8. Besides treatment modality (e.g., unconjugated mAb vs. BiAb), data with CD33-targeted therapeutics suggest the efficacy of Siglec-directed immunotherapy is also affected by the location of the protein domain to which it binds. Specifically, membrane-proximal binding enhances effector functions of CD3-directed BiAbs and CAR-modified T cells (Godwin 2021). To improve Siglec-8-directed immunotherapy, transgenic mice were used to raise a series of fully human anti-Siglec-8 mAbs. With these mAbs in hand, the role of membrane-proximal targeting as a way of enhancing the cytotoxicity of Siglec-8-targeted therapeutics was examined. Further, a series of BiAbs engaging T cells and CAR-modified NK cells were generated as novel treatment modalities for Siglec-8-positive disorders.

Materials and Methods. Generation of lentiviral expression vectors encoding human Siglec-8 variants. 6H-Siglec-8^{FL} [full-length Siglec-8] was generated using the endogenous human Siglec-8 signal peptide (amino acids [aa] 1-16), a 6-histidine tag, a glycine-serine-glycine linker, and the human Siglec-8 coding region including extracellular domain (ECD, aa 17-363), transmembrane domain (aa 364-384), and intracellular domain (aa 385-499). In the same way, Siglec-8^{FL} was also generated without the 6-histidine tag. A truncated Siglec-8 protein lacking exon-2 (Siglec-8^{ΔE2}) was generated using the endogenous Siglec-8 signal peptide (aa 1-16), the V-set domain (aa 17-151), aspartic acid at position 152, the site of the exon1/3 junction produced by exon 2-skipping, followed by the C2-set domain and all remaining Siglec-8 amino acids (aa 246-499). Another truncated Siglec-8 protein containing only the V-set domain in the extracellular portion (Siglec-8^{Vset only}) was generated using the endogenous Siglec-8 signal peptide (aa 1-16), the V-set domain (aa 17-152), the juxtamembrane region (aa 352-363) and all remaining Siglec-8 amino acids (aa 364-499). For mouse immunizations, Siglec-8^{FL} and/or chimeric mouse/human proteins including the V-set domain portion of mouse CD33 (aa 1-233) fused with the membrane proximal C2-set domain, transmembrane and intracellular domain of human Siglec-8 (aa 246-499), or the V-set portion of mouse Siglec-F (aa 1-227) fused to the membrane proximal C2-set domain of human Siglec-8 (aa 246-499) were used. cDNA was synthesized as gBlock (Integrated DNA Technologies, Coralville, Iowa) for cloning into pRRLsin.cPPT.MSCV lentivirus constructs containing an IRES-Enhanced Green Fluorescent Protein (EGFP) cassette. All lentiviral constructs were confirmed by Sanger sequencing.

Genetic deletion of Siglec-8. CRISPR/Cas9-editing was carried out by electroporating purified Cas9 protein (TrueCut Cas9 V2; ThermoFisher Scientific, Waltham, MA) complexed with a pool of 3 synthetic guide RNAs (sgRNA) targeting Siglec-8 (sequence 5′-CUGGGGGUAGGAGAAGGAGC-3′ (SEQ ID NO: 414), 5′-CUUGCUGCAAGUGCAGGAGC-3′ (SEQ ID NO: 415), and 5′-CCCCUUUGUCCCCCAGAGCA-3′ (SEQ ID NO: 416)) using the ECM 380 Square Wave Electroporation system (Harvard Apparatus, Cambridge, MA) as described (Humbert et al., Leukemia. 2019, 33(3):762-808; Godwin et al., Leukemia. 2020, 34(9):2479-2483 (“Godwin 2020a”)). Siglec-8-single cells were isolated via fluorescence-activated cell sorting (FACS), and genomic DNA amplified from individual clones using manufacturer's recommended primers (forward primer: GCTCCAAATGTCCCCAAGGA (SEQ ID NO: 417), reverse primer: ACTTTTCTGACTTCACCCCCA (SEQ ID NO: 418), sequencing primer: CTCCAAATGTCCCCAAGGAGTTG (SEQ ID NO: 419)) and analyzed by Sanger sequencing to confirm frame-shift mutation/gene disruption at all Siglec-8 alleles.

Parental and engineered human mast cells, human acute leukemia cells, and NK cell lines. LUVA cells were purchased from Kerafast, Inc. (Boston, MA) and maintained in StemPro™-34 (Thermo Fisher Scientific; Waltham, MA) serum-free medium (SFM) and 2 mM L-glutamine. The human mast cell line HMC-1.2 (Sigma Aldrich, St. Louis, MO) was cultured in Iscove's modified Dulbecco's medium supplemented with 25 mM HEPES, 3.024 g/L sodium bicarbonate, 10% FBS, 1% L-glutamine, and 0.1% β-mercaptoethanol (all from Gibco, Thermo Fisher Scientific). KHYG-1 cells (obtained from the German Collection of Microorganisms and Cell Cultures [DSMZ], Braunschweig, Germany) were cultured in Roswell Park Memorial Institute (RPMI)-1640 medium with 10% fetal bovine serum (FBS, Hyclone, Thermo Fisher Scientific) and 100 U/mL of recombinant IL-2 (Peprotech, Cranbury, NJ). Human myeloid HL-60, K562, ML-1, and TF-1 cells as well as human lymphoid RS4; 11 cells were cultured as previously described (Godwin 2021; Godwin 2020a). Human myeloid EOL-1 cells were cultured in RPMI-1640 medium with 10% FBS. Lentivirally-transduced sublines of various cell lines were generated at multiplicities of infection (MOI) of 1-25; EGFP-positive cells were isolated by FACS and re-cultured for further analysis/use. All cell lines were grown with 1% penicillin-streptomycin, routinely tested for mycoplasma contamination (MycoAlert Mycoplasma Detection Kit, Lonza, Basel, Switzerland), and authenticated using standard short tandem repeat (STR) combined DNA index system (CODIS) typing.

Primary human NK cells. Primary human NK cells were isolated from non-mobilized healthy donor leukapheresis product by CD56-positive selection using cliniMACS CD56 microbeads (Militenyi Biotec, Bergisch Gladbach, Germany) and then rested overnight before freezing down. Frozen purified NK cells were thawed 1 day before co-culture experiments in the ADCC assays. Primary human NK cells were cultured in RPMI-1640 medium with 10% heat-inactivated pooled human serum (Bloodworks Northwest, Seattle, WA), 3.5 mM glutamine (ThermoFisher Scientific), 44 μM β-mercaptoethanol, 100 μg/mL penicillin/streptomycin (ThermoFisher Scientific), and supplemented with 500 U/mL of recombinant IL-2 (Peprotech) when rested overnight at 37° C. in 5% CO₂.

Primary human eosinophils. Peripheral blood specimens from two patients with elevated eosinophil levels were obtained under protocols approved by the Fred Hutchinson Cancer Center Institutional Review Board. All patients provided written informed consent for sample collection and use for research. Eosinophils were isolated from peripheral blood using the Eosinophil Isolation Kit (Miltenyi Biotec) following manufacturer's instructions and the purity of enriched eosinophils (CCR3+, Siglec-8+) evaluated by flow cytometry was >90% post-isolation.

Generation of human Siglec-8 mAbs. Alloy ATX-GK BL/6, ATX-GK mix, and ATX-GL mice (Alloy Therapeutics, Waltham, MA) were subjected to 4 rounds of injections with combinations of mouse 3T3 cells expressing different Siglec-8 immunogens. Hybridoma screening was done flow cytometrically using K562 sublines overexpressing either Siglec-8^{FL} or chimeric mouse/human versions of Siglec-8 expressing only the juxtamembrane portion of the ECD of Siglec-8. Hybridomas with reactivity against Siglec-8^{FL} and/or reactivity against the juxtamembrane portion of the ECD of Siglec-8 were subcloned, and antibodies sequenced as described (Godwin 2020a).

Expression and purification of recombinant human Siglec-8 mAbs and negative control mAb. 5′ rapid amplification of cDNA ends (RACE) cloning of antibody sequences was performed as per company instructions (Alloy Therapeutics). Protein sequences were reverse-translated using human codons for cloning, cDNA was synthesized as gBlock (Integrated DNA Technologies, Coralville, Iowa) for cloning into pcDNA3.4 vectors, sequence verified by Sanger sequencing, and heavy chain and light chain pairs transiently transfected into Expi293 cells per manufacturer's instructions (Thermo Fisher Scientific, Waltham, MA). Secreted antibodies were then purified from conditioned media using protein A affinity chromatography on an AKTA pure system with MAbSelectSuRe column (Cytiva, Marlborough, MA), quantified via NanoDrop microvolume spectrophotometer (Thermo Fisher Scientific), and analyzed by SDS-PAGE. A negative control mAb, 13R4 (recognizing E. coli beta-galactosidase), was generated and purified in a similar fashion using published sequences (SEQ ID NO: 33 in European patent EP2134841B1) as shown in the following sequence: MAEVQLVESGGSLVKPGGSLRLSCAASGFTFSNYSMNWVRQAPGKGLEWISSISGSSRYIYYA DFVKGRFTISRDNATNSLYLQMNSLRAEDTAVYYCVRSSITIFGGGMDVWGRGTLVTVSSGGG GSGGGGSGGGGSQSVLTQPASVSGSPGQSITISCAGTSSDVGGYNYVSWYQQHPGKAPKLM IYEDSKRPSGVSNRFSGSKSGNTASLTISGLQAEDEADYYCSSYTTRSTRVFGGGTKLAVLGA AAEQKLISEEDLNGAAHHHHHH(SEQ ID NO: 328). Commercially-produced lirentelimab was obtained from MedChemExpress (Monmouth Junction, NJ).

Quantification of Siglec-8 expression. Expression of Siglec-8 was quantified by flow cytometry using a phycoerythrin (PE) directly labeled Siglec-8 antibody (clone 7C9; BD Biosciences; Franklin Lakes, NJ). QuantiBRITE PE beads were used to standardize quantitation of number of antibody molecules bound per cell as per manufacturer's instructions (BD Biosciences). To identify non-viable cells, samples were stained with 4′,6-diamidino-2-phenylindole (DAPI). 10,000 events were acquired on a flow cytometer, and DAPI-negative cells analyzed using FlowJo version 10 (BD Biosciences).

Construction, expression, and purification of Siglec-8/CD3- and CD19/CD3- directed BiAbs. T cell-engaging Siglec-8-directed (i.e., Siglec-8/CD3) BiAbs were constructed in the single chain variable fragment (scFv)-scFv format using published sequences (SEQ ID NO: 227 in US 2016/0317657A1; Godwin et al., Br J Haematol. 2020, 189(1):e9-e13 (“Godwin 2020b”)) where the CD33 binding scFv portion was replaced with Siglec-8 binding scFvs as shown in the following sequence:

(SEQ ID NO: 326)
QVQLVQSGAEVKKPGESVKVSCKASGYTFTNYGMNWVKQAPGQGLEWMGW
INTYTGEPTYADKFQGRVTMTTDTSTSTAYMEIRNLGGDDTAVYYCARWS
WSDGYYVYFDYWGQGTSVTVSSGGGGSGGGGSGGGGSDIVMTQSPDSLTV
SLGERTTINCKSSQSVLDSSTNKNSLAWYQQKPGQPPKLLLSWASTRESG
IPDRFSGSGSGTDFTLTIDSPQPEDSATYYCQQSAHFPITFGQGTRLEIK
SGGGGSEVQLVESGGGLVQPGGSLKLSCAASGFTFNKYAMNWVRQAPGKG
LEWVARIRSKYNNYATYYADSVKDRFTISRDDSKNTAYLQMNNLKTEDTA
VYYCVRHGNFGNSYISYWAYWGQGTLVTVSSGGGGSGGGGSGGGGSQTVV
TQEPSLTVSPGGTVTLTCGSSTGAVTSGNYPNWVQQKPGQAPRGLIGGTK
FLAPGTPARFSGSLLGGKAALTLSGVQPEDEAEYYCVLWYSNRWVFGGGT
KLTVLHHHHHH.

In particular embodiments, a CD19/CD3 BiAb was generated in the scFv-scFv (SEQ ID NO: 2 in U.S. Pat. No. 8,007,796 B2; Godwin 2020b) format including the sequence:

(SEQ ID NO: 327)
MGWSCIILFLVATATGVHSDIQLTQSPASLAVSLGQRATISCKASQSVDY
DGDSYLNWYQQIPGQPPKLLIYDASNLVSGIPPRFSGSGSGTDFTLNIHP
VEKVDAATYHCQQSTEDPWTFGGGTKLEIKGGGGSGGGGSGGGGSQVQLQ
QSGAELVRPGSSVKISCKASGYAFSSYWMNWVKQRPGQGLEWIGQIWPGD
GDTNYNGKFKGKATLTADESSSTAYMQLSSLASEDSAVYFCARRETTTVG
RYYYAMDYWGQGTTVTVSSGGGGSDIKLQQSGAELARPGASVKMSCKTSG
YTFTRYTMHWVKQRPGQGLEWIGYINPSRGYTNYNQKFKDKATLTTDKSS
STAYMQLSSLTSEDSAVYYCARYYDDHYCLDYWGQGTTLTVSSVEGGSGG
SGGSGGSGGVDDIQLTQSPAIMSASPGEKVTMTCRASSSVSYMNWYQQKS
GTSPKRWIYDTSKVASGVPYRFSGSGSGTSYSLTISSMEAEDAATYYCQQ
WSSNPLTFGAGTKLELKHHHHHH.

Protein sequences were reverse translated using human codons, cDNA synthesized as gBlock for cloning into pcDNA3.4 vectors, and sequences verified by Sanger sequencing. Proteins were expressed transiently as described above and purified using nickel affinity chromatography on an AKTA pure system with His Trap column (Cytiva).

Quantification of Siglec-8 internalization. To quantify Siglec-8 internalization, cells were stained with 2 μg/mL unlabeled Siglec-8 antibody in ice water for 20 min. After washing in phosphate-buffered saline (PBS), cells were split into aliquots and either left in ice water or incubated at 37° C. for various periods of time. Samples were then stained with PE-conjugated—goat anti-human IgG Fc secondary antibody (SouthernBiotech) to identify remaining antibody on the cell surface, and fluorescence quantified by flow cytometry as described above. Briefly, 10,000 events were acquired on a flow cytometer, and DAPI-negative cells analyzed using FlowJo version 10 (BD Biosciences).

CAR NK cell generation. CAR-modified KHYG-1 cells were generated through lentiviral transduction. Briefly, Siglec-8 scFvs were generated as gBlocks, cloned into the lentiviral vector pSLCAR-CD19-BBz (plasmid #135992, Addgene.org), to generate pSLCAR-Siglec-8-BBz constructs, and sequence verified by Sanger sequencing. pSLCAR encodes a scFv linked to a 60 aa hinge domain spacer, CD28 transmembrane domain, CD3zeta and 4-1 BB intracellular signaling domain and a P2A-EGFP reporter transduction marker. Lentivirally transduced cell sublines were identified and isolated as described above.

Quantification of Antibody-Dependent Cellular Cytotoxicity (ADCC), BiAb- and CAR NK cell-induced cytotoxicity. Cytotoxicity induced by BiAbs was determined flow cytometrically as described, using healthy donor T cells enriched from unstimulated peripheral blood mononuclear cells collected from healthy adult volunteers (Laszlo et al., Front Biosci (Landmark Ed). 2013, 18:1311-1334; Godwin 2020b; Laszlo et al., Blood. 2014, 123(4):554-561; Harrington et al., PLoS One. 2015, 10(8):e0135945 (“Harrington 2015”); Laszlo et al., Blood Cancer. 2015, 5:e340; Reusch et al., Clin Cancer Res. 2016, 22(23):5829-5838; Correnti et al., Leukemia. 2018, 32(5):1239-1243; Klupsch et al., Leukemia. 2019, 33(3):805-808; Laszlo et al., Haematologica. 2019, 104(2):e59-e62 (“Laszlo 2019”)). ADCC was determined flow cytometrically using healthy donor NK cells enriched from unstimulated peripheral blood mononuclear cells collected from healthy adult volunteers. Either the effector cells or target cells were first labeled with CellVue Burgundy Labeling Kit (ThermoFisher Scientific) before co-culturing to differentiate effector cells from target cells. For cell line experiments, cytotoxicity was quantified as a change in the percentage of dead cells as measured by DAPI staining, or in some cases as drug-specific cytotoxicity was quantified as previously described (Harrington 2015; and Laszlo 2019). For experiments involving purified primary human eosinophils, unlabeled primary human eosinophils were co-cultured with healthy donor NK cells or T cells or CAR-NK cells with or without increasing concentrations of the respective antibodies or BiAbs for 16-18 hours before staining with AF6470-labeled anti-CCR3 (Clone 5E8; BioLegend), PE-labeled anti-Siglec-8 (Clone 7C9; BioLegend, San Diego, CA) and APC-Cy7 labeled anti-CD16 (Clone 3G8; BD Biosciences), followed by DAPI. Cytotoxicity was quantified as a change in the number of viable human eosinophils (i.e., % Cytotoxicity=100×(1-live eosinophils treated/live eosinophils control)).

Statistical analysis. Comparisons of Siglec-8 expression levels and drug-induced cytotoxicity were performed with Prism versions 8 thru 10 (GraphPad Software, San Diego, CA) using repeat measure two-way ANOVA with multiple comparison testing.

Results. Production and determination of binding characteristics of new anti-human Siglec-8 mAbs. To raise new human mAbs as a basis for Siglec-8-directed immunotherapies, transgenic mice were immunized with mouse 3T3 cells expressing human Siglec-8^{FL} or a chimeric mouse/human protein including either the V-set domain of mouse CD33 fused with the membrane-proximal C2-set domain of human Siglec-8 or the V-set domain of mouse Siglec-F fused to the membrane-proximal C2-set domain of human Siglec-8 (FIG. 2). Hybridomas of interest were selected based on flow cytometric studies showing binding to K562 cells overexpressing either human Siglec-8^{FL} or truncated Siglec-8 proteins, and variable domain regions of underlying mAbs sequenced. In this experimental example, eleven unique mAbs recognizing human Siglec-8 and/or variants thereof were identified and produced in recombinant fashion with human IgG1 and IgG4 (S228P) frameworks for further characterization.

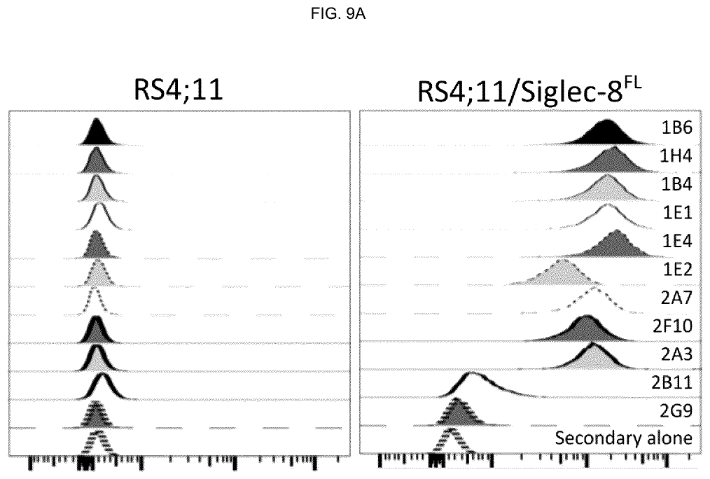

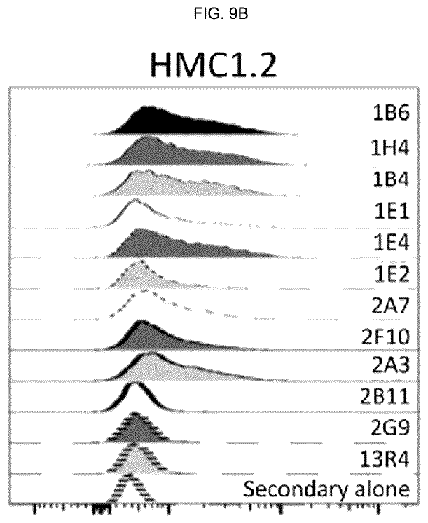

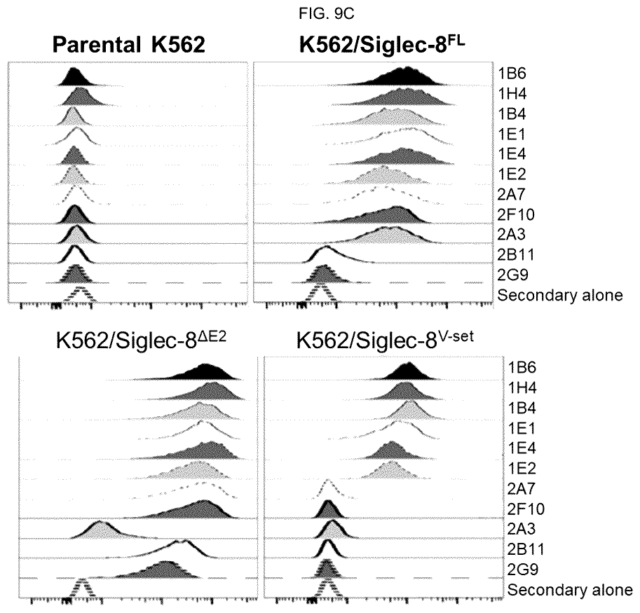

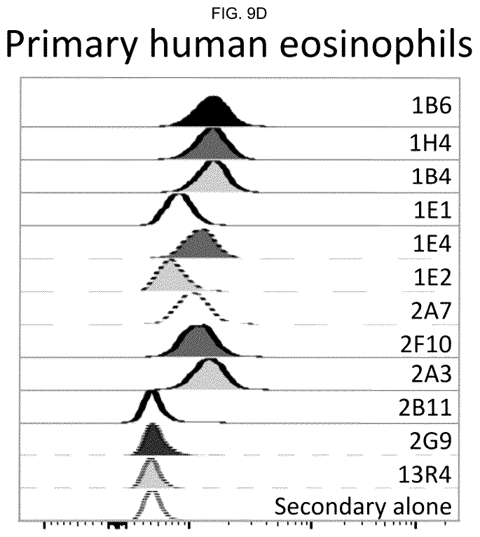

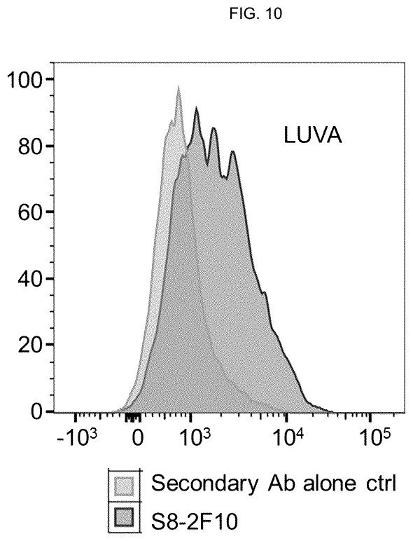

Nine of the anti-Siglec-8 mAbs recognized Siglec-8^{FL} expressed on genetically engineered human lymphoid RS4; 11 cells, and one additional mAb showed minimal binding to Siglec-8^{FL}(FIG. 9A). The same 9 mAbs recognized Siglec-8 endogenously expressed on a subset of HMC1.2 cells (FIG. 9B) and on primary human eosinophils isolated from patients with elevated eosinophil levels (FIG. 9D). Based on binding patterns to Siglec-8^{FL} and truncated variants of Siglec-8 containing the V-set domain and the membrane-proximal C2-set domain or only the V-set domain in the extracellular portion of the protein expressed on K562 cells (for cartoons of Siglec-8 proteins, see FIG. 1), 6 of these 11 mAbs (clones 1B6, 1H4, 1B4, 1E1, 1E4, and 1E2) recognized Siglec-8^{FL} via binding to the V-set domain, 1 mAb (clone 2A3) recognized Siglec-8^{FL} via binding to the membrane-distal C2-set domain, and 2 mAbs (clone 2A7 and 2F10) recognized Siglec-8^{FL} via binding to the membrane-proximal C2-set domain. Finally, 2 mAbs (clones 2B11 and 2G9) recognized a truncated Siglec-8 protein including the V-set domain and the membrane-proximal C2-set domain, indicating that they might bind an artificial epitope generated by the protein truncation (FIG. 9C).

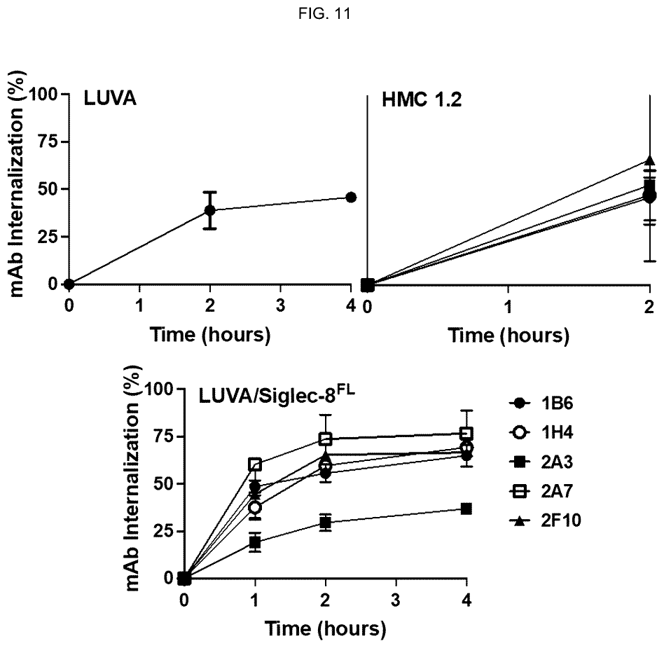

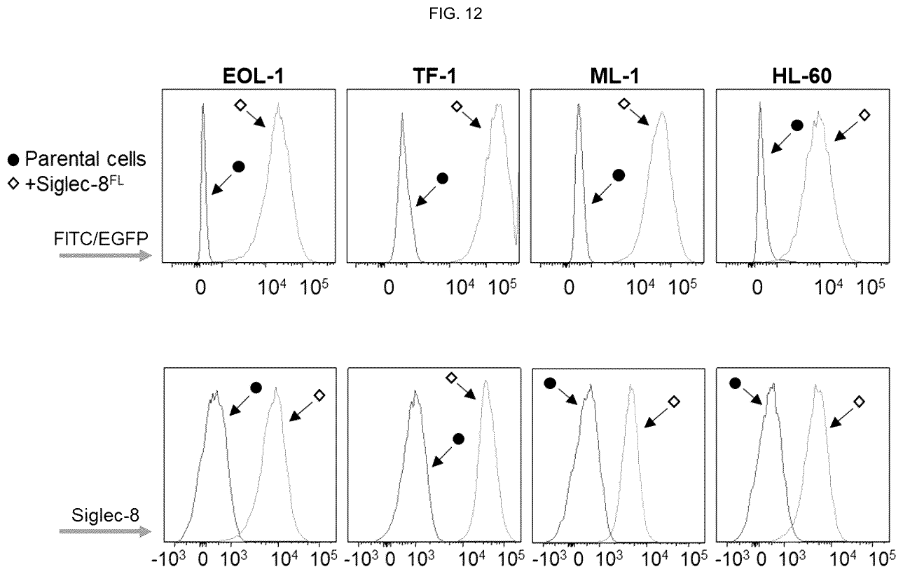

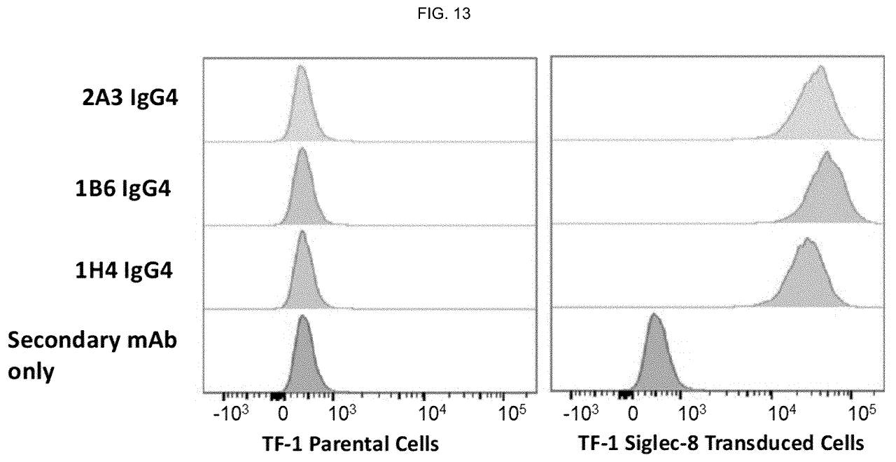

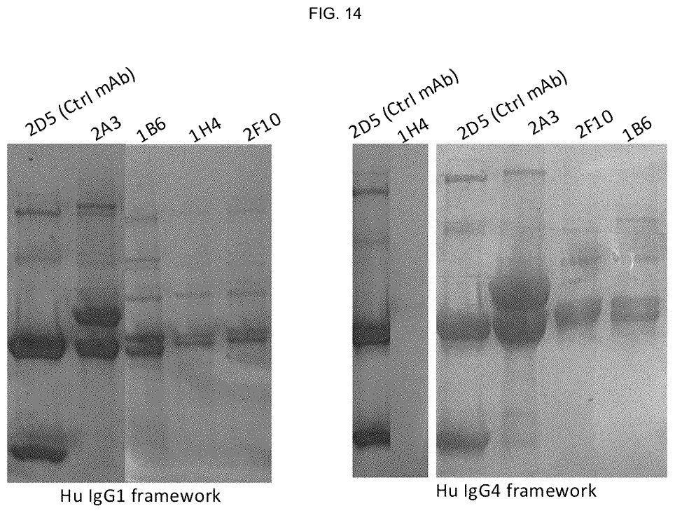

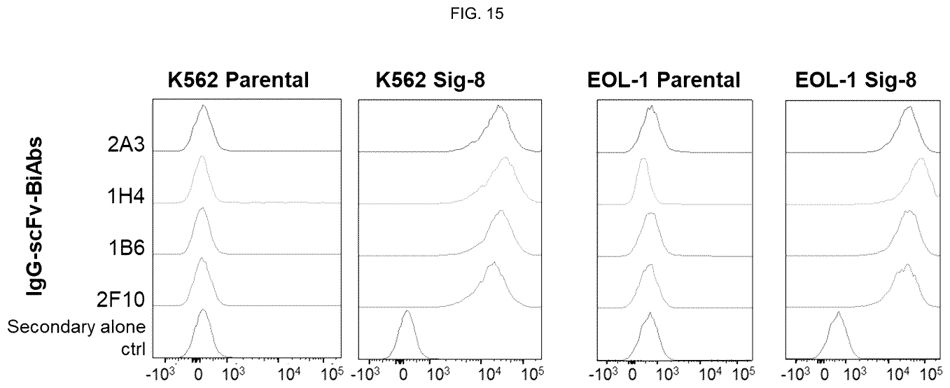

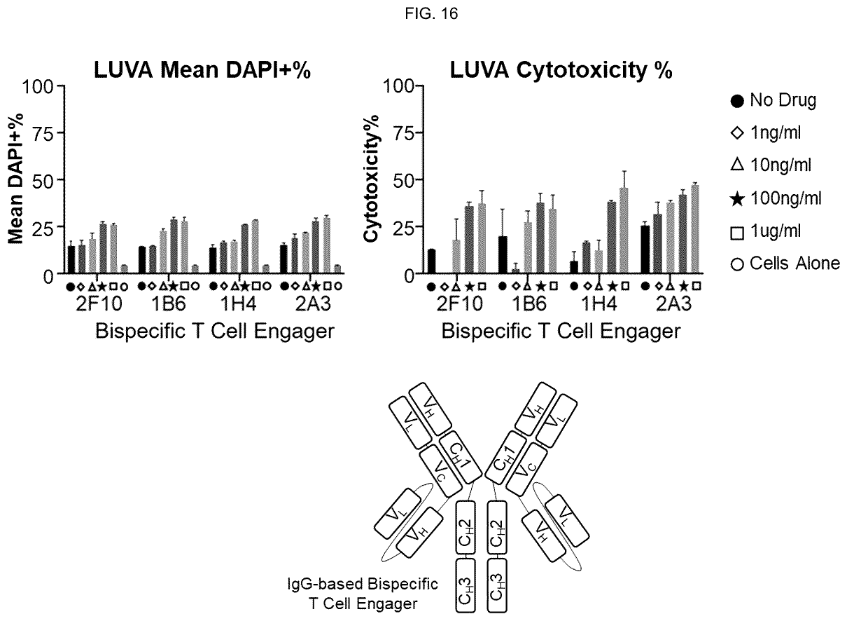

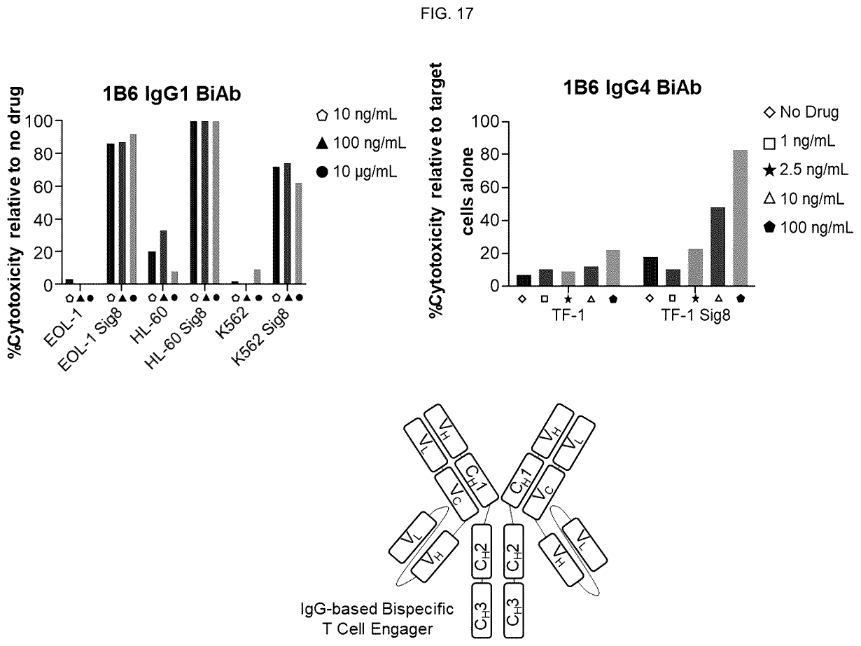

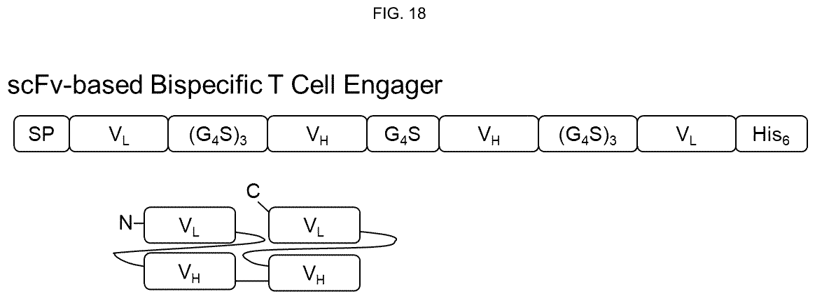

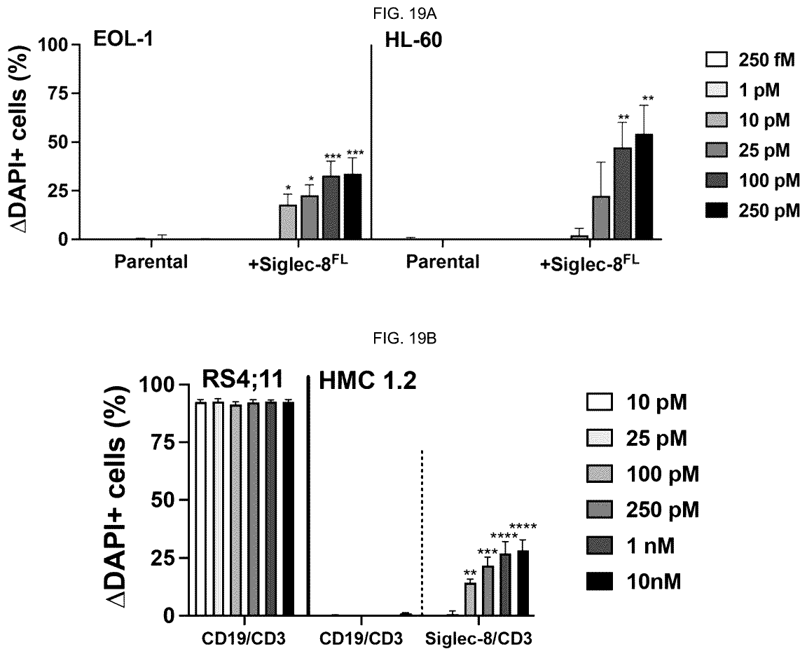

Internalization of Siglec-8 mAbs. Previous studies have indicated that Siglec-8 has endocytic properties (O'Sullivan et al., J Allergy Clin Immunol. 2018, 141(5):1774-1785 e1777 (“O'Sullivan 2018”)). To characterize the human Siglec-8 mAbs as carriers for cytotoxic payloads, flow cytometry-based internalization assays were performed. For these experiments, parental LUVA cells, which endogenously express very low levels of Siglec-8, a subline of LUVA cells in which Siglec-8^{FL} was overexpressed, and, as a negative control, a subline of LUVA cells in which the Siglec-8 gene was deleted by CRISPR/Cas9 were used. Additionally, internalization of Siglec-8 mAbs in HMC 1.2 cells was assessed. Since Siglec-8 was only expressed in a subset of HMC 1.2 cells, FACS was used to enrich HMC 1.2 cells for Siglec-8 expression for the internalization assays (FIG. 9B). All Siglec-8 mAbs tested were internalized time dependently by parental and Siglec-8 overexpressing LUVA cells as well as HMC1.2 cells (FIG. 11). In contrast, no measurable internalization of Siglec-8 mAbs was seen in Siglec-8 gene-edited LUVA cells (not shown), demonstrating that the mAb uptake seen in LUVA cells was dependent on Siglec-8 expression.

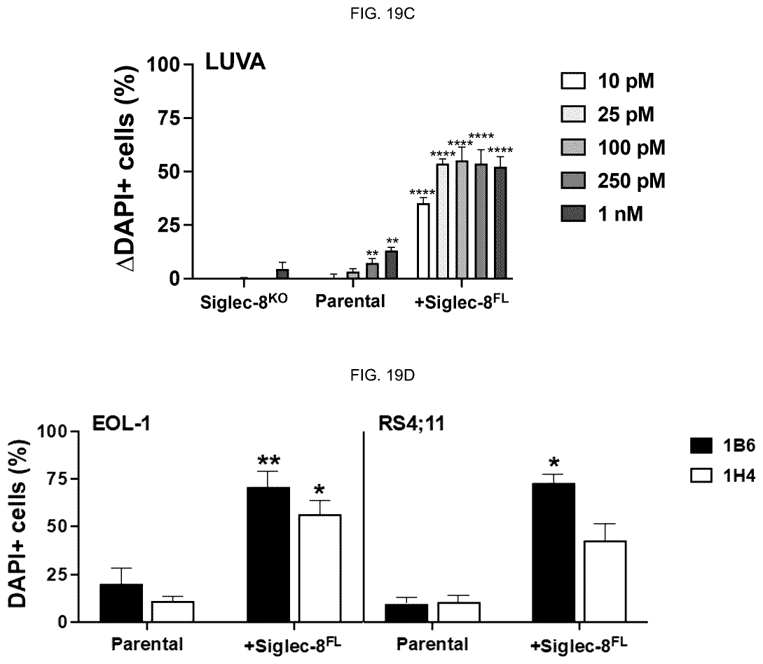

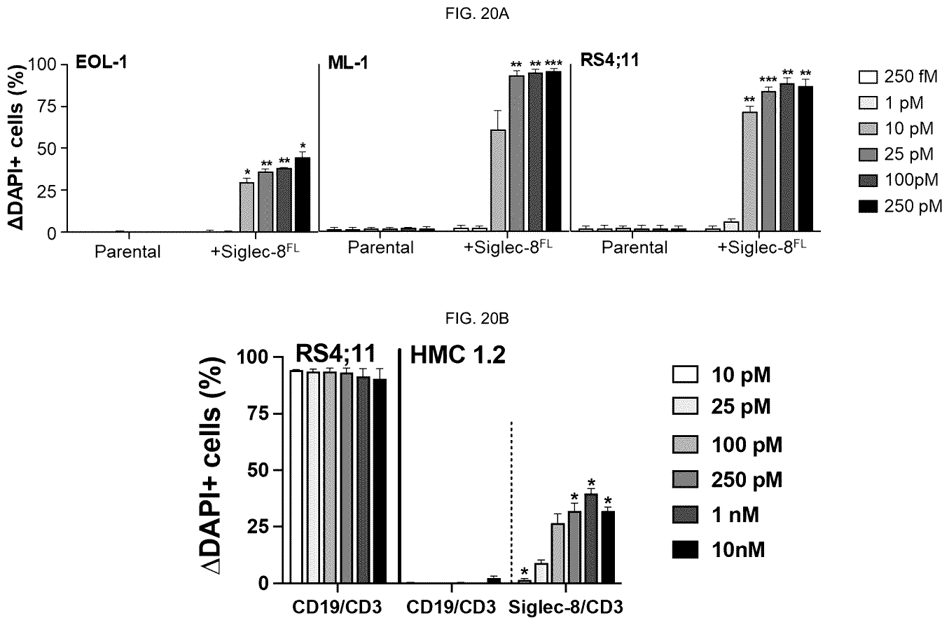

Efficacy of Siglec-8-directed immunotherapies engaging T or NK cells. So far, Siglec-8-directed immunotherapy has been largely pursued with unconjugated (“naked”) Siglec-8 mAbs. Assessment of Siglec-8-directed immunotherapies engaging T or NK cells was performed. For this purpose, T cell-engaging Siglec-8/CD3 BiAbs in the scFv-scFv (Bispecific T cell-Engager, “BiTE”) format as well as Siglec-8 CAR NK cells were generated, using binding sequences from Siglec-8^{V-set} mAbs. As shown in FIGS. 19A-19D, very low concentrations of a Siglec-8/CD3 BiAb built on the binding sequences of 1B6 elicited dose-dependent cytolytic activity against EOL-1 and HL-60 as well as LUVA cells transduced with human Siglec-8^{FL}. This BiAb also exerted Siglec-8-specific cytotoxicity against HMC 1.2 cells that were enriched for Siglec-8 expression as well as parental LUVA cells, which both express very minimal numbers of Siglec-8 molecules on the cell surface. Like Siglec-8/CD3 BiAbs, KHYG-1 cells transduced with a Siglec-8^{V-set}-directed 41 BB co-stimulatory domain-containing CAR generated from scFv sequences from either 1B6 or 1H4 induced significant cytotoxicity against EOL-1 and RS4; 11 cells transduced with Siglec-8^{FL}(FIGS. 19A-19D). Together, these studies demonstrate that Siglec-8-directed immunotherapies engaging T or NK cells can be highly effective against Siglec-8 expressing cells, even against cells displaying very low amounts of the target antigen.

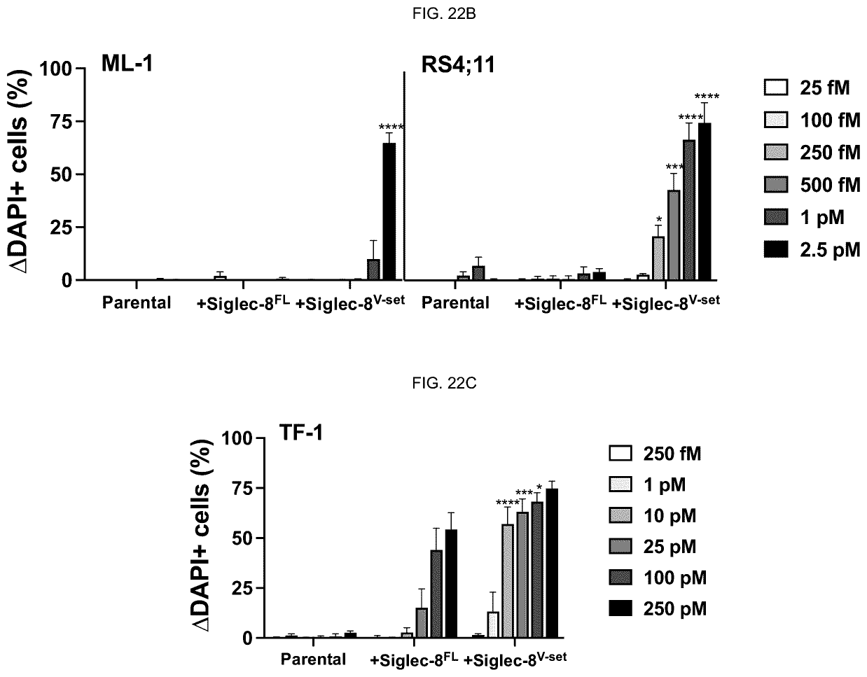

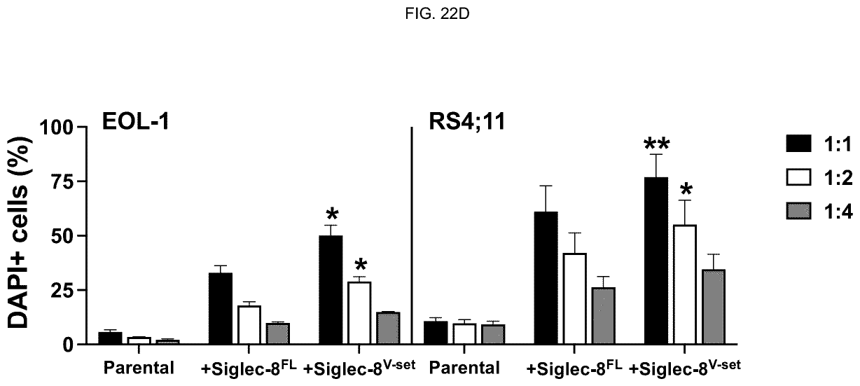

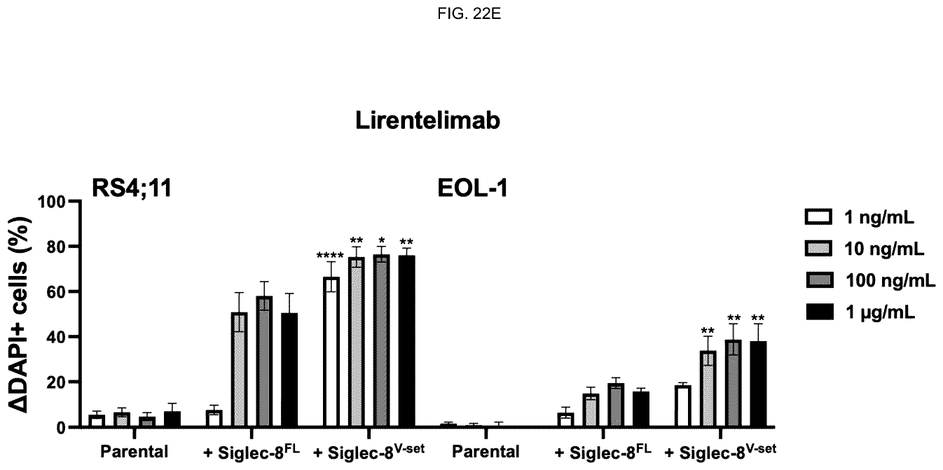

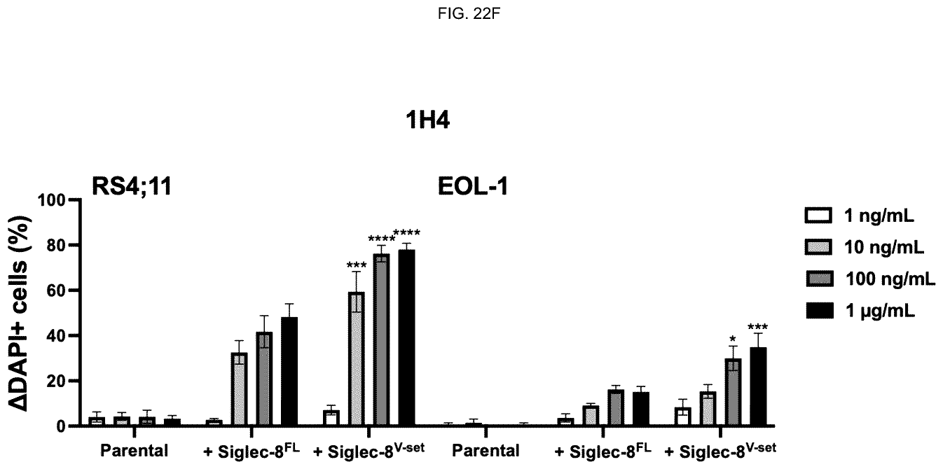

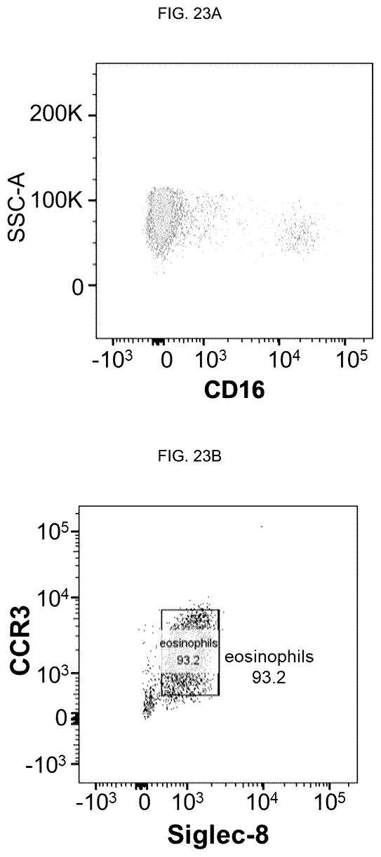

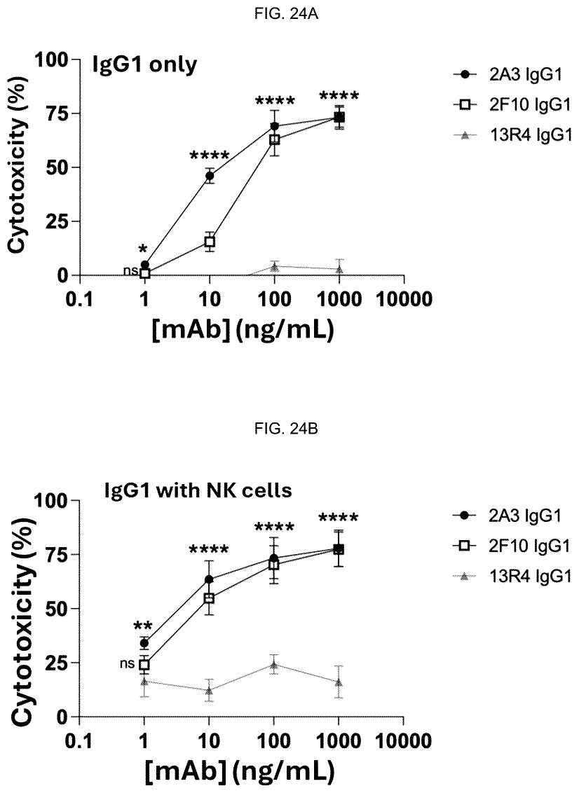

Binding distance from cell membrane correlates with efficacy of Siglec-8-directed immunotherapies engaging T or NK cells. In recent studies aimed at developing immunotherapies targeting Siglec-3 (CD33), it was found that membrane-proximal binding enhances effector functions of CD3-directed BiAbs and CAR-modified T cells (Godwin 2021). To examine the relationship between the efficacy of T cell and NK cell-engaging immunotherapies and the distance from the target epitope on Siglec-8 to the cell membrane, an experimental approach similar to the one used in studies on CD33 was employed (Godwin 2021). Specifically, human AML and ALL cell line pairs expressing comparable levels of either Siglec-8^{FL} or an artificial variant of Siglec-8 containing only the V-set domain on the extracellular portion of the protein (Siglec-8^{V-set}) to bring the V-set domain closer to the cell membrane (for cartoons, see FIG. 1) were engineered and average Siglec-8 molecules per cell assessed (FIG. 22A), and these cells were used as targets for Siglec-8^{V-set}-directed BiAbs and CAR-modified immune effector cells (FIGS. 22B-22D). ML-1, RS4; 11, and TF-1 cell line pairs were subjected to short-term in vitro cytotoxicity assays with various doses of the Siglec-8^{V-set}/CD3 BiAb and healthy human donor T cells as immune effector cells. As shown in FIGS. 22B-22C Siglec-8^{V-set}/CD3 BiAbs exerted greater cytotoxicity against AML and ALL cells expressing Siglec-8^{V-set} than cells expressing Siglec-8^{FL} despite similar or higher expression levels of Siglec-8^{FL} compared to Siglec-8^{V-set}. EOL-1 and RS4; 11 cell line pairs were then subjected to short-term in vitro cytotoxicity assays with Siglec-8^{V-set} CAR-modified KHYG-1 cells at various E:T cell ratios. Similar to results with Siglec-8^{V-set}/CD3 BiAbs, these CAR-modified NK cells exerted greater cytotoxicity against AML and ALL cells expressing Siglec-8^{V-set} than cells expressing Siglec-8^{FL} despite similar or higher expression levels of Siglec-8^{FL} compared to Siglec-8^{V-set} (FIG. 22D). To examine the relationship between the ADCC efficacy and the distance from the target epitope on Siglec-8 to the cell membrane, EOL-1 and RS4; 11 cell line pairs were subjected to short-term in vitro cytotoxicity assays with increasing concentrations of the Siglec-8^{V-set} mAbs (lirentelimab or 1H4) and healthy human donor NK cells as effector cells. Both lirentelimab (FIG. 22E) and 1H4 (FIG. 22F) exerted greater ADCC against cells expressing Siglec-8^{V-set} than cells expressing Siglec-8^{FL} despite similar or higher expression levels of Siglec-8^{FL} compared to Siglec-8^{V-set}, indicating that membrane-proximal binding of Siglec-8 enhances ADCC. Together, these data demonstrate altering the position of the Siglec-8 mAb binding epitope changes the effector functions of Siglec-8 mAb-derived therapies and suggest that membrane-proximal targeting of Siglec-8 via one of the C2-set domains can improve the efficacy of Siglec-8-targeted therapies that engage T or NK cells as immune effector cells.

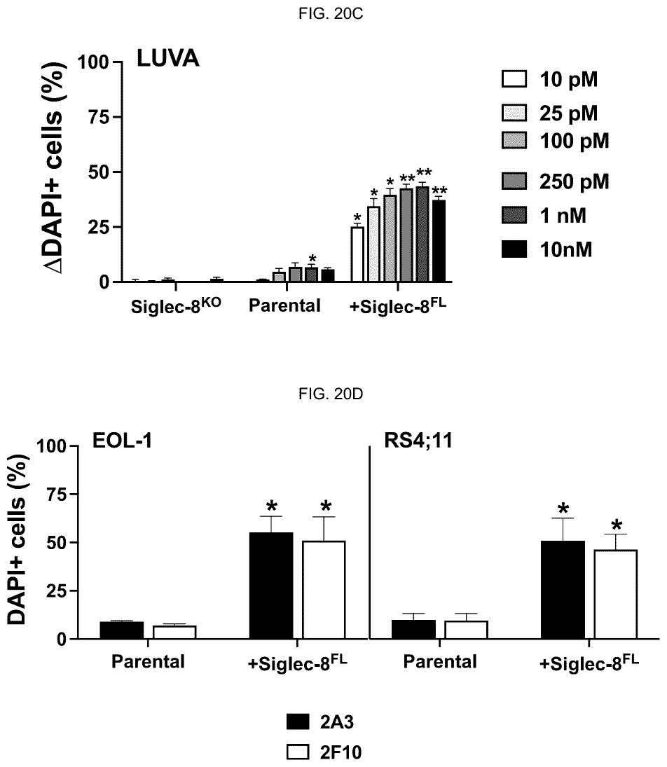

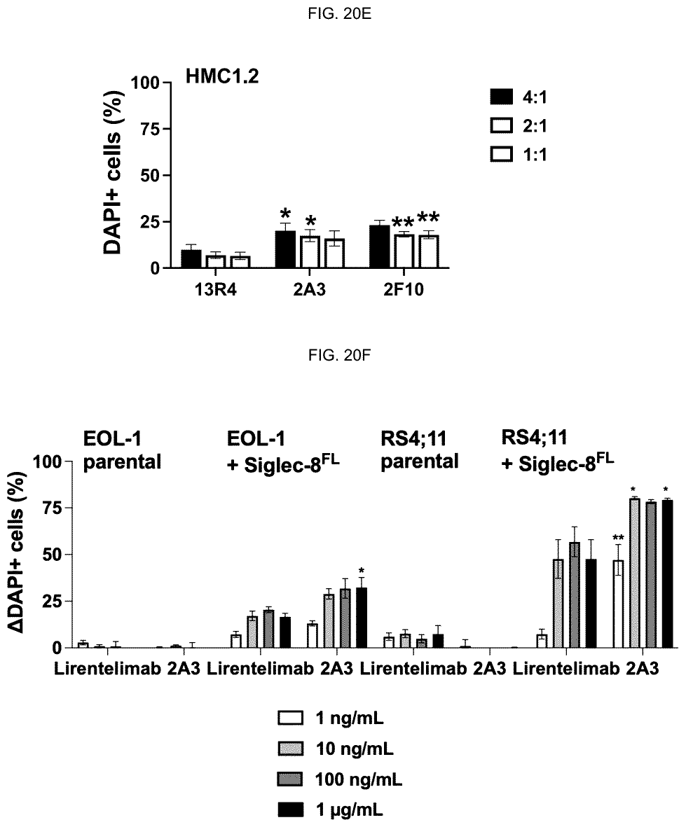

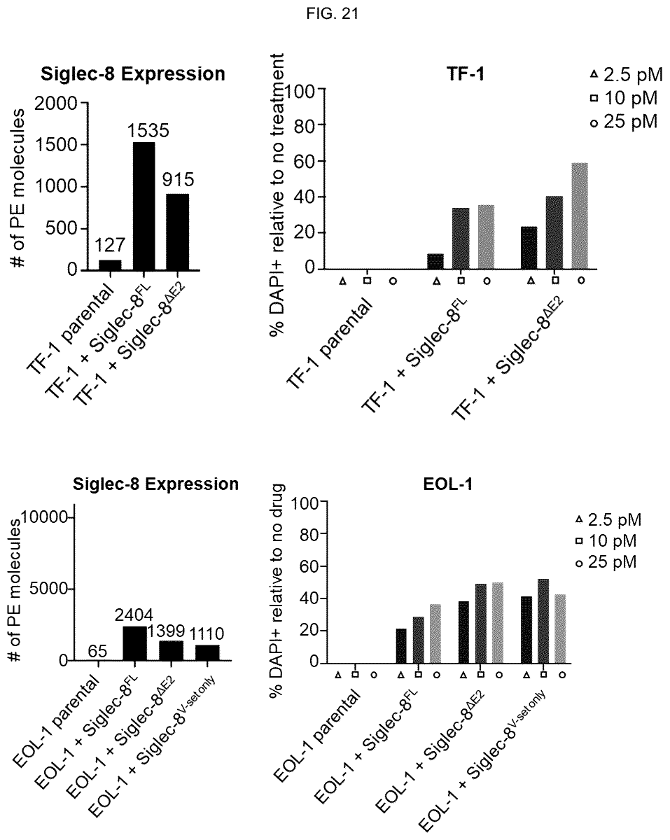

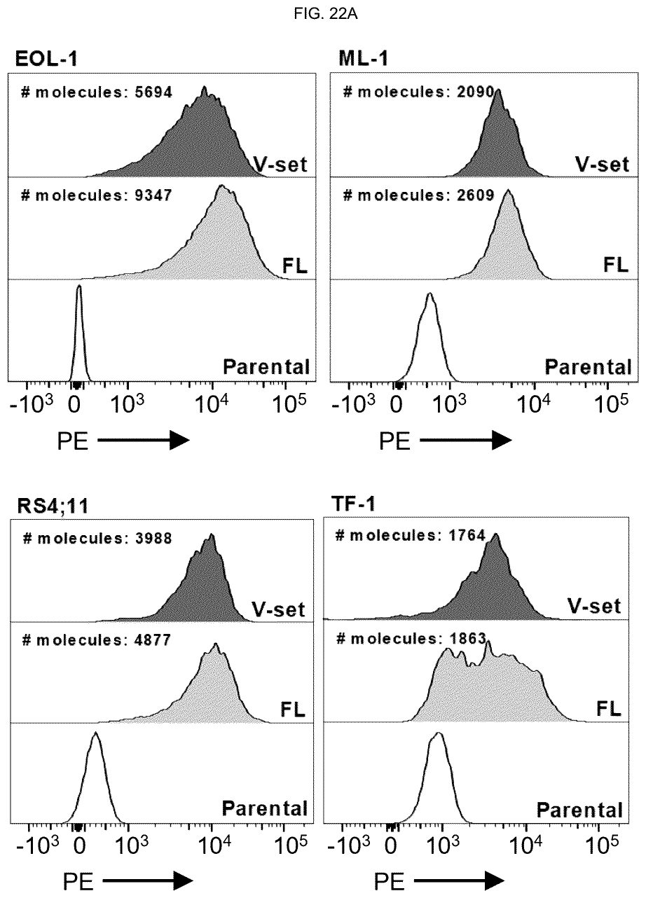

Generation of Siglec-8^{C2-set}/CD3 BiAb. In efforts toward immunization, the majority of identified mAbs recognized the V-set domain of Siglec-8, suggesting that immune-dominant epitopes are located on the outermost domain of Siglec-8. Three mAbs (clones 2A3, 2A7, and 2F10) bound a domain other than the V-set domain: for 2A3, the membrane-distal C2-set domain; for 2A7 and 2F10, the membrane-proximal C2-set domain. Next, tool constructs were generated demonstrating efficacy of Siglec-8 immunotherapeutics targeting C2-set domains. As an example of T-cell engaging immunotherapeutics, a Siglec-8^{C2-set}/CD3 BiAb in the scFv-scFv format was generated using binding sequences from 2A3. As shown as examples in FIGS. 20A-20E, Siglec-8^{C2-set}/CD3 BiAbs effectively killed EOL-1, ML-1, RS4; 11, and LUVA cells transduced with human Siglec-8^{FL} at very low concentrations. This BiAb also exerted Siglec-8-specific cytotoxicity against Siglec-8-enriched HMC 1.2 cells and parental LUVA cells, which both display Siglec-8 at very low copy number. To test whether such mAbs could also serve as basis for potent NK cell-engaging immunotherapeutics, their ADCC efficacy was assessed. 2A3 that binds the membrane-distal C2-set domain of Siglec-8 induced dose-dependent ADCC against RS4; 11 and EOL-1 cells transduced with human Siglec-8^{FL} but not Siglec-8-negative parental RS4; 11 and EOL-1 cells (FIG. 20F). Compared to lirentelimab, 2A3 showed higher ADCC efficacy against AML and ALL cell lines overexpressing Siglec-8^{FL} (FIG. 20F). No direct apoptotic effect was observed against leukemia cell lines transduced with Siglec-8^{FL} when Siglec-8 mAbs were incubated with the cell lines without NK effector cells present (data not shown). Likewise, KHYG-1 cells transduced with a Siglec-8^{C2-set}-directed CAR generated from scFv sequences from either 2A3 or 2F10 induced significant cytotoxicity against EOL-1 and RS4; 11 cells transduced with Siglec-8^{FL} (FIGS. 20A-20E). These CAR-modified KHGY-1 cells also exerted Siglec-8 specific cytotoxicity against Siglec-8-enriched HMC 1.2 cells. Together, these studies demonstrate that Siglec-8^{C2-set}-directed immunotherapies engaging T or NK cells can be highly effective, even against target cells expressing limited amounts of Siglec-8.

Efficacy of Siglec-8^{C2-set}-directed immunotherapies against primary human eosinophils. Like previously reported for lirentelimab (Youngblood 2019) unconjugated Siglec-8^{C2-set} antibodies induced direct apoptosis in purified primary human eosinophils. Shown in FIG. 24A, compared to the non-binding control antibody (13R4), the Siglec-8^{C2-set} antibodies (2A3 and 2F10) in IgG1 format induced dose-dependent apoptosis of eosinophils, even without prior activation of the eosinophils using IL-5. With additional allogeneic primary human NK cells present as effector cells, the Siglec-8^{C2-set} mAbs (2A3 and 2F10) induced even greater cytotoxicity than without the NK cells at low antibody concentrations (1-100 ng/mL) likely because of additional ADCC (FIG. 24B). Similarly, compared to the non-binding control BiAb (13R4), the Siglec-8^{C2-set}/CD3 BiAb using sequences from 2A3 exerted dose-dependent T-cell mediated cytotoxicity against the primary human eosinophils (FIG. 24C). Finally, both Siglec-8^{C2-set}-directed (2A3 and 2F10) CAR-modified KHYG-1 cells exerted dose-dependent cytotoxicity against the primary human eosinophils compared to the non-binding control CAR-NK cells (13R4) (FIG. 24D), demonstrating that Siglec-8^{C2-set}-directed immunotherapies engaging T or NK cells are potent against human eosinophils isolated from patients.

Discussion Siglecs are members of the immunoglobulin gene family implicated in promoting cell-cell interactions and regulating functions of cells in the innate and adaptive immune systems through glycan recognition (Crocker 2007). They are primarily found on hematopoietic and immune cells, mostly in a highly cell type-restricted pattern. This is true for Siglec-8, which is uniquely expressed on eosinophils, mast cells and, in some cases, basophils (Kiwamoto 2012; Floyd 2000; Kikly 2000; Liu et 2006). Displayed late in the eosinophil and mast cell maturation process, Siglec-8 is not found on hematopoietic stem cells. Unconjugated Siglec-8 mAbs cause caspase and/or reactive oxygen species-dependent apoptosis of eosinophils in vitro (Nutku et al., Blood. 2003, 101(12):5014-5020; Nutku et al., Biochem Biophys Res Commun. 2005, 336(3):918-924; Nutku-Bilir et al., Am J Respir Cell Mol Biol. 2008; 38(1):121-124). The benefit seen using the unconjugated anti-Siglec-8 mAb, lirentelimab, in some patients with eosinophilic gastritis and duodenitis (Dellon 2020) supports this notion but more potent therapeutic modalities are necessary for other, more aggressive diseases involving eosinophils and mast cells.

Taking advantage of a transgenic mouse platform, new, fully human anti-Siglec-8 mAbs were developed herein. Using several immunogens, including chimeric proteins which contain portions of the extracellular domain of human Siglec-8, a panel of mAbs was obtained with diverse epitopes to which they bind. Domain mapping studies indicated the majority of mAbs recognize the V-set domain of Siglec-8, suggesting this outermost domain contains the immune dominant epitopes of Siglec-8. This observation parallels what was seen with Siglec-3 (CD33), where most mAbs recognize the V-set domain. Nonetheless, a subset of the generated mAbs recognize epitopes on either the membrane-distal or membrane-proximal C2-set domain of Siglec-8. The mAb targeting diversity provided herein allows for tailoring or choosing the ideal mAb for the desired treatment modality.

Siglec-8 has endocytic properties (O'Sullivan 2018). Consistent with this notion, all tested fully human mAbs were internalized in a time- and Siglec-8-dependent fashion. While four of the five mAbs studied internalized with relatively similar kinetics, one mAb (2A3) internalized significantly more slowly, drawing attention to the possibility that some mAbs might be more suitable as carriers for toxic payloads (e.g. in the form of an antibody-drug conjugate or immunotoxin), whereas others might be better suited for applications for which limited internalization may be preferable (e.g. for targeted delivery of radioisotopes).

Because prior in vitro studies showed that unconjugated anti-Siglec-8 mAbs inhibit mast cell function but cannot induce mast cell apoptosis (Kolkhir2022; Youngblood 2019; Kerr 2020; Schanin 2021), this example focused on Siglec-8-directed immunotherapies with T cell-engaging BiAbs and CAR-modified immune effector cells. Experimental work demonstrates that both Siglec-8/CD3 BiAbs and Siglec-8-directed NK CAR cells can be highly effective, and highly specific, against Siglec-8-expressing hematopoietic cells. Importantly, these therapeutics induced Siglec-8-specific cytotoxicity even in cells with barely detectable Siglec-8 expression, indicating such approaches can be effective even in situations where target antigen expression is very low and other therapeutic modalities (e.g. radiolabeled or toxin-labeled mAbs) might fail. Together these data provide a foundation for the further development and characterization of Siglec-8/CD3 BiAbs and/or Siglec-8-directed CAR cells for clinical application for patients with aggressive diseases involving eosinophils and mast cells.

For several cell surface proteins, including 2 members of the Siglec family (CD22 and CD33), binding membrane proximal epitopes was shown to enhance the efficacy of mAbs, CD3-directed BiAbs, or CAR T cells (Godwin 2021; Bluemel et al., Cancer Immunol Immunother. 2010, 59(8):1197-1209; Lin Pharmgenomics Pers Med. 2010, 3:51-59; Haso et al., Blood. 2013, 121(7):1165-1174; Cleary et al., J Immunol. 2017, 198(10):3999-4011). To test this relationship with Siglec-8, a strategy was used that is based on natural and artificial proteins where the targeted domain's position relative to the cell membrane is varied. Specifically, a construct was generated where the V-set domain of Siglec-8 was positioned closer to the cell membrane. Further, several Siglec-8^{V-set}-directed therapeutics were used in comparative cytotoxicity experiments with cells expressing full-length Siglec-8. Together, the studies—done in cell line pairs that express well-matched levels of target antigens—demonstrate that membrane-proximal targeting of Siglec-8 enhances the cytolytic activities of both Siglec-8/CD3 BiAbs as well as Siglec-8-directed CAR NK cells. While based on an artificial protein, this approach allows use of single therapeutics for comparative assessments, thereby avoiding potential issues that could arise from mAbs with different affinities and/or the targeting of different binding epitopes. One approach to increase the efficacy of Siglec-8-targeting therapeutics could be to integrate binding sequences from mAbs that bind to membrane proximal regions of the Siglec-8 protein into therapeutics. To support the feasibility of this approach, several Siglec-8^{C2-set} directed therapeutics were generated and tested and demonstrated high anti-tumor activity both in the context of T cell-directed BiAbs and CAR NK cells.

Taken together, the current disclosure provides a series of diverse, fully human mAbs against Siglec-8. Experimental data demonstrates that Siglec-8-directed immunotherapies can be highly potent, supporting further development for use in patients with eosinophilic and mast cell disorders, particularly for those whom current treatments are insufficiently effective.

(xii) Closing Paragraphs. The nucleic acid and amino acid sequences provided herein are shown using letter abbreviations for nucleotide bases and amino acid residues, as defined in 37 C.F.R. § 1.831-1.835 and set forth in WIPO Standard ST.26 (implemented on Jul. 1, 2022). Only one strand of each nucleic acid sequence is shown, but the complementary strand is understood as included in embodiments where it would be appropriate.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (lie), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, lie, Phe, Met, and Trp; Group 11 (aliphatic): Gly, Ala, Val, Leu, and lie; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W. H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure, it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridize under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g., 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Binds” refers to an association of a binding domain (of, for example, a human Siglec-8 antibody) to its cognate binding molecule with an affinity or Kₐ (i.e., an equilibrium association constant of a particular binding interaction with units of 1/M) equal to or greater than 10⁵ M⁻¹, while not significantly associating with any other molecules or components in a relevant environment sample. Binding domains may be classified as “high affinity” or “low affinity”. In particular embodiments, “high affinity” binding domains refer to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹¹ M⁻¹. In particular embodiments, “low affinity” binding domains refer to those binding domains with a Kₐ of up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M). In certain embodiments, a binding domain may have “enhanced affinity,” which refers to a selected or engineered binding domains with stronger binding to a cognate binding molecule than a wild type (or parent) binding domain. For example, enhanced affinity may be due to a Kₐ (equilibrium association constant) for the cognate binding molecule that is higher than the reference binding domain or due to a K_{d} (dissociation constant) for the cognate binding molecule that is less than that of the reference binding domain, or due to an off-rate (K_{off}) for the cognate binding molecule that is less than that of the reference binding domain. A variety of assays are known for detecting binding domains that bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant decrease in binding affinity to Siglec-8 by an antibody disclosed herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

1.-125. (canceled)

126. An antibody or binding fragment thereof comprising a set of complementarity determining regions (CDRs):
according to North comprising:
a light chain (LC) CDR1 having the sequence as set forth in SEQ ID NO: 26, an LC CDR2 having the sequence as set forth in SEQ ID NO: 27, an LC CDR3 having the sequence as set forth in SEQ ID NO: 28, a heavy chain (HC) CDR1 having the sequence as set forth in SEQ ID NO: 29, an HC CDR2 having the sequence as set forth in SEQ ID NO: 30, and an HC CDR3 having the sequence as set forth in SEQ ID NO: 31;
an LC CDR1 having the sequence as set forth in SEQ ID NO: 34, an LC CDR2 having the sequence as set forth in SEQ ID NO: 35, an LC CDR3 having the sequence as set forth in SEQ ID NO: 28, an HC CDR1 having the sequence as set forth in SEQ ID NO: 36, an HC CDR2 having the sequence as set forth in SEQ ID NO: 37, and an HC CDR3 having the sequence as set forth in SEQ ID NO: 31;
an LC CDR1 having the sequence as set forth in SEQ ID NO: 38, an LC CDR2 having the sequence as set forth in SEQ ID NO: 39, an LC CDR3 having the sequence as set forth in SEQ ID NO: 40 an HC CDR1 having the sequence as set forth in SEQ ID NO:41, an HC CDR2 having the sequence as set forth in SEQ ID NO: 42, and an HC CDR3 having the sequence as set forth in SEQ ID NO: 43;
an LC CDR1 having the sequence as set forth in SEQ ID NO: 45, an LC CDR2 having the sequence as set forth in SEQ ID NO: 46, an LC CDR3 having the sequence as set forth in SEQ ID NO: 47, an HC CDR1 having the sequence as set forth in SEQ ID NO:48 an HC CDR2 having the sequence as set forth in SEQ ID NO: 49, and an HC CDR3 having the sequence as set forth in SEQ ID NO: 50;
an LC CDR1 having the sequence as set forth in SEQ ID NO: 51, an LC CDR2 having the sequence as set forth in SEQ ID NO: 52, an LC CDR3 having the sequence as set forth in SEQ ID NO: 53, an HC CDR1 having the sequence as set forth in SEQ ID NO:54, an HC CDR2 having the sequence as set forth in SEQ ID NO: 55, and an HC CDR3 having the sequence as set forth in SEQ ID NO: 56;
an LC CDR1 having the sequence as set forth in SEQ ID NO: 57, an LC CDR2 having the sequence as set forth in SEQ ID NO: 58, an LC CDR3 having the sequence as set forth in SEQ ID NO: 59, an HC CDR1 having the sequence as set forth in SEQ ID NO:60, an HC CDR2 having the sequence as set forth in SEQ ID NO: 61, and an HC CDR3 having the sequence as set forth in SEQ ID NO: 62;
an LC CDR1 having the sequence as set forth in SEQ ID NO: 63, an LC CDR2 having the sequence as set forth in SEQ ID NO: 64, an LC CDR3 having the sequence as set forth in SEQ ID NO: 65, an HC CDR1 having the sequence as set forth in SEQ ID NO:66, an HC CDR2 having the sequence as set forth in SEQ ID NO: 67, and an HC CDR3 having the sequence as set forth in SEQ ID NO: 68;
an LC CDR1 having the sequence as set forth in SEQ ID NO: 69, an LC CDR2 having the sequence as set forth in SEQ ID NO: 70, an LC CDR3 having the sequence as set forth in SEQ ID NO: 71, an HC CDR1 having the sequence as set forth in SEQ ID NO:72, an HC CDR2 having the sequence as set forth in SEQ ID NO: 73, and an HC CDR3 having the sequence as set forth in SEQ ID NO: 74;
an LC CDR1 having the sequence as set forth in SEQ ID NO: 75, an LC CDR2 having the sequence as set forth in SEQ ID NO: 76, an LC CDR3 having the sequence as set forth in SEQ ID NO: 77, an HC CDR1 having the sequence as set forth in SEQ ID NO:78, an HC CDR2 having the sequence as set forth in SEQ ID NO: 79, and an HC CDR3 having the sequence as set forth in SEQ ID NO: 80;
an LC CDR1 having the sequence as set forth in SEQ ID NO: 69, an LC CDR2 having the sequence as set forth in SEQ ID NO: 70, an LC CDR3 having the sequence as set forth in SEQ ID NO: 71, an HC CDR1 having the sequence as set forth in SEQ ID NO:72, an HC CDR2 having the sequence as set forth in SEQ ID NO: 73, and an HC CDR3 having the sequence as set forth in SEQ ID NO: 74; or
an LC CDR1 having the sequence as set forth in SEQ ID NO: 75, an LC CDR2 having the sequence as set forth in SEQ ID NO: 76, an LC CDR3 having the sequence as set forth in SEQ ID NO: 77, an HC CDR1 having the sequence as set forth in SEQ ID NO:81, an HC CDR2 having the sequence as set forth in SEQ ID NO: 82, and an HC CDR3 having the sequence as set forth in SEQ ID NO: 83;
according to IMGT comprising:
an LC CDR1 having the sequence as set forth in SEQ ID NO: 86, an LC CDR2 having the sequence ATS, an LC CDR3 having the sequence as set forth in SEQ ID NO: 28, an HC CDR1 having the sequence as set forth in SEQ ID NO:87, an HC CDR2 having the sequence as set forth in SEQ ID NO: 88, and an HC CDR3 having the sequence as set forth in SEQ ID NO: 31;
an LC CDR1 having the sequence as set forth in SEQ ID NO: 86, an LC CDR2 having the sequence ASS, an LC CDR3 having the sequence as set forth in SEQ ID NO: 28, an HC CDR1 having the sequence as set forth in SEQ ID NO:90, an HC CDR2 having the sequence as set forth in SEQ ID NO: 88, and an HC CDR3 having the sequence as set forth in SEQ ID NO: 31;
an LC CDR1 having the sequence as set forth in SEQ ID NO: 91, an LC CDR2 having the sequence LVS, an LC CDR3 having the sequence as set forth in SEQ ID NO: 40, an HC CDR1 having the sequence as set forth in SEQ ID NO:92, an HC CDR2 having the sequence as set forth in SEQ ID NO: 93, and an HC CDR3 having the sequence as set forth in SEQ ID NO: 43;
an LC CDR1 having the sequence as set forth in SEQ ID NO: 95, an LC CDR2 having the sequence GAS, an LC CDR3 having the sequence as set forth in SEQ ID NO: 47, an HC CDR1 having the sequence as set forth in SEQ ID NO:96, an HC CDR2 having the sequence as set forth in SEQ ID NO: 97, and an HC CDR3 having the sequence as set forth in SEQ ID NO: 50;
an LC CDR1 having the sequence as set forth in SEQ ID NO: 98, an LC CDR2 having the sequence AAS, an LC CDR3 having the sequence as set forth in SEQ ID NO: 53, an HC CDR1 having the sequence as set forth in SEQ ID NO:99, an HC CDR2 having the sequence as set forth in SEQ ID NO: 100, and an HC CDR3 having the sequence as set forth in SEQ ID NO: 56;
an LC CDR1 having the sequence as set forth in SEQ ID NO: 101, an LC CDR2 having the sequence GAS, an LC CDR3 having the sequence as set forth in SEQ ID NO: 59, an HC CDR1 having the sequence as set forth in SEQ ID NO:102, an HC CDR2 having the sequence as set forth in SEQ ID NO: 103 and an HC CDR3 having the sequence as set forth in SEQ ID NO: 62;
an LC CDR1 having the sequence as set forth in SEQ ID NO: 104, an LC CDR2 having the sequence NNN, an LC CDR3 having the sequence as set forth in SEQ ID NO: 65, an HC CDR1 having the sequence as set forth in SEQ ID NO:105, an HC CDR2 having the sequence as set forth in SEQ ID NO: 106, and an HC CDR3 having the sequence as set forth in SEQ ID NO: 68;
an LC CDR1 having the sequence as set forth in SEQ ID NO: 107, an LC CDR2 having the sequence AAS, an LC CDR3 having the sequence as set forth in SEQ ID NO: 71, an HC CDR1 having the sequence as set forth in SEQ ID NO:108, an HC CDR2 having the sequence as set forth in SEQ ID NO: 109, and an HC CDR3 having the sequence as set forth in SEQ ID NO: 74;
an LC CDR1 having the sequence as set forth in SEQ ID NO: 110, an LC CDR2 having the sequence KIS, an LC CDR3 having the sequence as set forth in SEQ ID NO: 77, an HC CDR1 having the sequence as set forth in SEQ ID NO:111, an HC CDR2 having the sequence as set forth in SEQ ID NO: 112, and an HC CDR3 having the sequence as set forth in SEQ ID NO: 80;
an LC CDR1 having the sequence as set forth in SEQ ID NO: 107, an LC CDR2 having the sequence AAS, an LC CDR3 having the sequence as set forth in SEQ ID NO: 71, an HC CDR1 having the sequence as set forth in SEQ ID NO:108, an HC CDR2 having the sequence as set forth in SEQ ID NO: 109, and an HC CDR3 having the sequence as set forth in SEQ ID NO: 74; or
an LC CDR1 having the sequence as set forth in SEQ ID NO: 110, an LC CDR2 having the sequence KIS, an LC CDR3 having the sequence as set forth in SEQ ID NO: 77, an HC CDR1 having the sequence as set forth in SEQ ID NO:113, an HC CDR2 having the sequence as set forth in SEQ ID NO: 114, and an HC CDR3 having the sequence as set forth in SEQ ID NO: 83;
according to Kabat comprising:
an LC CDR1 having the sequence as set forth in SEQ ID NO: 26, an LC CDR2 having the sequence as set forth in SEQ ID NO: 116, an LC CDR3 having the sequence as set forth in SEQ ID NO: 28, an HC CDR1 having the sequence as set forth in SEQ ID NO: 117, an HC CDR2 having the sequence as set forth in SEQ ID NO:118, and an HC CDR3 having the sequence as set forth in SEQ ID NO: 119;
an LC CDR1 having the sequence as set forth in SEQ ID NO: 34, an LC CDR2 having the sequence as set forth in SEQ ID NO: 121, an LC CDR3 having the sequence as set forth in SEQ ID NO: 28, an HC CDR1 having the sequence as set forth in SEQ ID NO: 117, an HC CDR2 having the sequence as set forth in SEQ ID NO:122, and an HC CDR3 having the sequence as set forth in SEQ ID NO: 119;
an LC CDR1 having the sequence as set forth in SEQ ID NO: 38, an LC CDR2 having the sequence as set forth in SEQ ID NO: 123, an LC CDR3 having the sequence as set forth in SEQ ID NO: 40, an HC CDR1 having the sequence as set forth in SEQ ID NO: 124, an HC CDR2 having the sequence as set forth in SEQ ID NO:125, and an HC CDR3 having the sequence as set forth in SEQ ID NO: 126;
an LC CDR1 having the sequence as set forth in SEQ ID NO: 45, an LC CDR2 having the sequence as set forth in SEQ ID NO: 127, an LC CDR3 having the sequence as set forth in SEQ ID NO: 47, an HC CDR1 having the sequence as set forth in SEQ ID NO: 128, an HC CDR2 having the sequence as set forth in SEQ ID NO:129, and an HC CDR3 having the sequence as set forth in SEQ ID NO: 130;
an LC CDR1 having the sequence as set forth in SEQ ID NO: 51, an LC CDR2 having the sequence as set forth in SEQ ID NO: 131, an LC CDR3 having the sequence as set forth in SEQ ID NO: 53, an HC CDR1 having the sequence as set forth in SEQ ID NO: 132, an HC CDR2 having the sequence as set forth in SEQ ID NO:133, and an HC CDR3 having the sequence as set forth in SEQ ID NO: 134;
an LC CDR1 having the sequence as set forth in SEQ ID NO: 57, an LC CDR2 having the sequence as set forth in SEQ ID NO: 135, an LC CDR3 having the sequence as set forth in SEQ ID NO: 59, an HC CDR1 having the sequence as set forth in SEQ ID NO: 136, an HC CDR2 having the sequence as set forth in SEQ ID NO:137, and an HC CDR3 having the sequence as set forth in SEQ ID NO: 138;
an LC CDR1 having the sequence as set forth in SEQ ID NO: 63, an LC CDR2 having the sequence as set forth in SEQ ID NO: 139, an LC CDR3 having the sequence as set forth in SEQ ID NO: 65, an HC CDR1 having the sequence as set forth in SEQ ID NO: 140, an HC CDR2 having the sequence as set forth in SEQ ID NO:141, and an HC CDR3 having the sequence as set forth in SEQ ID NO: 142;
an LC CDR1 having the sequence as set forth in SEQ ID NO: 69, an LC CDR2 having the sequence as set forth in SEQ ID NO: 143, an LC CDR3 having the sequence as set forth in SEQ ID NO: 71, an HC CDR1 having the sequence as set forth in SEQ ID NO: 144, an HC CDR2 having the sequence as set forth in SEQ ID NO:145, and an HC CDR3 having the sequence as set forth in SEQ ID NO: 146;
an LC CDR1 having the sequence as set forth in SEQ ID NO: 75, an LC CDR2 having the sequence as set forth in SEQ ID NO: 147, an LC CDR3 having the sequence as set forth in SEQ ID NO: 77, an HC CDR1 having the sequence as set forth in SEQ ID NO: 148, an HC CDR2 having the sequence as set forth in SEQ ID NO:149, and an HC CDR3 having the sequence as set forth in SEQ ID NO: 150;
an LC CDR1 having the sequence as set forth in SEQ ID NO: 69, an LC CDR2 having the sequence as set forth in SEQ ID NO: 143, an LC CDR3 having the sequence as set forth in SEQ ID NO: 71, an HC CDR1 having the sequence as set forth in SEQ ID NO: 144, an HC CDR2 having the sequence as set forth in SEQ ID NO:151, and an HC CDR3 having the sequence as set forth in SEQ ID NO: 146; or
an LC CDR1 having the sequence as set forth in SEQ ID NO: 75, an LC CDR2 having the sequence as set forth in SEQ ID NO: 147, an LC CDR3 having the sequence as set forth in SEQ ID NO: 77, an HC CDR1 having the sequence as set forth in SEQ ID NO: 148, an HC CDR2 having the sequence as set forth in SEQ ID NO:152, and an HC CDR3 having the sequence as set forth in SEQ ID NO: 153;
according to Chothia comprising:
an LC CDR1 the sequence as set forth in SEQ ID NO: 26, an LC CDR2 the sequence as set forth in SEQ ID NO: 116, an LC CDR3 having the sequence as set forth in SEQ ID NO: 28, an HC CDR1 having the sequence as set forth in SEQ ID NO: 156, an HC CDR2 having the sequence as set forth in SEQ ID NO:157, and an HC CDR3 having the sequence as set forth in SEQ ID NO: 119;
an LC CDR1 having the sequence as set forth in SEQ ID NO: 34, an LC CDR2 having the sequence as set forth in SEQ ID NO: 121, an LC CDR3 having the sequence as set forth in SEQ ID NO: 28, an HC CDR1 having the sequence as set forth in SEQ ID NO: 158, an HC CDR2 having the sequence as set forth in SEQ ID NO:157, and an HC CDR3 having the sequence as set forth in SEQ ID NO: 119;
an LC CDR1 having the sequence as set forth in SEQ ID NO: 38, an LC CDR2 having the sequence as set forth in SEQ ID NO: 123, an LC CDR3 having the sequence as set forth in SEQ ID NO: 40, an HC CDR1 having the sequence as set forth in SEQ ID NO: 159, an HC CDR2 having the sequence as set forth in SEQ ID NO:160, and an HC CDR3 having the sequence as set forth in SEQ ID NO: 126;
an LC CDR1 having the sequence as set forth in SEQ ID NO: 45, an LC CDR2 having the sequence as set forth in SEQ ID NO: 127, an LC CDR3 having the sequence as set forth in SEQ ID NO: 47, an HC CDR1 having the sequence as set forth in SEQ ID NO: 161, an HC CDR2 having the sequence as set forth in SEQ ID NO:162, and an HC CDR3 having the sequence as set forth in SEQ ID NO: 130;
an LC CDR1 having the sequence as set forth in SEQ ID NO: 51, an LC CDR2 having the sequence as set forth in SEQ ID NO: 131, an LC CDR3 having the sequence as set forth in SEQ ID NO: 53, an HC CDR1 having the sequence as set forth in SEQ ID NO: 163, an HC CDR2 having the sequence as set forth in SEQ ID NO:164, and an HC CDR3 having the sequence as set forth in SEQ ID NO: 134;
an LC CDR1 having the sequence as set forth in SEQ ID NO: 57, an LC CDR2 having the sequence as set forth in SEQ ID NO: 135, an LC CDR3 having the sequence as set forth in SEQ ID NO: 59, an HC CDR1 having the sequence as set forth in SEQ ID NO: 165, an HC CDR2 having the sequence as set forth in SEQ ID NO:166, and an HC CDR3 having the sequence as set forth in SEQ ID NO: 138;
an LC CDR1 having the sequence as set forth in SEQ ID NO: 63, an LC CDR2 having the sequence as set forth in SEQ ID NO: 139, an LC CDR3 having the sequence as set forth in SEQ ID NO: 65, an HC CDR1 having the sequence as set forth in SEQ ID NO: 167, an HC CDR2 having the sequence as set forth in SEQ ID NO:168, and an HC CDR3 having the sequence as set forth in SEQ ID NO: 142;
an LC CDR1 having the sequence as set forth in SEQ ID NO: 69, an LC CDR2 having the sequence as set forth in SEQ ID NO: 143, an LC CDR3 having the sequence as set forth in SEQ ID NO: 71, an HC CDR1 having the sequence as set forth in SEQ ID NO: 169, an HC CDR2 having the sequence as set forth in SEQ ID NO:170, and an HC CDR3 having the sequence as set forth in SEQ ID NO: 146;
an LC CDR1 having the sequence as set forth in SEQ ID NO: 75, an LC CDR2 having the sequence as set forth in SEQ ID NO: 147, an LC CDR3 having the sequence as set forth in SEQ ID NO: 77, an HC CDR1 having the sequence as set forth in SEQ ID NO: 171, an HC CDR2 having the sequence as set forth in SEQ ID NO:172, and an HC CDR3 having the sequence as set forth in SEQ ID NO: 150;
an LC CDR1 having the sequence as set forth in SEQ ID NO: 69, an LC CDR2 having the sequence as set forth in SEQ ID NO: 143, an LC CDR3 having the sequence as set forth in SEQ ID NO: 71, an HC CDR1 having the sequence as set forth in SEQ ID NO: 169, an HC CDR2 having the sequence as set forth in SEQ ID NO:170, and an HC CDR3 having the sequence as set forth in SEQ ID NO: 146; or
an LC CDR1 having the sequence as set forth in SEQ ID NO: 75, an LC CDR2 having the sequence as set forth in SEQ ID NO: 147, an LC CDR3 having the sequence as set forth in SEQ ID NO: 77, an HC CDR1 having the sequence as set forth in SEQ ID NO: 173, an HC CDR2 having the sequence as set forth in SEQ ID NO:174, and an HC CDR3 having the sequence as set forth in SEQ ID NO: 153;
according to Contact comprising:
an LC CDR1 having the sequence as set forth in SEQ ID NO: 175, an LC CDR2 having the sequence as set forth in SEQ ID NO: 176, an LC CDR3 having the sequence as set forth in SEQ ID NO: 177, an HC CDR1 having the sequence as set forth in SEQ ID NO: 178, an HC CDR2 having the sequence as set forth in SEQ ID NO:179, and an HC CDR3 having the sequence as set forth in SEQ ID NO: 180;
an LC CDR1 having the sequence as set forth in SEQ ID NO: 175, an LC CDR2 having the sequence as set forth in SEQ ID NO: 183, an LC CDR3 having the sequence as set forth in SEQ ID NO: 177, an HC CDR1 having the sequence as set forth in SEQ ID NO: 184, an HC CDR2 having the sequence as set forth in SEQ ID NO:185, and an HC CDR3 having the sequence as set forth in SEQ ID NO: 180;
an LC CDR1 having the sequence as set forth in SEQ ID NO: 186, an LC CDR2 having the sequence as set forth in SEQ ID NO: 187, an LC CDR3 having the sequence as set forth in SEQ ID NO: 188, an HC CDR1 having the sequence as set forth in SEQ ID NO: 189, an HC CDR2 having the sequence as set forth in SEQ ID NO:190, and an HC CDR3 having the sequence as set forth in SEQ ID NO: 191;
an LC CDR1 having the sequence as set forth in SEQ ID NO: 192, an LC CDR2 having the sequence as set forth in SEQ ID NO: 193, an LC CDR3 having the sequence as set forth in SEQ ID NO: 194, an HC CDR1 having the sequence as set forth in SEQ ID NO: 195, an HC CDR2 having the sequence as set forth in SEQ ID NO:196, and an HC CDR3 having the sequence as set forth in SEQ ID NO: 197;
an LC CDR1 having the sequence as set forth in SEQ ID NO: 198, an LC CDR2 having the sequence as set forth in SEQ ID NO: 199, an LC CDR3 having the sequence as set forth in SEQ ID NO: 200, an HC CDR1 having the sequence as set forth in SEQ ID NO: 201, an HC CDR2 having the sequence as set forth in SEQ ID NO:202, and an HC CDR3 having the sequence as set forth in SEQ ID NO: 203;
an LC CDR1 having the sequence as set forth in SEQ ID NO: 204, an LC CDR2 having the sequence as set forth in SEQ ID NO: 205, an LC CDR3 having the sequence as set forth in SEQ ID NO: 206, an HC CDR1 having the sequence as set forth in SEQ ID NO: 207, an HC CDR2 having the sequence as set forth in SEQ ID NO:208, and an HC CDR3 having the sequence as set forth in SEQ ID NO: 209;
an LC CDR1 having the sequence as set forth in SEQ ID NO: 210, an LC CDR2 having the sequence as set forth in SEQ ID NO: 211, an LC CDR3 having the sequence as set forth in SEQ ID NO: 212, an HC CDR1 having the sequence as set forth in SEQ ID NO: 140, an HC CDR2 having the sequence as set forth in SEQ ID NO:213, and an HC CDR3 having the sequence as set forth in SEQ ID NO: 214;
an LC CDR1 having the sequence as set forth in SEQ ID NO: 215, an LC CDR2 having the sequence as set forth in SEQ ID NO: 216, an LC CDR3 having the sequence as set forth in SEQ ID NO: 217, an HC CDR1 having the sequence as set forth in SEQ ID NO: 218, an HC CDR2 having the sequence as set forth in SEQ ID NO:219, and an HC CDR3 having the sequence as set forth in SEQ ID NO: 220;
an LC CDR1 having the sequence as set forth in SEQ ID NO: 221, an LC CDR2 having the sequence as set forth in SEQ ID NO: 222, an LC CDR3 having the sequence as set forth in SEQ ID NO: 223, an HC CDR1 having the sequence as set forth in SEQ ID NO: 224, an HC CDR2 having the sequence as set forth in SEQ ID NO:225, and an HC CDR3 having the sequence as set forth in SEQ ID NO: 226;
an LC CDR1 having the sequence as set forth in SEQ ID NO: 215, an LC CDR2 having the sequence as set forth in SEQ ID NO: 216, an LC CDR3 having the sequence as set forth in SEQ ID NO: 217, an HC CDR1 having the sequence as set forth in SEQ ID NO: 218, an HC CDR2 having the sequence as set forth in SEQ ID NO:219, and an HC CDR3 having the sequence as set forth in SEQ ID NO: 220; or
an LC CDR1 having the sequence as set forth in SEQ ID NO: 221, an LC CDR2 having the sequence as set forth in SEQ ID NO: 222, an LC CDR3 having the sequence as set forth in SEQ ID NO: 223, an HC CDR1 having the sequence as set forth in SEQ ID NO: 224, an HC CDR2 having the sequence as set forth in SEQ ID NO:227, and an HC CDR3 having the sequence as set forth in SEQ ID NO: 228.

127. The antibody or binding fragment thereof of claim 126 comprising:
a variable light chain having the sequence as set forth in SEQ ID NO: 4 and a variable heavy chain having the sequence as set forth in SEQ ID NO: 5 or a variable light chain having a sequence having at least 90% sequence identity to the sequence as set forth in SEQ ID NO: 4 and a variable heavy chain having a sequence having at least 90% sequence identity to the sequence as set forth in SEQ ID NO: 5;
a variable light chain having the sequence as set forth in SEQ ID NO: 13 and a variable heavy chain having the sequence as set forth in SEQ ID NO: 14 or a variable light chain having a sequence having at least 90% sequence identity to the sequence as set forth in SEQ ID NO: 13 and a variable heavy chain having a sequence having at least 90% sequence identity to the sequence as set forth in SEQ ID NO: 14;
a variable light chain having the sequence as set forth in SEQ ID NO: 15 and a variable heavy chain having the sequence as set forth in SEQ ID NO: 16 or a variable light chain having a sequence having at least 90% sequence identity to the sequence as set forth in SEQ ID NO: 15 and a variable heavy chain having a sequence having at least 90% sequence identity to the sequence as set forth in SEQ ID NO: 16;
a variable light chain having the sequence as set forth in SEQ ID NO: 17 and a variable heavy chain having the sequence as set forth in SEQ ID NO: 18 or a variable light chain having a sequence having at least 90% sequence identity to the sequence as set forth in SEQ ID NO: 17 and a variable heavy chain having a sequence having at least 90% sequence identity to the sequence as set forth in SEQ ID NO: 18;
a variable light chain having the sequence as set forth in SEQ ID NO: 9 and a variable heavy chain having the sequence as set forth in SEQ ID NO: 10 or a variable light chain having a sequence having at least 90% sequence identity to the sequence as set forth in SEQ ID NO: 9 and a variable heavy chain having a sequence having at least 90% sequence identity to the sequence as set forth in SEQ ID NO: 10;
a variable light chain having the sequence as set forth in SEQ ID NO: 1 and a variable heavy chain having the sequence as set forth in SEQ ID NO: 2 or a variable light chain having a sequence having at least 90% sequence identity to the sequence as set forth in SEQ ID NO: 1 and a variable heavy chain having a sequence having at least 90% sequence identity to the sequence as set forth in SEQ ID NO: 2;
a variable light chain having the sequence as set forth in SEQ ID NO: 6 and a variable heavy chain having the sequence as set forth in SEQ ID NO: 7 or a variable light chain having a sequence having at least 90% sequence identity to the sequence as set forth in SEQ ID NO: 6 and a variable heavy chain having a sequence having at least 90% sequence identity to the sequence as set forth in SEQ ID NO: 7;
a variable light chain having the sequence as set forth in SEQ ID NO: 19 and a variable heavy chain having the sequence as set forth in SEQ ID NO: 20 or a variable light chain having a sequence having at least 90% sequence identity to the sequence as set forth in SEQ ID NO: 19 and a variable heavy chain having a sequence having at least 90% sequence identity to the sequence as set forth in SEQ ID NO: 20;
a variable light chain having the sequence as set forth in SEQ ID NO: 19 and a variable heavy chain having the sequence as set forth in SEQ ID NO: 23 or a variable light chain having a sequence having at least 90% sequence identity to the sequence as set forth in SEQ ID NO: 19 and a variable heavy chain having a sequence having at least 90% sequence identity to the sequence as set forth in SEQ ID NO: 23;
a variable light chain having the sequence as set forth in SEQ ID NO: 21 and a variable heavy chain having the sequence as set forth in SEQ ID NO: 22 or a variable light chain having a sequence having at least 90% sequence identity to the sequence as set forth in SEQ ID NO: 21 and a variable heavy chain having a sequence having at least 90% sequence identity to the sequence as set forth in SEQ ID NO: 22; or
a variable light chain having the sequence as set forth in SEQ ID NO: 21 and a variable heavy chain having the sequence as set forth in SEQ ID NO: 24 or a variable light chain having a sequence having at least 90% sequence identity to the sequence as set forth in SEQ ID NO: 21 and a variable heavy chain having a sequence having at least 90% sequence identity to the sequence as set forth in SEQ ID NO: 24.

128. The antibody or binding fragment thereof of claim 126, further comprising a human light chain constant region selected from the group consisting of: a human Igκ light chain constant region and a human IgA light chain constant region.

129. The antibody or binding fragment thereof of claim 128, wherein the human light chain constant region comprises the sequence as set forth in SEQ ID NO: 337 or SEQ ID NO: 338.

130. The antibody or binding fragment thereof of claim 126, comprising a variable light chain and a human Igκ light chain constant region comprising the sequence as set forth in SEQ ID NOs: 281, 287, 293, 434, 443, 452, 461, 476, 295, 320, 323, 436, 445, 454, 463, or 478 or a sequence having at least 95% sequence identity to SEQ ID NOs: 281, 287, 293, 434, 443, 453, 461, 476, 295, 320, 323, 436, 445, 454, 463, or 478.

131. The antibody or binding fragment thereof of claim 126, comprising a variable light chain and a human Igκ light chain constant region encoded by the sequence as set forth in SEQ ID NOs: 282, 288, 294, 435, 444, 453, 462 or 477 or a sequence having at least 95% sequence identity to SEQ ID NO: 282, 288, 294, 435, 444, 453, 462 or 477.

132. The antibody or binding fragment thereof of claim 126, comprising a variable light chain and a human IgA light chain constant region comprising the sequence as set forth in SEQ ID NO: 275 or SEQ ID NO: 317 or a sequence having at least 95% sequence identity to SEQ ID NO: 275 or SEQ ID NO: 317.

133. The antibody or binding fragment thereof of claim 126, comprising a variable light chain and a human IgA light chain constant region encoded by the sequence as set forth in SEQ ID NO: 276 or a sequence having at least 95% sequence identity to SEQ ID NO: 276.

134. The antibody or binding fragment thereof of claim 126, further comprising a human heavy chain constant region selected from the group consisting of: an IgG heavy chain constant region, an IgA heavy chain constant region, an IgM heavy chain constant region, an IgD heavy chain constant region, and an IgE heavy chain constant region.

135. The antibody or binding fragment thereof of claim 134, wherein the IgG heavy chain constant region comprises an IgG1 heavy chain constant region, an IgG2 heavy chain constant region, an IgG3 heavy chain constant region, or an IgG4 heavy chain constant region.

136. The antibody or binding fragment thereof of claim 126, comprising a variable heavy chain and an IgG1 heavy chain constant region comprising the sequence as set forth in SEQ ID NOs: 271, 277, 283, 289, 296, 437, 446, 455, 464, 470, 479, 315, 318, 321, 324, 298, 439, 448, 457, 466, 472, or 481 or a sequence having at least 95% sequence identity to SEQ ID NOs: 271, 277, 283, 289, 296, 437, 446, 455, 464, 470, 479, 315, 318, 321, 324, 298, 439, 448, 457, 466, 472, or 481.

137. The antibody or binding fragment thereof of claim 126, comprising a variable heavy chain and an IgG1 heavy chain constant region encoded by the sequence as set forth in SEQ ID NOs: 272, 278, 284, 290, 297, 438, 447, 456, 465, 471, or 480 or a sequence having at least 95% sequence identity to SEQ ID NOs: 272, 278, 284, 290, 297, 438, 447, 456, 465, 471, or 480.

138. The antibody or binding fragment thereof of claim 126, comprising a variable heavy chain and an IgG4 heavy chain constant region comprising the sequence as set forth in SEQ ID NOs: 273, 279, 285, 291, 431, 440, 449, 458, 467, 473, 482, 316, 319, 322, 325, 433, 442, 451, 460, 469, 475, or 484 or a sequence having at least 95% sequence identity to SEQ ID NOs: 273, 279, 285, 291, 431, 440, 449, 458, 467, 473, 482, 316, 319, 322, 325, 433, 442, 451, 460, 469, 475, or 484.

139. The antibody or binding fragment thereof of claim 126, comprising a variable heavy chain and an IgG4 heavy chain constant region encoded by the sequence as set forth in SEQ ID NOs: 274, 280, 286, 292, 432, 441, 450, 459, 468, 474, or 483 or a sequence having at least 95% sequence identity to SEQ ID NOs: 274, 280, 286, 292, 432, 441, 450, 459, 468, 474, or 483.

140. The antibody or binding fragment thereof of claim 126, wherein the antibody or fragment thereof further comprises a signal peptide.

141. The antibody or binding fragment thereof of claim 126, wherein the antibody or binding fragment thereof is an scFv having the sequence as set forth in SEQ ID NOs: 421, 423, 425, 427, 429, 491, 492, 493, 494, or 495 or has a sequence with at least 90% sequence identity to a sequence as set forth in SEQ ID NOs: 421, 423, 425, 427, 429, 491, 492, 493, 494, or 495.

142. The antibody or binding fragment thereof of claim 141, wherein the scFv is encoded by the sequence set forth in SEQ ID NO: 422, 424, 426, 428, or 430, or a sequence having at least 90% sequence identity to SEQ ID NO: 422, 424, 426, 428, or 430.

143. A composition comprising the antibody or binding fragment thereof of claim 1 and a pharmaceutically acceptable carrier.

144. A method of treating a subject in need thereof comprising administering a therapeutically effective amount of the composition of claim 143, thereby treating the subject in need thereof.

145. The method of claim 144, wherein the therapeutically effective amount provides a prophylactic or a therapeutic treatment against a mast-cell or eosinophil-related disorder.
